(19) United States
(12) Patent Application Publication
(10) Pub. No.: US 2022/0289708 A1
(43) Pub. Date: Sep. 15, 2022

(54) SUBSTITUTED N-PHENYL URACILS, SALTS THEREOF AND THEIR USE AS HERBICIDAL AGENTS

(71) Applicant: Bayer Aktiengesellschaft, Leverkusen (DE)

(72) Inventors: Ines HEINEMANN, Hofheim (DE); Jens FRACKENPOHL, Frankfurt (DE); Lothar WILLMS, Hillscheid (DE); Harald JAKOBI, Frankfurt (DE); Hendrik HELMKE, Liederbach (DE); Christopher Hugh ROSINGER, Hofheim (DE); Elmar GATZWEILER, Bad Nauheim (DE); Elisabeth ASMUS, Hoesbach (DE)

(21) Appl. No.: 17/628,524

(22) PCT Filed: Jul. 20, 2020
(86) PCT No.: PCT/EP2020/070463

§ 371 Date: Jan. 19, 2022

(30) Foreign Application Priority Data
Jul. 22, 2019 (EP) 19187529.3

(51) Int. Cl. C07D 401/12; A01N 25/32; A01N 43/54; C07D 405/12; A01P 13/00; C07D 409/12; C07F 7/10; C07F 5/04; A01N 47/12; C07D 487/08; C07D 471/08; A01N 43/90; A01N 43/56; A01N 43/58; A01N 43/76; A01N 43/78; A01N 55/00; A01N 55/08; C07D 417/14; C07D 413/14; C07D 401/14

(52) U.S. Cl. CPC C07D 401/12; A01N 25/32; A01N 43/54; C07D 405/12; A01P 13/00; C07D 409/12; C07F 7/10; C07F 5/04; A01N 47/12; C07D 487/08; C07D 471/08; A01N 43/90; A01N 43/56; A01N 43/58; A01N 43/76; A01N 43/78; A01N 55/00; A01N 55/08; C07D 417/14; C07D 413/14; C07D 401/14

(57) ABSTRACT

The present invention relates to substituted N-phenyluracils of the general formula (I) or salts thereof,

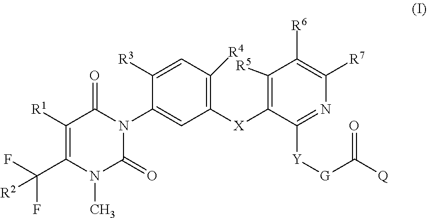

where the radicals in the general formula (I) correspond to the definitions given in the description,
and the use thereof as herbicides, in particular for controlling broad-leaved weeds and/or weed grasses in crops of useful plants and/or as plant growth regulators for influencing the growth of crops of useful plants.

The invention relates to the technical field of crop protection agents, in particular that of herbicides for the selective control of broad-leaved weeds and weed grasses in crops of useful plants.

Specifically, the present invention relates to substituted N-phenyluracils and to their salts, to processes for their preparation and to their use as herbicides, in particular for controlling broad-leaved weeds and/or weed grasses in crops of useful plants and/or as plant growth regulators for influencing the growth of crops of useful plants.

In their application, crop protection agents known to date for the selective control of harmful plants in crops of useful plants or active compounds for controlling unwanted vegetation sometimes have disadvantages, be it (a) that they have no or else insufficient herbicidal activity against particular harmful plants, (b) that the spectrum of harmful plants which can be controlled with an active compound is not wide enough, (c) that their selectivity in crops of useful plants is too low and/or (d) that they have a toxicologically unfavorable profile.

Furthermore, some active compounds which can be used as plant growth regulators for a number of useful plants cause unwanted reduced harvest yields in other useful plants or are not compatible with the crop plant, or only within a narrow application rate range. Some of the known active compounds cannot be produced economically on an industrial scale owing to precursors and reagents which are difficult to obtain, or they have only insufficient chemical stabilities. In the case of other active compounds, the activity is too highly dependent on environmental conditions, such as weather and soil conditions.

The herbicidal activity of these known compounds, in particular at low application rates, and/or their compatibility with crop plants remain in need of improvement.

From various publications, it is known that certain substituted N-attached aryluracils can be employed as herbicidally active compounds (cf. EP408382, EP473551, EP648749, U.S. Pat. Nos. 4,943,309, 5,084,084, 5,127,935, WO91/00278, WO95/29168, WO95/30661, WO96/35679, WO97/01541, WO98/25909, WO2001/39597). However, the known aryluracils have a number of activity gaps, in particular with regard to monocotyledonous weeds. A number of herbicidal active compound combinations based on N-attached aryluracils are also known (cf. DE4437197, EP714602, WO96/07323, WO96/08151, JP11189506). However, the properties of these active compound combinations were not entirely satisfactory.

Furthermore, it is known that certain N-phenyluracils having lactic acid groups, optionally with further substitution, can likewise be employed as herbicidally active compounds (cf. JP2000/302764, JP2001/172265, U.S. Pat. No. 6,403,534, EP408382). In addition, it is known that N-phenyluracils having thiolactic acid groups, optionally with further substitution, are likewise herbicidally active (cf. WO2010/038953, KR2011110420). Particular substituted tetrahydrofuryl esters of N-phenyluracils having thiolactic acid groups, optionally with further substitution, are described in JP09188676.

Also known are substituted uracils having an N-attached and further substituted diaryl ether group or a corresponding heteroaryl aryl ether radical (cf. U.S. Pat. Nos. 6,333,296, 6,121,201, WO2001/85907, EP1122244, EP1397958, EP1422227, WO 2002/098227). Furthermore, highly substituted N-phenyluracils having a carbonylalkyloxy group with specific substituted have been described (cf. WO2011/137088). Highly substituted N-pyridyluracils have also been described (cf. WO2017/202768), whereas WO2018/019842 describes the use of specifically substituted N-phenyluracils for controlling certain dicotyledonous weeds with specific resistance to established herbicides. Substituted 3-phenyl-5-alkyl-6-(trifluoromethyl)pyrimidine-2,4(1H,3H)-diones (cf. WO2019/101551) and related substituted 3-(pyridin-2-yl)-5-alkyl-6-(trifluoromethyl)pyrimidine-2,4(1H,3H)-diones (cf. WO2019/101513) are likewise known.

Surprisingly, it has now been found that certain substituted N-phenyluracils having a substituted alkyl ester side chain or salts thereof are suitable as herbicides and can be employed particularly advantageously for controlling monocotyledonous and dicotyledonous weeds in crop plant cultures.

Accordingly, the present invention provides substituted N-phenyluracils of the general formula (I) or salts thereof

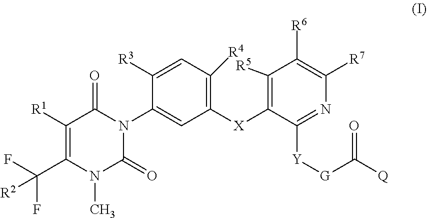

in which
R¹ represents hydrogen, (C₁-C₈)-haloalkyl,
R² represents hydrogen, fluorine, chlorine, bromine, trifluoromethyl, (C₁-C₈)-alkoxy,
R³ represents hydrogen, halogen, (C₁-C₈)-alkoxy,
R⁴ represents halogen, cyano, NO₂, C(O)NH₂, C(S)NH₂, (C₁-C₈)-haloalkyl, (C₂-C₈)-alkynyl,
R⁵, R⁶ and R⁷ independently of one another represent hydrogen, halogen, cyano, (C₁-C₈)-alkyl, (C₁-C₈)-haloalkyl, (C₁-C₈)-alkoxy, (C₁-C₈)-haloalkoxy,
G represents straight-chain or branched (C₁-C₈)-alkylene,
Q represents a radical of the formula

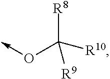

R⁸ represents hydrogen, (C₁-C₈)-alkyl, (C₁-C₈)-haloalkyl, aryl, aryl-(C₁-C₈)-alkyl, heteroaryl, (C₂-C₈)-alkynyl, (C₂-C₈)-alkenyl, C(O)R¹³, C(O)OR¹³, (C₁-C₈)-alkoxy-(C₁-C₈)-alkyl,
R⁹ represents hydrogen or (C₁-C₈)-alkyl,
R¹⁰ represents cyano, NO₂, heteroaryl, heteroaryl-(C₁-C₈)-alkyl, heterocyclyl, heterocyclyl-(C₁-C₈)-alkyl, R¹¹R¹²N—(C₁-C₈)-alkyl, R¹³O—(C₁-C₈)-alkyl, cyano-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, OR¹³, NR¹¹R¹², SR¹⁴, S(O)R¹⁴, SO₂R¹⁴, R¹⁴S—(C₁-C₈)-alkyl, R¹⁴(O)S—(C₁-C₈)-alkyl, R¹⁴O₂S—(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-alkyl]-bis-(aryl)silyl-(C₁-C₈)-alkyl, tris-[(C₁-C₈)-alkyl]silyl, bis-hydroxyboryl-(C₁-C₈)-alkyl, bis-[(C₁-C₈)-alkoxy]boryl-(C₁-C₈)-alkyl, tetramethyl-1,3,2-dioxaborolan-2-yl, tetramethyl-1,3,2-dioxaborolan-2-yl-(C₁-C₈)-alkyl, nitro-(C₁-C₈)-alkyl, C(O)R¹⁴, bis-(C₁-C₈)-alkoxymethyl, bis-(C₁-C₈)-alkoxymethyl-(C₁-C₈)-alkyl, or
R⁸ and R¹⁰ together with the carbon atom to which they are attached form a fully saturated or partially saturated 3- to 10-membered monocyclic or bicyclic heterocyclyl optionally having further substitution,
R¹¹ and R¹² are identical or different and independently of one another represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, COR¹³, SO₂R¹⁴, heterocyclyl, (C₁-C₈)-alkoxycarbonyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, heterocyclyl-(C₁-C₈)-alkyl, or
R¹¹ and R¹² together with the nitrogen atom to which they are attached form a fully saturated or partially saturated 3- to 10-membered monocyclic or bicyclic ring optionally interrupted by heteroatoms and optionally having further substitution,
R¹³ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkoxy-(C₁-C₈)-haloalkyl, (C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkoxy-(C₁-C₈)-alkyl, aryl, aryl-(C₁-C₈)-alkyl, aryl-(C₁-C₈)-alkoxy-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, bis-[(C₁-C₈)-alkyl]amino-(C₂-C₆)-alkyl, (C₁-C₈)-alkyl-amino-(C₂-C₆)-alkyl, aryl-(C₁-C₈)-alkyl-amino-(C₂-C₆)-alkyl, R¹⁴S—(C₁-C₈)-alkyl, R¹⁴(O)S—(C₁-C₈)-alkyl, R¹⁴O₂S—(C₁-C₈)-alkyl, hydroxycarbonyl-(C₁-C₈)-alkyl, heterocyclyl, heterocyclyl-(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-alkyl]-bis-(aryl)silyl-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, aryloxy-(C₁-C₈)-alkyl, heteroaryloxy-(C₁-C₈)-alkyl, (C₁-C₈)-alkoxycarbonyl,
R¹⁴ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]amino, (C₁-C₈)-alkyl-amino, aryl-(C₁-C₈)-amino, aryl-(C₁-C₆)-alkyl-amino, aryl-[(C₁-C₈)-alkyl]amino, (C₃-C₈)-cycloalkyl-amino, (C₃-C₈)-cycloalkyl-[(C₁-C₈)-alkyl]amino; N-azetidinyl, N-pyrrolidinyl, N-piperidinyl, N-morpholinyl,
   and
X and Y independently of one another represent O (oxygen) or S (sulfur).

The invention preferably provides compounds of the general formula (I) in which
R¹ represents hydrogen, (C₁-C₇)-haloalkyl,
R² represents hydrogen, fluorine, chlorine, bromine, trifluoromethyl, (C₁-C₇)-alkoxy,
R³ represents hydrogen, halogen, (C₁-C₇)-alkoxy,
R⁴ represents halogen, cyano, NO₂, C(O)NH₂, C(S)NH₂, (C₁-C₇)-haloalkyl, (C₂-C₇)-alkynyl,
R⁵, R⁶ and R⁷ independently of one another represent hydrogen, halogen, cyano, (C₁-C₇)-alkyl, (C₁-C₇)-haloalkyl, (C₁-C₇)-alkoxy, (C₁-C₇)-haloalkoxy,
G represents straight-chain or branched (C₁-C₇)-alkylene,
Q represents a radical of the formula

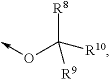

R⁸ represents hydrogen, (C₁-C₇)-alkyl, (C₁-C₇)-haloalkyl, aryl, aryl-(C₁-C₇)-alkyl, heteroaryl, (C₂-C₇)-alkynyl, (C₂-C₇)-alkenyl, C(O)R¹³, C(O)OR¹³, (C₁-C₇)-alkoxy-(C₁-C₇)-alkyl,
R⁹ represents hydrogen or (C₁-C₆)-alkyl,
R¹⁰ represents cyano, NO₂, heteroaryl, heteroaryl-(C₁-C₇)-alkyl, heterocyclyl, heterocyclyl-(C₁-C₇)-alkyl, R¹¹R¹²N—(C₁-C₇)-alkyl, R¹³O—(C₁-C₇)-alkyl, cyano-(C₁-C₇)-alkyl, (C₁-C₇)-alkylcarbonyloxy-(C₁-C₇)-alkyl, (C₃-C₇)-cycloalkylcarbonyloxy-(C₁-C₇)-alkyl, arylcarbonyloxy-(C₁-C₇)-alkyl, heteroarylcarbonyloxy-(C₁-C₇)-alkyl, heterocyclylcarbonyloxy-(C₁-C₇)-alkyl, OR¹³, NR¹¹R¹², SR¹⁴, S(O)R¹⁴, SO₂R¹⁴, R¹⁴S—(C₁-C₇)-alkyl, R¹⁴(O)S—(C₁-C₇)-alkyl, R¹⁴O₂S—(C₁-C₇)-alkyl, tris-[(C₁-C₇)-alkyl]silyl-(C₁-C₇)-alkyl, bis-[(C₁-C₇)-alkyl](aryl)silyl(C₁-C₇)-alkyl, [(C₁-C₇)-alkyl]-bis-(aryl)silyl-(C₁-C₇)-alkyl, tris-[(C₁-C₇)-alkyl]silyl, bis-hydroxyboryl-(C₁-C₇)-alkyl, bis-[(C₁-C₇)-alkoxy]boryl-(C₁-C₇)-alkyl, tetramethyl-1,3,2-dioxaborolan-2-yl, tetramethyl-1,3,2-dioxaborolan-2-yl-(C₁-C₇)-alkyl, nitro-(C₁-C₇)-alkyl, C(O)R¹⁴, bis-(C₁-C₇)-alkoxymethyl, bis-(C₁-C₇)-alkoxymethyl-(C₁-C₇)-alkyl, or
R⁸ and R¹⁰ together with the carbon atom to which they are attached form a fully saturated or partially saturated 3- to 10-membered monocyclic or bicyclic heterocyclyl optionally having further substitution,
R¹¹ and R¹² are identical or different and independently of one another represent hydrogen, (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₃-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-haloalkoxy-(C₁-C₇)-alkyl, (C₁-C₇)-alkylthio-(C₁-C₇)-alkyl, (C₁-C₇)-haloalkylthio-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, (C₃-C₇)-cycloalkyl-(C₁-C₇)-alkyl, (C₄-C₁₀)-cycloalkenyl-(C₁-C₇)-alkyl, COR¹³, SO₂R¹⁴, heterocyclyl, (C₁-C₇)-alkoxycarbonyl, bis-[(C₁-C₇)-alkyl]aminocarbonyl-(C₁-C₇)-alkyl, (C₁-C₇)-alkyl-aminocarbonyl-(C₁-C₇)-alkyl, aryl-(C₁-C₇)-alkyl-aminocarbonyl-(C₁-C₇)-alkyl, aryl-(C₁-C₇)-alkoxycarbonyl, heteroaryl-(C₁-C₇)-alkoxycarbonyl, (C₂-C₇)-alkenyloxycarbonyl, (C₂-C₇)-alkynyloxycarbonyl, heterocyclyl-(C₁-C₇)-alkyl, or
R¹¹ and R¹² together with the nitrogen atom to which they are attached form a fully saturated or partially saturated 3- to 10-membered monocyclic or bicyclic ring optionally interrupted by heteroatoms and optionally having further substitution,
   R¹³ represents hydrogen, (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₃-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-haloalkoxy-(C₁-C₇)-alkyl, (C₁-C₇)-alkoxy-(C₁-C₇)-haloalkyl, (C₁-C₇)-alkoxy-(C₁-C₇)-alkoxy-(C₁-C₇)-alkyl, (C₁-C₇)-alkoxy-(C₁-C₇)-alkoxy-(C₁-C₇)-alkoxy-(C₁-C₇)-alkyl, (C₁-C₇)-alkoxy-(C₁-C₇)-alkoxy-(C₁-C₇)-alkoxy-(C₁-C₇)-alkoxy-(C₁-C₇)-alkyl, aryl, aryl-(C₁-C₇)-alkyl, aryl-(C₁-C₇)-alkoxy-(C₁-C₇)-alkyl, heteroaryl, heteroaryl-(C₁-C₇)-alkyl, (C₃-C₇)-cycloalkyl-(C₁-C₇)-alkyl, (C₄-C₁₀)-cycloalkenyl-(C₁-C₇)-alkyl, bis-[(C₁-C₇)-alkyl]aminocarbonyl-(C₁-C₇)-alkyl, (C₁-C₇)-alkyl-aminocarbonyl-(C₁-C₇)-alkyl, aryl-(C₁-C₇)-alkyl-aminocarbonyl-(C₁-C₇)-alkyl, bis-[(C₁-C₇)-alkyl]amino-(C₂-C₅)-alkyl, (C₁-C₇)-alkyl-amino-(C₂-C₅)-alkyl, aryl-(C₁-C₇)-alkyl-amino-(C₂-C₅)-alkyl, R¹⁴S—(C₁-C₇)-alkyl, R¹⁴(O)S—(C₁-C₇)-alkyl, R¹⁴O₂S—(C₁-C₇)-alkyl, hydroxycarbonyl-(C₁-C₇)-alkyl, heterocyclyl, heterocyclyl-(C₁-C₇)-alkyl, tris-[(C₁-C₇)-alkyl]silyl-(C₁-C₇)-alkyl, bis-[(C₁-C₇)-alkyl](aryl)silyl(C₁-C₇)-alkyl, [(C₁-C₇)-alkyl]-bis-(aryl)silyl-(C₁-C₇)-alkyl, (C₁-C₇)-alkylcarbonyloxy-(C₁-C₇)-alkyl, (C₃-C₇)-cycloalkylcarbonyloxy-(C₁-C₇)-alkyl, arylcarbonyloxy-(C₁-C₇)-alkyl, heteroarylcarbonyloxy-(C₁-C₇)-alkyl, heterocyclylcarbonyloxy-(C₁-C₇)-alkyl, aryloxy-(C₁-C₇)-alkyl, heteroaryloxy-(C₁-C₇)-alkyl, (C₁-C₇)-alkoxycarbonyl,
   R¹⁴ represents hydrogen, (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₃-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, heterocyclyl-(C₁-C₇)-alkyl, (C₃-C₇)-cycloalkyl-(C₁-C₇)-alkyl, (C₄-C₁₀)-cycloalkenyl-(C₁-C₇)-alkyl, bis-[(C₁-C₇)-alkyl]amino, (C₁-C₇)-alkyl-amino, aryl-(C₁-C₇)-amino, aryl-(C₁-C₄)-alkyl-amino, aryl-[(C₁-C₇)-alkyl]amino, (C₃-C₇)-cycloalkyl-amino, (C₃-C₇)-cycloalkyl-[(C₁-C₇)-alkyl]amino; N-azetidinyl, N-pyrrolidinyl, N-piperidinyl, N-morpholinyl,
      and
X and Y independently of one another represent O (oxygen) or S (sulfur).

The invention particularly preferably provides compounds of the general formula (I), in which
R¹ represents hydrogen,
R² represents hydrogen, fluorine, chlorine, bromine, trifluoromethyl, (C₁-C₆)-alkoxy,
R³ represents hydrogen, halogen, (C₁-C₆)-alkoxy,
R⁴ represents halogen, cyano, NO₂, C(O)NH₂, C(S)NH₂, (C₁-C₆)-haloalkyl, (C₂-C₆)-alkynyl,
R⁵, R⁶ and R⁷ independently of one another represent hydrogen, halogen, cyano, (C₁-C₆)-alkyl, (C₁-C₆)-haloalkyl, (C₁-C₆)-alkoxy, (C₁-C₆)-haloalkoxy,
G represents straight-chain or branched (C₁-C₆)-alkylene,
Q represents a radical of the formula

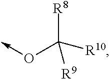

R⁸ represents hydrogen, (C₁-C₆)-alkyl, (C₁-C₆)-haloalkyl, aryl, aryl-(C₁-C₆)-alkyl, heteroaryl, (C₂-C₆)-alkynyl, (C₂-C₆)-alkenyl, C(O)R¹³, C(O)OR¹³, (C₁-C₆)-alkoxy-(C₁-C₆)-alkyl,
R⁹ represents hydrogen or (C₁-C₄)-alkyl,
R¹⁰ represents cyano, NO₂, heteroaryl, heteroaryl-(C₁-C₆)-alkyl, heterocyclyl, heterocyclyl-(C₁-C₆)-alkyl, R¹¹R¹²N—(C₁-C₆)-alkyl, R¹³O—(C₁-C₆)-alkyl, cyano-(C₁-C₆)-alkyl, (C₁-C₆)-alkylcarbonyloxy-(C₁-C₆)-alkyl, (C₃-C₆)-cycloalkylcarbonyloxy-(C₁-C₆)-alkyl, arylcarbonyloxy-(C₁-C₆)-alkyl, heteroarylcarbonyloxy-(C₁-C₆)-alkyl, heterocyclylcarbonyloxy-(C₁-C₆)-alkyl, OR¹³, NR¹¹R¹², SR¹⁴, S(O)R¹⁴, SO₂R¹⁴, R¹⁴S—(C₁-C₆)-alkyl, R¹⁴(O)S—(C₁-C₆)-alkyl, R¹⁴O₂S—(C₁-C₆)-alkyl, tris-[(C₁-C₆)-alkyl]silyl-(C₁-C₆)-alkyl, bis-[(C₁-C₆)-alkyl](aryl)silyl(C₁-C₆)-alkyl, [(C₁-C₆)-alkyl]-bis-(aryl)silyl-(C₁-C₆)-alkyl, tris-[(C₁-C₆)-alkyl]silyl, bis-hydroxyboryl-(C₁-C₆)-alkyl, bis-[(C₁-C₆)-alkoxy]boryl-(C₁-C₆)-alkyl, tetramethyl-1,3,2-dioxaborolan-2-yl, tetramethyl-1,3,2-dioxaborolan-2-yl-(C₁-C₆)-alkyl, nitro-(C₁-C₆)-alkyl, C(O)R¹³, bis-(C₁-C₆)-alkoxymethyl, bis-(C₁-C₆)-alkoxymethyl-(C₁-C₆)-alkyl,
R⁸ and R¹⁰ together with the carbon atom to which they are attached form a fully saturated or partially saturated 3- to 10-membered monocyclic or bicyclic heterocyclyl optionally having further substitution,
R¹¹ and R¹² are identical or different and independently of one another represent hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-haloalkoxy-(C₁-C₆)-alkyl, (C₁-C₆)-alkylthio-(C₁-C₆)-alkyl, (C₁-C₆)-haloalkylthio-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, COR¹³, SO₂R¹⁴, heterocyclyl, (C₁-C₆)-alkoxycarbonyl, bis-[(C₁-C₆)-alkyl]aminocarbonyl-(C₁-C₆)-alkyl, (C₁-C₆)-alkyl-aminocarbonyl-(C₁-C₆)-alkyl, aryl-(C₁-C₆)-alkyl-aminocarbonyl-(C₁-C₆)-alkyl, aryl-(C₁-C₆)-alkoxycarbonyl, heteroaryl-(C₁-C₆)-alkoxycarbonyl, (C₂-C₆)-alkenyloxycarbonyl, (C₂-C₆)-alkynyloxycarbonyl, heterocyclyl-(C₁-C₆)-alkyl, or
R¹¹ and R¹² together with the nitrogen atom to which they are attached form a fully saturated or partially saturated 3- to 10-membered monocyclic or bicyclic ring optionally interrupted by heteroatoms and optionally having further substitution,
R¹³ represents hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-haloalkoxy-(C₁-C₆)-alkyl, (C₁-C₆)-alkoxy-(C₁-C₆)-haloalkyl, (C₁-C₆)-alkoxy-(C₁-C₆)-alkoxy-(C₁-C₆)-alkyl, (C₁-C₆)-alkoxy-(C₁-C₆)-alkoxy-(C₁-C₆)-alkoxy-(C₁-C₆)-alkyl, (C₁-C₆)-alkoxy-(C₁-C₆)-alkoxy-(C₁-C₆)-alkoxy-(C₁-C₆)-alkoxy-(C₁-C₆)-alkyl, aryl, aryl-(C₁-C₆)-alkyl, aryl-(C₁-C₆)-alkoxy-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, bis-[(C₁-C₆)-alkyl]aminocarbonyl-(C₁-C₆)-alkyl, (C₁-C₆)-alkyl-aminocarbonyl-(C₁-C₆)-alkyl, aryl-(C₁-C₆)-alkyl-aminocarbonyl-(C₁-C₆)-alkyl, bis-[(C₁-C₆)-alkyl]amino-(C₂-C₄)-alkyl, (C₁-C₆)-alkyl-amino-(C₂-C₄)-alkyl, aryl-(C₁-C₆)-alkyl-amino-(C₂-C₄)-alkyl, R¹⁴S—(C₁-C₆)-alkyl, R¹⁴(O)S—(C₁-C₆)-alkyl, R¹⁴O₂S—(C₁-C₆)-alkyl, hydroxycarbonyl-(C₁-C₆)-alkyl, heterocyclyl, heterocyclyl-(C₁-C₆)-alkyl, tris-[(C₁-C₆)-alkyl]silyl-(C₁-C₆)-alkyl, bis-[(C₁-C₆)-alkyl](aryl)silyl(C₁-C₆)-alkyl, [(C₁-C₆)-alkyl]-bis-(aryl)silyl-(C₁-C₆)-alkyl, (C₁-C₆)-alkylcarbonyloxy-(C₁-C₆)-alkyl, (C₃-C₆)-cycloalkylcarbonyloxy-(C₁-C₆)-alkyl, arylcarbonyloxy-(C₁-C₆)-alkyl, heteroarylcarbonyloxy-(C₁-C₆)-alkyl, heterocyclylcarbonyloxy-(C₁-C₆)-alkyl, aryloxy-(C₁-C₆)-alkyl, heteroaryloxy-(C₁-C₆)-alkyl, (C₁-C₆)-alkoxycarbonyl,
R¹⁴ represents hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, heterocyclyl-(C₁-C₆)-alkyl, (C₃-C₆)-cycloalkyl-(C₁-C₆)-alkyl, (C₄-C₁₀)-cycloalkenyl-(C₁-C₆)-alkyl, bis-[(C₁-C₆)-alkyl]amino, (C₁-C₆)-alkyl-amino, aryl-(C₁-C₆)-amino, aryl-(C₁-C₂)-alkyl-amino, aryl-[(C₁-C₆)-alkyl]amino, (C₃-C₆)-cycloalkyl-amino, (C₃-C₆)-cycloalkyl-[(C₁-C₆)-alkyl]amino; N-azetidinyl, N-pyrrolidinyl, N-piperidinyl, N-morpholinyl,
   and
X and Y independently of one another represent O (oxygen) or S (sulfur).

The invention very particularly preferably provides compounds of the general formula (I) in which
R¹ represents hydrogen,
R² represents hydrogen, fluorine, chlorine, bromine, trifluoromethyl, methoxy, ethoxy, prop-1-yloxy, but-1-yloxy,
R³ represents hydrogen, fluorine, chlorine, bromine, methoxy, ethoxy, prop-1-yloxy, prop-2-yloxy, but-1-yloxy, but-2-yloxy, 2-methylprop-1-yloxy, 1,1-dimethyleth-1-yloxy,
R⁴ represents fluorine, chlorine, bromine, cyano, NO₂, C(O)NH₂, C(S)NH₂, trifluoromethyl, difluoromethyl, pentafluoroethyl, ethynyl, propyn-1-yl, 1-butyn-1-yl, pentyn-1-yl, hexyn-1-yl,
R⁵, R⁶ and R⁷ independently of one another represent hydrogen, fluorine, chlorine, bromine, iodine, cyano, methyl, ethyl, prop-1-yl, 1-methylethyl, but-1-yl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, difluoromethyl, pentafluoroethyl, 2,2-difluoroethyl, 2,2,2-trifluoroethyl, methoxy, ethoxy, prop-1-yloxy, prop-2-yloxy, but-1-yloxy, but-2-yloxy, 2-methylprop-1-yloxy, 1,1-dimethyleth-1-yloxy, difluoromethoxy, trifluoromethoxy, pentafluoroethoxy, 2,2-difluoroethoxy, 2,2,2-trifluoroethoxy,
G represents methylene, (methyl)methylene, (ethyl)methylene, (prop-1-yl)methylene, (prop-2-yl)methylene, (but-1-yl)methylene, (but-2-yl)methylene, (pent-1-yl)methylene, (pent-2-yl)methylene, (pent-3-yl)methylene, (dimethyl)methylene, (diethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, 1,1-dimethylethyl-1-ene, 2,2-dimethylethyl-1-ene, 1-ethylethyl-1-ene, 2-ethylethyl-1-ene, 1-(prop-1-yl)ethyl-1-ene, 2-(prop-1-yl)ethyl-1-ene, 1-(prop-2-yl)ethyl-1-ene, 2-(prop-2-yl)ethyl-1-ene, 1,1,2-trimethylethyl-1-ene, 1,2,2-trimethylethyl-1-ene, 1,1,2,2-tetramethylethyl-1-ene, n-pentylene, 1-methylbutyl-1-ene, 2-methylbutyl-1-ene, 3-methylbutyl-1-ene, 4-methylbutyl-1-ene, 1,1-dimethylpropyl-1-ene, 2,2-dimethylpropyl-1-ene, 3,3-dimethylpropyl-1-ene, 1,2-dimethylpropyl-1-ene, 1,3-dimethylpropyl-1-ene, 1-ethylpropyl-1-ene, n-hexylene, 1-methylpentyl-1-ene, 2-methylpentyl-1-ene, 3-methylpentyl-1-ene, 4-methylpentyl-1-ene, 1,1-dimethylbutyl-1-ene, 1,2-dimethylbutyl-1-ene, 1,3-dimethylbutyl-1-ene, 2,2-dimethylbutyl-1-ene, 2,3-dimethylbutyl-1-ene, 3,3-dimethylbutyl-1-ene, 1-ethylbutyl-1-ene, 2-ethylbutyl-1-ene, 1,1,2-trimethylpropyl-1-ene, 1,2,2-trimethylpropyl-1-ene, 1-ethyl-1-methylpropyl-1-ene, 1-ethyl-2-methylpropyl-1-ene,
X and Y independently of one another represent O (oxygen) or S (sulfur)
   and
Q represents one of the moieties Q-1 to Q-54, Q-56 to Q-57, Q-60 to Q-89, Q-91 to Q-129, Q-131 to Q-139, Q-141 to Q-144, Q-146 to Q-180, Q-182 to Q-185, Q-193 to Q-195, Q-200 to Q-208, Q-210 to Q-370, Q-395 to Q-440 specifically mentioned below:

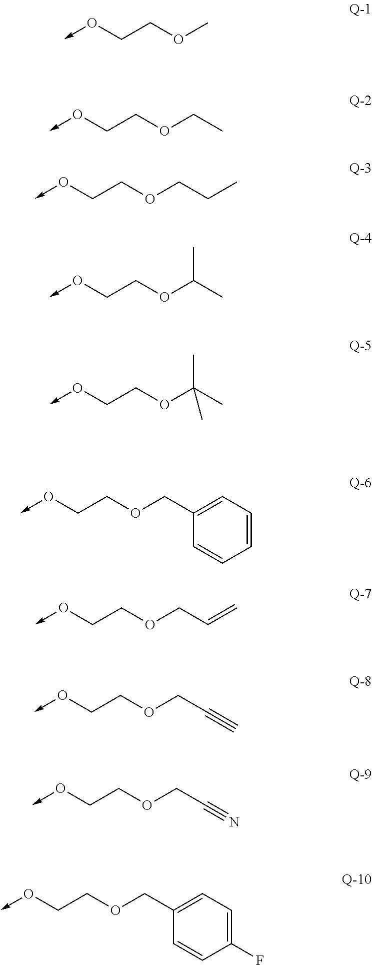

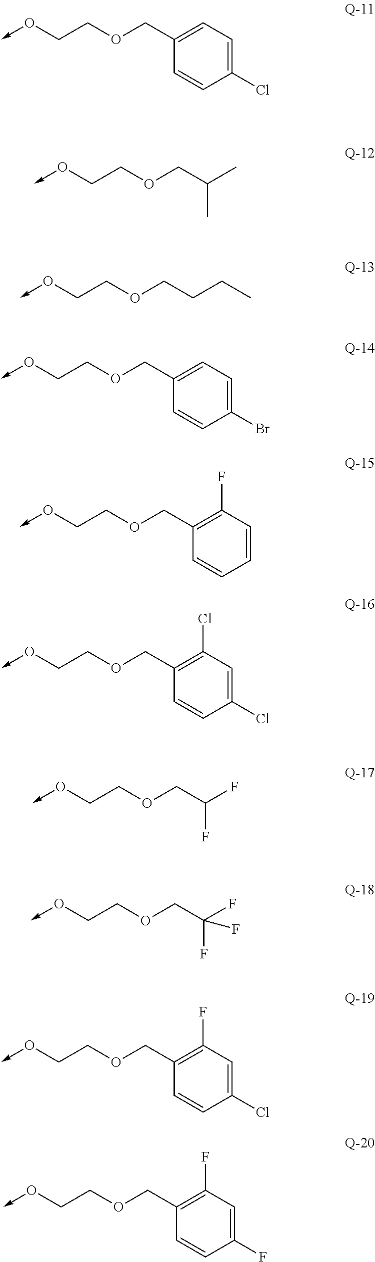

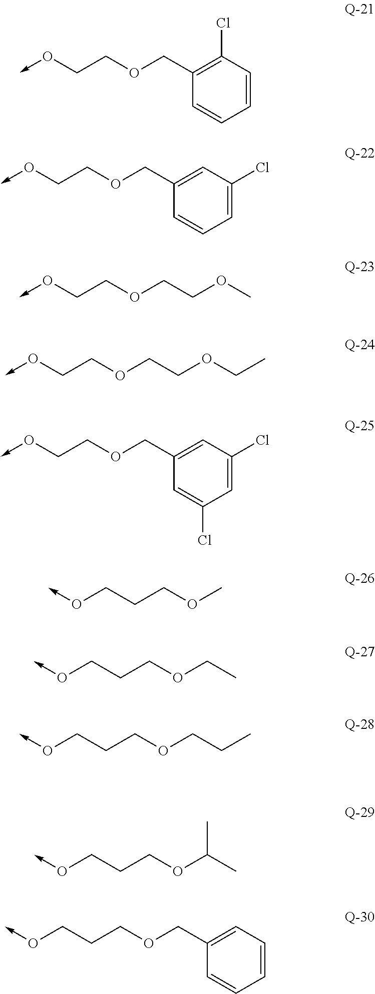

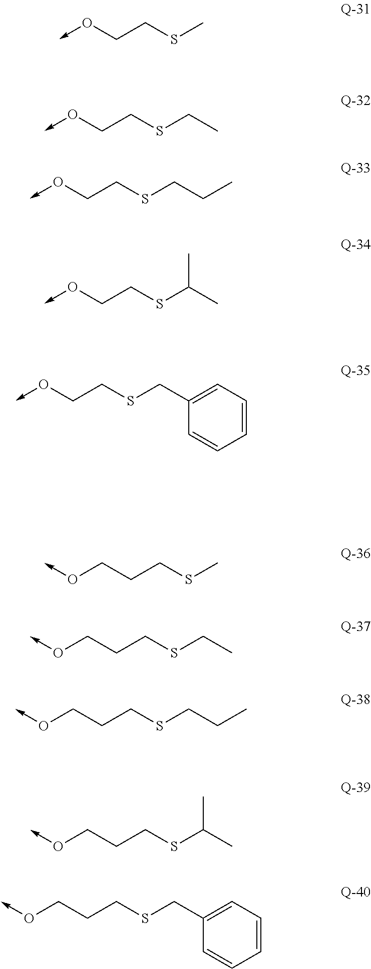

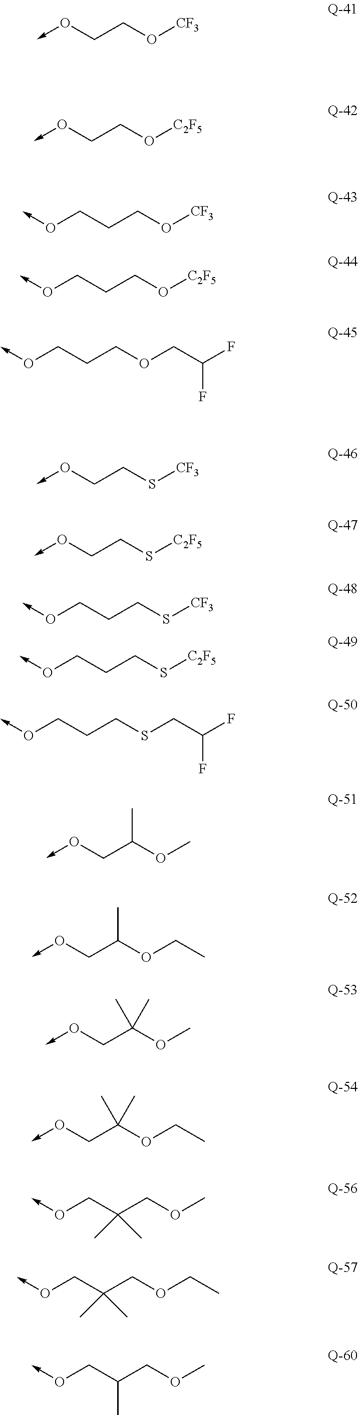

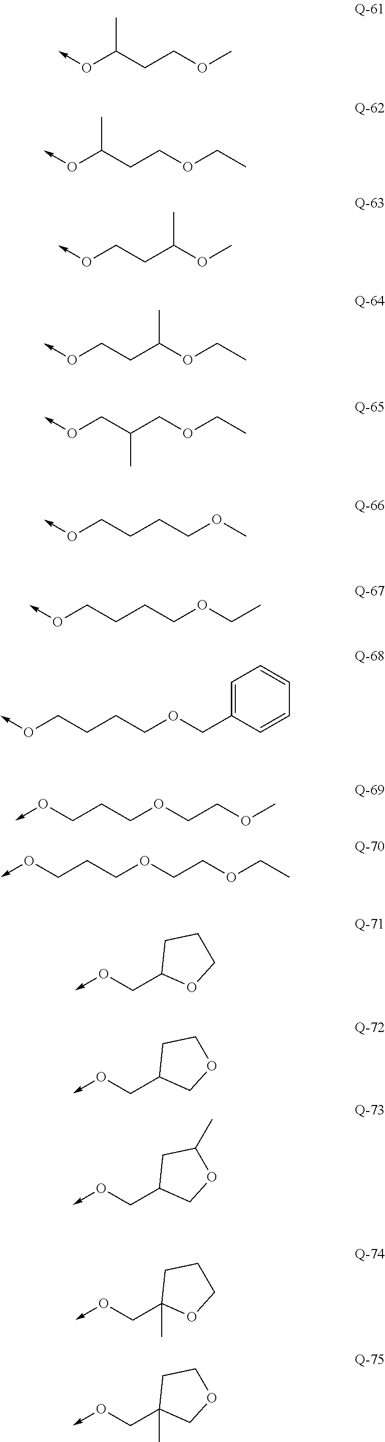

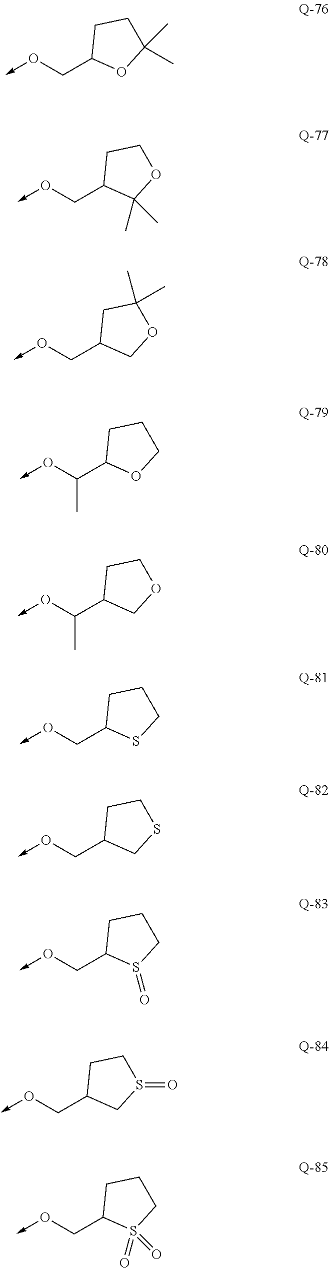

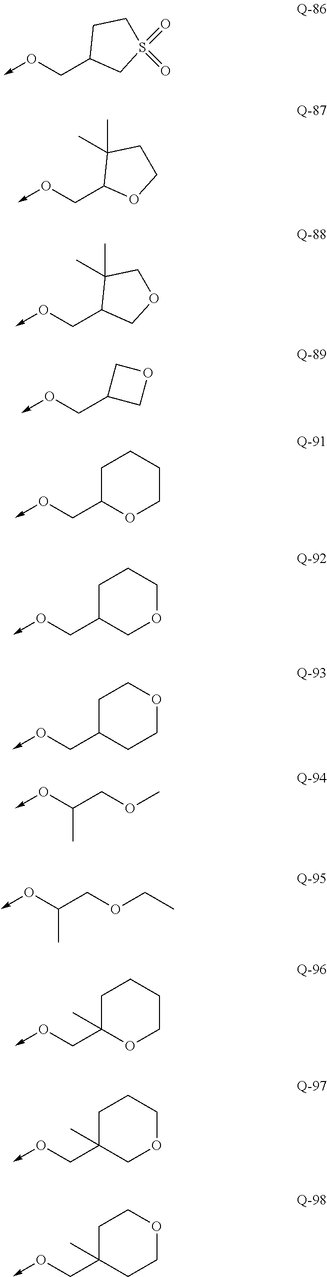

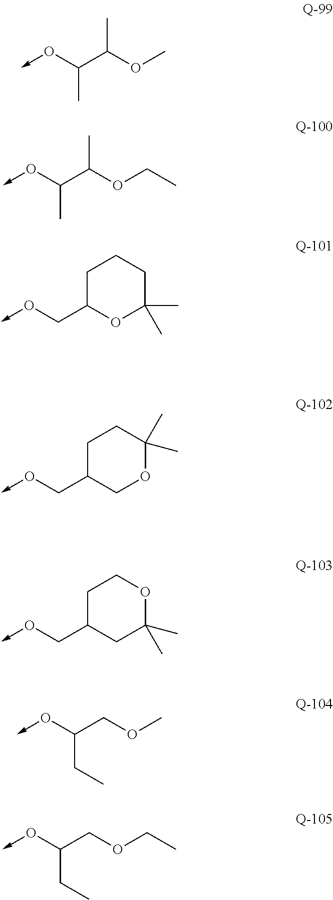

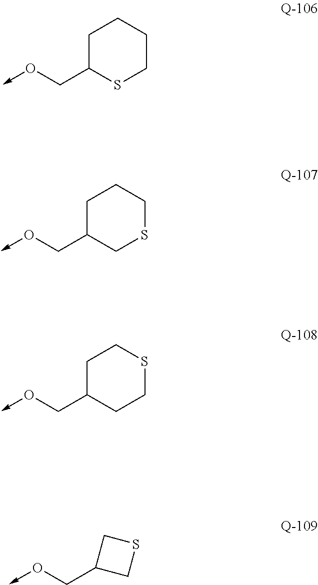

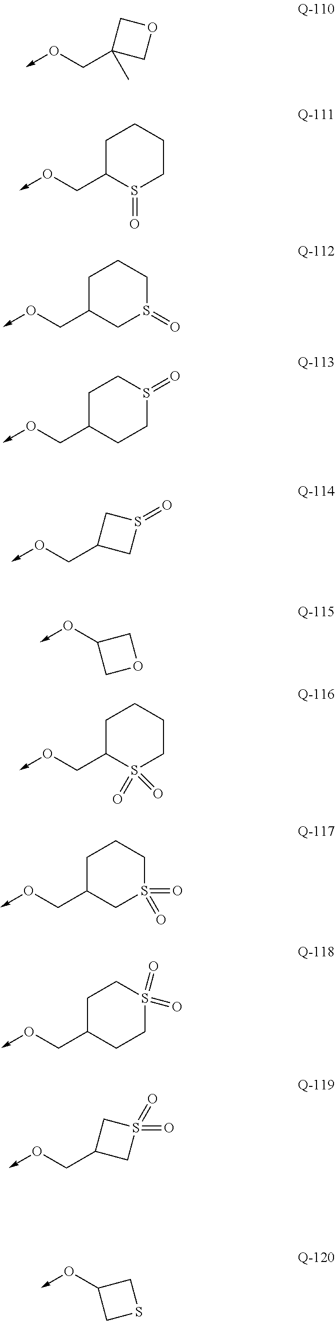

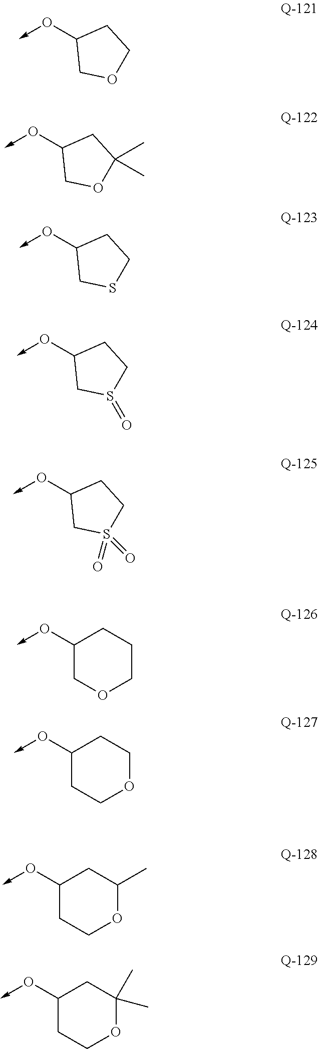

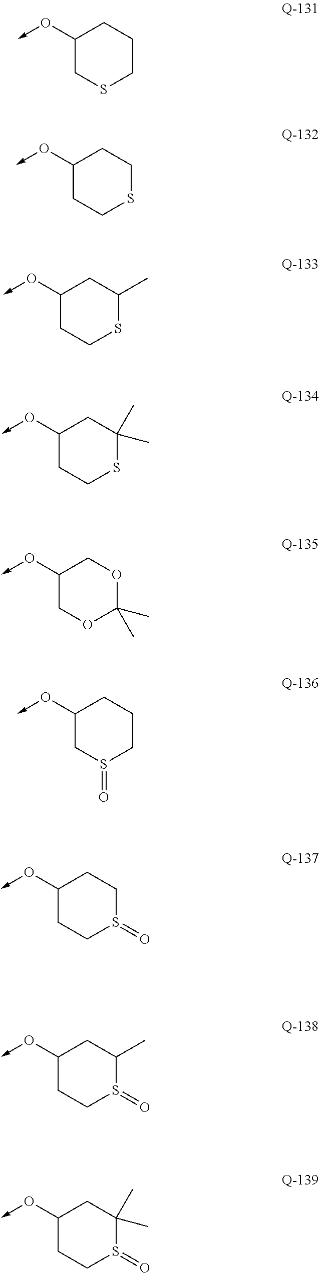

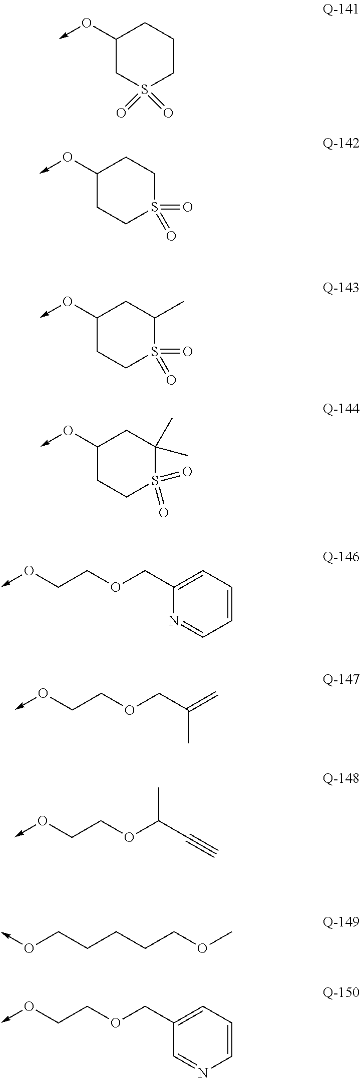

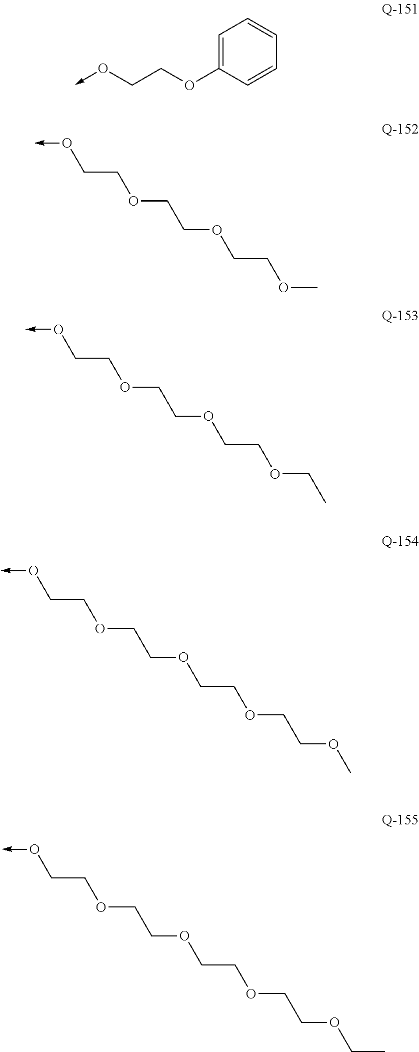

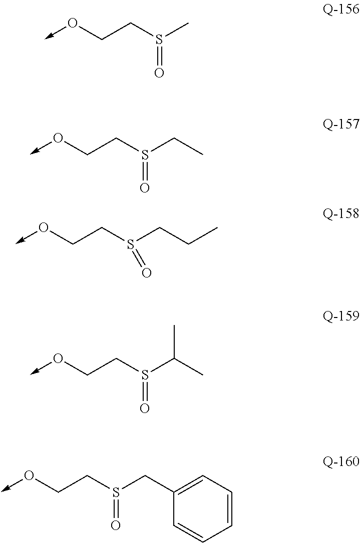

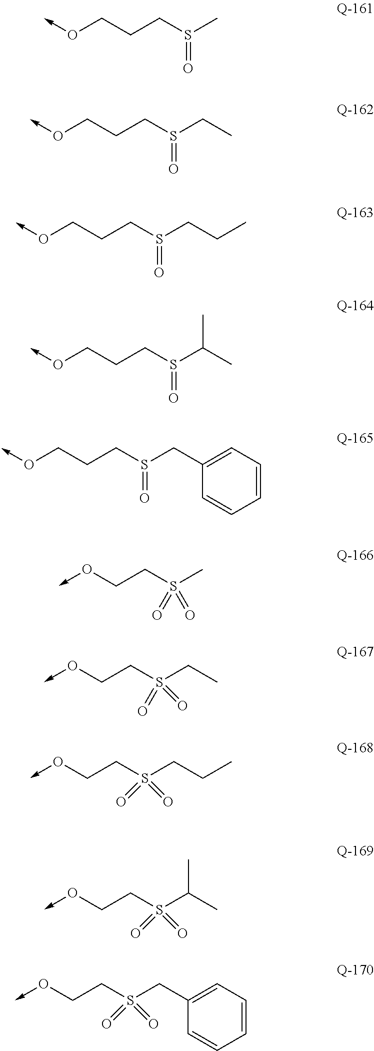

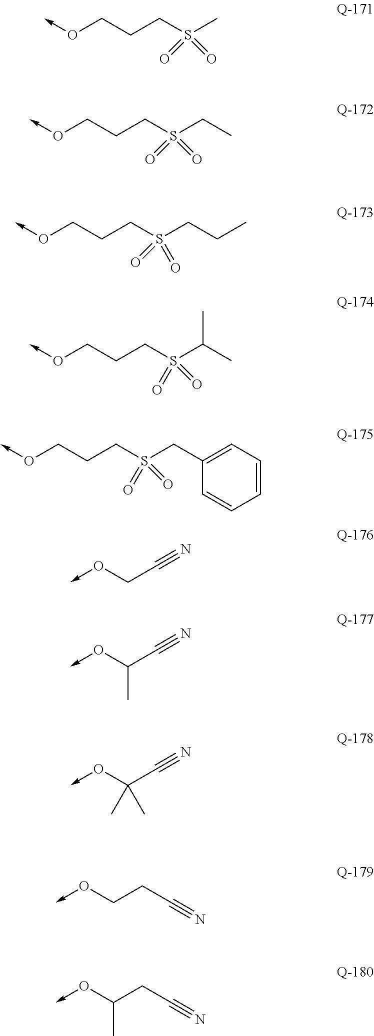

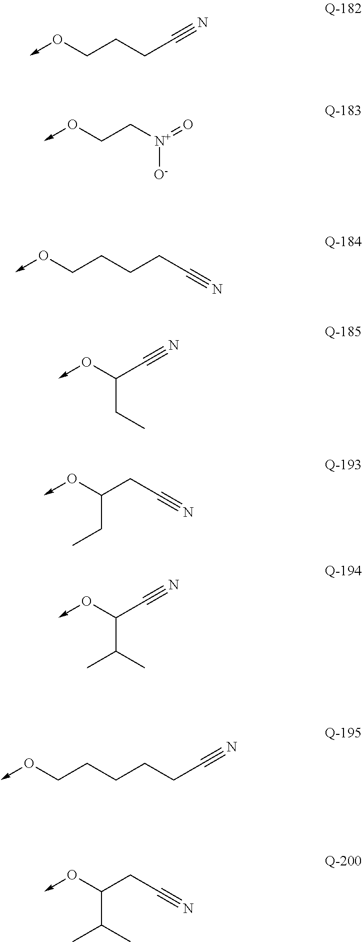

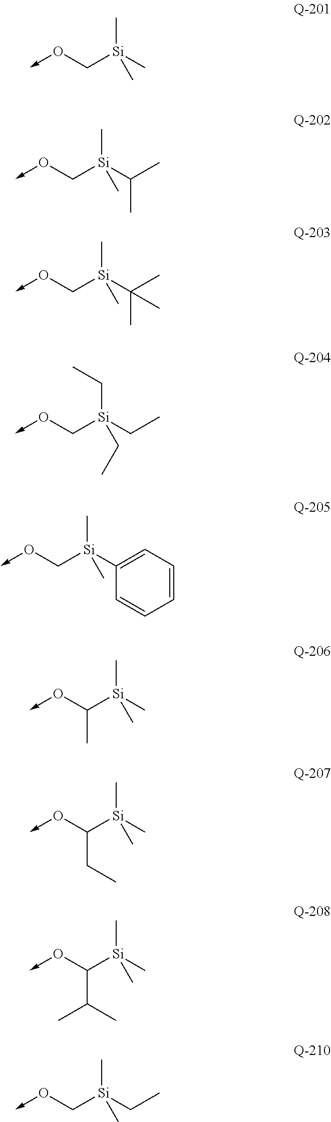

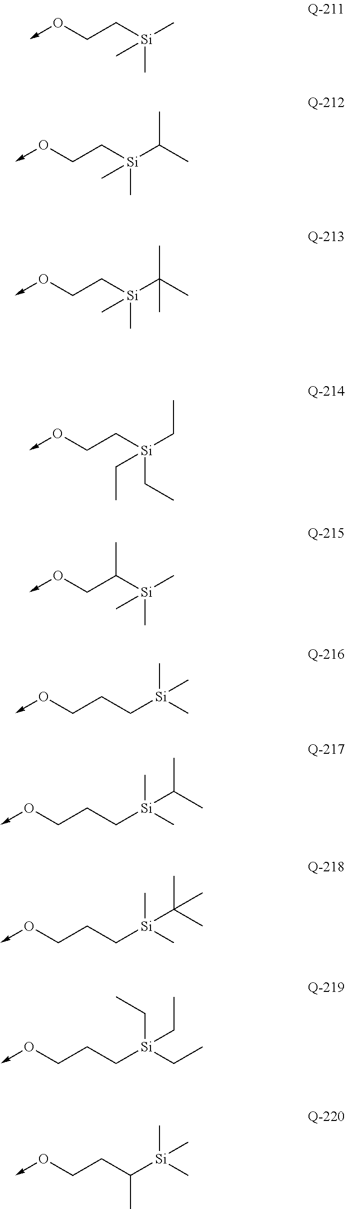

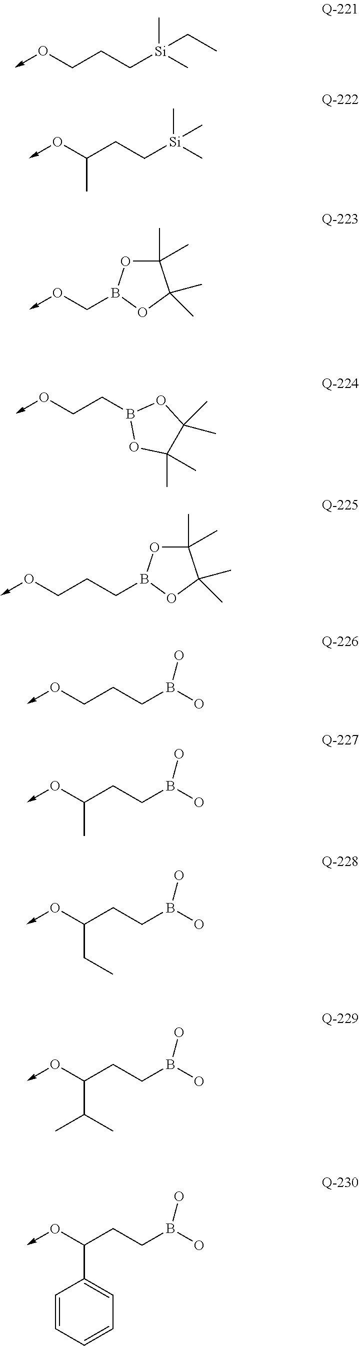

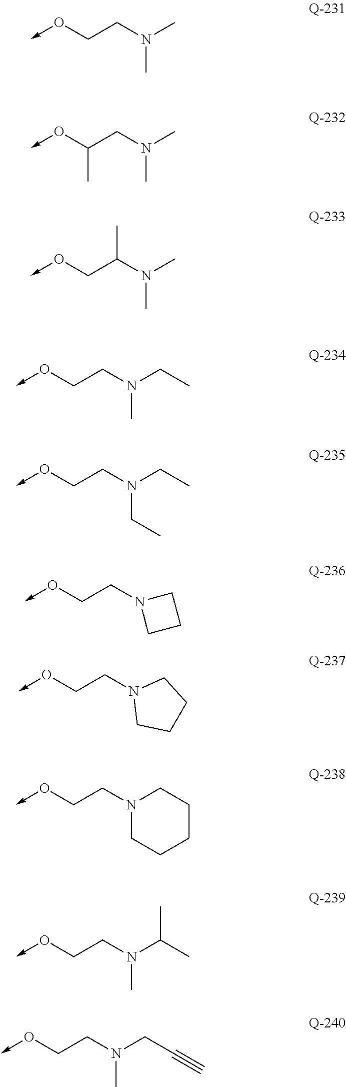

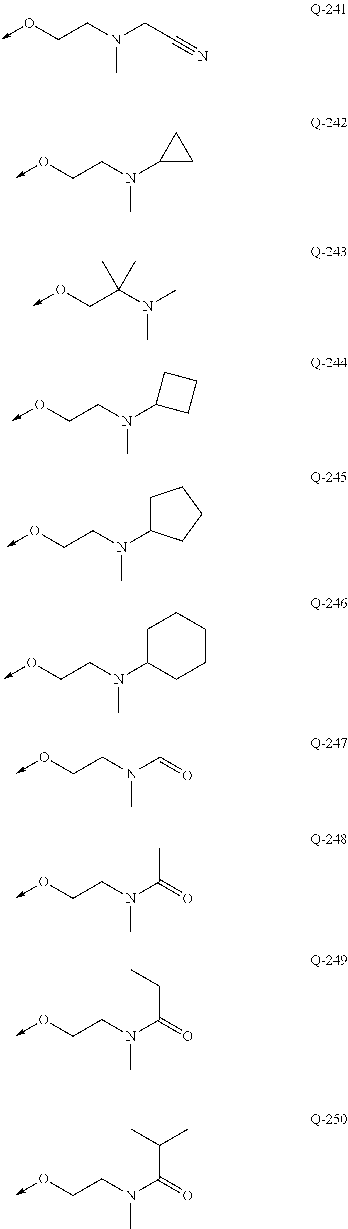

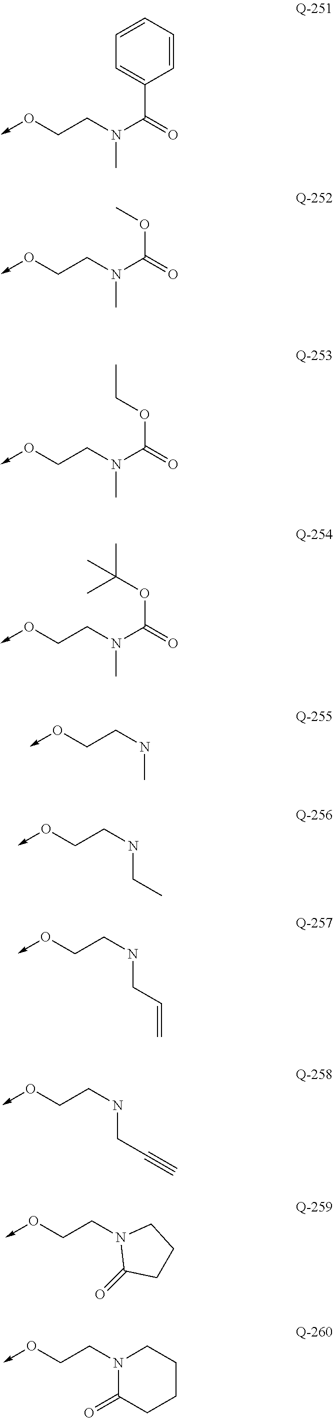

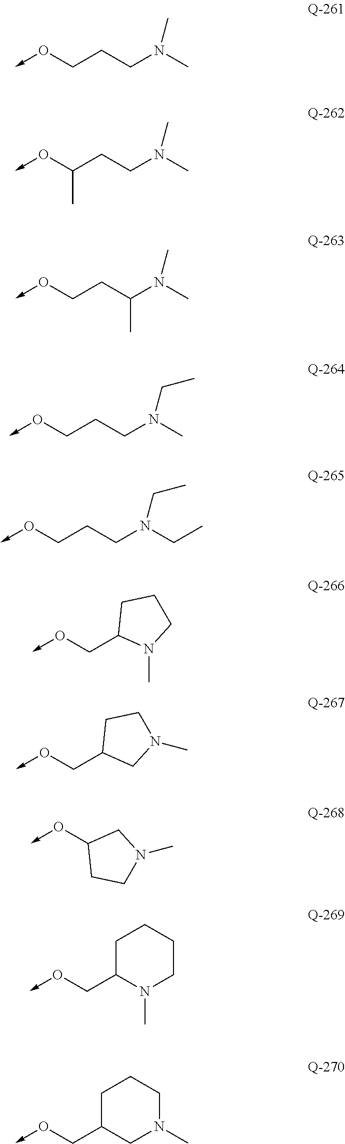

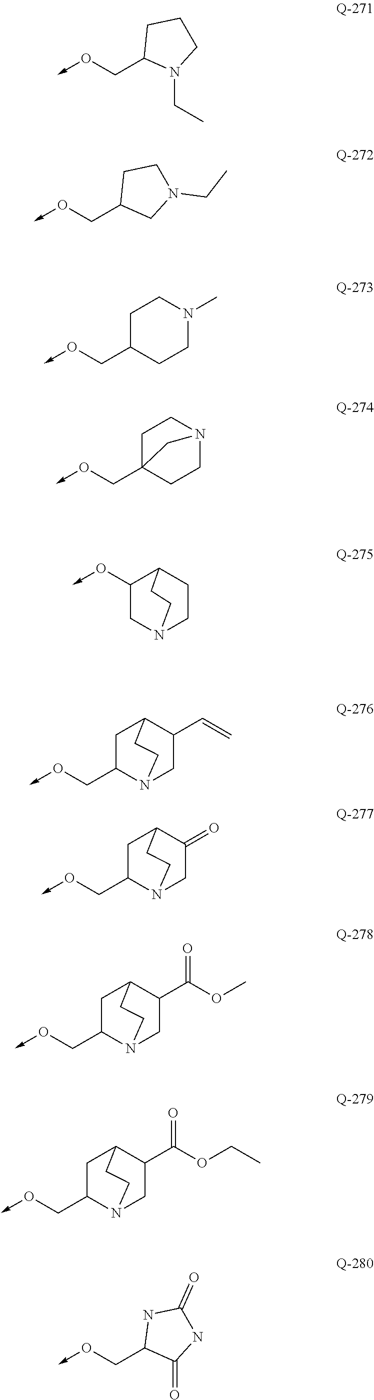

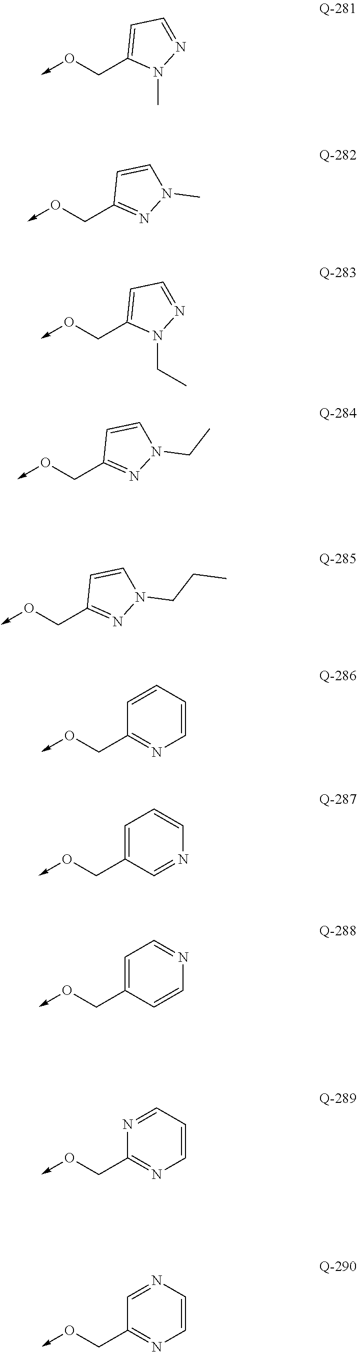

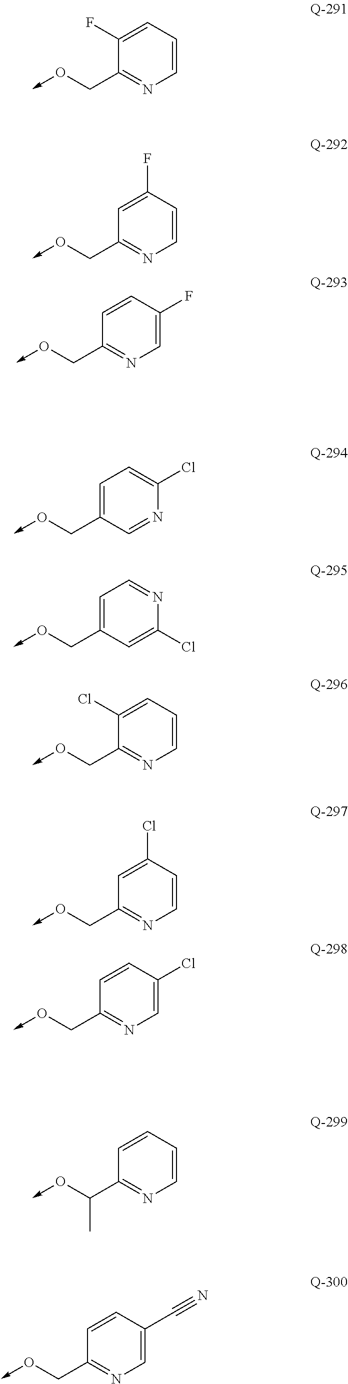

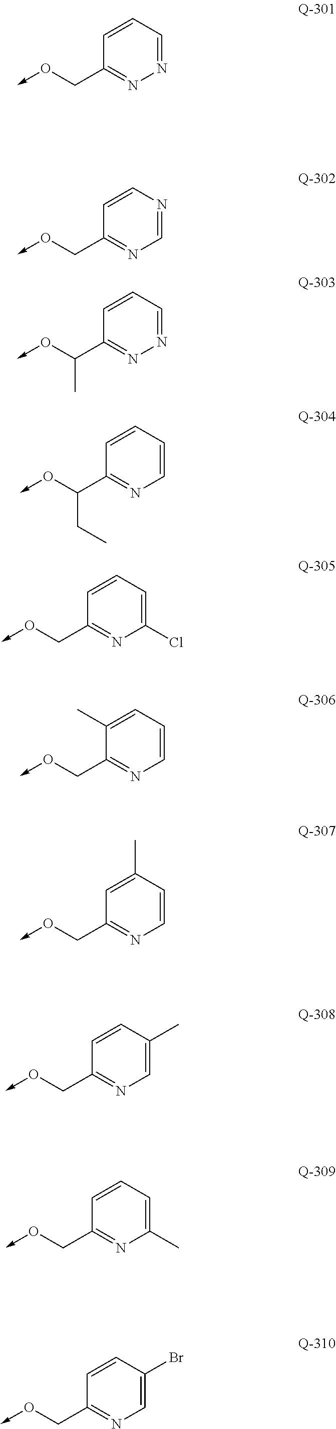

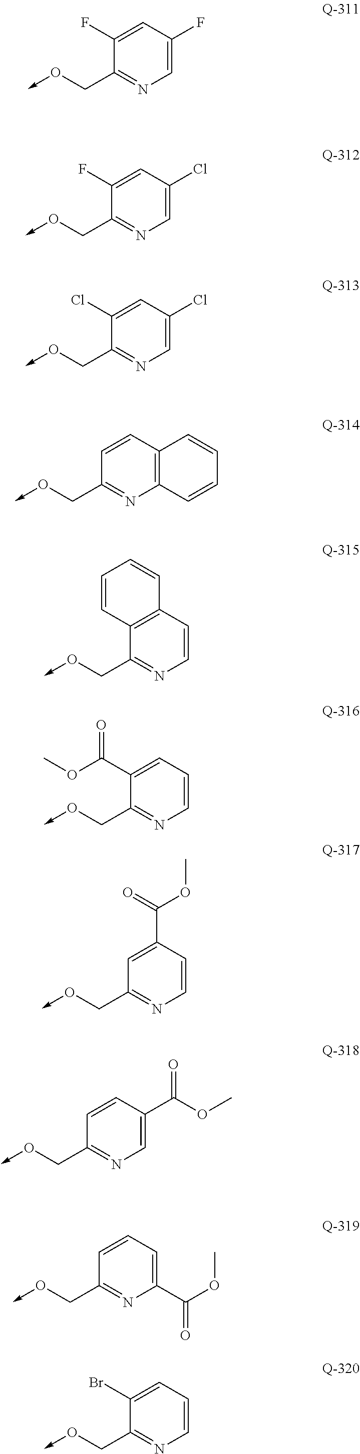

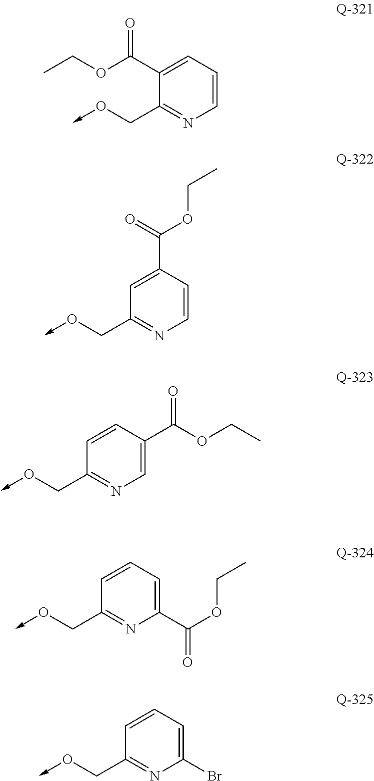

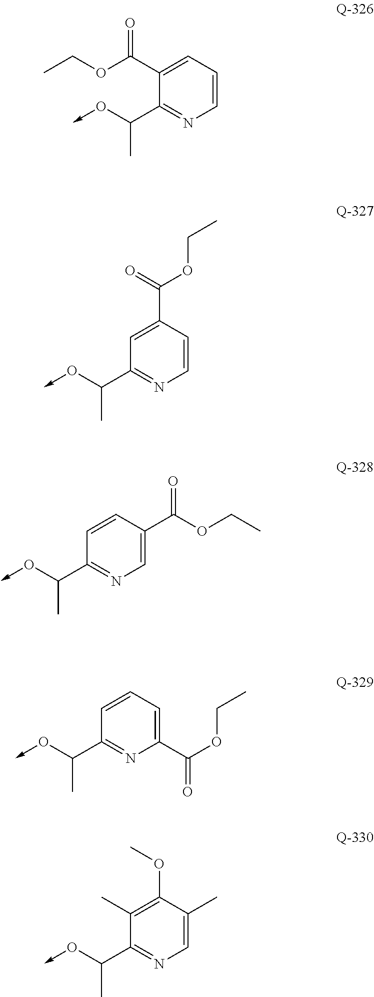

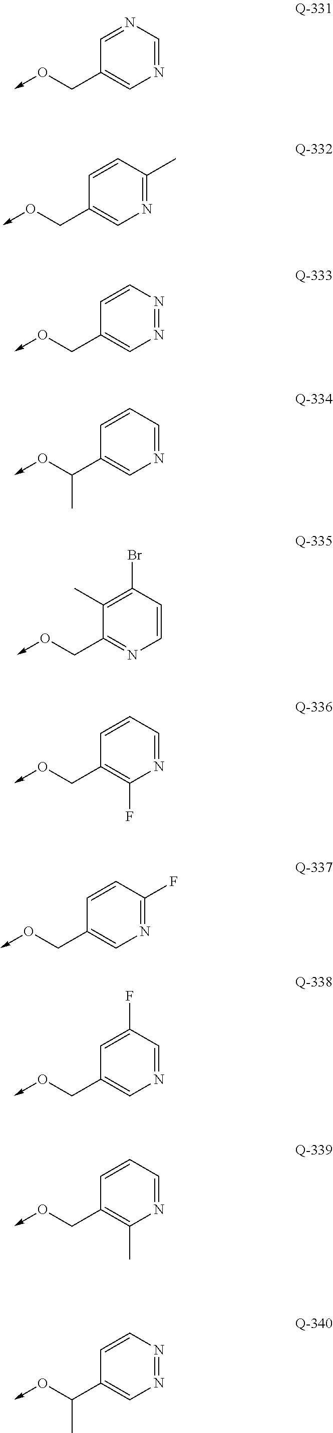

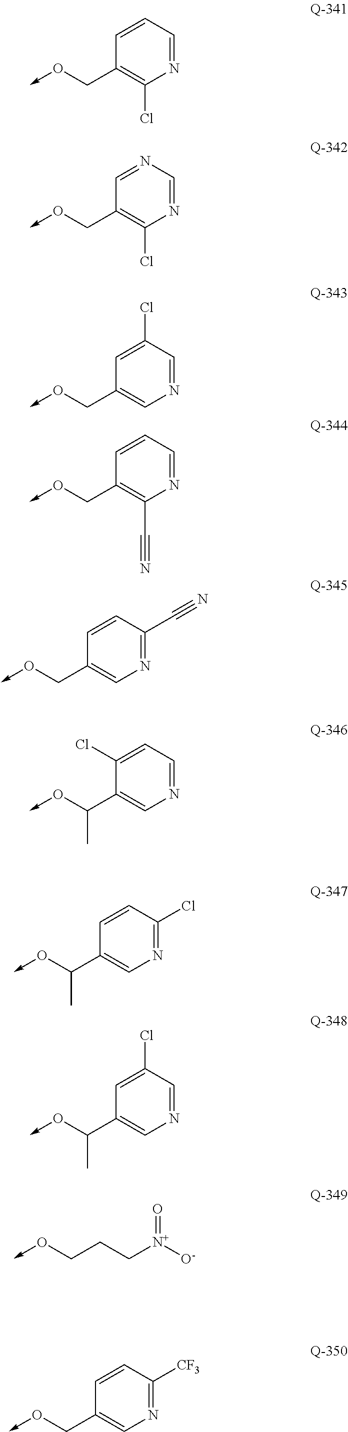

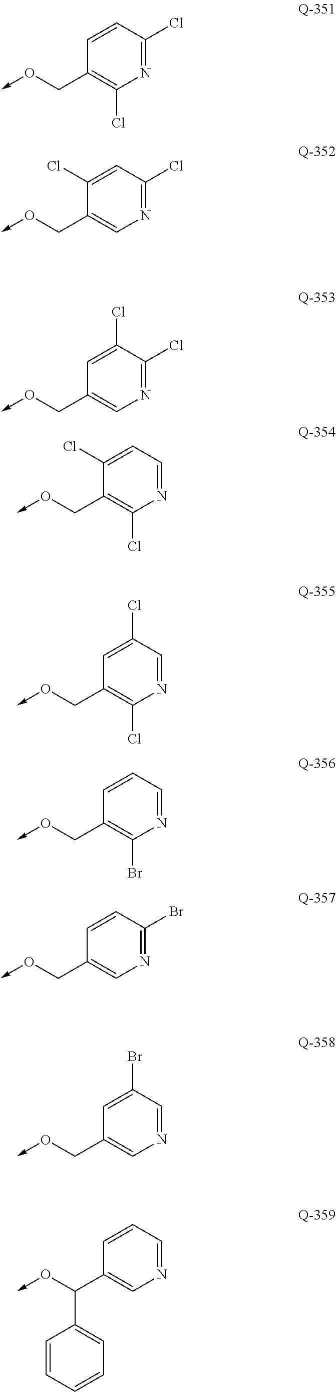

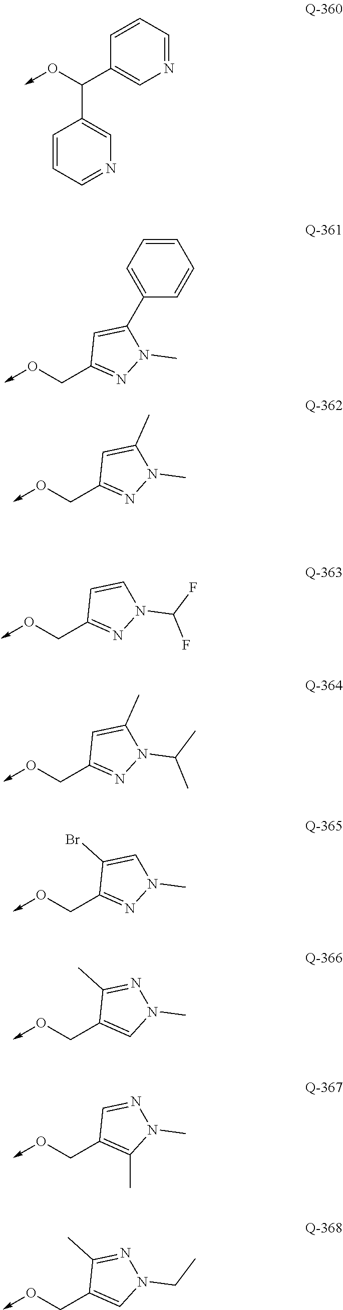

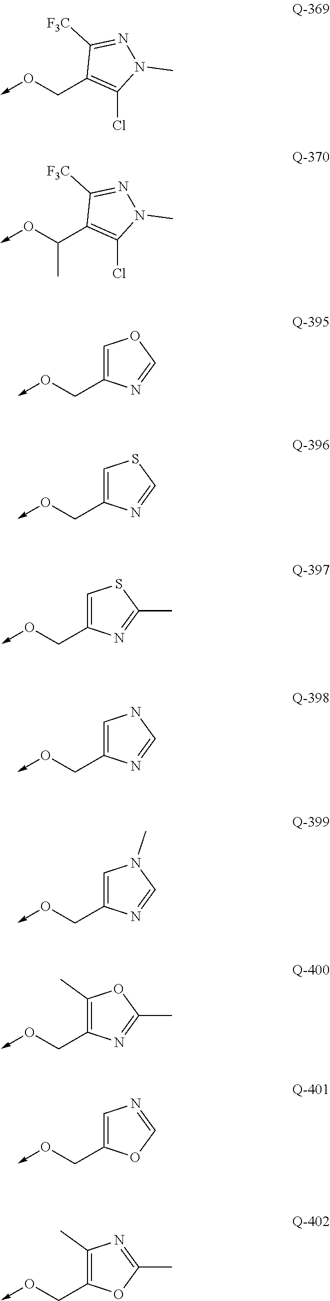

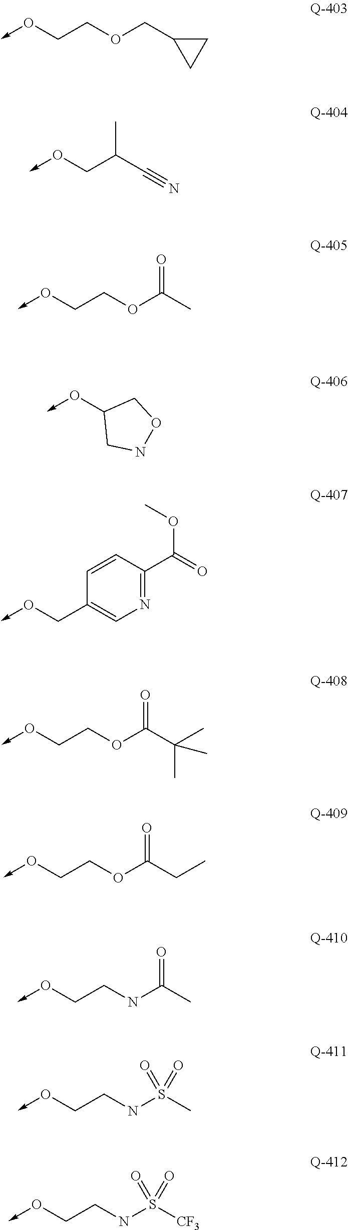

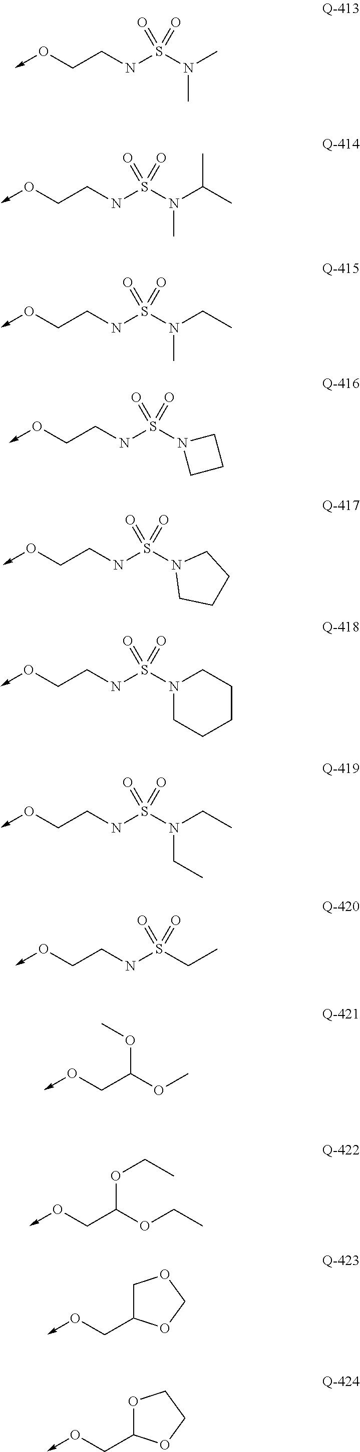

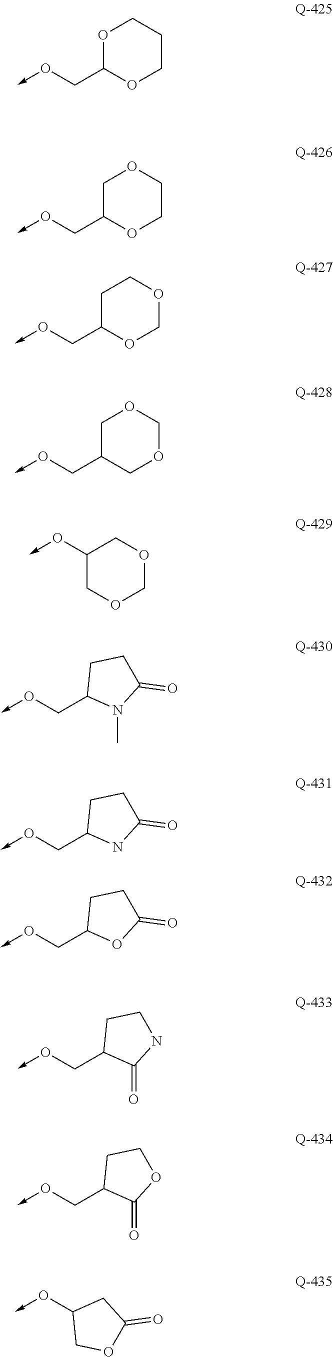

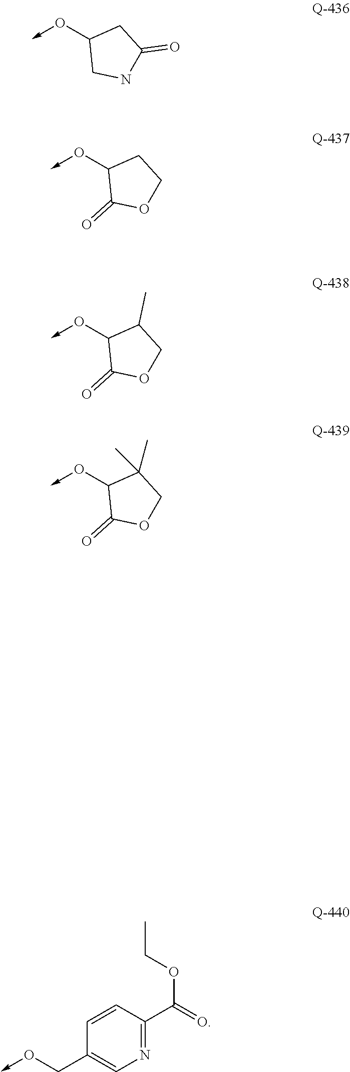

The invention especially provides compounds of the general formula (I) in which
R¹ represents hydrogen,
R² represents fluorine,
R³ represents hydrogen, fluorine, chlorine, bromine, methoxy,
R⁴ represents fluorine, chlorine, bromine, cyano, NO₂, C(O)NH₂, C(S)NH₂, trifluoromethyl, ethynyl, propyn-1-yl,
R⁵, R⁶ and R⁷ independently of one another represent hydrogen, fluorine, chlorine, bromine, iodine, cyano, methyl, ethyl, trifluoromethyl, difluoromethyl, methoxy, ethoxy, difluoromethoxy, trifluoromethoxy,
G represents methylene, (methyl)methylene, (ethyl)methylene, (dimethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, 1,1-dimethylethyl-1-ene, 2,2-dimethylethyl-1-ene, 1-ethylethyl-1-ene, 2-ethylethyl-1-ene, 1-(prop-1-yl)ethyl-1-ene, 2-(prop-1-yl)ethyl-1-ene, 1-(prop-2-yl)ethyl-1-ene, 2-(prop-2-yl)ethyl-1-ene, n-pentylene, 1-methylbutyl-1-ene, 2-methylbutyl-1-ene, 3-methylbutyl-1-ene, 4-methylbutyl-1-ene, 1,1-dimethylpropyl-1-ene, 2,2-dimethylpropyl-1-ene, 3,3-dimethylpropyl-1-ene, 1,2-dimethylpropyl-1-ene, 1,3-dimethylpropyl-1-ene, 1-ethylpropyl-1-ene, n-hexylene,
X and Y independently of one another represent O (oxygen) or S (sulfur)
   and
Q represents one of the moieties Q-1 to Q-54, Q-56 to Q-57, Q-60 to Q-89, Q-91 to Q-129, Q-131 to Q-139, Q-141 to Q-144, Q-146 to Q-180, Q-182 to Q-185, Q-193 to Q-195, Q-200 to Q-208, Q-210 to Q-370, Q-395 to Q-440 specifically mentioned above.

The invention very especially provides compounds of the general formula (I) in which
R¹ represents hydrogen,
R² represents fluorine,
R³ represents fluorine,
R⁴ represents chlorine, bromine, cyano, NO₂, C(O)NH₂, C(S)NH₂,
R⁵, R⁶ and R⁷ independently of one another represent hydrogen, fluorine, chlorine, bromine, cyano, methyl, trifluoromethyl, methoxy, trifluoromethoxy,
G represents methylene, (methyl)methylene, (ethyl)methylene, (dimethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, n-pentylene, n-hexylene,
X and Y independently of one another represent O (oxygen) or S (sulfur)
   and
Q represents one of the moieties Q-1 to Q-54, Q-56 to Q-57, Q-60 to Q-89, Q-91 to Q-129, Q-131 to Q-139, Q-141 to Q-144, Q-146 to Q-180, Q-182 to Q-185, Q-193 to Q-195, Q-200 to Q-208, Q-210 to Q-370, Q-395 to Q-440 specifically mentioned above.

The invention particularly especially provides compounds of the general formula (I) in which
R¹ represents hydrogen,
R² represents fluorine,
R³ represents fluorine,
R⁴ represents chlorine, bromine, cyano, NO₂,
R⁵ represents hydrogen,
R⁶ represents hydrogen, fluorine, chlorine, bromine,
R⁷ represents hydrogen,
G represents methylene,
X and Y independently of one another represent O (oxygen) or S (sulfur)
   and
Q represents one of the moieties Q-1 to Q-54, Q-56 to Q-57, Q-60 to Q-89, Q-91 to Q-129, Q-131 to Q-139, Q-141 to Q-144, Q-146 to Q-180, Q-182 to Q-185, Q-193 to Q-195, Q-200 to Q-208, Q-210 to Q-370, Q-395 to Q-440 specifically mentioned above.

The invention very particularly especially provides compounds of the general formula (I) in which
R¹ represents hydrogen,
R² represents fluorine,
R³ represents fluorine,
R⁴ represents chlorine, bromine, cyano, NO₂,
R⁵ represents hydrogen,
R⁶ represents hydrogen, fluorine,
R⁷ represents hydrogen,
G represents methylene,
X represents O (oxygen) or S (sulfur),
Y represents O (oxygen),
   and
Q represents one of the moieties Q-1 to Q-35, Q-41, Q-42, Q-71 to Q-80, Q-115, Q-120, Q-152 to Q-155, Q-166 to Q-170, Q-176 to Q-206, Q-211 to Q-214, Q-280 to Q-358, Q-362 to Q-370, Q-405, Q-408 to Q-410, Q-421 to Q-429 specifically mentioned above.

The invention very especially provides compounds of the general formula (I) in which
R¹ represents hydrogen,
R² represents fluorine,
R³ represents fluorine,
R⁴ represents chlorine, bromine, cyano, NO₂,
R⁵ represents hydrogen,
R⁶ represents hydrogen, fluorine,
R⁷ represents hydrogen,
G represents methylene,
X represents O (oxygen) or S (sulfur),
Y represents O (oxygen),
   and
Q represents one of the moieties Q-1, Q-2, Q-6, Q-23, Q-26, Q-31, Q-41, Q-71, Q-72, Q-115, Q-154, Q-166, Q-176, Q-201, Q-211, Q-280, Q-286, Q-288, Q-301, Q-350, Q-366, Q-367, Q-368, Q-405, Q-421, Q-422, Q-424 specifically mentioned above.

The abovementioned general or preferred radical definitions apply both to the end products of the general formula (I) and, correspondingly, to the starting materials or the intermediates required in each case for the preparation. These radical definitions can be combined with one another as desired, i.e. including combinations between the given preferred ranges.

Primarily for reasons of higher herbicidal activity, better selectivity and/or better producibility, inventive compounds of the abovementioned general formula (I) or their salts or their use according to the invention are of particular interest in which individual radicals have one of the preferred meanings already specified or specified below, or in particular those in which one or more of the preferred meanings already specified or specified below occur in combination.

If the compounds can form, through a hydrogen shift, tautomers whose structure is not formally covered by the general formula (I), these tautomers are nevertheless covered by the definition of the compounds of the general formula (I) according to the invention, unless a particular tautomer is under consideration. For example, many carbonyl compounds may be present both in the keto form and in the enol form, both forms being encompassed by the definition of the compound of the general formula (I).

Depending on the nature of the substituents and the manner in which they are attached, the compounds of the general formula (I) may be present as stereoisomers. The possible stereoisomers defined by the specific three-dimensional form thereof, such as enantiomers, diastereomers, Z and E isomers, are all encompassed by the general formula (I). If, for example, one or more alkenyl groups are present, diastereomers (Z and E isomers) may occur. If, for example, one or more asymmetric carbon atoms are present, enantiomers and diastereomers may occur. Stereoisomers can be obtained from the mixtures obtained in the preparation by customary separation methods. The chromatographic separation can be effected either on the analytical scale to find the enantiomeric excess or the diastereomeric excess, or else on the preparative scale to produce test specimens for biological testing. It is likewise possible to selectively prepare stereoisomers by using stereoselective reactions with use of optically active starting materials and/or auxiliaries. The invention thus also relates to all stereoisomers which are embraced by the general formula (I) but are not shown in their specific stereometric form, and to mixtures thereof.

If the compounds are obtained as solids, the purification can also be carried out by recrystallization or digestion. If individual compounds (I) cannot be obtained in a satisfactory manner by the routes described below, they can be prepared by derivatization of other compounds (I).

Suitable isolation methods, purification methods and methods for separating stereoisomers of compounds of the general formula (I) are methods generally known to the person skilled in the art from analogous cases, for example by physical processes such as crystallization, chromatographic methods, in particular column chromatography and HPLC (high pressure liquid chromatography), distillation, optionally under reduced pressure, extraction and other methods, any mixtures that remain can generally be separated by chromatographic separation, for example on chiral solid phases. Suitable for preparative amounts or on an industrial scale are processes such as crystallization, for example of diastereomeric salts which can be obtained from the diastereomer mixtures using optically active acids and, if appropriate, provided that acidic groups are present, using optically active bases.

With regard to the compounds according to the invention, the terms used above and further below will be elucidated. These are familiar to the person skilled in the art and especially have the definitions elucidated hereinafter:

Unless defined differently, names of chemical groups are generally to be understood such that attachment to the skeleton or the remainder of the molecule is via the structural element of the relevant chemical group mentioned last, i.e. for example in the case of (C₂-C₈)-alkenyloxy via the oxygen atom and in the case of heterocyclyl-(C₁-C₈)-alkyl or (C₁-C₆)-alkoxy-(C₁-C₆)-alkoxy-(C₁-C₆)-alkyl in each case via the carbon atom of the alkyl group.

According to the invention, “alkylsulfonyl”—alone or as part of a chemical group—refers to straight-chain or branched alkylsulfonyl, preferably having 1 to 8 or 1 to 6 carbon atoms, for example (but not limited to) (C₁-C₆)-alkylsulfonyl such as methylsulfonyl, ethylsulfonyl, propylsulfonyl, 1-methylethylsulfonyl, butylsulfonyl, 1-methylpropylsulfonyl, 2-methylpropylsulfonyl, 1,1-dimethylethylsulfonyl, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl.

According to the invention, “heteroarylsulfonyl” refers to optionally substituted pyridylsulfonyl, pyrimidinylsulfonyl, pyrazinylsulfonyl or optionally substituted polycyclic heteroarylsulfonyl, here in particular optionally substituted quinolinylsulfonyl, for example substituted by fluorine, chlorine, bromine, iodine, cyano, nitro, alkyl, haloalkyl, haloalkoxy, amino, alkylamino, alkylcarbonylamino, dialkylamino or alkoxy groups.

According to the invention, “alkylthio”—alone or as part of a chemical group—refers to straight-chain or branched S-alkyl, preferably having 1 to 8 or 1 to 6 carbon atoms, such as (C₁-C₁₀)-, (C₁-C₆)- or (C₁-C₄)-alkylthio, for example (but not limited to) (C₁-C₆)-alkylthio such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio.

According to the invention, “alkenylthio” denotes an alkenyl radical bonded via a sulfur atom, alkynylthio denotes an alkynyl radical bonded via a sulfur atom, cycloalkylthio denotes a cycloalkyl radical bonded via a sulfur atom, and cycloalkenylthio denotes a cycloalkenyl radical bonded via a sulfur atom.

According to the invention, “alkylsulfinyl (alkyl-S(═O)—)”, unless defined differently elsewhere, denotes alkyl radicals which are bonded to the skeleton via —S(═O)—, such as (C₁-C₁₀)-, (C₁-C₆)- or (C₁-C₄)-alkylsulfinyl, for example (but not limited to) (C₁-C₆)-alkylsulfinyl such as methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl.

Analogously, “alkenylsulfinyl” and “alkynylsulfinyl” are defined in accordance with the invention respectively as alkenyl and alkynyl radicals bonded to the skeleton via —S(═O)—, such as (C₂-C₁₀)-, (C₂-C₆)- or (C₂-C₄)-alkenylsulfinyl or (C₃-C₁₀)-, (C₃-C₆)- or (C₃-C₄)-alkynylsulfinyl.

Analogously, “alkenylsulfonyl” and “alkynylsulfonyl” are defined in accordance with the invention respectively as alkenyl and alkynyl radicals bonded to the skeleton via —S(═O)₂—, such as (C₂-C₁₀)-, (C₂-C₆)- or (C₂-C₄)-alkenylsulfonyl or (C₃-C₁₀)-, (C₃-C₆)- or (C₃-C₄)-alkynylsulfonyl.

“Alkoxy” denotes an alkyl radical attached via an oxygen atom, for example (but not limited to) (C₁-C₆)-alkoxy such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy. Alkenyloxy denotes an alkenyl radical attached via an oxygen atom, and alkynyloxy denotes an alkynyl radical attached via an oxygen atom, such as (C₂-C₁₀)-, (C₂-C₆)- or (C₂-C₄)-alkenoxy and (C₃-C₁₀)-, (C₃-C₆)- or (C₃-C₄)-alkynoxy.

“Cycloalkyloxy” denotes a cycloalkyl radical bonded via an oxygen atom and cycloalkenyloxy denotes a cycloalkenyl radical bonded via an oxygen atom.

According to the invention, “alkylcarbonyl” (alkyl-C(═O)—), unless defined differently elsewhere, represents alkyl radicals bonded to the skeleton via —C(═O)—, such as (C₁-C₁₀)-, (C₁-C₆)- or (C₁-C₄)-alkylcarbonyl. Here, the number of the carbon atoms refers to the alkyl radical in the alkylcarbonyl group.

Analogously, “alkenylcarbonyl” and “alkynylcarbonyl”, unless defined differently elsewhere, in accordance with the invention, respectively represent alkenyl and alkynyl radicals bonded to the skeleton via —C(═O)—, such as (C₂-C₁₀)-, (C₂-C₆)- or (C₂-C₄)-alkenylcarbonyl and (C₂-C₁₀)-, (C₂-C₆)- or (C₂-C₄)-alkynylcarbonyl. Here, the number of the carbon atoms refers to the alkenyl or alkynyl radical in the alkenylcarbonyl or alkynylcarbonyl group.

“Alkoxycarbonyl (alkyl-O—C(═O)—)”, unless defined differently elsewhere: alkyl radicals bonded to the skeleton via —O—C(═O)—, such as (C₁-C₁₀)-, (C₁-C₆)- or (C₁-C₄)-alkoxycarbonyl.

Here, the number of the carbon atoms refers to the alkyl radical in the alkoxycarbonyl group. Analogously, “alkenyloxycarbonyl” and “alkynyloxycarbonyl”, unless defined differently elsewhere, in accordance with the invention, respectively represent alkenyl and alkynyl radicals bonded to the skeleton via —O—C(═O)—, such as (C₂-C₁₀)-, (C₂-C₆)- or (C₂-C₄)-alkenyloxycarbonyl and (C₃-C₁₀)-, (C₃-C₆)- or (C₃-C₄)-alkynyloxycarbonyl. Here, the number of the carbon atoms refers to the alkenyl or alkynyl radical in the alkenyloxycarbonyl or alkynyloxycarbonyl group.

According to the invention, the term “alkylcarbonyloxy” (alkyl-C(═O)—O—), unless defined differently elsewhere, represents alkyl radicals bonded to the skeleton via the oxygen of a carbonyloxy group (—C(═O)—O—), such as (C₁-C₁₀)-, (C₁-C₆)- or (C₁-C₄)-alkylcarbonyloxy. Here, the number of the carbon atoms refers to the alkyl radical in the alkylcarbonyloxy group.

Analogously, “alkenylcarbonyloxy” and “alkynylcarbonyloxy” are defined in accordance with the invention respectively as alkenyl and alkynyl radicals bonded to the skeleton via the oxygen of (—C(═O)—O—), such as (C₂-C₁₀)-, (C₂-C₆)- or (C₂-C₄)-alkenylcarbonyloxy or (C₂-C₁₀)-, (C₂-C₆)- or (C₂-C₄)-alkynylcarbonyloxy. Here, the number of the carbon atoms refers to the alkenyl or alkynyl radical in the alkenyl- or alkynylcarbonyloxy group respectively.

In short forms such as C(O)R¹³, C(O)OR¹³, OC(O)NR¹¹R¹² or C(O)NR¹¹R¹², the short form O shown in brackets represents an oxygen atom attached to the adjacent carbon atom via a double bond.

In short forms such as OC(S)OR¹³, OC(S)SR¹⁴, OC(S)NR¹¹R¹², the short form S shown in brackets represents a sulfur atom attached to the adjacent carbon atom via a double bond.

The term “aryl” denotes an optionally substituted mono-, bi- or polycyclic aromatic system having preferably 6 to 14, especially 6 to 10, ring carbon atoms, for example phenyl, naphthyl, anthryl, phenanthrenyl and the like, preferably phenyl.

The term “optionally substituted aryl” also includes polycyclic systems, such as tetrahydronaphthyl, indenyl, indanyl, fluorenyl, biphenylyl, where the bonding site is on the aromatic system. In systematic terms, “aryl” is generally also encompassed by the term “optionally substituted phenyl”. Preferred aryl substituents here are, for example, hydrogen, halogen, alkyl, cycloalkyl, cycloalkylalkyl, cycloalkenyl, halocycloalkyl, alkenyl, alkynyl, aryl, arylalkyl, arylalkenyl, heteroaryl, heteroarylalkyl, heterocyclyl, heterocyclylalkyl, alkoxyalkyl, alkylthio, haloalkylthio, haloalkyl, alkoxy, haloalkoxy, cycloalkoxy, cycloalkylalkoxy, aryloxy, heteroraryloxy, alkoxyalkoxy, alkynylalkoxy, alkenyloxy, bisalkylaminoalkoxy, tris[alkyl]silyl, bis[alkyl]arylsilyl, bis[alkyl]alkylsilyl, tris[alkyl]silylalkynyl, arylalkynyl, heteroarylalkynyl, alkylalkynyl, cycloalkylalkynyl, haloalkylalkynyl, heterocyclyl-N-alkoxy, nitro, cyano, amino, alkylamino, bis-alkylamino, alkylcarbonylamino, cycloalkylcarbonylamino, arylcarbonylamino, alkoxycarbonylamino, alkoxycarbonylalkylamino, arylalkoxycarbonylalkylamino, hydroxycarbonyl, alkoxycarbonyl, aminocarbonyl, alkylaminocarbonyl, cycloalkylaminocarbonyl, bisalkylaminocarbonyl, heteroarylalkoxy, arylalkoxy.

A heterocyclic radical (heterocyclyl) contains at least one heterocyclic ring (=carbocyclic ring in which at least one carbon atom has been replaced by a heteroatom, preferably by a heteroatom from the group of N, O, S, P) which is saturated, unsaturated, partially saturated or heteroaromatic and may be unsubstituted or substituted, in which case the bonding site is localized on a ring atom. If the heterocyclyl radical or the heterocyclic ring is optionally substituted, it may be fused to other carbocyclic or heterocyclic rings. In the case of optionally substituted heterocyclyl, polycyclic systems are also included, for example 8-azabicyclo[3.2.1]octanyl, 8-azabicyclo[2.2.2]octanyl or 1-azabicyclo[2.2.1]heptyl. Optionally substituted heterocyclyl also includes spirocyclic systems, such as, for example, 1-oxa-5-azaspiro[2.3]hexyl. Unless defined differently, the heterocyclic ring preferably contains 3 to 9 ring atoms, especially 3 to 6 ring atoms, and one or more, preferably 1 to 4, especially 1, 2 or 3, heteroatoms in the heterocyclic ring, preferably from the group of N, O and S, but no two oxygen atoms should be directly adjacent, for example with one heteroatom from the group of N, O and S: 1- or 2- or 3-pyrrolidinyl, 3,4-dihydro-2H-pyrrol-2- or -3-yl, 2,3-dihydro-1H-pyrrol-1- or -2- or -3- or -4- or -5-yl; 2,5-dihydro-1H-pyrrol-1- or -2- or -3-yl, 1- or 2- or 3- or 4-piperidinyl; 2,3,4,5-tetrahydropyridin-2- or -3- or -4- or -5-yl or -6-yl; 1,2,3,6-tetrahydropyridin-1- or -2- or -3- or -4- or -5- or -6-yl; 1,2,3,4-tetrahydropyridin-1- or -2- or -3- or -4- or -5- or -6-yl; 1,4-dihydropyridin-1- or -2- or -3- or -4-yl; 2,3-dihydropyridin-2- or -3- or -4- or -5- or -6-yl; 2,5-dihydropyridin-2- or -3- or -4- or -5- or -6-yl, 1- or 2- or 3- or 4-azepanyl; 2,3,4,5-tetrahydro-1H-azepin-1- or -2- or -3- or -4- or -5- or -6- or -7-yl; 2,3,4,7-tetrahydro-1H-azepin-1- or -2- or -3- or -4- or -5- or -6- or -7-yl; 2,3,6,7-tetrahydro-1H-azepin-1- or -2- or -3- or -4-yl; 3,4,5,6-tetrahydro-2H-azepin-2- or -3- or -4- or -5- or -6- or -7-yl; 4,5-dihydro-1H-azepin-1- or -2- or -3- or -4-yl; 2,5-dihydro-1H-azepin-1- or -2- or -3- or -4- or -5- or -6- or -7-yl; 2,7-dihydro-1H-azepin-1- or -2- or -3- or -4-yl; 2,3-dihydro-1H-azepin-1- or -2- or -3- or -4- or -5- or -6- or -7-yl; 3,4-dihydro-2H-azepin-2- or -3- or -4- or -5- or -6- or -7-yl; 3,6-dihydro-2H-azepin-2- or -3- or -4- or -5- or -6- or -7-yl; 5,6-dihydro-2H-azepin-2- or -3- or -4- or -5- or -6- or -7-yl; 4,5-dihydro-3H-azepin-2- or -3- or -4- or -5- or -6- or -7-yl; 1H-azepin-1- or -2- or -3- or -4- or -5- or -6- or -7-yl; 2H-azepin-2- or -3- or -4- or -5- or -6- or -7-yl; 3H-azepin-2- or -3- or -4- or -5- or -6- or -7-yl; 4H-azepin-2- or -3- or -4- or -5- or -6- or -7-yl, 2- or 3-oxolanyl (=2- or 3-tetrahydrofuranyl); 2,3-dihydrofuran-2- or -3- or -4- or -5-yl; 2,5-dihydrofuran-2- or -3-yl, 2- or 3- or 4-oxanyl (=2- or 3- or 4-tetrahydropyranyl); 3,4-dihydro-2H-pyran-2- or -3- or -4- or -5- or -6-yl; 3,6-dihydro-2H-pyran-2- or -3- or -4- or -5- or -6-yl; 2H-pyran-2- or -3- or -4- or -5- or -6-yl; 4H-pyran-2- or -3- or -4-yl, 2- or 3- or 4-oxepanyl; 2,3,4,5-tetrahydrooxepin-2- or -3- or -4- or -5- or -6- or -7-yl; 2,3,4,7-tetrahydrooxepin-2- or -3- or -4- or -5- or -6- or -7-yl; 2,3,6,7-tetrahydrooxepin-2- or -3- or -4-yl; 2,3-dihydrooxepin-2- or -3- or -4- or -5- or -6- or -7-yl; 4,5-dihydrooxepin-2- or -3- or -4-yl; 2,5-dihydrooxepin-2- or -3- or -4- or -5- or -6- or -7-yl; oxepin-2- or -3- or -4- or -5- or -6- or -7-yl; 2- or 3-tetrahydrothiophenyl; 2,3-dihydrothiophen-2- or -3- or -4- or -5-yl; 2,5-dihydrothiophen-2- or -3-yl; tetrahydro-2H-thiopyran-2- or -3- or -4-yl; 3,4-dihydro-2H-thiopyran-2- or -3- or -4- or -5- or -6-yl; 3,6-dihydro-2H-thiopyran-2- or -3- or -4- or -5- or -6-yl; 2H-thiopyran-2- or -3- or -4- or -5- or -6-yl; 4H-thiopyran-2- or -3- or -4-yl. Preferred 3-membered and 4-membered heterocycles are, for example, 1- or 2-aziridinyl, oxiranyl, thiiranyl, 1- or 2- or 3-azetidinyl, 2- or 3-oxetanyl, 2- or 3-thietanyl, 1,3-dioxetan-2-yl. Further examples of “heterocyclyl” are a partially or fully hydrogenated heterocyclic radical having two heteroatoms from the group consisting of N, O and S, such as, for example, 1- or 2- or 3- or 4-pyrazolidinyl; 4,5-dihydro-3H-pyrazol-3- or -4- or -5-yl; 4,5-dihydro-1H-pyrazol-1- or -3- or -4- or -5-yl; 2,3-dihydro-1H-pyrazol-1- or -2- or -3- or -4- or -5-yl; 1- or 2- or 3- or 4-imidazolidinyl; 2,3-dihydro-1H-imidazol-1- or -2- or -3- or -4-yl; 2,5-dihydro-1H-imidazol-1- or -2- or -4- or -5-yl; 4,5-dihydro-1H-imidazol-1- or -2- or -4- or -5-yl; hexahydropyridazin-1- or -2- or -3- or -4-yl; 1,2,3,4-tetrahydropyridazin-1- or -2- or -3- or -4- or -5- or -6-yl; 1,2,3,6-tetrahydropyridazin-1- or -2- or -3- or -4- or -5- or -6-yl; 1,4,5,6-tetrahydropyridazin-1- or -3- or -4- or -5- or -6-yl; 3,4,5,6-tetrahydropyridazin-3- or -4- or -5-yl; 4,5-dihydropyridazin-3- or -4-yl; 3,4-dihydropyridazin-3- or -4- or -5- or -6-yl; 3,6-dihydropyridazin-3- or -4-yl; 1,6-dihydropyriazin-1- or -3- or -4- or -5- or -6-yl; hexahydropyrimidin-1- or -2- or -3- or -4-yl; 1,4,5,6-tetrahydropyrimidin-1- or -2- or -4- or -5- or -6-yl; 1,2,5,6-tetrahydropyrimidin-1- or -2- or -4- or -5- or -6-yl; 1,2,3,4-tetrahydropyrimidin-1- or -2- or -3- or -4- or -5- or -6-yl; 1,6-dihydropyrimidin-1- or -2- or -4- or -5- or -6-yl; 1,2-dihydropyrimidin-1- or -2- or -4- or -5- or -6-yl; 2,5-dihydropyrimidin-2- or -4- or -5-yl; 4,5-dihydropyrimidin-4- or -5- or -6-yl; 1,4-dihydropyrimidin-1- or -2- or -4- or -5- or -6-yl; 1- or 2- or 3-piperazinyl; 1,2,3,6-tetrahydropyrazin-1- or -2- or -3- or -5- or -6-yl; 1,2,3,4-tetrahydropyrazin-1- or -2- or -3- or -4- or -5- or -6-yl; 1,2-dihydropyrazin-1- or -2- or -3- or -5- or -6-yl; 1,4-dihydropyrazin-1- or -2- or -3-yl; 2,3-dihydropyrazin-2- or -3- or -5- or -6-yl; 2,5-dihydropyrazin-2- or -3-yl; 1,3-dioxolan-2- or -4- or -5-yl; 1,3-dioxol-2- or -4-yl; 1,3-dioxan-2- or -4- or -5-yl; 4H-1,3-dioxin-2- or -4- or -5- or -6-yl; 1,4-dioxan-2- or -3- or -5- or -6-yl; 2,3-dihydro-1,4-dioxin-2- or -3- or -5- or -6-yl; 1,4-dioxin-2- or -3-yl; 1,2-dithiolan-3- or -4-yl; 3H-1,2-dithiol-3- or -4- or -5-yl; 1,3-dithiolan-2- or -4-yl; 1,3-dithiol-2- or -4-yl; 1,2-dithian-3- or -4-yl; 3,4-dihydro-1,2-dithiin-3- or -4- or -5- or -6-yl; 3,6-dihydro-1,2-dithiin-3- or -4-yl; 1,2-dithiin-3- or -4-yl; 1,3-dithian-2- or -4- or -5-yl; 4H-1,3-dithiin-2- or -4- or -5- or -6-yl; isoxazolidin-2- or -3- or -4- or -5-yl; 2,3-dihydroisoxazol-2- or -3- or -4- or -5-yl; 2,5-dihydroisoxazol-2- or -3- or -4- or -5-yl; 4,5-dihydroisoxazol-3- or -4- or -5-yl; 1,3-oxazolidin-2- or -3- or -4- or -5-yl; 2,3-dihydro-1,3-oxazol-2- or -3- or -4- or -5-yl; 2,5-dihydro-1,3-oxazol-2- or -4- or -5-yl; 4,5-dihydro-1,3-oxazol-2- or -4- or -5-yl; 1,2-oxazinan-2- or -3- or -4- or -5- or -6-yl; 3,4-dihydro-2H-1,2-oxazin-2- or -3- or -4- or -5- or -6-yl; 3,6-dihydro-2H-1,2-oxazin-2- or -3- or -4- or -5- or -6-yl; 5,6-dihydro-2H-1,2-oxazin-2- or -3- or -4- or -5- or -6-yl; 5,6-dihydro-4H-1,2-oxazin-3- or -4- or -5- or -6-yl; 2H-1,2-oxazin-2- or -3- or -4- or -5- or -6-yl; 6H-1,2-oxazin-3- or -4- or -5- or -6-yl; 4H-1,2-oxazin-3- or -4- or -5- or -6-yl; 1,3-oxazinan-2- or -3- or -4- or -5- or -6-yl; 3,4-dihydro-2H-1,3-oxazin-2- or -3- or -4- or -5- or -6-yl; 3,6-dihydro-2H-1,3-oxazin-2- or -3- or -4- or -5- or -6-yl; 5,6-dihydro-2H-1,3-oxazin-2- or -4- or -5- or -6-yl; 5,6-dihydro-4H-1,3-oxazin-2- or -4- or -5- or -6-yl; 2H-1,3-oxazin-2- or -4- or -5- or -6-yl; 6H-1,3-oxazin-2- or -4- or -5- or -6-yl; 4H-1,3-oxazin-2- or -4- or -5- or -6-yl; morpholin-2- or -3- or -4-yl; 3,4-dihydro-2H-1,4-oxazin-2- or -3- or -4- or -5- or -6-yl; 3,6-dihydro-2H-1,4-oxazin-2- or -3- or -5- or -6-yl; 2H-1,4-oxazin-2- or -3- or -5- or -6-yl; 4H-1,4-oxazin-2- or -3-yl; 1,2-oxazepan-2- or -3- or -4- or -5- or -6- or -7-yl; 2,3,4,5-tetrahydro-1,2-oxazepin-2- or -3- or -4- or -5- or -6- or -7-yl; 2,3,4,7-tetrahydro-1,2-oxazepin-2- or -3- or -4- or -5- or -6- or -7-yl; 2,3,6,7-tetrahydro-1,2-oxazepin-2- or -3- or -4- or -5- or -6- or -7-yl; 2,5,6,7-tetrahydro-1,2-oxazepin-2- or -3- or -4- or -5- or -6- or -7-yl; 4,5,6,7-tetrahydro-1,2-oxazepin-3- or -4- or -5- or -6- or -7-yl; 2,3-dihydro-1,2-oxazepin-2- or -3- or -4- or -5- or -6- or -7-yl; 2,5-dihydro-1,2-oxazepin-2- or -3- or -4- or -5- or -6- or -7-yl; 2,7-dihydro-1,2-oxazepin-2- or -3- or -4- or -5- or -6- or -7-yl; 4,5-dihydro-1,2-oxazepin-3- or -4- or -5- or -6- or -7-yl; 4,7-dihydro-1,2-oxazepin-3- or -4- or -5- or -6- or -7-yl; 6,7-dihydro-1,2-oxazepin-3- or -4- or -5- or -6- or -7-yl; 1,2-oxazepin-3- or -4- or -5- or -6- or -7-yl; 1,3-oxazepan-2- or -3- or -4- or -5- or -6- or -7-yl; 2,3,4,5-tetrahydro-1,3-oxazepin-2- or -3- or -4- or -5- or -6- or -7-yl; 2,3,4,7-tetrahydro-1,3-oxazepin-2- or -3- or -4- or -5- or -6- or -7-yl; 2,3,6,7-tetrahydro-1,3-oxazepin-2- or -3- or -4- or -5- or -6- or -7-yl; 2,5,6,7-tetrahydro-1,3-oxazepin-2- or -4- or -5- or -6- or -7-yl; 4,5,6,7-tetrahydro-1,3-oxazepin-2- or -4- or -5- or -6- or -7-yl; 2,3-dihydro-1,3-oxazepin-2- or -3- or -4- or -5- or -6- or -7-yl; 2,5-dihydro-1,3-oxazepin-2- or -4- or -5- or -6- or -7-yl; 2,7-dihydro-1,3-oxazepin-2- or -4- or -5- or -6- or -7-yl; 4,5-dihydro-1,3-oxazepin-2- or -4- or -5- or -6- or -7-yl; 4,7-dihydro-1,3-oxazepin-2- or -4- or -5- or -6- or -7-yl; 6,7-dihydro-1,3-oxazepin-2- or -4- or -5- or -6- or -7-yl; 1,3-oxazepin-2- or -4- or -5- or -6- or -7-yl; 1,4-oxazepan-2- or -3- or -5- or -6- or -7-yl; 2,3,4,5-tetrahydro-1,4-oxazepin-2- or -3- or -4- or -5- or -6- or -7-yl; 2,3,4,7-tetrahydro-1,4-oxazepin-2- or -3- or -4- or -5- or -6- or -7-yl; 2,3,6,7-tetrahydro-1,4-oxazepin-2- or -3- or -5- or -6- or -7-yl; 2,5,6,7-tetrahydro-1,4-oxazepin-2- or -3- or -5- or -6- or -7-yl; 4,5,6,7-tetrahydro-1,4-oxazepin-2- or -3- or -4- or -5- or -6- or -7-yl; 2,3-dihydro-1,4-oxazepin-2- or -3- or -5- or -6- or -7-yl; 2,5-dihydro-1,4-oxazepin-2- or -3- or -5- or -6- or -7-yl; 2,7-dihydro-1,4-oxazepin-2- or -3- or -5- or -6- or -7-yl; 4,5-dihydro-1,4-oxazepin-2- or -3- or -4- or -5- or -6- or -7-yl; 4,7-dihydro-1,4-oxazepin-2- or -3- or -4- or -5- or -6- or -7-yl; 6,7-dihydro-1,4-oxazepin-2- or -3- or -5- or -6- or -7-yl; 1,4-oxazepin-2- or -3- or -5- or -6- or -7-yl; isothiazolidin-2- or -3- or -4- or -5-yl; 2,3-dihydroisothiazol-2- or -3- or -4- or -5-yl; 2,5-dihydroisothiazol-2- or -3- or -4- or -5-yl; 4,5-dihydroisothiazol-3- or -4- or -5-yl; 1,3-thiazolidin-2- or -3- or -4- or -5-yl; 2,3-dihydro-1,3-thiazol-2- or -3- or -4- or -5-yl; 2,5-dihydro-1,3-thiazol-2- or -4- or -5-yl; 4,5-dihydro-1,3-thiazol-2- or -4- or -5-yl; 1,3-thiazinan-2- or -3- or -4- or -5- or -6-yl; 3,4-dihydro-2H-1,3-thiazin-2- or -3- or -4- or -5- or -6-yl; 3,6-dihydro-2H-1,3-thiazin-2- or -3- or -4- or -5- or -6-yl; 5,6-dihydro-2H-1,3-thiazin-2- or -4- or -5- or -6-yl; 5,6-dihydro-4H-1,3-thiazin-2- or -4- or -5- or -6-yl; 2H-1,3-thiazin-2- or -4- or -5- or -6-yl; 6H-1,3-thiazin-2- or -4- or -5- or -6-yl; 4H-1,3-thiazin-2- or -4- or -5- or -6-yl. Further examples of “heterocyclyl” are a partially or fully hydrogenated heterocyclic radical having 3 heteroatoms from the group of N, O and S, for example 1,4,2-dioxazolidin-2- or -3- or -5-yl; 1,4,2-dioxazol-3- or -5-yl; 1,4,2-dioxazinan-2- or -3- or -5- or -6-yl; 5,6-dihydro-1,4,2-dioxazin-3- or -5- or -6-yl; 1,4,2-dioxazin-3- or -5- or -6-yl; 1,4,2-dioxazepan-2- or -3- or -5- or -6- or -7-yl; 6,7-dihydro-5H-1,4,2-dioxazepin-3- or -5- or -6- or -7-yl; 2,3-dihydro-7H-1,4,2-dioxazepin-2- or -3- or -5- or -6- or -7-yl; 2,3-dihydro-5H-1,4,2-dioxazepin-2- or -3- or -5- or -6- or -7-yl; 5H-1,4,2-dioxazepin-3- or -5- or -6- or -7-yl; 7H-1,4,2-dioxazepin-3- or -5- or -6- or -7-yl. Structural examples of heterocycles which are optionally substituted further are also listed below:

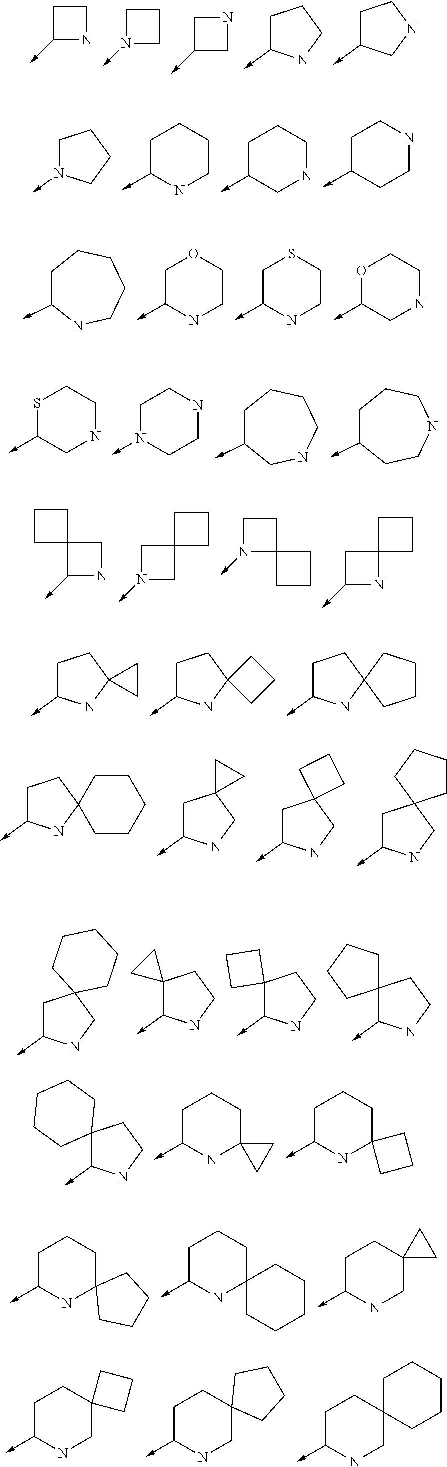

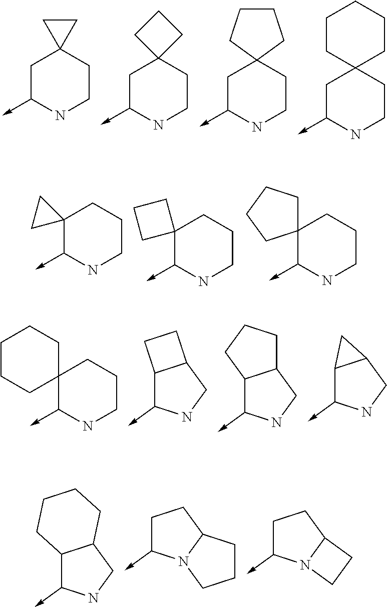

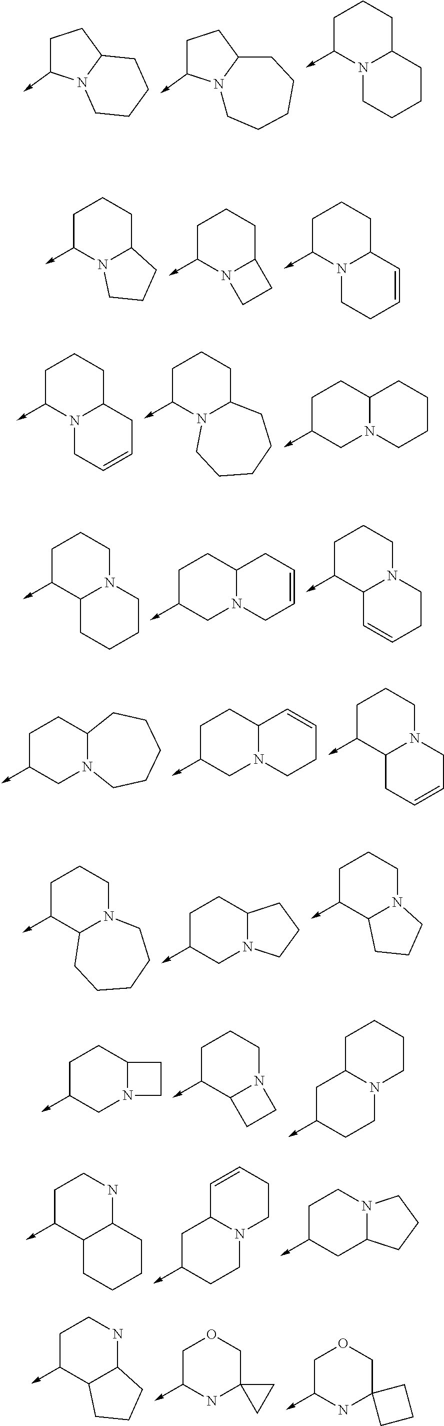

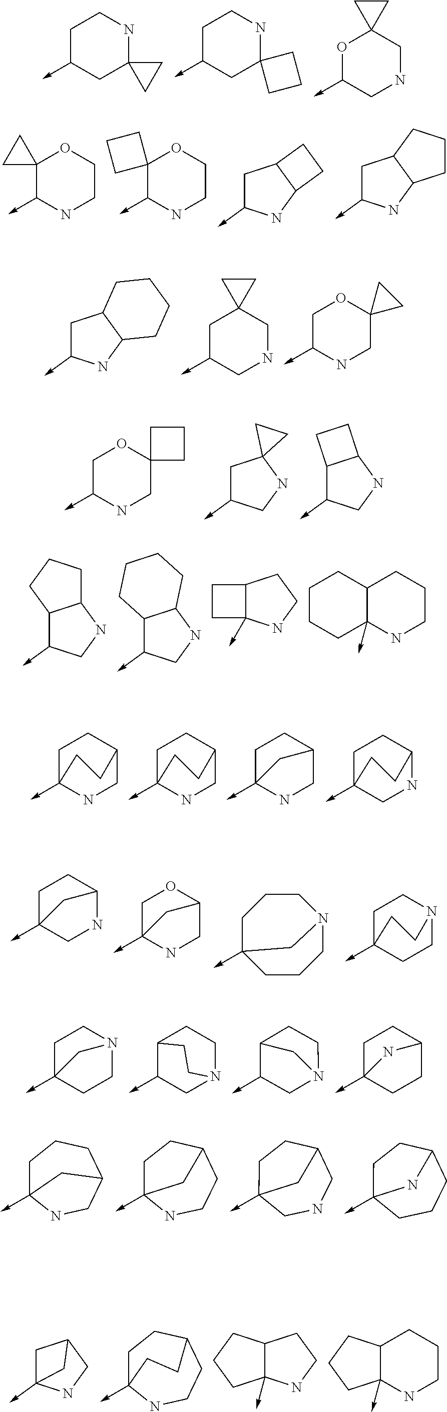

The heterocycles listed above are preferably substituted, for example, by hydrogen, halogen, alkyl, haloalkyl, hydroxyl, alkoxy, cycloalkoxy, aryloxy, alkoxyalkyl, alkoxyalkoxy, cycloalkyl, halocycloalkyl, aryl, arylalkyl, heteroaryl, heterocyclyl, alkenyl, alkylcarbonyl, cycloalkylcarbonyl, arylcarbonyl, heteroarylcarbonyl, alkoxycarbonyl, hydroxycarbonyl, cycloalkoxycarbonyl, cycloalkylalkoxycarbonyl, alkoxycarbonylalkyl, arylalkoxycarbonyl, arylalkoxycarbonylalkyl, alkynyl, alkynylalkyl, alkylalkynyl, trisalkylsilylalkynyl, nitro, amino, cyano, haloalkoxy, haloalkylthio, alkylthio, hydrothio, hydroxyalkyl, oxo, heteroarylalkoxy, arylalkoxy, heterocyclylalkoxy, heterocyclylalkylthio, heterocyclyloxy, heterocyclylthio, heteroaryloxy, bisalkylamino, alkylamino, cycloalkylamino, hydroxycarbonylalkylamino, alkoxycarbonylalkylamino, arylalkoxycarbonylalkylamino, alkoxycarbonylalkyl(alkyl)amino, aminocarbonyl, alkylaminocarbonyl, bisalkylaminocarbonyl, cycloalkylaminocarbonyl, hydroxycarbonylalkylaminocarbonyl, alkoxycarbonylalkylaminocarbonyl, arylalkoxycarbonylalkylaminocarbonyl.

When a base structure is substituted “by one or more radicals” from a list of radicals (=group) or a generically defined group of radicals, this in each case includes simultaneous substitution by a plurality of identical and/or structurally different radicals.

In the case of a partially or fully saturated nitrogen heterocycle, this may be joined to the remainder of the molecule either via carbon or via the nitrogen.

Suitable substituents for a substituted heterocyclic radical are the substituents specified further down, and additionally also oxo and thioxo. The oxo group as a substituent on a ring carbon atom is then, for example, a carbonyl group in the heterocyclic ring. As a result, lactones and lactams are preferably also included. The oxo group may also occur on the ring heteroatoms, which may exist in different oxidation states, for example in the case of N and S, and in that case form, for example, the divalent —N(O)—, —S(O)— (also SO for short) and —S(O)₂— (also SO₂ for short) groups in the heterocyclic ring. In the case of —N(O)— and —S(O)— groups, both enantiomers in each case are included.

According to the invention, the expression “heteroaryl” refers to heteroaromatic compounds, i.e. fully unsaturated aromatic heterocyclic compounds, preferably 5- to 7-membered rings having 1 to 4, preferably 1 or 2, identical or different heteroatoms, preferably O, S or N. Inventive heteroaryls are, for example, 1H-pyrrol-1-yl; 1H-pyrrol-2-yl; 1H-pyrrol-3-yl; furan-2-yl; furan-3-yl; thien-2-yl; thien-3-yl, 1H-imidazol-1-yl; 1H-imidazol-2-yl; 1H-imidazol-4-yl; 1H-imidazol-5-yl; 1H-pyrazol-1-yl; 1H-pyrazol-3-yl; 1H-pyrazol-4-yl; 1H-pyrazol-5-yl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 1H-1,2,4-triazol-1-yl, 1H-1,2,4-triazol-3-yl, 4H-1,2,4-triazol-4-yl, 1,2,4-oxadiazol-3-yl, 1,2,4-oxadiazol-5-yl, 1,3,4-oxadiazol-2-yl, 1,2,3-oxadiazol-4-yl, 1,2,3-oxadiazol-5-yl, 1,2,5-oxadiazol-3-yl, azepinyl, pyridin-2-yl, pyridin-3-yl, pyridin-4-yl, pyrazin-2-yl, pyrazin-3-yl, pyrimidin-2-yl, pyrimidin-4-yl, pyrimidin-5-yl, pyridazin-3-yl, pyridazin-4-yl, 1,3,5-triazin-2-yl, 1,2,4-triazin-3-yl, 1,2,4-triazin-5-yl, 1,2,4-triazin-6-yl, 1,2,3-triazin-4-yl, 1,2,3-triazin-5-yl, 1,2,4-, 1,3,2-, 1,3,6- and 1,2,6-oxazinyl, isoxazol-3-yl, isoxazol-4-yl, isoxazol-5-yl, 1,3-oxazol-2-yl, 1,3-oxazol-4-yl, 1,3-oxazol-5-yl, isothiazol-3-yl, isothiazol-4-yl, isothiazol-5-yl, 1,3-thiazol-2-yl, 1,3-thiazol-4-yl, 1,3-thiazol-5-yl, oxepinyl, thiepinyl, 1,2,4-triazolonyl and 1,2,4-diazepinyl, 2H-1,2,3,4-tetrazol-5-yl, 1H-1,2,3,4-tetrazol-5-yl, 1,2,3,4-oxatriazol-5-yl, 1,2,3,4-thiatriazol-5-yl, 1,2,3,5-oxatriazol-4-yl, 1,2,3,5-thiatriazol-4-yl. The heteroaryl groups of the invention may also be substituted by one or more identical or different radicals. If two adjacent carbon atoms are part of a further aromatic ring, the systems are fused heteroaromatic systems, such as benzofused or polyannelated heteroaromatics. Preferred examples are quinolines (e.g. quinolin-2-yl, quinolin-3-yl, quinolin-4-yl, quinolin-5-yl, quinolin-6-yl, quinolin-7-yl, quinolin-8-yl); isoquinolines (e.g. isoquinolin-1-yl, isoquinolin-3-yl, isoquinolin-4-yl, isoquinolin-5-yl, isoquinolin-6-yl, isoquinolin-7-yl, isoquinolin-8-yl); quinoxaline; quinazoline; cinnoline; 1,5-naphthyridine; 1,6-naphthyridine; 1,7-naphthyridine; 1,8-naphthyridine; 2,6-naphthyridine; 2,7-naphthyridine; phthalazine; pyridopyrazines; pyridopyrimidines; pyridopyridazines; pteridines; pyrimidopyrimidines. Examples of heteroaryl are also 5- or 6-membered benzofused rings from the group of 1H-indol-1-yl, 1H-indol-2-yl, 1H-indol-3-yl, 1H-indol-4-yl, 1H-indol-5-yl, 1H-indol-6-yl, 1H-indol-7-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, 2H-indazol-7-yl, 2H-isoindol-2-yl, 2H-isoindol-1-yl, 2H-isoindol-3-yl, 2H-isoindol-4-yl, 2H-isoindol-5-yl, 2H-isoindol-6-yl; 2H-isoindol-7-yl, 1H-benzimidazol-1-yl, 1H-benzimidazol-2-yl, 1H-benzimidazol-4-yl, 1H-benzimidazol-5-yl, 1H-benzimidazol-6-yl, 1H-benzimidazol-7-yl, 1,3-benzoxazol-2-yl, 1,3-benzoxazol-4-yl, 1,3-benzoxazol-5-yl, 1,3-benzoxazol-6-yl, 1,3-benzoxazol-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,2-benzisoxazol-3-yl, 1,2-benzisoxazol-4-yl, 1,2-benzisoxazol-5-yl, 1,2-benzisoxazol-6-yl, 1,2-benzisoxazol-7-yl, 1,2-benzisothiazol-3-yl, 1,2-benzisothiazol-4-yl, 1,2-benzisothiazol-5-yl, 1,2-benzisothiazol-6-yl, 1,2-benzisothiazol-7-yl.

The term “halogen” denotes, for example, fluorine, chlorine, bromine or iodine. If the term is used for a radical, “halogen” denotes, for example, a fluorine, chlorine, bromine or iodine atom.

According to the invention, “alkyl” means a straight-chain or branched open-chain, saturated hydrocarbon radical which is optionally mono- or polysubstituted, and in the latter case is referred to as “substituted alkyl”. Preferred substituents are halogen atoms, alkoxy, haloalkoxy, cyano, alkylthio, haloalkylthio, amino or nitro groups, particular preference being given to methoxy, methyl, fluoroalkyl, cyano, nitro, fluorine, chlorine, bromine or iodine. The prefix “bis” also includes the combination of different alkyl radicals, e.g. methyl(ethyl) or ethyl(methyl).

“Haloalkyl”, “-alkenyl” and “-alkynyl” respectively denote alkyl, alkenyl and alkynyl partially or fully substituted by identical or different halogen atoms, for example monohaloalkyl such as CH₂CH₂Cl, CH₂CH₂Br, CHClCH₃, CH₂Cl, CH₂F; perhaloalkyl such as CCl₃, CClF₂, CFCl₂, CF₂CClF₂, CF₂CClFCF₃; polyhaloalkyl such as CH₂CHFCl, CF₂CClFH, CF₂CBrFH, CH₂CF₃; the term perhaloalkyl also encompasses the term perfluoroalkyl.

“Partially fluorinated alkyl” denotes a straight-chain or branched, saturated hydrocarbon which is mono- or polysubstituted by fluorine, where the fluorine atoms in question may be present as substituents on one or more different carbon atoms of the straight-chain or branched hydrocarbon chain, for example CHFCH₃, CH₂CH₂F, CH₂CH₂CF₃, CHF₂, CH₂F, CHFCF₂CF₃.

“Partially fluorinated haloalkyl” denotes a straight-chain or branched, saturated hydrocarbon which is substituted by different halogen atoms with at least one fluorine atom, where any other halogen atoms optionally present are selected from the group consisting of fluorine, chlorine or bromine, iodine. The corresponding halogen atoms may be present as substituents on one or more different carbon atoms of the straight-chain or branched hydrocarbon chain. Partially fluorinated haloalkyl also includes full substitution of the straight or branched chain by halogen including at least one fluorine atom.

“Haloalkoxy” is, for example, OCF₃, OCHF₂, OCH₂F, OCF₂CF₃, OCH₂CF₃ and OCH₂CH₂Cl; this applies correspondingly to haloalkenyl and other halogen-substituted radicals.

The expression “(C₁-C₄)-alkyl” mentioned here by way of example is a brief notation for straight-chain or branched alkyl having one to 4 carbon atoms according to the range stated for carbon atoms, i.e. encompasses the methyl, ethyl, 1-propyl, 2-propyl, 1-butyl, 2-butyl, 2-methylpropyl or tert-butyl radicals. General alkyl radicals with a larger specified range of carbon atoms, e.g. “(C₁-C₆)-alkyl”, correspondingly also encompass straight-chain or branched alkyl radicals with a greater number of carbon atoms, i.e. according to the example also the alkyl radicals having 5 and 6 carbon atoms.

Unless stated specifically, preference is given to the lower carbon skeletons, for example having from 1 to 6 carbon atoms, or having from 2 to 6 carbon atoms in the case of unsaturated groups, in the case of the hydrocarbon radicals such as alkyl, alkenyl and alkynyl radicals, including in composite radicals. Alkyl radicals, including in composite radicals such as alkoxy, haloalkyl, etc., are, for example, methyl, ethyl, n-propyl or i-propyl, n-, i-, t- or 2-butyl, pentyls, hexyls such as n-hexyl, i-hexyl and 1,3-dimethylbutyl, heptyls such as n-heptyl, 1-methylhexyl and 1,4-dimethylpentyl; alkenyl and alkynyl radicals are defined as the possible unsaturated radicals corresponding to the alkyl radicals, where at least one double bond or triple bond is present. Preference is given to radicals having one double bond or triple bond.

The term “alkenyl” also includes, in particular, straight-chain or branched open-chain hydrocarbon radicals having more than one double bond, such as 1,3-butadienyl and 1,4-pentadienyl, but also allenyl or cumulenyl radicals having one or more cumulated double bonds, for example allenyl (1,2-propadienyl), 1,2-butadienyl and 1,2,3-pentatrienyl. Alkenyl denotes, for example, vinyl which may optionally be substituted by further alkyl radicals, for example (but not limited thereto) (C₂-C₆)-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “alkynyl” also includes, in particular, straight-chain or branched open-chain hydrocarbon radicals having more than one triple bond, or else having one or more triple bonds and one or more double bonds, for example 1,3-butatrienyl or 3-penten-1-yn-1-yl. (C₂-C₆)-Alkynyl denotes, for example, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl.

The term “cycloalkyl” refers to a carbocyclic saturated ring system having preferably 3-8 ring carbon atoms, for example cyclopropyl, cyclobutyl, cyclopentyl or cyclohexyl, which optionally has further substitution, preferably by hydrogen, alkyl, alkoxy, cyano, nitro, alkylthio, haloalkylthio, halogen, alkenyl, alkynyl, haloalkyl, amino, alkylamino, bisalkylamino, alkoxycarbonyl, hydroxycarbonyl, arylalkoxycarbonyl, aminocarbonyl, alkylaminocarbonyl, cycloalkylaminocarbonyl. In the case of optionally substituted cycloalkyl, cyclic systems with substituents are included, also including substituents with a double bond on the cycloalkyl radical, for example an alkylidene group such as methylidene. In the case of optionally substituted cycloalkyl, polycyclic aliphatic systems are also included, for example bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl and adamantan-2-yl, but also systems such as 1,1′-bi(cyclopropyl)-1-yl, 1,1′-bi(cyclopropyl)-2-yl, for example. The term “(C₃-C₇)-cycloalkyl” is a brief notation for cycloalkyl having three to 7 carbon atoms, corresponding to the range specified for carbon atoms.

In the case of substituted cycloalkyl, spirocyclic aliphatic systems are also included, for example spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl.

“Cycloalkenyl” denotes a carbocyclic, nonaromatic, partially unsaturated ring system having preferably 4-8 carbon atoms, e.g. 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl, also including substituents with a double bond on the cycloalkenyl radical, for example an alkylidene group such as methylidene. In the case of optionally substituted cycloalkenyl, the elucidations for substituted cycloalkyl apply correspondingly.

The term “alkylidene”, also, for example, in the form (C₁-C₁₀)-alkylidene, denotes the radical of a straight-chain or branched open-chain hydrocarbon radical which is attached via a double bond. Possible bonding sites for alkylidene are naturally only positions on the base structure where two hydrogen atoms can be replaced by the double bond; radicals are, for example, ═CH₂, ═CH—CH₃, ═C(CH₃)—CH₃, ═C(CH₃)—C₂H₅ or ═C(C₂H₅)—C₂H₅. Cycloalkylidene denotes a carbocyclic radical bonded via a double bond.

The term “alkylene”, also, for example, in the form (C₁-C₈)-alkylene, denotes the radical of a straight-chain or branched open-chain hydrocarbon radical which is attached at two positions to further groups.

“Cycloalkylalkyloxy” denotes a cycloalkylalkyl radical bonded via an oxygen atom and “arylalkyloxy” denotes an arylalkyl radical bonded via an oxygen atom.

“Alkoxyalkyl” represents an alkoxy radical bonded via an alkyl group and “alkoxyalkoxy” denotes an alkoxyalkyl radical bonded via an oxygen atom, for example (but not limited thereto) methoxymethoxy, methoxyethoxy, ethoxyethoxy, methoxy-n-propyloxy.

“Alkylthioalkyl” represents an alkylthio radical bonded via an alkyl group and “alkylthioalkylthio” denotes an alkylthioalkyl radical bonded via an oxygen atom.

“Arylalkoxyalkyl” represents an aryloxy radical bonded via an alkyl group and “heteroaryloxyalkyl” denotes a heteroaryloxy radical bonded via an alkyl group.

“Haloalkoxyalkyl” represents a haloalkoxy radical and “haloalkylthioalkyl” denotes a haloalkylthio radical, bonded via an alkyl group.

“Arylalkyl” represents an aryl radical bonded via an alkyl group, “heteroarylalkyl” denotes a heteroaryl radical bonded via an alkyl group, and “heterocyclylalkyl” denotes a heterocyclyl radical bonded via an alkyl group.

“Cycloalkylalkyl” represents a cycloalkyl radical bonded via an alkyl group, for example (but not limited thereto) cyclopropylmethyl, cyclobutylmethyl, cyclopentylmethyl, cyclohexylmethyl, 1-cyclopropyleth-1-yl, 2-cyclopropyleth-1-yl, 1-cyclopropylprop-1-yl, 3-cyclopropylprop-1-yl.

“Arylalkenyl” represents an aryl radical bonded via an alkenyl group, “heteroarylalkenyl” denotes a heteroaryl radical bonded via an alkenyl group, and “heterocyclylalkenyl” denotes a heterocyclyl radical bonded via an alkenyl group.

“Arylalkynyl” represents an aryl radical bonded via an alkynyl group, “heteroarylalkynyl” denotes a heteroaryl radical bonded via an alkynyl group, and “heterocyclylalkynyl” denotes a heterocyclyl radical bonded via an alkynyl group.

According to the invention, “haloalkylthio”—on its own or as constituent part of a chemical group—represents straight-chain or branched S-haloalkyl, preferably having 1 to 8, or having 1 to 6 carbon atoms, such as (C₁-C₈)-, (C₁-C₆)- or (C₁-C₄)-haloalkylthio, for example (but not limited thereto) trifluoromethylthio, pentafluoroethylthio, difluoromethyl, 2,2-difluoroeth-1-ylthio, 2,2,2-difluoroeth-1-ylthio, 3,3,3-prop-1-ylthio.

“Halocycloalkyl” and “halocycloalkenyl” denote cycloalkyl and cycloalkenyl, respectively, which are partially or fully substituted by identical or different halogen atoms, such as F, Cl and Br, or by haloalkyl, such as trifluoromethyl or difluoromethyl, for example 1-fluorocycloprop-1-yl, 2-fluorocycloprop-1-yl, 2,2-difluorocycloprop-1-yl, 1-fluorocyclobut-1-yl, 1-trifluoromethylcycloprop-1-yl, 2-trifluoromethylcycloprop-1-yl, 1-chlorocycloprop-1-yl, 2-chlorocycloprop-1-yl, 2,2-dichlorocycloprop-1-yl, 3,3-difluorocyclobutyl.

According to the invention, “trialkylsilyl”—on its own or as constituent part of a chemical group—represents straight-chain or branched Si-alkyl, preferably having 1 to 8, or having 1 to 6 carbon atoms, such as tri[(C₁-C₈)-, (C₁-C₆)- or (C₁-C₄)-alkyl]silyl, for example (but not limited thereto) trimethylsilyl, triethylsilyl, tri(n-propyl)silyl, tri(isopropyl)silyl, tri(n-butyl)silyl, tri(1-methylprop-1-yl)silyl, tri(2-methylprop-1-yl)silyl, tri(1,1-dimethyleth-1-yl)silyl, tri(2,2-dimethyleth-1-yl)silyl.

“Trialkylsilylalkynyl” represents a trialkylsilyl radical bonded via an alkynyl group.

### Synthesis of Substituted N-Phenyluracils of the General Formula (I).

The substituted N-phenyluracils of the general formula (I) according to the invention can be prepared using known processes. The synthesis routes used and examined proceed from commercially available or easily preparable heteroaromatic amines and of correspondingly substituted hydroxy esters. In the schemes which follow, the moieties G, Q, R¹, R², R³, R⁴, R⁵, R⁶, R⁷, X and Y of the general formula (I) have the meanings defined above, unless exemplary, but not limiting, definitions are given. As a first key intermediate for the synthesis of the compounds of the general formula (Ia) according to the invention in which X represents sulfur (S) and Y represents oxygen (O), a mercaptophenyl-1H-pyrimidine-2,4-dione, which is optionally substituted further, is prepared. By way of example, but not by way of limitation, this is illustrated by the synthesis of 3-(4-chloro-2-fluoro-5-mercaptophenyl)-1-methyl-6-trifluoromethyl-1H-pyrimidine-2,4-dione (IIa) (Scheme 1). To this end, a suitable substituted aniline, by way of example, but not by way of limitation, 2-fluoro-4-chloroaniline, is converted with a suitable reagent (e.g. triphosgene) in a suitable polar aprotic solvent (e.g. dichloromethane) into the corresponding isocyanate which, in the next step, is converted by reaction with a suitable aminoacrylic ester using a suitable base (e.g. sodium hydride or potassium tert-butoxide) in a suitable polar aprotic solvent (e.g. N,N-dimethylformamide) into the corresponding pyrimidine-2,4-dione, which is optionally substituted further, by way of example, but not by way of limitation, 3-(4-chloro-2-fluorophenyl)-1-methyl-6-trifluoromethyl-1H-pyrimidine-2,4-dione (Scheme 1). By subsequent sulfochlorination with a suitable reagent (e.g. chlorosulfonic acid) followed by reduction with a suitable reducing agent (e.g. Zn in EtOH and HCl, tin(II) chloride hydrate or triphenylphosphine), it is possible to prepare the desired further-substituted mercaptophenyl-1H-pyrimidine-2,4-dione, by way of example, but not by way of limitation, 3-(4-chloro-2-fluoro-5-mercaptophenyl)-1-methyl-6-trifluoromethyl-1H-pyrimidine-2,4-dione (IIa) (cf. KR1345394; EP1122244; EP408382; WO 2003/029226; WO2010/038953; US2011/0224083; KR2011/110420). In Scheme 1 below, R¹, by way of example, but not by way of limitation, represents hydrogen, R² and R³, by way of example, but not by way of limitation, represent fluorine, R⁴, by way of example, but not by way of limitation, represents chlorine, and X, by way of example, but not by way of limitation, represents sulfur.

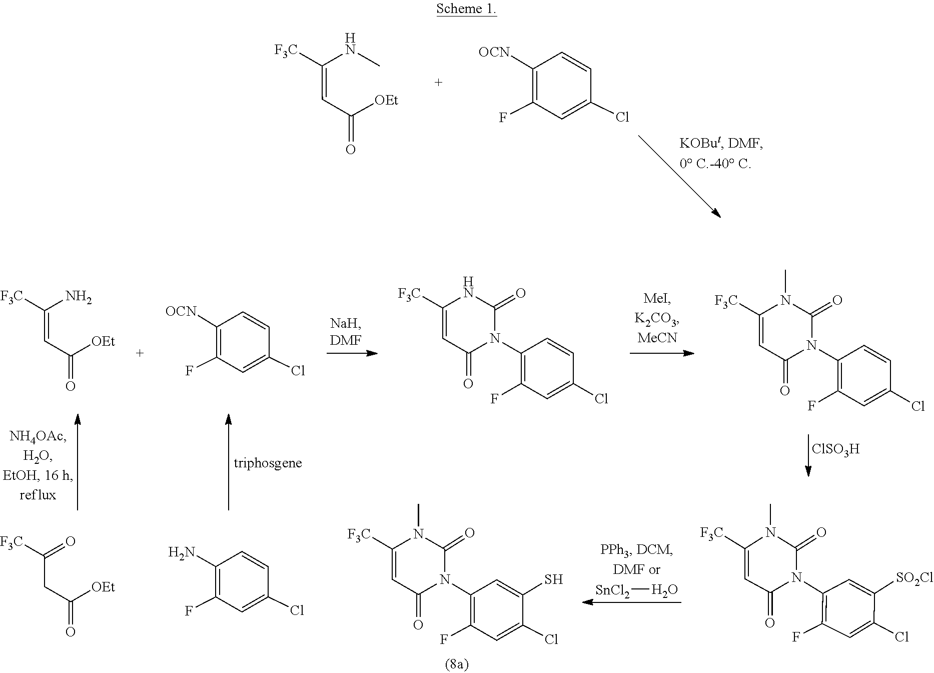

The synthesis of the key intermediate (IIa) described in Scheme 1 can also be applied to the preparation of similar intermediates, e.g. 3-(4-chloro-2-fluoro-5-mercaptophenyl)-1-methyl-5,6-ditrifluoromethyl-1H-pyrimidine-2,4-dione (IIb). Here, by way of example, but not by way of limitation, the starting material used is ethyl 4,4,4-trifluoro-3-oxo-2-(trifluoromethyl)butanoate (cf. Journal of Fluorine Chemistry (2016), 181, 1-6). In Scheme 1 below, R¹, by way of example, but not by way of limitation, represents CF₃, R² and R³, by way of example, but not by way of limitation, represent fluorine, R⁴, by way of example, but not by way of limitation, represents chlorine, and X, by way of example, but not by way of limitation, represents sulfur.

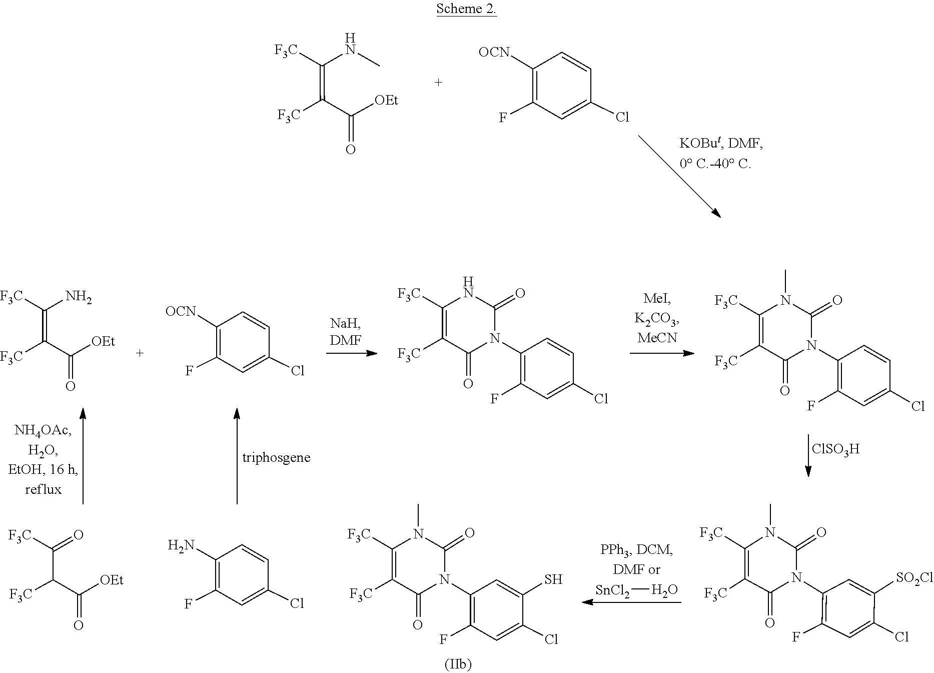

The respective further-substituted N-methyl-5-mercaptophenyl-1H-pyrimidine-2,4-dione intermediates (II) can then be converted by various routes into the desired compounds of the general formula (Ia) according to the invention in which X represents sulfur (S) and Y represents oxygen (O) (Scheme 3), after converting the compounds (II) in a first step with the aid of a suitable optionally further-substituted iodopyridone using a suitable base or using a suitable transition metal catalyst (e.g. tris(dibenzylideneacetone)dipalladium(0)) with a suitable ligand (e.g. 4,5-bis(diphenylphosphino)-9,9-dimethylxanthene) and a suitable base (e.g. diispropyl(ethyl)amine) in a suitable polar-aprotic solvent (e.g. dioxane) into intermediates (III). In Scheme 3 below, Q, R¹, R², R³ and R⁴ have the above meanings according to the invention. Furthermore, R⁵, R⁶, R⁷, by way of example, but not by way of limitation, represent hydrogen, X, by way of example, but not by way of limitation, represents sulfur, Y, by way of example, but not by way of limitation, represents oxygen and G, by way of example, but not by way of limitation, represents CH₂. The corresponding intermediate (II) described by way of example, but not by way of limitation, in Scheme 2 can be converted by reaction with a suitable optionally further-substituted iodoalkanoic ester (in Schema 3 by way of example, but not by way of limitation, an iodoacetic ester) using a suitable base (e.g. silver(I) carbonate) in a suitable polar-aprotic solvent (e.g. n-hexane or cyclohexane) at elevated temperature (e.g. under microwave conditions) into a corresponding oxyalkanoic ester intermediate (IVa, IVb) or the desired target compounds of the general formula (Ia) (cf. Synthesis 2009, 2725). The corresponding iodoalkanoic esters can be prepared by routes known from the literature (cf Eur. J. Org. Chem., 2006, 71, 8459; WO2012037573; Organometallics, 2009, 28, 132).

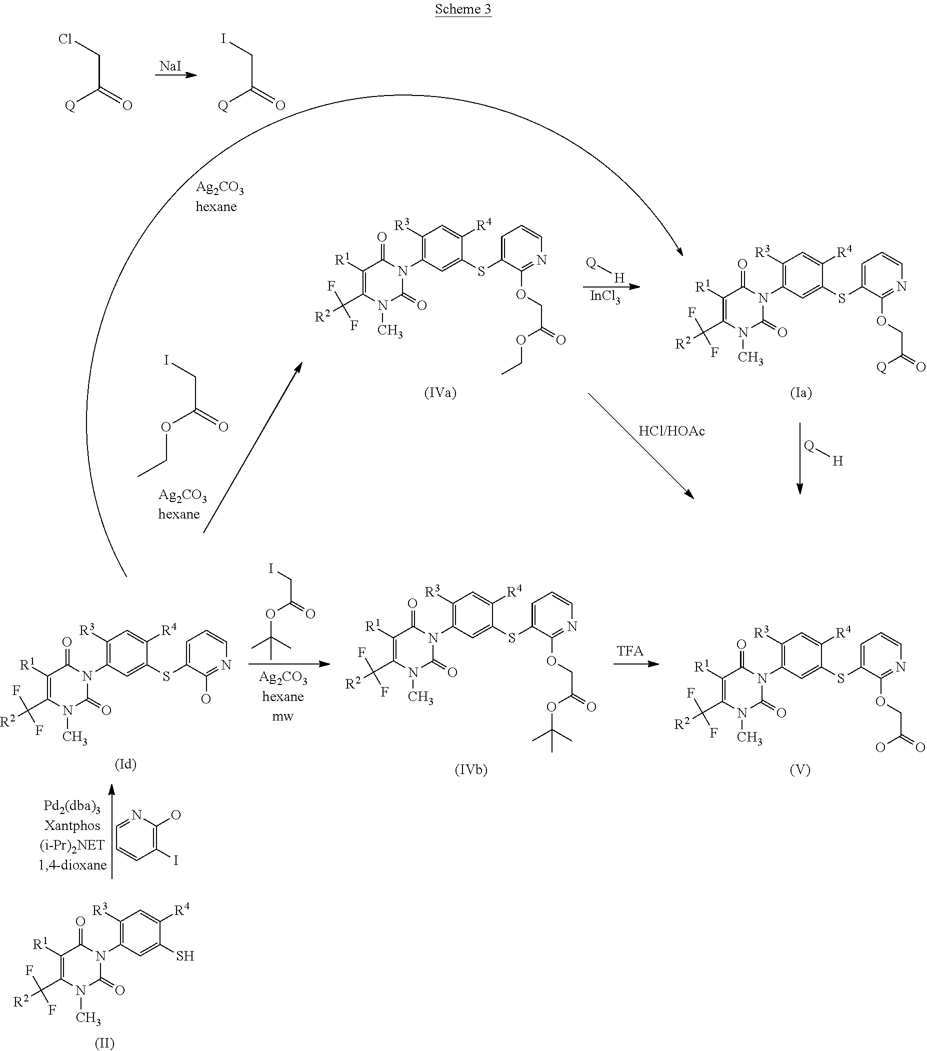

The ethyl ester (IVa) and tert-butyl ester (IVb) intermediates can then be converted under suitable reaction conditions [use of a suitable acid such as hydrochloric acid or acetic acid in the case of (IVa) or trifluoroacetic acid (TFA) in the case of (IVb)] into the corresponding free acid (V). By reaction of the corresponding acid intermediate (V) with a suitable compound Q-H with mediation by suitable coupling reagents (e.g. HOBt=1-hydroxybenzotriazole, EDC=1-ethyl-3-(3-dimethylaminopropyl)carbodiimide, HATU=O-(7-azabenzotriazol-1-yl)-N,N,N′,N′-tetramethyluronium hexafluorophosphate, T3P=2,4,6-tripropyl-1,3,5,2,4,6-trioxatriphosphorinane 2,4,6-trioxide) and suitable bases (e.g. diisopropylethylamine, triethylamine) in a suitable polar-aprotic solvent (e.g. dichloromethane, chloroform), it is possible to prepare the desired substituted N-phenyluracils of the general formula (Ia). Alternatively, the ethyl ester (IVa) can be converted by coupling with a suitable compound Q-H with mediation by a suitable Lewis acid (e.g. indium(III) chloride) into the corresponding desired substituted N-phenyluracil of the general formula (Ia) (cf. WO2011/1307088).

The preparation of the compounds of the general formula (I) in which X and Y, by way of example, but not by way of limitation, represent oxygen (O) proceeds via the synthesis of key intermediates (VI) having a fluorine substituent at position 5, such as 3-(2,5-difluoro-4-nitro)-1-methyl-6-trifluoromethyl-1H-pyrimidine-2,4-dione (VIa). To this end, a suitable substituted aniline, by way of example, but not by way of limitation, 2,5-difluoroaniline, is converted with a suitable reagent (e.g. triphosgene) in a suitable polar aprotic solvent (e.g. dichloromethane) into the corresponding isocyanate which, in the next step, is converted by reaction with a suitable aminoacrylic ester using a suitable base (e.g. sodium hydride or potassium tert-butoxide) in a suitable polar aprotic solvent (e.g. N,N-dimethylformamide) into the corresponding pyrimidine-2,4-dione, which is optionally substituted further, here, by way of example, but not by way of limitation, 3-(2,5-difluorophenyl)-6-trifluoromethyl-1H-pyrimidine-2,4-dione (Scheme 4). Nitration with a suitable nitration reagent and subsequent N-methylation with a suitable methylating reagent affords the desired intermediate, here, by way of example, but not by way of limitation, 3-(2,5-difluoro-4-nitro)-1-methyl-6-trifluoromethyl-1H-pyrimidine-2,4-dione (VIa). In Scheme 4 below, R¹, by way of example, but not by way of limitation, represents hydrogen, R², by way of example, but not by way of limitation, represents fluorine, R³, by way of example, but not by way of limitation, represents fluorine, and R⁴, by way of example, but not by way of limitation, represents nitro.

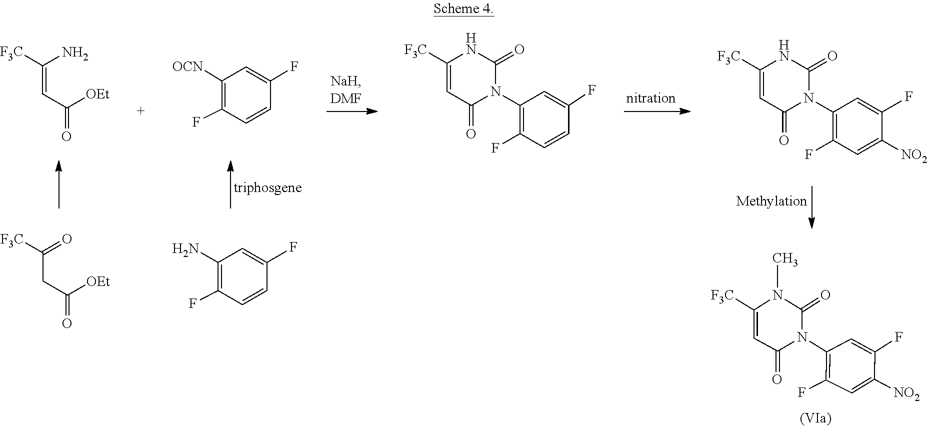

Intermediate (VI), e.g. compound (VIa), obtained in the manner described above, can then be converted with a suitable substituted 2-carbonylalkyloxy-3-hydroxypyridine (VII) using a suitable base (e.g. potassium carbonate) in a suitable polar-aprotic solvent (e.g. N,N-dimethylformamide (DMF)) into a desired substituted N-phenyluracil (Ib, R⁴=nitro). The intermediate (VII) used for this purpose can be obtained by a multi-step synthesis starting with commercially available 2-chloro-3-nitropyridine, via (i) base-mediated coupling (e.g. with sodium hydride) with a suitable substituted hydroxyalkylcarbonyl reagent in a suitable polar-aprotic solvent (e.g. tetrahydrofuran or dioxane), (ii) reduction of the nitro group with a suitable reducing agent (e.g. hydrogen, palladium on carbon in a suitable polar-protic solvent), (iii) diazotization (with a suitable diazotization reagent, e.g. tert-butyl nitrite (t-BuONO), boron trifluoride etherate (BF₃-OEt₂) in suitable polar-aprotic solvents (e.g. dichloromethane (DCM), dimethoxyethane), (iv) reaction with acetic anhydride and (v) release of the hydroxy group by removal of the acetyl protective group (e.g. base-mediated with potassium carbonate in a polar-protic solvent). The nitro group in compound (Ib) can then be converted by reduction and subsequent Sandmeyer reaction into a halogen substituent (e.g. chlorine, bromine), such that the desired substituted N-phenyluracil (Ic) can be obtained in this manner. In Scheme 5 below, Q, R¹ and R² have the above meanings according to the invention. Furthermore, R³, by way of example, but not by way of limitation, represents fluorine, R⁴, by way of example, but not by way of limitation, represents chlorine or nitro, R⁵, R⁶, R⁷, by way of example, but not by way of limitation, represent hydrogen, X and Y, by way of example, but not by way of limitation, represent oxygen and G, by way of example, but not by way of limitation, represents CH₂.

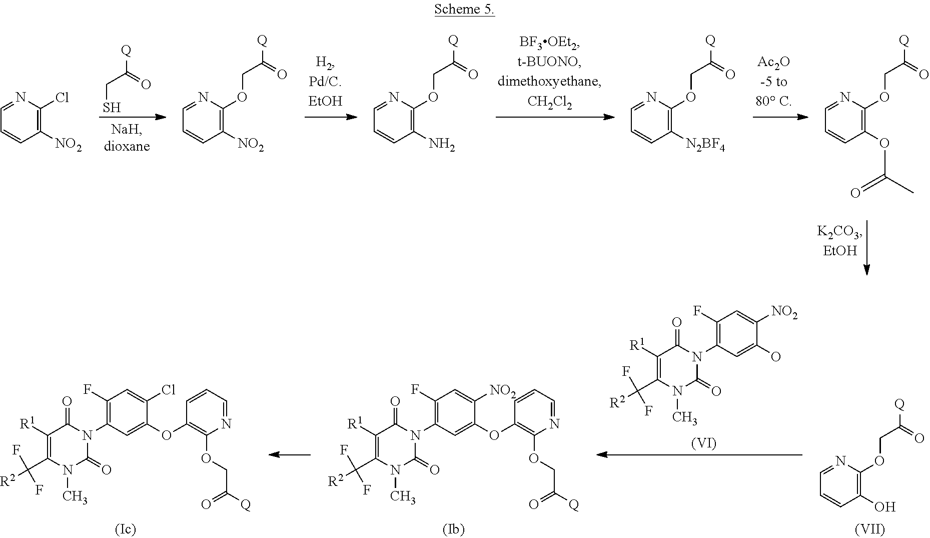

Correspondingly, intermediate (VI), obtained in the manner described above, can be converted with a suitable substituted 2-carbonylalkylthio-3-hydroxypyridine (VIII) using a suitable base (e.g. potassium carbonate) in a suitable polar-aprotic solvent (e.g. N,N-dimethylformamide (DMF)) into a desired substituted N-phenyluracil (Id, R⁴=nitro) where X═O (oxygen) and Y═S (sulfur). The intermediate (VIII) used for this purpose can be obtained by a multi-step synthesis analogously to the synthesis of intermediate (VII) described in Scheme 5 starting with commercially available 2-chloro-3-nitropyridine. The nitro group in compound (Id) can then be converted by reduction and subsequent Sandmeyer reaction into a halogen substituent (e.g. chlorine, bromine), such that the desired substituted N-phenyluracil (le) can be obtained in this manner. In Scheme 6 below, Q, R¹ and R² have the above meanings according to the invention.

Furthermore, R³, by way of example, but not by way of limitation, represents fluorine, R⁴, by way of example, but not by way of limitation, represents chlorine or nitro, R⁵, R⁶, R⁷, by way of example, but not by way of limitation, represent hydrogen, X, by way of example, but not by way of limitation, represents oxygen, Y, by way of example, but not by way of limitation, represents sulfur and G, by way of example, but not by way of limitation, represents CH₂.

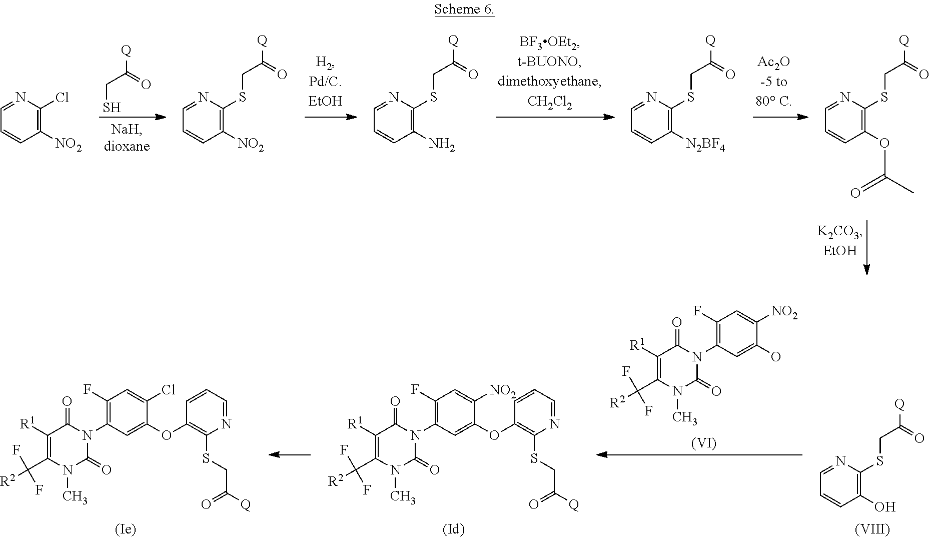

The further-substituted N-methyl-5-mercaptophenyl-1H-pyrimidine-2,4-dione intermediates (II) can also be converted into the desired compounds of the general formula (If) according to the invention in which X and Y represent sulfur (S) (Scheme 7), after converting the compounds (III) in a first step with the aid of a suitable optionally further-substituted iodothiopyridine using a suitable base or using a suitable transition metal catalyst (e.g. tris(dibenzylideneacetone)dipalladium(0)) with a suitable ligand (e.g. 4,5-bis(diphenylphosphino)-9,9-dimethylxanthene) and a suitable base (e.g. diispropyl(ethyl)amine) in a suitable polar-aprotic solvent (e.g. dioxane) into intermediates of type (IX). The intermediates (IX) can then be reacted with haloalkanecarboxylic acids having various substitutions using suitable bases, to afford the desired compounds of the general formula (If). In Scheme 6 below, Q, R¹, R², R³, and R⁴ have the above meanings according to the invention. Furthermore, R⁵, R⁶, R⁷, by way of example, but not by way of limitation, represent hydrogen, X and Y, by way of example, but not by way of limitation, represent sulfur and G, by way of example, but not by way of limitation, represents CH₂. For clarity, the reaction paths are furthermore described in Scheme 7 below, by way of example, but not by way of limitation, using iodoacetic esters. Also suitable for coupling with intermediate (IX) are comparable haloalkanecarboxylic acids (halogen=bromine or chlorine).

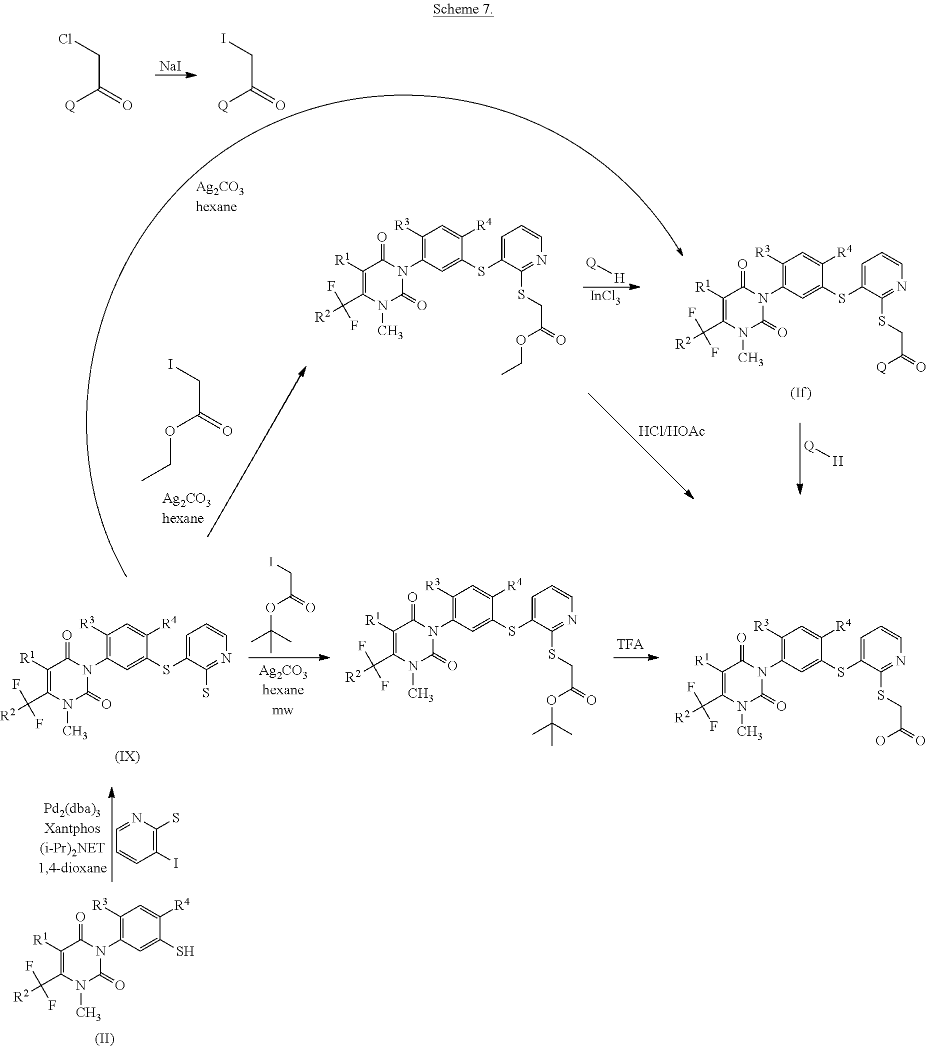

Selected detailed synthesis examples for the compounds of the general formula (I) according to the invention are given below. The example numbers mentioned correspond to the numbering scheme in Tables I.1 to I.34 below. The ¹H NMR, ¹³C-NMR and ¹⁹F-NMR spectroscopy data reported for the chemical examples described in the sections which follow (400 MHz for ¹H NMR and 150 MHz for ¹³C-NMR and 375 MHz for ¹⁹F-NMR, solvent CDCl₃, CD₃OD or d₆-DMSO, internal standard: tetramethylsilane δ=0.00 ppm) were obtained on a Bruker instrument, and the signals listed have the meanings given below: br=broad; s=singlet, d=doublet, t=triplet, dd=doublet of doublets, ddd=doublet of a doublet of doublets, m=multiplet, q=quartet, quint=quintet, sext=sextet, sept=septet, dq=doublet of quartets, dt=doublet of triplets. In the case of diastereomer mixtures, either the significant signals for each of the two diastereomers are reported or the characteristic signal of the main diastereomer is reported. The abbreviations used for chemical groups have, for example, the following meanings: Me=CH₃, Et=CH₂CH₃, t-Hex=C(CH₃)₂CH(CH₃)₂, t-Bu=C(CH₃)₃, n-Bu=unbranched butyl, n-Pr=unbranched propyl, i-Pr=branched propyl, c-Pr=cyclopropyl, c-Hex=cyclohexyl.

### SYNTHESIS EXAMPLES

### No. I.1-1: 2-Methoxyethyl {[3-({2-chloro-4-fluoro-5-[3-methyl-2,6-dioxo-4-(trifluoromethyl)-3,6-dihydropyrimidin-1(2H)-yl]phenyl}sulfanyl)pyridin-2-yl]oxy}acetate

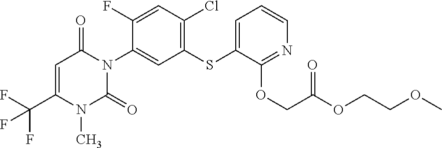 [SMILES: COCCOC(=O)COc1ncccc1Sc1cc(-n2c(=O)cc(C(F)(F)F)n(C)c2=O)c(F)cc1Cl]

Successively, 2-fluoro-4-chloroaniline (145 g, 996 mmol) and triethylamine (202 g, 2000 mmol) were added carefully to a solution of triphosgene (119 g, 401 mmol) in abs. dichloromethane (1000 ml) such that the temperature of the resulting reaction mixture remained below 20° C. After the addition had ended, the reaction mixture was stirred at room temperature overnight and then washed with water (3×500 ml) and TN hydrochloric acid (500 ml), dried over sodium sulfate, filtered and concentrated under reduced pressure. The resulting 2-fluoro-4-chlorophenyl isocyanate was used in the next stage without further purification. Sodium hydride (5.60 g, 140 mmol, 60% dispersion in mineral oil) was suspended in abs. N,N-dimethylformamide, and ethyl (2E)-3-amino-4,4,4-trifluorobut-2-enoate (14.2 g, 77.5 mmol) was added. The reaction mixture was stirred at room temperature for 1 h and then cooled to a temperature of −30° C., and 2-fluoro-4-chlorophenyl isocyanate (12.0 g, 70.0 mmol) was added. After the addition had ended, the resulting reaction mixture was stirred at room temperature for a further 4 h and then added to ice-water. After addition of ethyl acetate and acidification with TN hydrochloric acid, the aqueous phase was extracted thoroughly with ethyl acetate. The combined organic phases were washed with water, dried over sodium sulfate, filtered and concentrated under reduced pressure. This gave 3-(4-chloro-2-fluorophenyl)-6-(trifluoromethyl)pyrimidine-2,4(1H,3H)-dione (15.2 g, 50.2 mmol, 65%), which was used in the next stage without further purification. This reaction step was also repeated successfully on a larger scale. 3-(4-Chloro-2-fluorophenyl)-6-(trifluoromethyl)pyrimidine-2,4(1H,3H)-dione (238 g, 770 mmol) was dissolved in abs. N,N-dimethylformamide (800 ml), and potassium carbonate (117 g, 850 mmol) was added. A solution of methyl iodide (120 g, 850 mmol) in abs. N,N-dimethylformamide (100 ml) was then added and the resulting reaction mixture was stirred at room temperature for a further 1 h. After complete conversion, the reaction mixture was cooled to a temperature of 0° C., water (2000 ml) was added carefully and the mixture was then extracted thoroughly with dichloromethane. The combined organic phases were dried over sodium sulfate, filtered and concentrated under reduced pressure. This gave 3-(4-chloro-2-fluorophenyl)-1-methyl-6-(trifluoromethyl)pyrimidine-2,4(1H,3H)-dione (241 g, 747 mmol, 97% of theory), which was reacted in the next stage without further purification. 3-(4-Chloro-2-fluorophenyl)-1-methyl-6-(trifluoromethyl)pyrimidine-2,4(1H,3H)-dione (100 g, 310 mmol) was then added a little at a time to chlorosulfonic acid in a round-bottom flask which had been dried by heating. The resulting reaction mixture was then stirred at a temperature of 110° C. for 20 h and, after cooling to room temperature, added to ice-water and extracted repeatedly with ethyl acetate (3×300 ml). The combined organic phases were dried over sodium sulfate, filtered and concentrated under reduced pressure. This gave 2-chloro-4-fluoro-5-[3-methyl-2,6-dioxo-4-(trifluoromethyl)-3,6-dihydropyrimidin-1(2H)-yl]benzenesulfonyl chloride (75.0 g, 178 mmol, 57% of theory), which was used in the next stage without further purification. 2-Chloro-4-fluoro-5-[3-methyl-2,6-dioxo-4-(trifluoromethyl)-3,6-dihydropyrimidin-1(2H)-yl]sulfonyl chloride (100.0 g, 237 mmol) was initially charged in a round-bottom flask, and hydrochloric acid (500 ml), acetic acid (500 ml) and tin dichloride dihydrate (270 g, 1197 mmol) were added in succession. The resulting reaction mixture was stirred at a temperature of 100° C. for 10 h and, after cooling to room temperature, added to ice-water and extracted thoroughly with dichloromethane (3×400 ml). The combined organic phases were dried over sodium sulfate, filtered and concentrated under reduced pressure. Final purification by column chromatography gave 3-(4-chloro-2-fluoro-5-sulfanylphenyl)-1-methyl-6-(trifluoromethyl)pyrimidine-2,4(1H,3H)-dione (73.0 g, 206 mmol, 83% of theory) in the form of a colorless solid. Under argon, 3-(4-chloro-2-fluoro-5-sulfanylphenyl)-1-methyl-6-(trifluoromethyl)pyrimidine-2,4(1H,3H)-dione (1.69 mmol, 1 equiv.) was dissolved in dioxane (16 ml) in a microwave vessel and, after degassing of the solvent, tris(dibenzylideneacetone)dipalladium (0.04 mmol), 4,5-bis(diphenylphosphino)-9,9-dimethylxanthene (0.08 mmol), N,N-diisopropylethylamine (3.37 mmol) and 3-bromo-2-hydroxypyridine (1.86 mmol) were added. The resulting reaction mixture was stirred under microwave conditions at a temperature of 160° C. for 2 h. After cooling to room temperature, the reaction mixture was filtered and the filtrate was concentrated. Purification of the resulting crude product by column chromatography gave 3-{4-chloro-2-fluoro-5-[(2-hydroxypyridin-3-yl)sulfanyl]phenyl}-1-methyl-6-(trifluoromethyl)pyrimidine-2,4(1H,3H)-dione (720 mg, 86% of theory) in the form of a colourless solid. In a microwave vessel and under argon, n-hexane (17 ml) was added to 3-{4-chloro-2-fluoro-5-[(2-hydroxypyridin-3-yl)sulfanyl]phenyl}-1-methyl-6-(trifluoromethyl)pyrimidine-2,4(1H,3H)-dione (300 mg, 0.67 mmol). Silver(I) carbonate (223 mg, 0.80 mmol) and ethyl 2-iodoacetate (0.16 ml, 1.34 mmol) were then added. The reaction mixture was stirred at a temperature of 140° C. under microwave conditions for 30 minutes. After cooling to room temperature, the reaction mixture was filtered and the filtrate was concentrated under reduced pressure. Purification of the resulting crude product by column chromatography gave ethyl {[3-({2-chloro-4-fluoro-5-[3-methyl-2,6-dioxo-4-(trifluoromethyl)-3,6-dihydropyrimidin-1(2H)-yl]phenyl}sulfanyl)pyridin-2-yl]oxy}acetate (84 mg, 34% of theory) in the form of a colorless solid. In a round-bottom flask, acetic acid (2 ml) and conc. HCl (0.3 ml) were added to ethyl {[3-({2-chloro-4-fluoro-5-[3-methyl-2,6-dioxo-4-(trifluoromethyl)-3,6-dihydropyrimidin-1(2H)-yl]phenyl}sulfanyl)pyridin-2-yl]oxy}acetate (118 mg, 0.22 mmol). The resulting reaction mixture was then stirred at a temperature of 50° C. for 2 h and, after cooling to room temperature, water (5 ml) and dichloromethane were added and the mixture was extracted. The combined organic phases were dried over sodium sulfate, filtered and concentrated under reduced pressure. Final purification of the resulting crude product by preparative HPLC gave {[3-({2-chloro-4-fluoro-5-[3-methyl-2,6-dioxo-4-(trifluoromethyl)-3,6-dihydropyrimidin-1(2H)-yl]phenyl}sulfanyl)pyridin-2-yl]oxy}acetic acid (60 mg, 51% of theory) in the form of a colorless solid. {[3-({2-Chloro-4-fluoro-5-[3-methyl-2,6-dioxo-4-(trifluoromethyl)-3,6-dihydropyrimidin-1(2H)-yl]phenyl}sulfanyl)pyridin-2-yl]oxy}acetic acid (30 mg, 0.06 mmol) was dissolved in dichloromethane, and 1-hydroxy-1H-benzotriazole hydrate (12 mg, 0.08 mmol), 1-ethyl-3-(3-dimethylaminopropyl)carbodiimide (15 mg, 0.08 mmol), 4-dimethylaminopyridine (2 mg) and 2-methoxyethanol (6 mg, 0.08 mmol) were added. The resulting reaction mixture was then stirred at room temperature for 2 h and concentrated. Final purification of the resulting crude product by column chromatography gave 2-methoxyethyl {[3-({2-chloro-4-fluoro-5-[3-methyl-2,6-dioxo-4-(trifluoromethyl)-3,6-dihydropyrimidin-1(2H)-yl]phenyl}sulfanyl)pyridin-2-yl]oxy}acetate (22 mg, 64% of theory) in the form of a colorless solid. ¹H-NMR (CDCl₃ δ, ppm) 8.08 (d, 1H), 7.65 (m, 1H), 7.35 (d, 1H), 7.25 (d, 1H), 6.92 (m, 1H), 6.29 (s, 1H), 5.00-4.89 (dd, 2H), 4.24-4.20 (m, 2H), 3.56 (m, 2H), 3.50 (s, 3H), 3.35 (s, 3H).

### No. I.15-26: 3-Methoxypropyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate

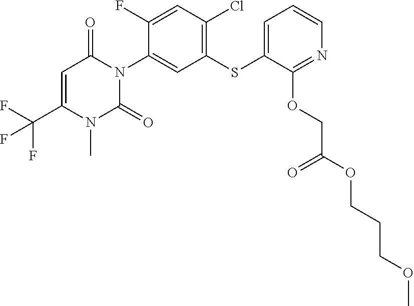 [SMILES: COCCCOC(=O)COc1ncccc1Sc1cc(-n2c(=O)cc(C(F)(F)F)n(C)c2=O)c(F)cc1Cl]

Ethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate (2.00 g, 3.9 mmol) was dissolved in 50 ml glacial acetic acid and 6 N aqueous hydrochloric acid (5.34 ml, 32.1 mmol) was added. The reaction was stirred at 50° C. for 6 h, allowed to stand at RT overnight, stirred at 50° C. for a further 6 h and cooled to RT, and dichloromethane and water were added. The aqueous phase was separated off. The organic phase was washed with water and dried over magnesium sulfate, and the solvent was removed under reduced pressure. The residue was purified by column chromatography (gradient ethyl acetate/n-heptane) and [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetic acid (1.07 g, 2.10 mmol, 57% of theory) was obtained in the form of a light-beige solid. ¹H-NMR (CDCl₃ δ, ppm) 7.94-7.96 (m, 1H), 7.26-7.39 (m, 2H), 6.96-6.99 (m, 1H), 6.78 (d, 1H), 6.32 (s, 1H), 4.91-5.00 (m, 2H), 3.51 (s, 3H). [(3-{2-Chloro-4-fluoro-5-[3-methyl-2,6-dioxo-4-(trifluoromethyl)-3,6-dihydropyrimidin-1(2H)-yl]phenoxy}pyridin-2-yl)oxy]acetic acid (60 mg, 0.12 mmol) was added to a solution of 3-methoxy-1-propanol (14 mg, 0.16 mmol) in 5 ml of dichloromethane, followed by 1-hydroxy-1H-benzotriazole hydrate (24 mg, 0.16 mmol), 1-(3-dimethylaminopropyl)-3-ethylcarbodiimide hydrochloride (31 mg, 0.16 mmol) and 4-dimethylaminopyridine (10 mol %). The reaction was stirred at RT for 2 h and allowed to stand at RT for 4 d, and 0.25 equivalents each of 1-hydroxy-1H-benzotriazole hydrate, 1-(3-dimethylaminopropyl)-3-ethylcarbodiimide hydrochloride and 3-methoxy-1-propanol were added. The reaction was stirred at RT for 6 h and allowed to stand at RT overnight, and the solvent was removed. The residue was purified by column chromatography (gradient ethyl acetate/n-heptane) and 3-methoxypropyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate (51 mg, purity: 96%, 71% of theory) was obtained in the form of a colorless solid. ¹H-NMR (CDCl₃ δ, ppm) 7.91-7.92 (m, 1H), 7.37 (d, 1H), 7.31-7.33 (m, 1H), 6.91-6.94 (m, 2H), 6.30 (s, 1H), 4.87-4.98 (m, 2H), 4.17-4.21 (m, 2H), 3.50-3.51 (m, 3H), 3.37 (t, 2H), 3.29 (s, 3H), 1.83-1.91 (m, 2H).

### No. I.15-72: Tetrahydrofuran-3-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate

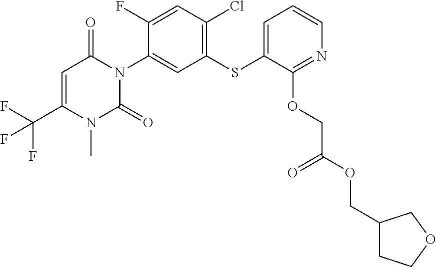 [SMILES: Cn1c(C(F)(F)F)cc(=O)n(-c2cc(Sc3cccnc3OCC(=O)OCC3CCOC3)c(Cl)cc2F)c1=O]

[(3-{2-Chloro-4-fluoro-5-[3-methyl-2,6-dioxo-4-(trifluoromethyl)-3,6-dihydropyrimidin-1(2H)-yl]phenoxy}pyridin-2-yl)oxy]acetic acid (60 mg, 0.12 mmol) was added to a solution of tetrahydro-3-furanmethanol (16 mg, 0.16 mmol) in 5 ml of dichloromethane, followed by 1-hydroxy-1H-benzotriazole hydrate (24 mg, 0.16 mmol), 1-(3-dimethylaminopropyl)-3-ethylcarbodiimide hydrochloride (31 mg, 0.16 mmol) and 4-dimethylaminopyridine (10 mol %). The reaction was stirred at RT for 2 h and allowed to stand at RT overnight, and 0.20 equivalents each of 1-hydroxy-1H-benzotriazole hydrate, 1-(3-dimethylaminopropyl)-3-ethylcarbodiimide hydrochloride and 3-methoxy-1-propanol were added. The reaction was stirred at RT for 6 h and allowed to stand at RT overnight, and the solvent was removed under reduced pressure. The residue was purified by column chromatography (gradient ethyl acetate/n-heptane) and tetrahydrofuran-3-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate (60 mg, purity: 95%, 81% of theory) was obtained. ¹H-NMR (CDCl₃ δ, ppm) 7.90-7.91 (m, 1H), 7.38 (d, 1H), 7.29-7.31 (m, 1H), 6.89-6.94 (m, 2H), 6.30 (d, 1H), 4.87-4.99 (m, 2H), 3.97-4.16 (m, 2H), 3.68-3.84 (m, 3H), 3.51 (s, 3H), 3.46-3.51 (m, 1H), 2.50-2.58 (m, 1H), 1.94-2.03 (m, 1H), 1.51-1.60 (m, 1H).

### No. I.15-350: [6-(Trifluoromethyl)pyridin-3-yl]methyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate

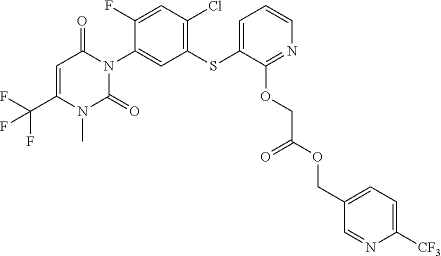 [SMILES: Cn1c(C(F)(F)F)cc(=O)n(-c2cc(Sc3cccnc3OCC(=O)OCc3ccc(C(F)(F)F)nc3)c(Cl)cc2F)c1=O]

[(3-{2-Chloro-4-fluoro-5-[3-methyl-2,6-dioxo-4-(trifluoromethyl)-3,6-dihydropyrimidin-1(2H)-yl]phenoxy}pyridin-2-yl)oxy]acetic acid (120 mg, 0.25 mmol) was added to a solution of [6-(trifluoromethyl)pyridin-3-yl]methanol (61 mg, 0.34 mmol) in 5 ml of dichloromethane, followed by 1-hydroxy-1H-benzotriazole hydrate (49 mg, 0.32 mmol), 1-(3-dimethylaminopropyl)-3-ethylcarbodiimide hydrochloride (61 mg, 0.32 mmol) and 4-dimethylaminopyridine (10 mol %). The reaction was stirred at RT for 6 h and allowed to stand at RT overnight, and the solvent was removed. The residue was purified by preparative HPLC and [6-(trifluoromethyl)pyridin-3-yl]methyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate (95 mg, purity: 98%, 59% of theory) was obtained. ¹H-NMR (CDCl₃ δ, ppm) 8.65 (s, 1H), 7.80-7.84 (m, 2H), 7.66 (d, 1H), 7.37 (d, 1H), 7.27-7.30 (m, 1H), 6.91-6.94 (m, 1H), 6.85 (d, 1H), 6.29 (s, 1H), 5.26 (m, 2H), 4.93-5.04 (m, 2H), 3.51 (m, 3H).

### No. I.31-23: 2-(2-Methoxyethoxy)ethyl {[3-({5-[3-methyl-2,6-dioxo-4-(trifluoromethyl)-3,6-dihydropyrimidin-1(2H)-yl]-2-chloro-4-fluorophenyl}sulfanyl)-5-fluoropyridin-2-yl]oxy}acetate

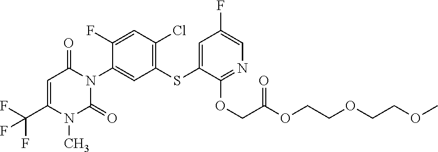 [SMILES: COCCOCCOC(=O)COc1ncc(F)cc1Sc1cc(-n2c(=O)cc(C(F)(F)F)n(C)c2=O)c(F)cc1Cl]

Under argon, 3-(4-chloro-2-fluoro-5-sulfanylphenyl)-1-methyl-6-(trifluoromethyl)pyrimidine-2,4(1H,3H)-dione (1.69 mmol, 1 equiv.) was dissolved in dioxane (16 ml) in a microwave vessel and, after degassing of the solvent, tris(dibenzylideneacetone)dipalladium (0.04 mmol), 4,5-bis(diphenylphosphino)-9,9-dimethylxanthene (0.08 mmol), N,N-diisopropylethylamine (3.37 mmol) and 3-bromo-5-fluoro-2-hydroxypyridine (1.86 mmol) were added. The resulting reaction mixture was stirred under microwave conditions at a temperature of 160° C. for 2 h. After cooling to room temperature, the reaction mixture was filtered and the filtrate was concentrated. Purification of the resulting crude product by column chromatography gave 3-{4-chloro-2-fluoro-5-[(5-fluoro-2-hydroxypyridin-3-yl)sulfanyl]phenyl}-1-methyl-6-(trifluoromethyl)pyrimidine-2,4(1H,3H)-dione (600 mg, 76% of theory) in the form of a colorless solid. In a microwave vessel and under argon, n-hexane (17 ml) was added to 3-{4-chloro-2-fluoro-5-[(5-fluoro-2-hydroxypyridin-3-yl)sulfanyl]phenyl}-1-methyl-6-(trifluoromethyl)pyrimidine-2,4(1H,3H)-dione (300 mg, 0.64 mmol). Silver(I) carbonate (213 mg, 0.77 mmol) and 2-(2-methoxyethoxy)ethyl iodoacetate (371 mg, 1.29 mmol) were then added. The reaction mixture was stirred at a temperature of 140° C. under microwave conditions for 48 minutes. After cooling to room temperature, the reaction mixture was filtered and the filtrate was concentrated under reduced pressure. Purification of the resulting crude product by column chromatography gave 2-(2-methoxyethoxy)ethyl {[3-({5-[3-methyl-2,6-dioxo-4-(trifluoromethyl)-3,6-dihydropyrimidin-1(2H)-yl]-2-chloro-4-fluorophenyl}sulfanyl)-5-fluoropyridin-2-yl]oxy}acetate (72 mg, 18% of theory) in the form of a colorless solid. ¹H-NMR (CDCl₃ δ, ppm) 7.85 (m, 1H), 7.43-7.39 (m, 2H), 7.24 (m, 1H), 6.33 (s, 1H), 4.95 (d, 1H), 4.91 (d, 1H), 4.29-4.25 (m, 2H), 3.72-3.66 (m, 2H), 3.64-3.61 (m, 2H), 3.58-3.53 (m, 5H), 3.39 (s, 3H).

In analogy to the preparation examples cited above and recited at the appropriate point, and taking account of the general details relating to the preparation of substituted N-heterocyclyl- and N-heteroaryltetrahydropyrimidinones, the compounds cited below are obtained. If in Table 1 a structural element is defined by a structural formula containing a broken line, this broken line means that at this position the group in question is attached to the remainder of the molecule. If in Table 1 a structural element is defined by a structural formula containing an arrow, the arrow represents a bond of the respective group Q to the carbonyl group in the general formula (I).

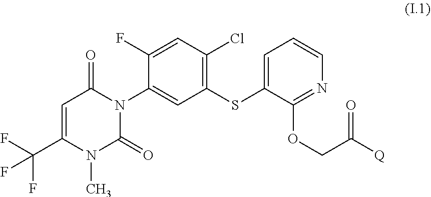

Table I.1: Preferred compounds of the formula (I.1) are the compounds I.1-1 to I.1-440 in which Q has the meanings of Table 1 indicated in the respective row. Thus, the compounds I.1-1 to I.1-440 of Table I.1 are defined by the meaning of the respective entries Nos. 1 to 440 for Q of Table 1.

TABLE 1

| No. | Q |
|---|---|
| 1 | Q-1 |
| 2 | Q-2 |
| 3 | Q-3 |
| 4 | Q-4 |
| 5 | Q-5 |
| 6 | Q-6 |
| 7 | Q-7 |
| 8 | Q-8 |
| 9 | Q-9 |
| 10 | Q-10 |
| 11 | Q-11 |
| 12 | Q-12 |
| 13 | Q-13 |
| 14 | Q-14 |
| 15 | Q-15 |
| 16 | Q-16 |
| 17 | Q-17 |
| 18 | Q-18 |
| 19 | Q-19 |
| 20 | Q-20 |
| 21 | Q-21 |
| 22 | Q-22 |
| 23 | Q-23 |
| 24 | Q-24 |
| 25 | Q-25 |
| 26 | Q-26 |
| 27 | Q-27 |
| 28 | Q-28 |
| 29 | Q-29 |
| 30 | Q-30 |
| 31 | Q-31 |
| 32 | Q-32 |
| 33 | Q-33 |
| 34 | Q-34 |
| 35 | Q-35 |
| 36 | Q-36 |
| 37 | Q-37 |
| 38 | Q-38 |
| 39 | Q-39 |
| 40 | Q-40 |
| 41 | Q-41 |
| 42 | Q-42 |
| 43 | Q-43 |
| 44 | Q-44 |
| 45 | Q-45 |
| 46 | Q-46 |
| 47 | Q-47 |
| 48 | Q-48 |
| 49 | Q-49 |
| 50 | Q-50 |
| 51 | Q-51 |
| 52 | Q-52 |
| 53 | Q-53 |
| 54 | Q-54 |
| 56 | Q-56 |
| 57 | Q-57 |
| 60 | Q-60 |
| 61 | Q-61 |
| 62 | Q-62 |
| 63 | Q-63 |
| 64 | Q-64 |
| 65 | Q-65 |
| 66 | Q-66 |
| 67 | Q-67 |
| 68 | Q-68 |
| 69 | Q-69 |
| 70 | Q-70 |
| 71 | Q-71 |
| 72 | Q-72 |
| 73 | Q-73 |
| 74 | Q-74 |
| 75 | Q-75 |
| 76 | Q-76 |
| 77 | Q-77 |
| 78 | Q-78 |
| 79 | Q-79 |
| 80 | Q-80 |
| 81 | Q-81 |
| 82 | Q-82 |
| 83 | Q-83 |
| 84 | Q-84 |
| 85 | Q-85 |
| 86 | Q-86 |
| 87 | Q-87 |
| 88 | Q-88 |
| 89 | Q-89 |
| 91 | Q-91 |
| 92 | Q-92 |
| 93 | Q-93 |
| 94 | Q-94 |
| 95 | Q-95 |
| 96 | Q-96 |
| 97 | Q-97 |
| 98 | Q-98 |
| 99 | Q-99 |
| 100 | Q-100 |
| 101 | Q-101 |
| 102 | Q-102 |
| 103 | Q-103 |
| 104 | Q-104 |
| 105 | Q-105 |
| 106 | Q-106 |
| 107 | Q-107 |
| 108 | Q-108 |
| 109 | Q-109 |
| 110 | Q-110 |
| 111 | Q-111 |
| 112 | Q-112 |
| 113 | Q-113 |
| 114 | Q-114 |
| 115 | Q-115 |
| 116 | Q-116 |
| 117 | Q-117 |
| 118 | Q-118 |
| 119 | Q-119 |
| 120 | Q-120 |
| 121 | Q-121 |
| 122 | Q-122 |
| 123 | Q-123 |
| 124 | Q-124 |
| 125 | Q-125 |
| 126 | Q-126 |
| 127 | Q-127 |
| 128 | Q-128 |
| 129 | Q-129 |
| 131 | Q-131 |
| 132 | Q-132 |
| 133 | Q-133 |
| 134 | Q-134 |
| 135 | Q-135 |
| 136 | Q-136 |
| 137 | Q-137 |
| 138 | Q-138 |
| 139 | Q-139 |
| 141 | Q-141 |
| 142 | Q-142 |
| 143 | Q-143 |
| 144 | Q-144 |
| 146 | Q-146 |
| 147 | Q-147 |
| 148 | Q-148 |
| 149 | Q-149 |
| 150 | Q-150 |
| 151 | Q-151 |
| 152 | Q-152 |
| 153 | Q-153 |
| 154 | Q-154 |
| 155 | Q-155 |
| 156 | Q-156 |
| 157 | Q-157 |
| 158 | Q-158 |
| 159 | Q-159 |
| 160 | Q-160 |
| 161 | Q-161 |
| 162 | Q-162 |
| 163 | Q-163 |
| 164 | Q-164 |
| 165 | Q-165 |
| 166 | Q-166 |
| 167 | Q-167 |
| 168 | Q-168 |
| 169 | Q-169 |
| 170 | Q-170 |
| 171 | Q-171 |
| 172 | Q-172 |
| 173 | Q-173 |
| 174 | Q-174 |
| 175 | Q-175 |
| 176 | Q-176 |
| 177 | Q-177 |
| 178 | Q-178 |
| 179 | Q-179 |
| 180 | Q-180 |
| 182 | Q-182 |
| 183 | Q-183 |
| 184 | Q-184 |
| 185 | Q-185 |
| 193 | Q-193 |
| 194 | Q-194 |
| 195 | Q-195 |
| 200 | Q-200 |
| 201 | Q-201 |
| 202 | Q-202 |
| 203 | Q-203 |
| 204 | Q-204 |
| 205 | Q-205 |
| 206 | Q-206 |
| 207 | Q-207 |
| 208 | Q-208 |
| 210 | Q-210 |
| 211 | Q-211 |
| 212 | Q-212 |
| 213 | Q-213 |
| 214 | Q-214 |
| 215 | Q-215 |
| 216 | Q-216 |
| 217 | Q-217 |
| 218 | Q-218 |
| 219 | Q-219 |
| 220 | Q-220 |
| 221 | Q-221 |
| 222 | Q-222 |
| 223 | Q-223 |
| 224 | Q-224 |
| 225 | Q-225 |
| 226 | Q-226 |
| 227 | Q-227 |
| 228 | Q-228 |
| 229 | Q-229 |
| 230 | Q-230 |
| 231 | Q-231 |
| 232 | Q-232 |
| 233 | Q-233 |
| 234 | Q-234 |
| 235 | Q-235 |
| 236 | Q-236 |
| 237 | Q-237 |
| 238 | Q-238 |
| 239 | Q-239 |
| 240 | Q-240 |
| 241 | Q-241 |
| 242 | Q-242 |
| 243 | Q-243 |
| 244 | Q-244 |
| 245 | Q-245 |
| 246 | Q-246 |
| 247 | Q-247 |
| 248 | Q-248 |
| 249 | Q-249 |
| 250 | Q-250 |
| 251 | Q-251 |
| 252 | Q-252 |
| 253 | Q-253 |
| 254 | Q-254 |
| 255 | Q-255 |
| 256 | Q-256 |
| 257 | Q-257 |
| 258 | Q-258 |
| 259 | Q-259 |
| 260 | Q-260 |
| 261 | Q-261 |
| 262 | Q-262 |
| 263 | Q-263 |
| 264 | Q-264 |
| 265 | Q-265 |
| 266 | Q-266 |
| 267 | Q-267 |
| 268 | Q-268 |
| 269 | Q-269 |
| 270 | Q-270 |
| 271 | Q-271 |
| 272 | Q-272 |
| 273 | Q-273 |
| 274 | Q-274 |
| 275 | Q-275 |
| 276 | Q-276 |
| 277 | Q-277 |
| 278 | Q-278 |
| 279 | Q-279 |
| 280 | Q-280 |
| 281 | Q-281 |
| 282 | Q-282 |
| 283 | Q-283 |
| 284 | Q-284 |
| 285 | Q-285 |
| 286 | Q-286 |
| 287 | Q-287 |
| 288 | Q-288 |
| 289 | Q-289 |
| 290 | Q-290 |
| 291 | Q-291 |
| 292 | Q-292 |
| 293 | Q-293 |
| 294 | Q-294 |
| 295 | Q-295 |
| 296 | Q-296 |
| 297 | Q-297 |
| 298 | Q-298 |
| 299 | Q-299 |
| 300 | Q-300 |
| 301 | Q-301 |
| 302 | Q-302 |
| 303 | Q-303 |
| 304 | Q-304 |
| 305 | Q-305 |
| 306 | Q-306 |
| 307 | Q-307 |
| 308 | Q-308 |
| 309 | Q-309 |
| 310 | Q-310 |
| 311 | Q-311 |
| 312 | Q-312 |
| 313 | Q-313 |
| 314 | Q-314 |
| 315 | Q-315 |
| 316 | Q-316 |
| 317 | Q-317 |
| 318 | Q-318 |
| 319 | Q-319 |
| 320 | Q-320 |
| 321 | Q-321 |
| 322 | Q-322 |
| 323 | Q-323 |
| 324 | Q-324 |
| 325 | Q-325 |
| 326 | Q-326 |
| 327 | Q-327 |
| 328 | Q-328 |
| 329 | Q-329 |
| 330 | Q-330 |
| 331 | Q-331 |
| 332 | Q-332 |
| 333 | Q-333 |
| 334 | Q-334 |
| 335 | Q-335 |
| 336 | Q-336 |
| 337 | Q-337 |
| 338 | Q-338 |
| 339 | Q-339 |
| 340 | Q-340 |
| 341 | Q-341 |
| 342 | Q-342 |
| 343 | Q-343 |
| 344 | Q-344 |
| 345 | Q-345 |
| 346 | Q-346 |
| 347 | Q-347 |
| 348 | Q-348 |
| 349 | Q-349 |
| 350 | Q-350 |
| 351 | Q-351 |
| 352 | Q-352 |
| 353 | Q-353 |
| 354 | Q-354 |
| 355 | Q-355 |
| 356 | Q-356 |
| 357 | Q-357 |
| 358 | Q-358 |
| 359 | Q-359 |
| 360 | Q-360 |
| 361 | Q-361 |
| 362 | Q-362 |
| 363 | Q-363 |
| 364 | Q-364 |
| 365 | Q-365 |
| 366 | Q-366 |
| 367 | Q-367 |
| 368 | Q-368 |
| 369 | Q-369 |
| 370 | Q-370 |
| 395 | Q-395 |
| 396 | Q-396 |
| 397 | Q-397 |
| 398 | Q-398 |
| 399 | Q-399 |
| 400 | Q-400 |
| 401 | Q-401 |
| 402 | Q-402 |
| 403 | Q-403 |
| 404 | Q-404 |
| 405 | Q-405 |
| 406 | Q-406 |
| 407 | Q-407 |
| 408 | Q-408 |
| 409 | Q-409 |
| 410 | Q-410 |
| 411 | Q-411 |
| 412 | Q-412 |
| 413 | Q-413 |
| 414 | Q-414 |
| 415 | Q-415 |
| 416 | Q-416 |
| 417 | Q-417 |
| 418 | Q-418 |
| 419 | Q-419 |
| 420 | Q-420 |
| 421 | Q-421 |
| 422 | Q-422 |
| 423 | Q-423 |
| 424 | Q-424 |
| 425 | Q-425 |
| 426 | Q-426 |
| 427 | Q-427 |
| 428 | Q-428 |
| 429 | Q-429 |
| 430 | Q-430 |
| 431 | Q-431 |
| 432 | Q-432 |
| 433 | Q-433 |
| 434 | Q-434 |
| 435 | Q-435 |
| 436 | Q-436 |
| 437 | Q-437 |
| 438 | Q-438 |
| 439 | Q-439 |
| 440 | Q-440 |

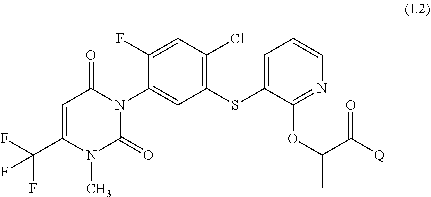

Table I.2: Preferred compounds of the formula (I.2) are the compounds I.2-1 to I.2-440 in which Q has the meanings of Table 1 indicated in the respective row. Thus, the compounds I.2-1 to I.2-440 of Table I.2 are defined by the meaning of the respective entries Nos. 1 to 440 for Q of Table 1.

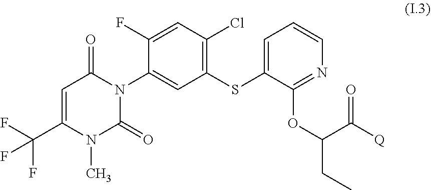

Table I.3: Preferred compounds of the formula (I.3) are the compounds I.3-1 to I.3-440 in which Q has the meanings of Table 1 indicated in the respective row. Thus, the compounds I.3-1 to I.3-440 of Table I.3 are defined by the meaning of the respective entries Nos. 1 to 440 for Q of Table 1.

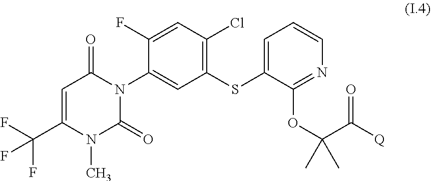

Table I.4: Preferred compounds of the formula (I.4) are the compounds I.4-1 to I.4-440 in which Q has the meanings of Table 1 indicated in the respective row. Thus, the compounds I.4-1 to I.4-440 of Table I.4 are defined by the meaning of the respective entries Nos. 1 to 440 for Q of Table 1.

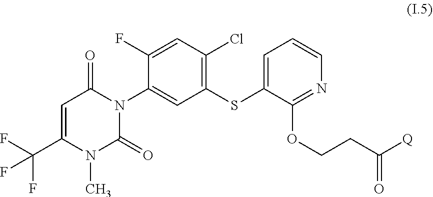

Table I.5: Preferred compounds of the formula (I.5) are the compounds I.5-1 to I.5-440 in which Q has the meanings of Table 1 indicated in the respective row. Thus, the compounds I.5-1 to I.5-440 of Table I.5 are defined by the meaning of the respective entries Nos. 1 to 440 for Q of Table 1.

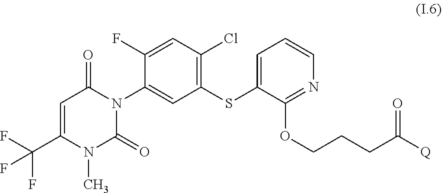

Table I.6: Preferred compounds of the formula (I.6) are the compounds I.6-1 to I.6-440 in which Q has the meanings of Table 1 indicated in the respective row. Thus, the compounds I.6-1 to I.6-440 of Table I.6 are defined by the meaning of the respective entries Nos. 1 to 440 for Q of Table 1.

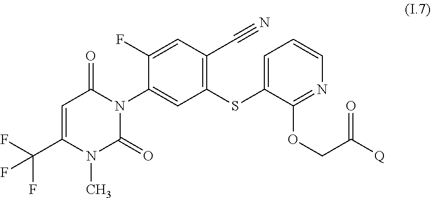

Table I.7: Preferred compounds of the formula (I.7) are the compounds I.7-1 to I.7-440 in which Q has the meanings of Table 1 indicated in the respective row. Thus, the compounds I.7-1 to I.7-440 of Table I.7 are defined by the meaning of the respective entries Nos. 1 to 440 for Q of Table 1.

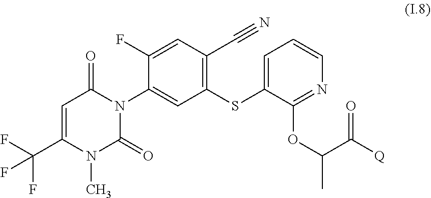

Table I.8: Preferred compounds of the formula (I.8) are the compounds I.8-1 to I.8-440 in which Q has the meanings of Table 1 indicated in the respective row. Thus, the compounds I.8-1 to I.8-440 of Table I.8 are defined by the meaning of the respective entries Nos. 1 to 440 for Q of Table 1.

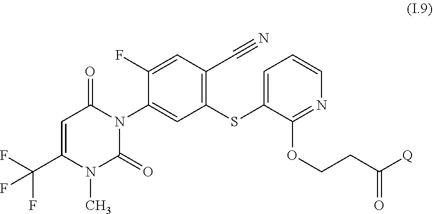

Table I.9: Preferred compounds of the formula (I.9) are the compounds I.9-1 to I.9-440 in which Q has the meanings of Table 1 indicated in the respective row. Thus, the compounds I.9-1 to I.9-440 of Table I.9 are defined by the meaning of the respective entries Nos. 1 to 440 for Q of Table 1.

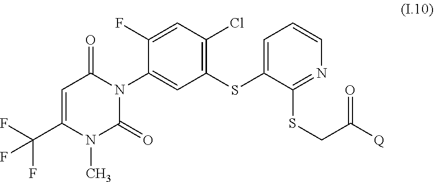

Table I.10: Preferred compounds of the formula (I.10) are the compounds I.10-1 to I.10-440 in which Q has the meanings of Table 1 indicated in the respective row. Thus, the compounds I.10-1 to I.10-440 of Table I.10 are defined by the meaning of the respective entries Nos. 1 to 440 for Q of Table 1.

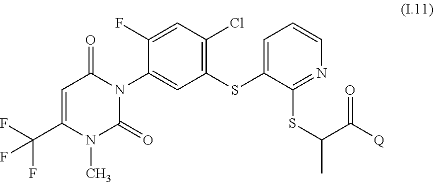

Table I.11: Preferred compounds of the formula (I.11) are the compounds I.11-1 to I.11-440 in which Q has the meanings of Table 1 indicated in the respective row. Thus, the compounds I.11-1 to I.11-440 of Table I.11 are defined by the meaning of the respective entries Nos. 1 to 440 for Q of Table 1.

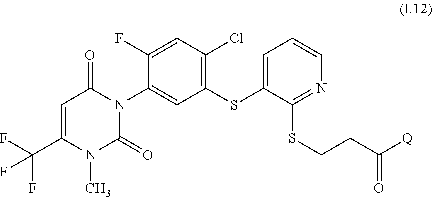

Table I.12: Preferred compounds of the formula (I.12) are the compounds I.12-1 to I.12-440 in which Q has the meanings of Table 1 indicated in the respective row. Thus, the compounds I.12-1 to I.12-440 of Table I.12 are defined by the meaning of the respective entries Nos. 1 to 440 for Q of Table 1.

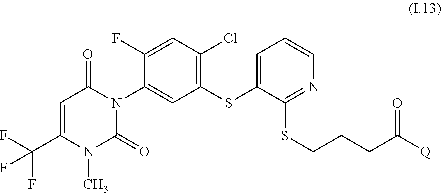

Table I.13: Preferred compounds of the formula (I.13) are the compounds I.13-1 to I.13-440 in which Q has the meanings of Table 1 indicated in the respective row. Thus, the compounds I.13-1 to I.13-440 of Table I.13 are defined by the meaning of the respective entries Nos. 1 to 440 for Q of Table 1.

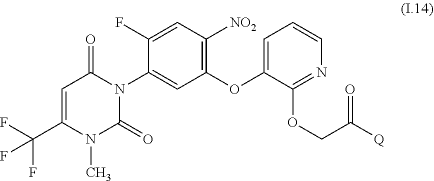

Table I.14: Preferred compounds of the formula (I.14) are the compounds I.14-1 to I.14-440 in which Q has the meanings of Table 1 indicated in the respective row. Thus, the compounds I.14-1 to I.14-440 of Table I.14 are defined by the meaning of the respective entries Nos. 1 to 440 for Q of Table 1.

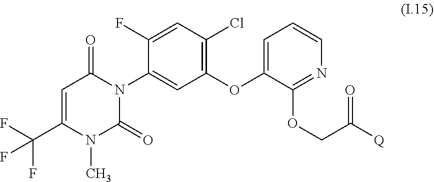

Table I.15: Preferred compounds of the formula (I.15) are the compounds I.15-1 to I.15-440 in which Q has the meanings of Table 1 indicated in the respective row. Thus, the compounds I.15-1 to I.15-440 of Table I.15 are defined by the meaning of the respective entries Nos. 1 to 440 for Q of Table 1.

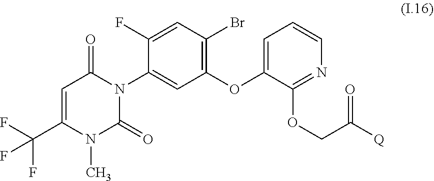

Table I.16: Preferred compounds of the formula (I.16) are the compounds I.16-1 to I.16-440 in which Q has the meanings of Table 1 indicated in the respective row. Thus, the compounds I.16-1 to I.16-440 of Table I.16 are defined by the meaning of the respective entries Nos. 1 to 440 for Q of Table 1.

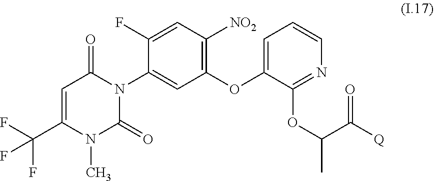

Table I.17: Preferred compounds of the formula (I.17) are the compounds I.17-1 to I.17-440 in which Q has the meanings of Table 1 indicated in the respective row. Thus, the compounds I.17-1 to I.17-440 of Table I.17 are defined by the meaning of the respective entries Nos. 1 to 440 for Q of Table 1.

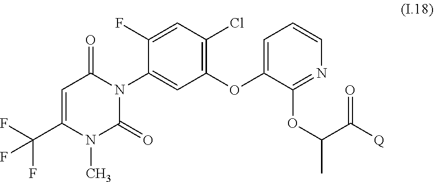

Table I.18: Preferred compounds of the formula (I.18) are the compounds I.18-1 to I.18-440 in which Q has the meanings of Table 1 indicated in the respective row. Thus, the compounds I.18-1 to I.18-440 of Table I.18 are defined by the meaning of the respective entries Nos. 1 to 440 for Q of Table 1.

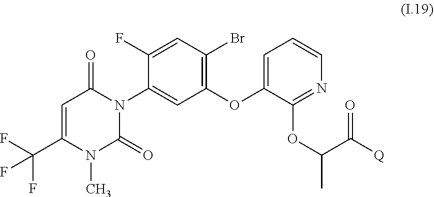

Table I.19: Preferred compounds of the formula (I.19) are the compounds I.19-1 to I.19-440 in which Q has the meanings of Table 1 indicated in the respective row. Thus, the compounds I.19-1 to I.19-440 of Table I.19 are defined by the meaning of the respective entries Nos. 1 to 440 for Q of Table 1.

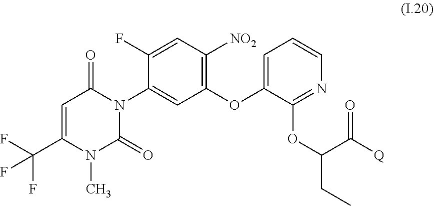

Table I.20: Preferred compounds of the formula (I.20) are the compounds I.20-1 to I.20-440 in which Q has the meanings of Table 1 indicated in the respective row. Thus, the compounds I.20-1 to I.20-440 of Table I.20 are defined by the meaning of the respective entries Nos. 1 to 440 for Q of Table 1.

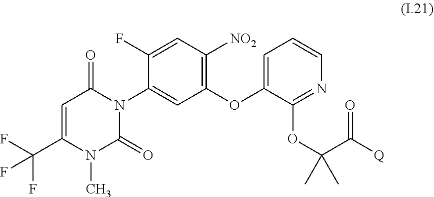

Table I.21: Preferred compounds of the formula (I.21) are the compounds I.21-1 to I.21-440 in which Q has the meanings of Table 1 indicated in the respective row. Thus, the compounds I.21-1 to I.21-440 of Table I.21 are defined by the meaning of the respective entries Nos. 1 to 440 for Q of Table 1.

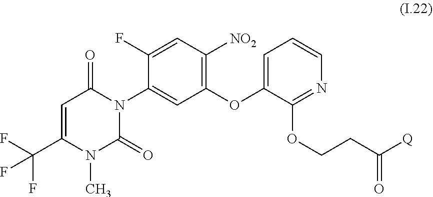

Table I.22: Preferred compounds of the formula (I.22) are the compounds I.22-1 to I.22-440 in which Q has the meanings of Table 1 indicated in the respective row. Thus, the compounds I.22-1 to I.22-440 of Table I.22 are defined by the meaning of the respective entries Nos. 1 to 440 for Q of Table 1.

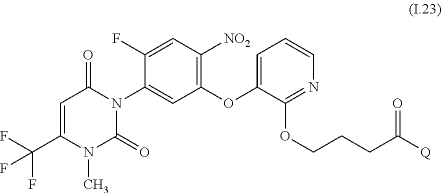

Table I.23: Preferred compounds of the formula (I.23) are the compounds I.23-1 to I.23-440 in which Q has the meanings of Table 1 indicated in the respective row. Thus, the compounds I.23-1 to I.23-440 of Table I.23 are defined by the meaning of the respective entries Nos. 1 to 440 for Q of Table 1.

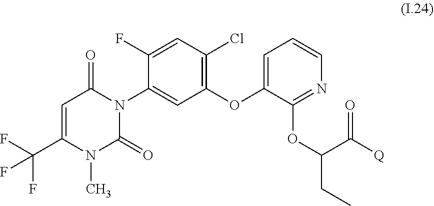

Table I.24: Preferred compounds of the formula (I.24) are the compounds I.24-1 to I.24-440 in which Q has the meanings of Table 1 indicated in the respective row. Thus, the compounds I.24-1 to I.24-440 of Table I.24 are defined by the meaning of the respective entries Nos. 1 to 440 for Q of Table 1.

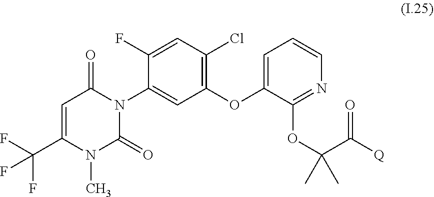

Table I.25: Preferred compounds of the formula (I.25) are the compounds I.25-1 to I.25-440 in which Q has the meanings of Table 1 indicated in the respective row. Thus, the compounds I.25-1 to I.25-440 of Table I.25 are defined by the meaning of the respective entries Nos. 1 to 440 for Q of Table 1.

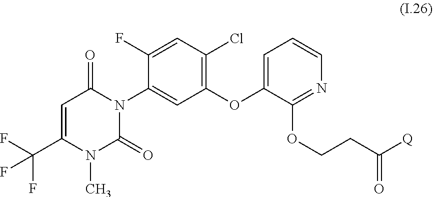

Table I.26: Preferred compounds of the formula (I.26) are the compounds I.26-1 to I.26-440 in which Q has the meanings of Table 1 indicated in the respective row. Thus, the compounds I.26-1 to I.26-440 of Table I.26 are defined by the meaning of the respective entries Nos. 1 to 440 for Q of Table 1.

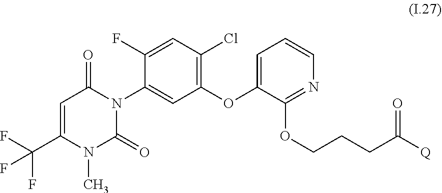

Table I.27: Preferred compounds of the formula (I.27) are the compounds I.27-1 to I.27-440 in which Q has the meanings of Table 1 indicated in the respective row. Thus, the compounds I.27-1 to I.27-440 of Table I.27 are defined by the meaning of the respective entries Nos. 1 to 440 for Q of Table 1.

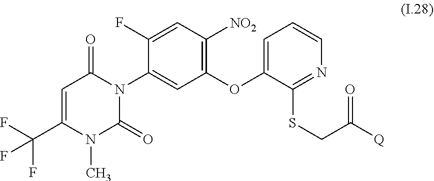

Table I.28: Preferred compounds of the formula (I.28) are the compounds I.28-1 to I.28-440 in which Q has the meanings of Table 1 indicated in the respective row. Thus, the compounds I.28-1 to I.28-440 of Table I.28 are defined by the meaning of the respective entries Nos. 1 to 440 for Q of Table 1.

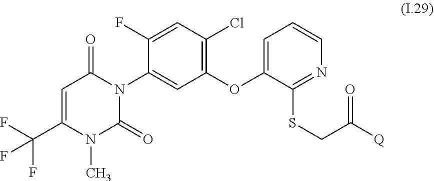

Table I.29: Preferred compounds of the formula (I.29) are the compounds I.29-1 to I.29-440 in which Q has the meanings of Table 1 indicated in the respective row. Thus, the compounds I.29-1 to I.29-440 of Table I.29 are defined by the meaning of the respective entries Nos. 1 to 440 for Q of Table 1.

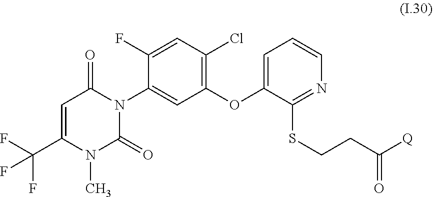

Table I.30: Preferred compounds of the formula (I.30) are the compounds I.30-1 to I.30-440 in which Q has the meanings of Table 1 indicated in the respective row. Thus, the compounds I.30-1 to I.30-440 of Table I.30 are defined by the meaning of the respective entries Nos. 1 to 440 for Q of Table 1.

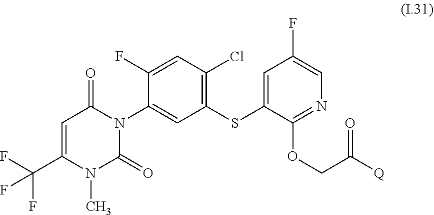

Table I.31: Preferred compounds of the formula (I.31) are the compounds I.31-1 to I.31-440 in which Q has the meanings of Table 1 indicated in the respective row. Thus, the compounds I.31-1 to I.31-440 of Table I.31 are defined by the meaning of the respective entries Nos. 1 to 440 for Q of Table 1.

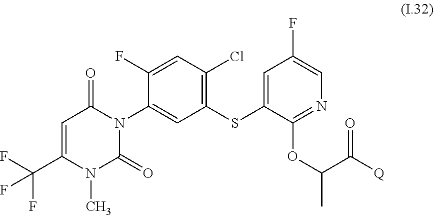

Table I.32: Preferred compounds of the formula (I.32) are the compounds I.32-1 to I.32-440 in which Q has the meanings of Table 1 indicated in the respective row. Thus, the compounds I.32-1 to I.32-440 of Table I.32 are defined by the meaning of the respective entries Nos. 1 to 440 for Q of Table 1.

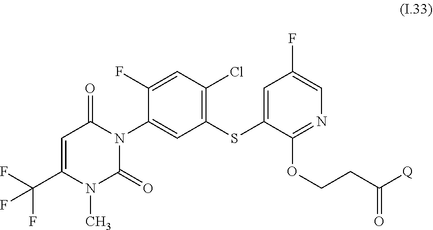

Table I.33: Preferred compounds of the formula (I.33) are the compounds I.33-1 to I.33-440 in which Q has the meanings of Table 1 indicated in the respective row. Thus, the compounds I.33-1 to I.33-440 of Table I.33 are defined by the meaning of the respective entries Nos. 1 to 440 for Q of Table 1.

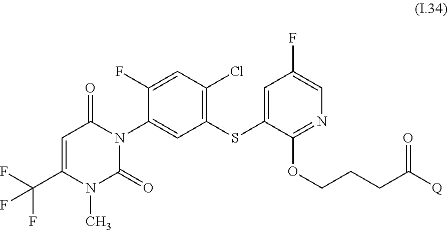

Table I.34: Preferred compounds of the formula (I.34) are the compounds I.34-1 to I.34-440 in which Q has the meanings of Table 1 indicated in the respective row. Thus, the compounds I.34-1 to I.34-440 of Table I.34 are defined by the meaning of the respective entries Nos. 1 to 440 for Q of Table 1.

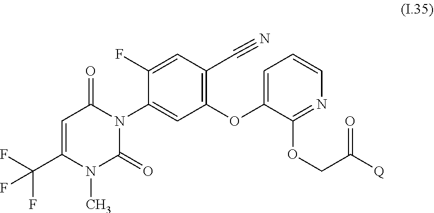

Table I.35: Preferred compounds of the formula (I.35) are the compounds I.35-1 to I.35-440 in which Q has the meanings of Table 1 indicated in the respective row. Thus, the compounds I.35-1 to I.35-440 of Table I.35 are defined by the meaning of the respective entries Nos. 1 to 440 for Q of Table 1.

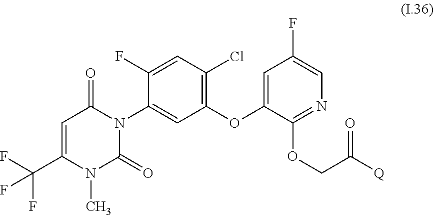

Table I.36: Preferred compounds of the formula (I.36) are the compounds I.36-1 to I.36-440 in which Q has the meanings of Table 1 indicated in the respective row. Thus, the compounds I.36-1 to I.36-440 of Table I.36 are defined by the meaning of the respective entries Nos. 1 to 440 for Q of Table 1.

NMR data of selected examples: The ¹H NMR data of selected examples of compounds of the general formula (I) are stated in two different ways, namely (a) conventional NMR interpretation or (b) in the form of 1H NMR peak lists according to the method described below.

### a) Conventional NMR Interpretation

### Example No. I.1-115

¹H-NMR (CDCl₃ δ, ppm) 8.06 (d, 1H), 7.61 (m, 1H), 7.37 (d, 1H), 7.21 (m, 1H), 6.95-6.91 (m, 1H), 6.30 (s, 1H), 5.45-5.42 (m, 1H), 4.99-4.96 (d, 1H), 4.93-4.89 (d, 1H), 4.85 (m, 2H), 4.63 (m, 2H), 3.52 (s, 3H).

### Example No. I.1-176

¹H-NMR (CDCl₃ δ, ppm) 8.08 (d, 1H), 7.68 (m, 1H), 7.38 (d, 1H), 7.18 (d, 1H), 6.98-6.95 (m, 1H), 6.32 (s, 1H), 5.02-4.98 (d, 1H), 4.95-4.91 (d, 1H), 4.74 (s, 2H), 3.53 (s, 3H).

### Example No. I.1-286

¹H-NMR (CDCl₃ δ, ppm) 8.57 (m, 1H), 8.06 (d, 1H), 7.70-7.63 (m, 2H), 7.34-7.28 (m, 2H), 7.26-7.21 (m, 2H), 6.94-6.91 (m, 1H), 6.23 (s, 1H), 5.29-5.26 (d, 1H), 5.24-5.20 (d, 1H), 5.07-5.03 (d, 1H), 5.01-4.97 (d, 1H), 3.48 (s, 3H).

### Example No. I.14-1

¹H-NMR (CDCl₃ δ, ppm): 7.99 (dd, 1H), 7.87 (d, 1H), 7.51 (dd, 1H), 7.10 (d, 1H), 7.00 (dd, 1H), 6.28 (s, 1H), 4.94 (q, 1H), 4.21-4.18 (m, 2H), 3.52-3.50 (m, 5H), 3.30 (s, 3H).

### Example No. I.14-115

¹H-NMR (CDCl₃ δ, ppm): 7.98 (dd, 1H), 7.88 (d, 1H), 7.50 (dd, 1H), 7.02-6.99 (m, 2H), 6.28 (s, 1H), 5.43-5.40 (quintett, 1H), 4.94 (q, 1H), 4.86-4.81 (m, 2H), 4.64-4.60 (m, 2H), 3.51 (s, 3H).

### Example No. I.15-2

¹H-NMR (CDCl₃ δ, ppm) 7.92-7.90 (m, 1H), 7.37 (d, 1H), 7.34-7.31 (m, 1H), 6.94-6.91 (m, 2H), 6.29 (s, 1H), 5.02-4.91 (m, 2H), 4.26-4.23 (m, 2H), 3.60-3.57 (m, 2H), 3.51 (s, 3H), 3.47 (q, 2H), 1.17 (t, 3H).

### Example No. I.15-71

¹H-NMR (CDCl₃ δ, ppm) 7.90-7.92 (m, 1H), 7.37 (d, 1H), 7.31-7.34 (m, 1H), 6.90-6.94 (m, 2H), 6.29 (m, 1H), 4.90-5.04 (m, 2H), 4.03-4.17 (m, 3H), 3.70-3.80 (m, 2H), 3.50 (m, 3H), 1.81-1.98 (m, 2H), 1.53-1.57 (m, 1H).

### Example No. I.15-211

¹H-NMR (CDCl₃ δ, ppm) 7.89-7.87 (m, 1H), 7.34 (d, 1H), 7.29 (d, 1H), 6.93-6.87 (m, 2H), 6.25 (s, 1H), 4.94-4.90 (d, 1H), 4.84-4.80 (d, 1H), 4.19-4.13 (m, 2H), 3.47 (s, 3H), 0.98-0.94 (m, 2H), -0.02 (s, 9H).

### Example No. I.15-280

¹H-NMR (CDCl₃ δ, ppm) 7.95 (m, 1H), 7.38-7.35 (m, 2H), 6.98-6.96 (m, 1H), 6.84-6.78 (d, 1H), 6.50/6.32 (s, 1H), 5.99/5.73 (s, 1H), 5.07-4.98 (m, 1H), 4.88-4.80 (m, 1H), 4.68-4.55 (m, 1H), 4.35-4.24 (m, 1H), 4.23 (br. m, 1H, NH), 4.12 (br. s, 1H, NH), 3.51 (s, 3H).

### Example No. I.15-288

¹H-NMR (CDCl₃ δ, ppm) 8.57 (d, 1H), 7.88-7.90 (m, 1H), 7.36 (d, 1H), 7.30-7.32 (m, 1H), 7.17 (d, 1H), 6.94-6.96 (m, 1H), 6.84 (d, 1H), 6.25 (s, 1H), 5.17-5.21 (m, 2H), 5.03 (q, 2H), 3.49 (s, 3H).

### Example No. I.15-350

¹H-NMR (CDCl₃ δ, ppm) 8.65 (d, 1H), 7.85-7.80 (m, 2H), 7.67 (d, 1H), 7.38 (d, 1H), 7.30-7.28 (m, 1H), 6.94-6.92 (m, 1H), 6.86 (d, 1H), 6.29 (s, 1H), 5.26 (s, 2H), 5.04-4.93 (q, 2H), 3.51 (s, 3H).

### Example No. I.15-366

¹H-NMR (CDCl₃ δ, ppm) 7.87-7.85 (m, 1H), 7.37 (d, 1H), 7.29 (s, 1H), 7.28-7.26 (m, 1H), 6.92-6.87 (m, 2H), 6.30 (s, 1H), 4.99 (s, 2H), 4.90 (dd, 2H), 3.79 (s, 3H), 3.52 (s, 3H), 2.18 (s, 3H).

### Example No. I.15-367

¹H-NMR (CDCl₃ δ, ppm) 7.88-7.86 (m, 1H), 7.38-7.35 (m, 2H), 7.28-7.26 (m, 1H), 6.92-6.88 (m, 2H), 6.30 (s, 1H), 4.99 (s, 2H), 4.90 (dd, 2H), 3.76 (s, 3H), 3.52 (s, 3H), 2.21 (s, 3H).

### Example No. I.15-368

¹H-NMR (CDCl₃ δ, ppm) 7.86-7.85 (m, 1H), 7.36 (d, 1H), 7.33 (s, 1H), 7.28-7.26 (m, 1H), 6.92-6.88 (m, 2H), 6.30 (s, 1H), 5.00 (s, 2H), 4.91 (dd, 2H), 4.06 (q, 2H), 3.52 (s, 3H), 2.20 (s, 3H), 1.45 (t, 3H).

### Example No. I.15-421

¹H-NMR (CDCl₃ δ, ppm) 7.93-7.91 (m, 1H), 7.38-7.33 (m, 2H), 6.95-6.92 (m, 1H), 6.90-6.87 (m, 1H), 6.29 (s, 1H), 5.02 (d, 1H), 4.96 (d, 1H), 4.46-4.44 (m, 1H), 4.12-4.08 (m, 2H), 3.50 (s, 3H), 3.33 (s, 3H), 3.32 (s, 3H).

### Example No. I.15-422

¹H-NMR (CDCl₃ δ, ppm) 7.92-7.90 (m, 1H), 7.38-7.31 (m, 2H), 6.95-6.88 (m, 2H), 6.29 (s, 1H), 5.02 (d, 1H), 4.96 (d, 1H), 4.63-4.60 (m, 1H), 4.12-4.07 (m, 2H), 3.69-3.61 (m, 2H), 3.55-3.48 (m, 5H), 1.19 (t, 3H).

### Example No. I.16-1

¹H-NMR (CDCl₃ δ, ppm) 7.92-7.90 (m, 1H), 7.53 (d, 1H), 7.34-7.31 (m, 1H), 6.94-6.92 (m, 1H), 6.88 (d, 1H), 6.28 (s, 1H), 5.01 (d, 1H), 4.94 (d, 1H), 4.27-4.20 (m, 2H), 3.55-3.51 (m, 2H), 3.50 (s, 3H), 3.31 (s, 3H).

### Example No. I.16-2

¹H-NMR (CDCl₃ δ, ppm) 7.92-7.90 (m, 1H), 7.54 (d, 1H), 7.34-7.31 (m, 1H), 6.94-6.87 (m, 2H), 6.29 (s, 1H), 5.02 (d, 1H), 4.95 (d, 1H), 4.26-4.23 (m, 2H), 3.60-3.58 (m, 2H), 3.50 (s, 3H), 3.47 (q, 2H), 1.17 (t, 3H).

### Example No. I.16-23

¹H-NMR (CDCl₃ δ, ppm) 7.92-7.90 (m, 1H), 7.54 (d, 1H), 7.34-7.31 (m, 1H), 6.94-6.87 (m, 2H), 6.29 (s, 1H), 5.02 (d, 1H), 4.94 (d, 1H), 4.27-4.25 (m, 2H), 3.69-3.66 (m, 2H), 3.62-3.59 (m, 2H), 3.53-3.51 (m, 2H), 3.50 (s, 3H), 3.37 (s, 3H).

### Example No. I.16-41

¹H-NMR (CDCl₃ δ, ppm): 7.91 (dd, 1H), 7.54 (d, 1H), 7.32 (dd, 1H), 6.93 (dd, 1H), 6.79 (d, 1H), 6.31 (s, 1H), 4.96 (q, 2H), 4.38-4.20 (m, 2H), 4.11 (t, 2H), 3.50 (s, 3H).

### Example No. I.16-71

¹H-NMR (CDCl₃ δ, ppm): 7.92 (dd, 1H), 7.53 (d, 1H), 7.33 (dd, 1H), 6.94-6.87 (m, 2H), 6.28 (s, 1H), 4.97 (pseudo quintett, 2H), 4.20-4.12 (m, 1H), 4.10-4.00 (m, 2H), 3.81-3.68 (m, 2H), 3.50 (s, 3H), 1.98-1180 (m, 3H), 1.60-1.50 (m, 1H).

### Example No. I.16-115

¹H-NMR (CDCl₃ δ, ppm): 7.91 (dd, 1H), 7.53 (d, 1H), 7.32 (dd, 1H), 6.94 (dd, 1H), 6.82 (d, 1H), 6.29 (s, 1H), 5.49-5.43 (quintett, 1H), 4.96 (q, 1H), 4.84 (m, 2H), 4.62 (m, 2H), 3.51 (s, 3H).

### Example No. I.16-176

¹H-NMR (CDCl₃ δ, ppm) 7.94-7.92 (m, 1H), 7.56 (d, 1H), 7.38-7.35 (m, 1H), 6.99-6.95 (m, 1H), 6.77-6.74 (m, 1H), 6.31 (s, 1H), 5.04-5.00 (d, 1H), 4.97-4.93 (d, 1H), 4.74 (s, 2H), 3.51 (s, 3H).

### Example No. I.16-286

¹H-NMR (CDCl₃ δ, ppm): 8.53 (d, 1H), 7.90 (dd, 1H), 7.67 (dt, 1H), 7.52 (d, 1H), 7.34 (dd, 1H), 7.29-7.28 (d, 1H), 7.22 (dd, 1H), 6.92 (dd, 1H), 6.85 (d, 1H), 6.23 (s, 1H), 5.26 (pseudo t, 2H), 5.09-4.99 (q, 2H), 3.48 (s, 3H).

### Example No. I.16-301

¹H-NMR (CDCl₃ δ, ppm): 9.14 (dd, 1H), 7.89 (dd, 1H), 7.54-7.45 (m, 3H), 7.33 (dd, 1H), 6.939 (dd, 1H), 6.78 (d, 1H), 6.26 (s, 1H), 5.50 (q, 2H), 5.04 (q, 2H), 3.50 (s, 3H).

### Example No. I.16-421

¹H-NMR (CDCl₃ δ, ppm) 7.93-7.91 (m, 1H), 7.54 (d, 1H), 7.35-7.33 (m, 2H), 6.95-6.92 (m, 1H), 6.86 (d, 1H), 6.29 (s, 1H), 5.00 (d, 1H), 4.95 (d, 1H), 4.46-4.44 (m, 1H), 4.12-4.06 (m, 2H), 3.50 (s, 3H), 3.33 (s, 3H), 3.32 (s, 3H).

### Example No. I.16-424

¹H-NMR (CDCl₃ δ, ppm): 7.92 (dd, 1H), 7.53 (d, 1H), 7.34 (dd, 1H), 6.93 (dd, 1H), 6.87 (d, 1H), 6.29 (s, 1H), 5.07 (t, 1H), 4.99 (q, 2H), 4.15 (pseudo q, 2H), 3.96-3.86 (m, 4H), 3.50 (s, 3H).

### Example No. I.31-1

¹H-NMR (CDCl₃ δ, ppm) 7.85 (m, 1H), 7.42-7.38 (m, 2H), 7.27 (m, 1H), 6.32 (s, 1H), 4.95 (d, 1H), 4.91 (d, 1H), 4.28-4.23 (m, 2H), 3.59-3.55 (m, 2H), 3.53 (s, 3H), 3.36 (s, 3H).

### Example No. I.35-23

¹H-NMR (CDCl₃ δ, ppm): 8.00 (dd, 1H), 7.54-7.49 (m, 2H), 7.00 (dd, 1H), 6.97 (d, 1H), 6.28 (s, 1H), 4.93 (q, 1H), 4.23-4.20 (m, 2H), 3.67-3.65 (m, 2H), 3.61-3.59 (m, 2H), 3.54-3.49 (m, 5H), 3.37 (s, 3H).

### Example No. I.35-41

¹H-NMR (DMSO-D₆ δ, ppm): 8.19 (d, 1H), 8.06 (dd, 1H), 7.80 (dd, 1H), 7.17 (dd, 1H), 7.06 (d, 1H), 6.56 (s, 1H), 4.94 (ps q, 2H), 4.33-4.29 (m, 2H), 4.26-4.22 (m, 2H), 3.36 (s, 3H).

### Example No. I.35-176

¹H-NMR (CDCl₃ δ, ppm): 8.02 (dd, 1H), 7.57-7.52 (m, 2H), 7.05 (dd, 1H), 6.80 (d, 1H), 6.28 (s, 1H), 4.96 (q, 2H), 4.72 (s, 2H), 3.51 (s, 3H).

### Example No. I.35-286

¹H-NMR (CDCl₃ δ, ppm): 8.53 (d, 1H), 7.96 (dd, 1H), 7.72 (dt, 1H), 7.53 (dd, 1H), 7.49 (d, 1H), 7.30-7.22 (m, 2H), 6.99 (dd, 1H), 6.93 (d, 1H), 6.20 (s, 1H), 5.23 (m, 2H), 5.08-4.95 (q, 2H), 3.46 (s, 3H).

### Example No. I.36-176

¹H-NMR (CDCl₃ δ, ppm) 7.78 (m, 1H), 7.41 (d, 1H), 7.14 (m, 1H), 6.91 (d, 1H), 6.33 (s, 1H), 4.97 (dd, 2H), 4.76 (s, 2H), 3.53 (s, 3H).

### Example No. I.36-286

¹H-NMR (CDCl₃ δ, ppm) 8.55 (m, 1H), 7.74 (m, 1H), 7.71-7.67 (m, 1H), 7.38 (d, 1H), 7.29 (m, 1H), 7.24-7.21 (m, 1H), 7.11 (m, 1H), 6.98 (d, 1H), 6.26 (s, 1H), 5.25 (s, 2H), 5.02 (dd, 2H), 3.50 (s, 3H).

### b) NMR Peak List Method

The ¹H NMR data of selected examples may also be stated in the form of ¹H NMR peak lists. For each signal peak, first the δ value in ppm and then the signal intensity in round brackets are listed. The δ-value/signal intensity number pairs for different signal peaks are listed with separation from one another by semicolons. The peak list for one example therefore takes the form of:

δ₁ (intensity₁); δ₂ (intensity₂); . . . ; δᵢ (intensityᵢ); . . . ; δₙ (intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum. For calibration of the chemical shift of 1H NMR spectra we use tetramethylsilane and/or the chemical shift of the solvent, particularly in the case of spectra measured in DMSO. Therefore, the tetramethylsilane peak may but need not occur in NMR peak lists. The lists of the ¹H NMR peaks are similar to the conventional ¹H NMR printouts and thus usually contain all peaks listed in a conventional NMR interpretation. In addition, like conventional ¹H NMR printouts, they may show solvent signals, signals of stereoisomers of the compounds which are likewise provided by the invention, and/or peaks of impurities. In the reporting of compound signals within the delta range of solvents and/or water, our lists of ¹H NMR peaks show the standard solvent peaks, for example peaks of DMSO in DMSO-D₆ and the peak of water, which usually have a high intensity on average. The peaks of stereoisomers of the target compounds and/or peaks of impurities usually have a lower intensity on average than the peaks of the target compounds (for example with a purity of >90%). Such stereoisomers and/or impurities may be typical of the particular preparation process. Their peaks can thus help in this case to identify reproduction of our preparation process with reference to “by-product fingerprints”. An expert calculating the peaks of the target compounds by known methods (MestreC, ACD simulation, but also with empirically evaluated expected values) can, if required, isolate the peaks of the target compounds, optionally using additional intensity filters. This isolation would be similar to the relevant peak picking in conventional 1H NMR interpretation. Further details of ¹H NMR peak lists can be found in the Research disclosure Database Number 564025.

### Example No. I.1-1

¹H-NMR (400.0 MHz, CDCl₃): δ=8.0832 (1.2); 8.0787 (1.2); 8.0709 (1.2); 8.0664 (1.2); 7.6537 (1.2); 7.6492 (1.2); 7.6350 (1.4); 7.6305 (1.2); 7.3536 (1.8); 7.3311 (1.8); 7.2603 (75.4); 7.2524 (1.9); 7.2338 (1.8); 6.9368 (1.3); 6.9245 (1.3); 6.9181 (1.2); 6.9058 (1.2); 6.2898 (2.9); 4.9977 (0.8); 4.9579 (2.8); 4.9334 (2.8); 4.8936 (0.8); 4.2431 (0.8); 4.2387 (0.8); 4.2324 (1.5); 4.2258 (1.4); 4.2195 (0.9); 4.2150 (0.8); 3.5728 (1.9); 3.5620 (1.8); 3.5490 (1.7); 3.5066 (3.9); 3.5036 (3.9); 3.3484 (16.0); 1.5437 (2.3); 1.2596 (0.6); 0.8821 (0.9); 0.0080 (1.4); -0.0002 (45.2); -0.0085 (1.2)

### Example No. I.1-71

¹H-NMR (400.0 MHz, CDCl₃): δ=8.0782 (4.2); 8.0738 (4.5); 8.0659 (4.5); 8.0614 (4.4); 7.6474 (2.6); 7.6454 (2.9); 7.6430 (3.0); 7.6409 (2.8); 7.6287 (3.0); 7.6267 (3.2); 7.6243 (3.0); 7.6223 (2.8); 7.5193 (1.2); 7.3537 (7.5); 7.3312 (7.5); 7.2682 (8.4); 7.2605 (215.6); 7.2497 (7.6); 6.9964 (1.2); 6.9336 (5.2); 6.9212 (5.1); 6.9149 (5.0); 6.9026 (4.9); 6.2955 (6.4); 6.2897 (6.3); 5.0047 (1.9); 4.9945 (1.0); 4.9649 (5.7); 4.9548 (6.3); 4.9423 (6.3); 4.9300 (5.5); 4.9025 (1.1); 4.8902 (1.9); 4.1749 (1.5); 4.1661 (2.2); 4.1582 (1.6); 4.1483 (2.1); 4.1399 (3.8); 4.1334 (1.4); 4.1131 (1.1); 4.1083 (0.6); 4.0986 (1.2); 4.0917 (1.5); 4.0830 (1.4); 4.0742 (1.8); 4.0715 (2.0); 4.0663 (2.7); 4.0582 (1.7); 4.0447 (3.0); 4.0419 (2.2); 4.0294 (1.6); 4.0181 (1.8); 4.0028 (1.2); 3.8697 (0.8); 3.8662 (0.8); 3.8526 (1.6); 3.8489 (2.4); 3.8453 (1.4); 3.8322 (2.6); 3.8156 (1.4); 3.7948 (1.0); 3.7878 (1.0); 3.7777 (1.9); 3.7705 (1.9); 3.7613 (1.3); 3.7545 (1.4); 3.7403 (0.6); 3.7332 (0.6); 3.5073 (15.8); 3.5048 (16.0); 2.0452 (3.5); 1.9850 (0.6); 1.9685 (0.9); 1.9522 (1.4); 1.9438 (1.1); 1.9398 (1.0); 1.9317 (0.9); 1.9196 (1.8); 1.9158 (1.6); 1.9012 (2.9); 1.8850 (3.6); 1.8820 (3.5); 1.8655 (2.2); 1.8499 (0.8); 1.6072 (0.8); 1.5914 (1.4); 1.5746 (1.7); 1.5684 (1.4); 1.5499 (4.4); 1.3032 (0.9); 1.2844 (1.5); 1.2773 (2.2); 1.2642 (4.4); 1.2597 (5.1); 1.2416 (1.5); 0.8988 (2.3); 0.8820 (7.9); 0.8643 (3.1); 0.0080 (3.8); -0.0002 (129.8); -0.0085 (3.6)

### Example No. I.1-72

¹H-NMR (400.0 MHz, CDCl₃): δ=8.3787 (3.2); 8.3747 (3.3); 8.3668 (3.4); 8.3626 (3.3); 7.6161 (2.7); 7.6120 (2.7); 7.5969 (3.0); 7.5929 (2.8); 7.5194 (0.8); 7.3712 (5.4); 7.3488 (5.4); 7.2605 (145.8); 7.0431 (3.4); 7.0310 (3.4); 7.0238 (3.1); 7.0118 (3.1); 6.9964 (0.8); 6.9610 (2.9); 6.9581 (2.9); 6.9428 (2.9); 6.9398 (2.8); 6.2954 (7.1); 4.1487 (1.2); 4.1326 (1.3); 4.1212 (2.0); 4.1092 (1.7); 4.1061 (1.6); 4.0934 (1.5); 4.0140 (1.5); 3.9968 (1.8); 3.9872 (1.1); 3.9766 (1.6); 3.9698 (1.5); 3.9496 (1.2); 3.9270 (16.0); 3.8336 (0.9); 3.8201 (1.0); 3.8129 (2.0); 3.7992 (2.0); 3.7923 (1.4); 3.7787 (1.4); 3.7625 (1.2); 3.7566 (1.2); 3.7446 (1.5); 3.7393 (3.1); 3.7217 (2.9); 3.7010 (1.9); 3.6806 (0.9); 3.5046 (15.4); 3.4912 (2.8); 3.4828 (2.2); 3.4691 (2.2); 2.5756 (0.9); 2.5597 (1.1); 2.5411 (0.9); 2.0451 (2.5); 2.0275 (0.6); 2.0137 (0.7); 2.0081 (0.9); 1.9952 (1.2); 1.9869 (0.7); 1.9826 (0.8); 1.9738 (1.1); 1.9622 (1.0); 1.9558 (0.6); 1.9416 (0.6); 1.6146 (0.7); 1.5957 (1.6); 1.5637 (4.8); 1.2773 (1.2); 1.2596 (2.6); 1.2415 (0.8); 0.8988 (1.0); 0.8821 (2.5); 0.8642 (1.0); 0.0079 (3.2); -0.0002 (83.2); -0.0085 (2.6)

### Example No. I.10-1

¹H-NMR (400.0 MHz, CDCl₃): δ=8.3904 (1.2); 8.3863 (1.2); 8.3784 (1.3); 8.3743 (1.2); 7.6278 (1.2); 7.6236 (1.2); 7.6086 (1.4); 7.6045 (1.3); 7.3619 (2.0); 7.3395 (2.0); 7.2605 (44.2); 7.0349 (1.3); 7.0229 (1.3); 7.0157 (1.2); 7.0038 (1.2); 6.9348 (1.8); 6.9166 (1.8); 6.2894 (3.2); 4.2524 (1.6); 4.2412 (1.4); 4.2374 (1.1); 4.2288 (1.8); 3.9761 (7.6); 3.5802 (2.1); 3.5739 (0.7); 3.5712 (1.1); 3.5683 (1.9); 3.5566 (1.9); 3.5043 (4.5); 3.5015 (4.5); 3.3425 (16.0); 1.5626 (1.0); 1.2595 (0.7); 0.8821 (0.8); 0.0079 (0.8); -0.0002 (25.1); -0.0085 (0.8)

### Example No. I.10-26

¹H-NMR (400.0 MHz, CDCl₃): δ=8.3833 (1.8); 8.3796 (1.8); 8.3715 (1.8); 7.6166 (1.8); 7.6131 (1.6); 7.5974 (1.8); 7.5939 (1.6); 7.3661 (2.5); 7.3437 (2.4); 7.2610 (18.3); 7.0345 (1.6); 7.0225 (1.6); 7.0155 (1.5); 7.0035 (1.4); 6.9688 (2.3); 6.9507 (2.3); 6.2963 (4.5); 4.1962 (2.4); 4.1802 (4.5); 4.1642 (2.3); 3.9380 (8.9); 3.5065 (8.9); 3.4117 (2.7); 3.3961 (5.0); 3.3804 (2.6); 3.2978 (16.0); 1.9106 (0.8); 1.8950 (2.6); 1.8791 (3.6); 1.8632 (2.4); 1.8474 (0.7); 1.5720 (1.0); 1.2562 (0.9); -0.0002 (10.6)

### Example No. I.10-71

¹H-NMR (400.0 MHz, CDCl₃): δ=8.3861 (4.4); 8.3819 (4.5); 8.3741 (4.7); 8.3700 (4.4); 7.6193 (4.1); 7.6151 (4.1); 7.6001 (4.5); 7.5960 (4.2); 7.3625 (7.0); 7.3401 (7.0); 7.2613 (71.1); 7.0311 (4.7); 7.0192 (4.6); 7.0120 (4.4); 7.0000 (4.4); 6.9498 (5.2); 6.9316 (5.2); 6.2929 (6.5); 6.2899 (6.2); 4.1877 (1.7); 4.1859 (1.6); 4.1788 (2.2); 4.1604 (2.3); 4.1515 (3.7); 4.1344 (0.6); 4.1178 (1.6); 4.1090 (1.1); 4.1009 (1.7); 4.0926 (1.6); 4.0837 (0.8); 4.0758 (0.9); 4.0600 (2.8); 4.0510 (2.7); 4.0449 (1.5); 4.0353 (2.0); 4.0331 (2.0); 4.0238 (2.2); 4.0179 (1.2); 4.0083 (1.2); 3.9848 (9.0); 3.9828 (9.4); 3.9797 (12.9); 3.9389 (0.8); 3.8776 (1.4); 3.8607 (2.4); 3.8566 (2.4); 3.8441 (1.6); 3.8401 (3.1); 3.8236 (1.7); 3.7976 (1.0); 3.7946 (1.0); 3.7797 (2.2); 3.7770 (2.0); 3.7595 (1.7); 3.7434 (0.6); 3.5057 (16.0); 3.5028 (15.6); 1.9867 (0.7); 1.9840 (0.7); 1.9738 (0.7); 1.9703 (1.1); 1.9661 (1.0); 1.9621 (0.8); 1.9530 (1.1); 1.9487 (1.2); 1.9410 (1.2); 1.9373 (1.0); 1.9268 (1.4); 1.9202 (1.6); 1.9121 (1.6); 1.9020 (2.4); 1.8925 (1.8); 1.8850 (2.4); 1.8802 (1.8); 1.8642 (1.5); 1.8476 (0.6); 1.6307 (0.6); 1.6222 (0.6); 1.6132 (1.0); 1.6050 (1.3); 1.6005 (1.4); 1.5917 (2.0); 1.5879 (1.8); 1.5831 (2.6); 1.5748 (2.2); 1.5657 (1.6); 1.5563 (1.1); 1.3333 (0.7); 1.2845 (1.0); 1.2555 (1.3); 1.1078 (0.6); 0.0080 (1.5); -0.0002 (41.8); -0.0085 (1.2)

### Example No. I.10-72

¹H-NMR (400.0 MHz, CDCl₃): δ=8.3787 (3.2); 8.3747 (3.3); 8.3668 (3.4); 8.3626 (3.3); 7.6161 (2.7); 7.6120 (2.7); 7.5969 (3.0); 7.5929 (2.8); 7.5194 (0.8); 7.3712 (5.4); 7.3488 (5.4); 7.2605 (145.8); 7.0431 (3.4); 7.0310 (3.4); 7.0238 (3.1); 7.0118 (3.1); 6.9964 (0.8); 6.9610 (2.9); 6.9581 (2.9); 6.9428 (2.9); 6.9398 (2.8); 6.2954 (7.1); 4.1487 (1.2); 4.1326 (1.3); 4.1212 (2.0); 4.1092 (1.7); 4.1061 (1.6); 4.0934 (1.5); 4.0140 (1.5); 3.9968 (1.8); 3.9872 (1.1); 3.9766 (1.6); 3.9698 (1.5); 3.9496 (1.2); 3.9270 (16.0); 3.8336 (0.9); 3.8201 (1.0); 3.8129 (2.0); 3.7992 (2.0); 3.7923 (1.4); 3.7787 (1.4); 3.7625 (1.2); 3.7566 (1.2); 3.7446 (1.5); 3.7393 (3.1); 3.7217 (2.9); 3.7010 (1.9); 3.6806 (0.9); 3.5046 (15.4); 3.4912 (2.8); 3.4828 (2.2); 3.4691 (2.2); 2.5756 (0.9); 2.5597 (1.1); 2.5411 (0.9); 2.0451 (2.5); 2.0275 (0.6); 2.0137 (0.7); 2.0081 (0.9); 1.9952 (1.2); 1.9869 (0.7); 1.9826 (0.8); 1.9738 (1.1); 1.9622 (1.0); 1.9558 (0.6); 1.9416 (0.6); 1.6146 (0.7); 1.5957 (1.6); 1.5637 (4.8); 1.2773 (1.2); 1.2596 (2.6); 1.2415 (0.8); 0.8988 (1.0); 0.8821 (2.5); 0.8642 (1.0); 0.0079 (3.2); -0.0002 (83.2); -0.0085 (2.6)

### Example No. I.10-115

¹H-NMR (400.0 MHz, CDCl₃): δ=8.3922 (2.9); 8.3881 (2.8); 8.3802 (3.0); 8.3761 (2.7); 7.6202 (2.6); 7.6161 (2.6); 7.6010 (2.9); 7.5969 (2.7); 7.3767 (4.8); 7.3543 (4.8); 7.2606 (39.3); 7.0575 (3.0); 7.0455 (2.8); 7.0383 (2.8); 7.0263 (2.6); 6.9318 (4.2); 6.9136 (4.2); 6.3121 (0.6); 6.3078 (0.7); 6.2980 (6.8); 5.4498 (1.1); 5.4471 (1.1); 5.4340 (2.2); 5.4208 (1.2); 5.4181 (1.2); 5.4050 (0.6); 4.8709 (2.5); 4.8523 (4.0); 4.8339 (2.7); 4.6447 (2.6); 4.6315 (2.6); 4.6286 (2.4); 4.6259 (2.2); 4.6125 (2.0); 3.9469 (16.0); 3.9379 (0.8); 3.9306 (1.3); 3.5409 (0.8); 3.5269 (0.5); 3.5110 (11.4); 3.5081 (10.8); 1.5532 (0.9); 1.2546 (2.2); 1.2321 (0.7); 0.0080 (1.4); -0.0002 (51.6); -0.0085 (1.5)

### Example No. I.10-176

¹H-NMR (400.0 MHz, CDCl₃): δ=8.4351 (2.5); 8.4310 (2.5); 8.4230 (2.6); 8.4189 (2.5); 7.6834 (2.5); 7.6793 (2.5); 7.6642 (2.7); 7.6601 (2.6); 7.3732 (4.0); 7.3509 (4.0); 7.2603 (85.1); 7.0863 (2.6); 7.0742 (2.6); 7.0671 (2.5); 7.0550 (2.4); 6.8332 (3.6); 6.8151 (3.6); 6.2986 (6.6); 4.7467 (16.0); 4.1309 (0.8); 4.1130 (0.8); 3.9803 (0.9); 3.9396 (7.6); 3.9306 (7.6); 3.8899 (0.9); 3.5099 (9.8); 3.5071 (9.9); 2.0450 (3.7); 1.5506 (1.0); 1.2772 (1.3); 1.2594 (2.7); 1.2415 (1.1); 0.8989 (0.5); 0.8820 (1.5); 0.8642 (0.6); 0.0079 (1.9); -0.0002 (50.7); -0.0085 (1.8)

### Example No. I.14-2

¹H-NMR (400.0 MHz, CDCl₃): δ=7.9990 (0.7); 7.9949 (0.7); 7.9866 (0.8); 7.9826 (0.7); 7.8814 (1.0); 7.8600 (1.0); 7.5167 (0.7); 7.5127 (0.7); 7.4973 (0.8); 7.4932 (0.8); 7.2613 (38.0); 7.1365 (1.0); 7.1216 (1.0); 7.0022 (0.8); 6.9899 (0.8); 6.9828 (0.8); 6.9704 (0.7); 6.2800 (2.0); 4.9673 (0.7); 4.9275 (1.6); 4.8679 (1.6); 4.8280 (0.7); 4.1333 (0.8); 4.1280 (0.8); 4.1154 (0.9); 4.1103 (0.8); 3.5016 (2.8); 3.4988 (2.8); 1.5546 (16.0); 1.2673 (2.1); 1.2495 (4.4); 1.2316 (2.0); 0.0080 (0.6); -0.0002 (22.3); -0.0085 (0.6)

### Example No. I.14-23

¹H-NMR (600.0 MHz, CDCl₃): δ=7.9951 (2.2); 7.9926 (2.3); 7.9869 (2.3); 7.9844 (2.3); 7.8763 (3.2); 7.8620 (3.2); 7.5115 (2.2); 7.5090 (2.3); 7.4986 (2.4); 7.4961 (2.3); 7.2615 (13.0); 7.1335 (3.0); 7.1236 (3.0); 6.9959 (2.2); 6.9876 (2.2); 6.9829 (2.1); 6.9747 (2.0); 6.2797 (6.7); 5.2994 (2.0); 4.9573 (2.7); 4.9308 (4.6); 4.8640 (4.6); 4.8375 (2.6); 4.1494 (0.5); 4.1436 (0.8); 4.1367 (1.2); 4.1316 (2.4); 4.1246 (2.6); 4.1197 (2.6); 4.1127 (2.5); 4.1075 (1.2); 4.1008 (0.8); 4.0948 (0.4); 3.4998 (12.0); 2.1710 (0.5); 2.0445 (2.1); 1.5652 (50.0); 1.3012 (0.5); 1.2900 (0.6); 1.2800 (0.6); 1.2709 (1.3); 1.2608 (7.3); 1.2489 (12.4); 1.2370 (6.0); 0.8935 (1.2); 0.8821 (2.6); 0.8701 (1.3); -0.0001 (0.6)

### Example No. I.14-422

¹H-NMR (400.0 MHz, CDCl₃): δ=9.3524 (0.6); 7.9993 (1.1); 7.9951 (1.1); 7.9869 (1.2); 7.9828 (1.1); 7.8815 (1.4); 7.8602 (1.4); 7.5169 (1.1); 7.5128 (0.9); 7.4974 (1.2); 7.4936 (1.1); 7.2606 (47.9); 7.1371 (1.3); 7.1222 (1.4); 7.0020 (1.0); 6.9896 (1.2); 6.9825 (1.0); 6.9704 (1.0); 6.2797 (2.7); 4.9679 (1.1); 4.9281 (2.2); 4.8677 (2.2); 4.8278 (1.1); 4.1330 (1.2); 4.1276 (1.3); 4.1148 (1.3); 4.1097 (1.2); 3.5016 (4.4); 1.5456 (16.0); 1.2672 (2.8); 1.2494 (5.7); 1.2315 (2.7); 0.0079 (3.3); -0.0002 (50.9)

### Example No. I.15-1

¹H-NMR (400.0 MHz, CDCl₃): δ=7.9244 (1.5); 7.9208 (1.4); 7.9122 (1.6); 7.9085 (1.4); 7.3766 (2.3); 7.3545 (2.3); 7.3351 (1.5); 7.3314 (1.4); 7.3157 (1.7); 7.3120 (1.4); 7.2603 (13.9); 6.9412 (1.4); 6.9288 (1.5); 6.9217 (1.5); 6.9147 (2.4); 6.9094 (1.5); 6.8984 (2.2); 6.2869 (4.2); 5.0204 (1.1); 4.9806 (3.6); 4.9480 (3.5); 4.9082 (1.1); 4.2528 (1.2); 4.2474 (1.4); 4.2421 (2.2); 4.2349 (2.2); 4.2294 (1.4); 4.2241 (1.2); 3.5455 (2.5); 3.5340 (3.8); 3.5220 (2.4); 3.5032 (7.5); 3.3128 (16.0); 1.5779 (0.6); 1.2652 (0.8); 0.8821 (0.8); -0.0002 (15.5)

### Example No. I.15-6

¹H-NMR (400.0 MHz, CDCl₃): δ=7.8993 (2.4); 7.8875 (2.6); 7.3746 (3.4); 7.3521 (3.9); 7.3422 (3.5); 7.3251 (5.9); 7.3065 (9.2); 7.2598 (59.3); 6.9252 (2.2); 6.9124 (4.7); 6.8947 (4.8); 6.2696 (6.3); 5.0238 (1.6); 4.9842 (5.3); 4.9545 (5.4); 4.9141 (1.6); 4.5134 (12.4); 4.2951 (3.4); 4.2833 (4.2); 4.2719 (3.4); 3.6576 (3.6); 3.6457 (4.3); 3.6337 (3.2); 3.4829 (12.3); 1.5365 (16.0); 1.2650 (2.2); 0.8826 (2.1); 0.8649 (1.0); -0.0002 (80.1)

### Example No. I.15-23

¹H-NMR (400.0 MHz, CDCl₃): δ=7.9176 (1.2); 7.9136 (1.3); 7.9053 (1.3); 7.9013 (1.2); 7.3775 (1.8); 7.3555 (1.9); 7.3209 (1.2); 7.3170 (1.2); 7.3015 (1.4); 7.2976 (1.4); 7.2600 (32.7); 6.9364 (1.3); 6.9241 (1.4); 6.9182 (2.1); 6.9046 (1.4); 6.9022 (1.8); 6.2930 (3.0); 5.0193 (0.9); 4.9794 (2.7); 4.9453 (2.7); 4.9055 (0.9); 4.2753 (1.0); 4.2665 (1.5); 4.2530 (1.0); 3.6895 (1.8); 3.6773 (2.0); 3.6653 (1.6); 3.6114 (1.2); 3.6007 (1.7); 3.5957 (1.6); 3.5890 (2.5); 3.5255 (2.5); 3.5188 (1.7); 3.5062 (4.7); 3.5034 (5.4); 3.3722 (16.0); 1.5413 (11.6); 0.8822 (0.6); 0.0080 (1.8); -0.0002 (42.1); -0.0085 (1.8)

### Example No. I.15-26

¹H-NMR (400.0 MHz, CDCl₃): δ=7.9215 (1.2); 7.9176 (1.4); 7.9092 (1.4); 7.9053 (1.2); 7.3787 (1.8); 7.3565 (1.8); 7.3288 (1.2); 7.3248 (1.3); 7.3094 (1.5); 7.3054 (1.4); 7.2605 (23.1); 6.9403 (1.4); 6.9276 (2.6); 6.9209 (1.4); 6.9105 (1.9); 6.2996 (3.1); 4.9825 (0.9); 4.9428 (2.9); 4.9133 (2.8); 4.8736 (0.9); 4.2066 (0.8); 4.1991 (0.8); 4.1905 (1.5); 4.1831 (1.5); 4.1741 (0.9); 4.1669 (0.8); 3.5076 (4.5); 3.5047 (4.7); 3.3808 (1.7); 3.3652 (3.7); 3.3496 (1.8); 3.2912 (16.0); 2.6149 (2.5); 2.0451 (0.5); 1.8839 (1.6); 1.8679 (2.4); 1.8520 (1.6); 1.5463 (15.7); 1.2595 (0.8); 0.8821 (1.0); 0.0080 (1.2); -0.0002 (29.4); -0.0084 (1.5)

### Example No. I.15-31

¹H-NMR (400.0 MHz, CDCl₃): δ=7.9224 (1.1); 7.9184 (1.2); 7.9100 (1.2); 7.9060 (1.2); 7.3842 (1.6); 7.3621 (1.6); 7.3219 (1.1); 7.3179 (1.1); 7.3025 (1.3); 7.2985 (1.2); 7.2604 (18.4); 6.9442 (1.3); 6.9319 (1.2); 6.9248 (1.2); 6.9125 (1.2); 6.9030 (1.5); 6.8867 (1.5); 6.2985 (2.6); 5.3000 (3.8); 4.9964 (0.9); 4.9567 (2.6); 4.9237 (2.6); 4.8840 (0.9); 4.2939 (0.7); 4.2857 (0.8); 4.2769 (1.3); 4.2677 (1.1); 4.2588 (0.8); 4.2508 (0.7); 3.5130 (3.6); 3.5100 (3.7); 2.7105 (1.9); 2.6929 (2.3); 2.6756 (1.7); 2.1718 (1.7); 2.1221 (16.0); 1.5411 (5.2); 0.0080 (0.7); -0.0002 (26.6); -0.0085 (0.7)

### Example No. I.15-41

¹H-NMR (400.0 MHz, CDCl₃): δ=7.9150 (2.3); 7.9111 (2.5); 7.9027 (2.6); 7.8987 (2.5); 7.3888 (3.4); 7.3669 (3.4); 7.3301 (2.4); 7.3262 (2.5); 7.3107 (2.8); 7.3067 (2.6); 7.2601 (56.7); 7.2561 (1.0); 7.2553 (0.9); 6.9502 (2.8); 6.9379 (2.7); 6.9308 (2.6); 6.9184 (2.6); 6.8452 (3.2); 6.8290 (3.3); 6.2886 (5.6); 5.0254 (2.2); 4.9855 (5.4); 4.9409 (5.4); 4.9010 (2.2); 4.3622 (0.8); 4.3505 (2.4); 4.3384 (2.5); 4.3270 (0.9); 4.1224 (3.9); 4.1107 (6.3); 4.0989 (3.0); 3.5073 (7.7); 3.5043 (7.9); 1.5377 (16.0); 1.2628 (0.6); 0.8820 (1.1); 0.0079 (2.1); 0.0054 (0.5); 0.0046 (0.6); -0.0002 (79.4); -0.0027 (3.6); -0.0044 (1.5); -0.0052 (1.2); -0.0061 (1.0); -0.0069 (1.0); -0.0085 (2.5)

### Example No. I.15-72

¹H-NMR (400.0 MHz, CDCl₃): δ=7.9148 (1.2); 7.9109 (1.3); 7.9024 (1.3); 7.8986 (1.3); 7.3861 (1.8); 7.3641 (1.9); 7.3114 (1.0); 7.2932 (1.1); 7.2605 (19.8); 6.9428 (1.3); 6.9305 (1.4); 6.9234 (1.3); 6.9110 (1.3); 6.9066 (1.9); 6.8900 (1.8); 6.3145 (1.8); 6.2923 (1.8); 4.9908 (0.6); 4.9511 (1.7); 4.9474 (1.7); 4.9106 (1.7); 4.9075 (1.8); 4.8678 (0.6); 4.1307 (0.8); 4.1128 (1.1); 4.0947 (0.8); 4.0783 (0.6); 4.0492 (0.7); 4.0290 (0.7); 4.0150 (0.6); 3.9946 (0.6); 3.8131 (0.8); 3.7993 (0.9); 3.7926 (0.6); 3.7786 (0.6); 3.7352 (1.3); 3.7167 (1.5); 3.6981 (0.9); 3.5086 (5.9); 3.4945 (1.1); 3.4801 (0.6); 3.4724 (0.8); 3.4581 (0.5); 2.6148 (2.1); 2.5470 (0.5); 2.0450 (2.6); 1.5476 (16.0); 1.2771 (1.6); 1.2595 (3.1); 1.2417 (1.0); 0.8988 (1.2); 0.8820 (3.5); 0.8645 (1.5); -0.0002 (24.9); -0.0085 (1.2)

### Example No. I.15-115

¹H-NMR (400.0 MHz, CDCl₃): δ=7.9152 (1.8); 7.9112 (1.7); 7.9029 (2.0); 7.8989 (1.8); 7.3915 (2.6); 7.3693 (2.7); 7.3214 (1.8); 7.3174 (1.8); 7.3019 (2.0); 7.2979 (1.9); 7.2599 (44.3); 6.9556 (1.8); 6.9433 (1.8); 6.9362 (1.7); 6.9238 (1.8); 6.8632 (2.5); 6.8469 (2.5); 6.2904 (4.1); 5.4784 (0.7); 5.4625 (1.4); 5.4493 (0.9); 5.0216 (1.4); 4.9817 (3.9); 4.9452 (3.9); 4.9053 (1.4); 4.8642 (1.5); 4.8450 (2.4); 4.8284 (1.6); 4.6402 (0.9); 4.6327 (1.1); 4.6199 (1.6); 4.6095 (0.9); 4.6027 (0.8); 4.1309 (1.2); 4.1130 (1.2); 3.5113 (6.4); 3.5084 (6.3); 2.0448 (5.6); 1.5398 (16.0); 1.2772 (2.2); 1.2594 (4.4); 1.2415 (1.7); 0.8990 (0.9); 0.8821 (2.8); 0.8643 (1.1); 0.0080 (2.1); -0.0002 (58.6); -0.0085 (2.4)

### Example No. I.15-154

¹H-NMR (400.0 MHz, CDCl₃): δ=7.9187 (1.1); 7.9147 (1.2); 7.9064 (1.2); 7.9024 (1.1); 7.3787 (1.6); 7.3567 (1.6); 7.3205 (1.1); 7.3165 (1.1); 7.3011 (1.2); 7.2971 (1.2); 7.2617 (13.9); 6.9375 (1.3); 6.9252 (1.3); 6.9192 (1.9); 6.9057 (1.4); 6.9033 (1.6); 6.2936 (2.8); 5.0159 (0.8); 4.9762 (2.5); 4.9453 (2.5); 4.9056 (0.8); 4.2647 (1.0); 4.2552 (1.0); 4.2533 (1.1); 4.2493 (0.9); 4.2404 (1.1); 3.6882 (1.5); 3.6810 (0.6); 3.6783 (1.1); 3.6760 (1.6); 3.6640 (1.5); 3.6551 (16.0); 3.6487 (0.6); 3.6447 (1.6); 3.6427 (1.7); 3.6373 (1.4); 3.6302 (2.6); 3.6193 (7.0); 3.6166 (2.9); 3.6097 (0.6); 3.5573 (2.2); 3.5501 (1.3); 3.5453 (1.5); 3.5427 (1.3); 3.5345 (1.1); 3.5070 (3.7); 3.5041 (3.7); 3.3750 (15.9); 1.5863 (2.5); -0.0002 (15.2)

### Example No. I.15-166

¹H-NMR (400.0 MHz, CDCl₃): δ=7.9136 (2.0); 7.9097 (2.2); 7.9013 (2.3); 7.8973 (2.2); 7.3992 (3.0); 7.3773 (3.0); 7.3403 (2.0); 7.3364 (2.2); 7.3209 (2.4); 7.3169 (2.3); 7.2603 (64.0); 6.9791 (2.5); 6.9668 (2.4); 6.9597 (2.2); 6.9473 (2.2); 6.8460 (2.8); 6.8298 (2.9); 6.3297 (5.0); 5.3001 (16.0); 5.0149 (2.1); 4.9751 (4.7); 4.9210 (4.6); 4.8811 (2.1); 4.6101 (0.7); 4.5937 (1.2); 4.5792 (1.8); 4.5686 (1.2); 4.5654 (1.2); 4.5544 (1.9); 4.5399 (1.2); 4.5237 (0.7); 3.5179 (6.7); 3.5149 (7.0); 3.3072 (1.5); 3.2938 (2.9); 3.2799 (1.4); 2.9219 (14.0); 2.1719 (4.2); 1.5391 (15.6); 0.0079 (2.4); 0.0055 (0.7); -0.0002 (86.7); -0.0068 (1.0); -0.0085 (2.8)

### Example No. I.15-176

¹H-NMR (400.0 MHz, CDCl₃): δ=7.9361 (2.0); 7.9321 (2.1); 7.9238 (2.2); 7.9198 (2.2); 7.5193 (0.6); 7.3994 (3.3); 7.3774 (3.3); 7.3695 (2.1); 7.3655 (2.2); 7.3500 (2.4); 7.3460 (2.3); 7.2604 (104.1); 6.9964 (0.6); 6.9875 (2.4); 6.9752 (2.3); 6.9680 (2.2); 6.9557 (2.1); 6.8072 (3.1); 6.7909 (3.1); 6.3085 (5.4); 5.2999 (5.2); 5.0404 (1.6); 5.0002 (5.7); 4.9740 (5.6); 4.9338 (1.6); 4.7514 (16.0); 3.5163 (7.6); 3.5134 (7.8); 2.0074 (7.0); 1.5403 (7.8); 1.2536 (0.6); 0.0080 (2.1); -0.0002 (62.9); -0.0084 (1.8)

### Example No. I.15-201

¹H-NMR (400.0 MHz, CDCl₃): δ=7.8749 (2.8); 7.8709 (3.1); 7.8626 (3.1); 7.8586 (3.1); 7.3501 (4.1); 7.3279 (4.2); 7.2690 (2.9); 7.2650 (3.0); 7.2495 (3.4); 7.2456 (3.2); 7.2308 (52.3); 6.9191 (3.9); 6.9028 (4.0); 6.8941 (3.4); 6.8818 (3.3); 6.8747 (3.1); 6.8624 (3.1); 6.2636 (6.8); 4.9483 (1.2); 4.9090 (7.1); 4.8955 (7.1); 4.8562 (1.3); 3.8108 (0.5); 3.7757 (8.2); 3.7715 (8.2); 3.7364 (0.5); 3.4811 (9.2); 3.4781 (9.6); 1.9781 (8.2); 1.5108 (16.0); 0.0081 (3.4); -0.0002 (120.4); -0.0087 (3.4); -0.0215 (2.1); -0.0240 (0.6); -0.0248 (0.6); -0.0296 (71.6); -0.0354 (0.8); -0.0363 (0.7); -0.0379 (2.0)

### Example No. I.15-286

¹H-NMR (400.0 MHz, CDCl₃): δ=8.5414 (1.5); 8.5293 (1.5); 7.9055 (2.6); 7.9015 (2.8); 7.8931 (2.8); 7.8891 (2.8); 7.6904 (1.2); 7.6860 (1.2); 7.6712 (2.2); 7.6668 (2.2); 7.6519 (1.3); 7.6475 (1.3); 7.3678 (4.1); 7.3457 (4.2); 7.3396 (2.8); 7.3356 (2.8); 7.3202 (3.0); 7.3161 (2.8); 7.2847 (2.2); 7.2604 (56.0); 7.2243 (1.1); 7.2123 (1.1); 7.2058 (1.1); 7.1935 (1.0); 6.9416 (3.0); 6.9292 (2.9); 6.9221 (2.8); 6.9098 (2.8); 6.8922 (3.9); 6.8759 (4.0); 6.2342 (7.0); 5.6484 (0.6); 5.2610 (10.3); 5.0933 (1.5); 5.0535 (6.9); 5.0358 (6.9); 4.9959 (1.5); 4.1487 (1.1); 4.1309 (3.4); 4.1130 (3.4); 4.0952 (1.2); 3.4802 (9.5); 3.4774 (9.7); 2.0451 (16.0); 1.5511 (12.0); 1.3031 (0.7); 1.2773 (5.4); 1.2595 (10.9); 1.2416 (4.6); 0.8989 (1.9); 0.8820 (6.7); 0.8643 (2.6); 0.0080 (1.9); −0.0002 (73.3); -0.0085 (2.1)

### Example No. I.15-301

¹H-NMR (400.0 MHz, CDCl₃): δ=9.1426 (1.6); 9.1377 (1.6); 9.1308 (1.6); 9.1260 (1.6); 7.8939 (2.5); 7.8899 (2.7); 7.8816 (2.7); 7.8776 (2.6); 7.5174 (0.8); 7.5125 (0.9); 7.4962 (3.0); 7.4913 (2.8); 7.4819 (3.5); 7.4701 (3.1); 7.4607 (0.9); 7.4489 (1.1); 7.3757 (3.5); 7.3537 (3.4); 7.3342 (2.5); 7.3302 (2.6); 7.3148 (2.9); 7.3108 (2.8); 7.2655 (0.6); 7.2646 (0.8); 7.2613 (42.1); 7.2581 (0.9); 7.2573 (0.7); 6.9537 (3.0); 6.9413 (2.9); 6.9342 (2.8); 6.9219 (2.7); 6.8236 (3.3); 6.8073 (3.3); 6.2693 (5.8); 5.5450 (0.9); 5.5109 (4.8); 5.4960 (4.6); 5.4619 (0.9); 5.3001 (16.0); 5.0952 (1.8); 5.0554 (5.9); 5.0284 (5.8); 4.9885 (1.8); 3.5065 (7.8); 3.5035 (8.0); 1.5663 (3.8); 0.0079 (1.4); 0.0046 (0.6); 0.0037 (0.8); -0.0002 (52.6); -0.0028 (2.2); -0.0044 (0.9); -0.0053 (0.7); -0.0060 (0.6); -0.0069 (0.5); -0.0085 (1.6)

### Example No. I.15-405

¹H-NMR (400.0 MHz, CDCl₃): δ=7.9199 (1.2); 7.9159 (1.3); 7.9075 (1.3); 7.9036 (1.3); 7.3857 (1.9); 7.3637 (1.9); 7.3232 (1.2); 7.3192 (1.3); 7.3037 (1.4); 7.2998 (1.4); 7.2607 (28.3); 6.9457 (1.4); 6.9334 (1.4); 6.9263 (1.3); 6.9140 (1.3); 6.8899 (1.7); 6.8736 (1.7); 6.3012 (3.1); 5.3001 (2.7); 5.0213 (1.1); 4.9814 (2.9); 4.9422 (2.9); 4.9024 (1.1); 4.3201 (0.6); 4.3123 (0.7); 4.3084 (1.2); 4.3012 (1.1); 4.2994 (1.3); 4.2932 (1.1); 4.2904 (1.2); 4.2474 (1.2); 4.2445 (1.4); 4.2409 (0.5); 4.2323 (1.6); 4.2225 (0.7); 4.2206 (0.7); 3.5112 (4.1); 3.5083 (4.3); 2.1720 (2.0); 2.0313 (16.0); 1.5481 (13.3); 0.0079 (1.0); -0.0002 (33.4); -0.0085 (0.9)

### Example No. I.15-422

¹H-NMR (400.0 MHz, CDCl₃): δ=7.9188 (2.6); 7.9148 (2.8); 7.9065 (2.8); 7.9025 (2.8); 7.3773 (4.2); 7.3552 (4.2); 7.3374 (2.7); 7.3335 (2.7); 7.3180 (3.1); 7.3140 (2.9); 7.2606 (35.3); 6.9440 (3.0); 6.9316 (2.9); 6.9245 (2.8); 6.9100 (4.4); 6.8935 (4.0); 6.2922 (7.2); 5.0229 (1.7); 4.9830 (6.8); 4.9609 (6.7); 4.9209 (1.7); 4.6318 (2.0); 4.6181 (4.5); 4.6046 (2.2); 4.1122 (3.9); 4.1051 (4.0); 4.0989 (3.9); 4.0913 (3.6); 4.0767 (0.6); 3.6973 (0.5); 3.6847 (0.6); 3.6797 (1.6); 3.6738 (0.7); 3.6671 (1.6); 3.6618 (2.2); 3.6561 (2.2); 3.6494 (1.7); 3.6437 (2.6); 3.6385 (2.3); 3.6319 (0.6); 3.6259 (2.2); 3.6208 (0.8); 3.6083 (0.7); 3.5556 (0.7); 3.5496 (0.7); 3.5379 (2.1); 3.5320 (2.7); 3.5261 (0.7); 3.5202 (2.4); 3.5142 (4.4); 3.5072 (10.8); 3.5042 (10.8); 3.4968 (2.9); 3.4908 (1.8); 3.4791 (0.6); 3.4732 (0.5); 1.5462 (12.7); 1.2545 (1.0); 1.2368 (1.4); 1.2191 (0.7); 1.1998 (7.6); 1.1920 (7.8); 1.1822 (16.0); 1.1744 (15.8); 1.1645 (7.7); 1.1567 (7.5); 0.0079 (1.3); -0.0002 (49.0); -0.0085 (1.5)

### Example No. I.15-424

¹H-NMR (400.0 MHz, CDCl₃): δ=7.9299 (4.0); 7.9259 (4.2); 7.9176 (4.3); 7.9136 (4.2); 7.3754 (6.4); 7.3533 (6.6); 7.3462 (4.2); 7.3422 (4.1); 7.3267 (4.6); 7.3228 (4.5); 7.2603 (58.8); 6.9472 (4.7); 6.9348 (4.5); 6.9278 (4.3); 6.9154 (4.2); 6.9092 (6.2); 6.8929 (6.2); 6.2952 (11.0); 5.2998 (0.6); 5.0871 (3.5); 5.0778 (7.9); 5.0685 (3.8); 5.0365 (2.9); 4.9965 (10.4); 4.9707 (10.4); 4.9307 (2.9); 4.1579 (7.2); 4.1549 (7.3); 4.1488 (7.2); 4.1455 (6.8); 4.1162 (0.5); 3.9596 (1.5); 3.9428 (4.6); 3.9393 (4.1); 3.9350 (4.4); 3.9300 (4.4); 3.9267 (1.9); 3.9237 (4.7); 3.9135 (1.7); 3.9100 (2.0); 3.9049 (2.1); 3.9012 (1.6); 3.8900 (3.8); 3.8851 (4.1); 3.8800 (4.3); 3.8754 (4.0); 3.8723 (4.3); 3.8704 (4.3); 3.8564 (1.4); 3.5045 (15.0); 3.5016 (15.3); 1.5428 (16.0); 1.2640 (2.8); 0.8990 (1.4); 0.8820 (4.7); 0.8642 (1.9); 0.0693 (0.6); 0.0080 (2.1); -0.0002 (77.5); -0.0085 (2.4)

### Example No. I.16-422

¹H-NMR (400.0 MHz, CDCl₃): δ=7.9223 (1.2); 7.9183 (1.3); 7.9100 (1.3); 7.9060 (1.2); 7.5398 (1.8); 7.5184 (1.8); 7.3462 (1.2); 7.3422 (1.2); 7.3268 (1.4); 7.3228 (1.3); 7.2618 (9.6); 6.9456 (1.5); 6.9333 (1.4); 6.9262 (1.2); 6.9138 (1.2); 6.8774 (2.0); 6.8612 (1.9); 6.2889 (3.9); 5.0223 (0.8); 4.9823 (2.8); 4.9778 (1.0); 4.9589 (2.8); 4.9478 (0.9); 4.9190 (0.8); 4.6311 (0.8); 4.6174 (1.9); 4.6084 (0.7); 4.6040 (0.9); 4.1127 (0.6); 4.1097 (1.6); 4.1020 (1.6); 4.0963 (2.1); 4.0881 (1.7); 4.0827 (0.6); 4.0727 (0.6); 3.6794 (0.8); 3.6665 (0.7); 3.6617 (1.0); 3.6559 (1.1); 3.6488 (0.8); 3.6431 (1.2); 3.6383 (1.2); 3.6254 (1.0); 3.5379 (1.0); 3.5319 (1.2); 3.5203 (1.1); 3.5143 (1.8); 3.5122 (1.0); 3.5043 (5.4); 3.5016 (5.6); 3.4908 (0.8); 2.1718 (2.0); 2.0454 (1.7); 1.5713 (16.0); 1.2773 (1.0); 1.2645 (1.7); 1.2596 (2.0); 1.2546 (1.0); 1.2417 (0.7); 1.2369 (0.9); 1.1999 (4.3); 1.1918 (3.9); 1.1823 (8.9); 1.1742 (7.8); 1.1646 (4.2); 1.1566 (3.7); 0.8989 (0.9); 0.8820 (3.4); 0.8643 (1.2); -0.0002 (8.6)

### Example No. I.31-23

¹H-NMR (400.0 MHz, CDCl₃): δ=7.8484 (5.9); 7.8438 (6.0); 7.4184 (4.6); 7.4100 (5.4); 7.4062 (5.2); 7.3951 (4.7); 7.2604 (33.9); 7.2515 (3.2); 7.2435 (3.1); 7.2389 (2.7); 6.3259 (9.4); 4.9567 (2.3); 4.9300 (7.8); 4.9096 (7.6); 4.8830 (2.3); 4.7410 (0.4); 4.3105 (0.4); 4.3025 (0.4); 4.2880 (0.4); 4.2760 (2.9); 4.2735 (3.2); 4.2688 (5.1); 4.2648 (5.1); 4.2601 (3.4); 4.2576 (3.1); 4.2453 (0.4); 3.7282 (1.8); 3.7202 (0.6); 3.7124 (0.5); 3.7007 (5.2); 3.6927 (7.5); 3.6846 (4.9); 3.6749 (0.4); 3.6696 (0.4); 3.6621 (0.5); 3.6539 (0.6); 3.6306 (4.6); 3.6233 (6.3); 3.6200 (4.6); 3.6152 (6.5); 3.5696 (0.5); 3.5596 (0.8); 3.5542 (1.0); 3.5424 (6.9); 3.5374 (5.8); 3.5299 (19.2); 3.3931 (2.5); 3.3829 (35.6); 3.3650 (0.5); 2.1637 (0.4); 1.5493 (50.0); 1.2551 (1.1); -0.0001 (46.3)

### Example No. I.35-1

¹H-NMR (400.0 MHz, CDCl₃): δ=8.0118 (1.3); 8.0077 (1.3); 7.9994 (1.3); 7.9954 (1.3); 7.5491 (1.3); 7.5450 (1.3); 7.5297 (1.4); 7.5256 (1.4); 7.5110 (2.0); 7.4901 (2.0); 7.2635 (2.2); 7.0158 (1.4); 7.0035 (1.4); 6.9964 (1.4); 6.9841 (1.3); 6.9637 (1.9); 6.9492 (1.9); 6.2719 (3.6); 5.0022 (1.4); 4.9624 (2.8); 4.8951 (2.8); 4.8552 (1.4); 4.2026 (0.9); 4.1916 (2.5); 4.1794 (2.5); 4.1680 (0.8); 3.5152 (2.2); 3.5034 (4.1); 3.4903 (5.8); 3.4872 (5.1); 3.2949 (16.0); 1.6014 (1.0); -0.0002 (3.2)

### Example No. I.36-1

¹H-NMR (400.0 MHz, CDCl₃): δ=7.7738 (2.0); 7.7672 (2.1); 7.3975 (1.9); 7.3755 (1.9); 7.2607 (9.8); 7.1175 (1.2); 7.1109 (1.1); 7.0974 (1.2); 7.0909 (1.1); 7.0004 (1.8); 6.9842 (1.8); 6.3063 (3.2); 5.0009 (0.8); 4.9609 (3.0); 4.9349 (3.0); 4.8950 (0.8); 4.2622 (1.0); 4.2591 (1.0); 4.2517 (1.4); 4.2457 (1.3); 4.2387 (1.0); 4.2357 (1.0); 3.5625 (2.0); 3.5507 (2.2); 3.5389 (1.9); 3.5202 (4.5); 3.5175 (4.7); 3.3369 (16.0); 2.0450 (1.1); 1.5511 (4.4); 1.2772 (0.5); 1.2595 (1.1); 0.8820 (1.2); -0.0002 (12.9)

### Example No. I.36-23

¹H-NMR (400.0 MHz, CDCl₃): δ=7.7686 (1.5); 7.7620 (1.5); 7.3987 (1.4); 7.3767 (1.4); 7.2601 (36.0); 7.1037 (0.8); 7.0972 (0.8); 7.0835 (0.9); 7.0769 (0.8); 7.0058 (1.3); 6.9895 (1.3); 6.3117 (2.3); 4.9964 (0.6); 4.9566 (2.1); 4.9299 (2.1); 4.8899 (0.6); 4.2824 (0.9); 4.2726 (1.0); 4.2586 (1.0); 3.6951 (1.3); 3.6829 (1.4); 3.6710 (1.2); 3.6192 (0.8); 3.6090 (1.2); 3.6035 (1.0); 3.5968 (1.9); 3.5318 (1.9); 3.5196 (4.4); 3.5090 (0.9); 3.3750 (11.3); 1.5380 (16.0); 0.0080 (1.2); -0.0002 (47.6); -0.0085 (1.5)

The present invention furthermore provides the use of one or more compounds of the general formula (I) according to the invention and/or salts thereof, as defined above, preferably in one of the embodiments identified as preferred or particularly preferred, in particular one or more compounds of the formulae (I.1) to (I.36) and/or salts thereof, in each case as defined above, as herbicide and/or plant growth regulator, preferably in crops of useful plants and/or ornamental plants.

The present invention further provides a method for controlling harmful plants and/or for regulating the growth of plants, characterized in that an effective amount
of one or more compounds of the general formula (I) according to the invention and/or salts thereof, as defined above, preferably in one of the embodiments identified as preferred or particularly preferred, in particular one or more compounds of the formulae (I.1) to (I.36) and/or salts thereof, in each case as defined above, or
of a composition according to the invention, as defined below, is applied to the (harmful) plants, seeds of (harmful) plants, the soil in which or on which the (harmful) plants grow or the area under cultivation.

The present invention also provides a method for controlling unwanted plants, preferably in crops of useful plants, characterized in that an effective amount
of one or more compounds of the general formula (I) and/or salts thereof, as defined above, preferably in one of the embodiments identified as preferred or particularly preferred, in particular one or more compounds of the formulae (I.1) to (I.36) and/or salts thereof, in each case as defined above, or
of a composition according to the invention, as defined below, is applied to unwanted plants (for example harmful plants such as mono- or dicotyledonous weeds or unwanted crop plants), the seed of the unwanted plants (i.e. plant seeds, for example grains, seeds or vegetative propagation organs such as tubers or shoot parts with buds), the soil in which or on which the unwanted plants grow (for example the soil of crop land or non-crop land) or the area under cultivation (i.e. the area on which the unwanted plants will grow).

The present invention also further provides methods for controlling for regulating the growth of plants, preferably of useful plants, characterized in that an effective amount
of one or more compounds of the general formula (I) and/or salts thereof, as defined above, preferably in one of the embodiments identified as preferred or particularly preferred, in particular one or more compounds of the formulae (I.1) to (I.36) and/or salts thereof, in each case as defined above, or
of a composition according to the invention, as defined below, is applied to the plant, the seed of the plant (i.e. plant seeds, for example grains, seeds or vegetative propagation organs such as tubers or shoot parts with buds), the soil in which or on which the plants grow (for example the soil of crop land or non-crop land) or the area under cultivation (i.e. the area on which the plants will grow).

In this context, the compounds according to the invention or the compositions according to the invention can be applied for example by pre-sowing (if appropriate also by incorporation into the soil), pre-emergence and/or post-emergence processes. Specific examples of some representatives of the monocotyledonous and dicotyledonous weed flora which can be controlled by the compounds according to the invention are as follows, though there is no intention to restrict the enumeration to particular species.

In a method according to the invention for controlling harmful plants or for regulating the growth of plants, one or more compounds of the general formula (I) and/or salts thereof are preferably employed for controlling harmful plants or for regulating growth in crops of useful plants or ornamental plants, where in a preferred embodiment the useful plants or ornamental plants are transgenic plants.

The compounds of the general formula (I) according to the invention and/or their salts are suitable for controlling the following genera of monocotyledonous and dicotyledonous harmful plants:

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.
Dicotyledonous harmful plants of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

When the compounds according to the invention are applied to the soil surface before germination of the harmful plants (weed grasses and/or broad-leaved weeds) (pre-emergence method), either the seedlings of the weed grasses or broad-leaved weeds are prevented completely from emerging or they grow until they have reached the cotyledon stage, but then stop growing and eventually, after three to four weeks have elapsed, die completely.

If the active compounds are applied post-emergence to the green parts of the plants, growth stops after the treatment, and the harmful plants remain at the growth stage at the time of application, or they die completely after a certain time, so that in this manner competition by the weeds, which is harmful to the crop plants, is eliminated very early and in a sustained manner.

Although the compounds according to the invention display an outstanding herbicidal activity against monocotyledonous and dicotyledonous weeds, crop plants of economically important crops, for example dicotyledonous crops of the genera Arachis, Beta, Brassica, Cucumis, Cucurbita, Helianthus, Daucus, Glycine, Gossypium, Ipomoea, Lactuca, Linum, Lycopersicon, Miscanthus, Nicotiana, Phaseolus, Pisum, Solanum, Vicia, or monocotyledonous crops of the genera Allium, Ananas, Asparagus, Avena, Hordeum, Oryza, Panicum, Saccharum, Secale, Sorghum, Triticale, Triticum, Zea, are damaged only to an insignificant extent, or not at all, depending on the structure of the respective compound according to the invention and its application rate. For these reasons, the present compounds are very suitable for selective control of unwanted plant growth in plant crops such as agriculturally useful plants or ornamental plants.

In addition, the compounds of the invention (depending on their particular structure and the application rate deployed) have outstanding growth-regulating properties in crop plants. They intervene in the plants' own metabolism with regulatory effect, and can thus be used for the controlled influencing of plant constituents and to facilitate harvesting, for example by triggering desiccation and stunted growth. Furthermore, they are also suitable for the general control and inhibition of unwanted vegetative growth without killing the plants in the process.

Inhibition of vegetative growth plays a major role for many mono- and dicotyledonous crops since, for example, this can reduce or completely prevent lodging.

By virtue of their herbicidal and plant growth regulatory properties, the active compounds can also be used to control harmful plants in crops of genetically modified plants or plants modified by conventional mutagenesis. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material.

It is preferred with a view to transgenic crops to use the compounds according to the invention and/or their salts in economically important transgenic crops of useful plants and ornamentals, for example of cereals such as wheat, barley, rye, oats, millet, rice and corn or else crops of sugar beet, cotton, soybean, oilseed rape, potato, tomato, peas and other vegetables.

It is preferred to employ the compounds according to the invention as herbicides in crops of useful plants which are resistant, or have been made resistant by recombinant means, to the phytotoxic effects of the herbicides.

By virtue of their herbicidal and plant growth regulatory properties, the active compounds can also be used to control harmful plants in crops of genetically modified plants which are known or are yet to be developed. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material. Further special properties may be tolerance or resistance to abiotic stressors, for example heat, cold, drought, salinity and ultraviolet radiation.

Preference is given to the use of the compounds of the general formula (I) according to the invention or salts thereof in economically important transgenic crops of useful plants and ornamentals, for example of cereals such as wheat, barley, rye, oats, triticale, millet, rice, cassava and corn, or else crops of sugar beet, cotton, soybean, oilseed rape, potatoes, tomatoes, peas and other vegetables.

It is preferable to employ the compounds of the general formula (I) as herbicides in crops of useful plants which are resistant, or have been made resistant by recombinant means, to the phytotoxic effects of the herbicides.

Conventional ways of producing novel plants which have modified properties in comparison to existing plants consist, for example, in traditional cultivation methods and the generation of mutants. Alternatively, novel plants with altered properties can be generated with the aid of recombinant methods.

A large number of molecular-biological techniques by means of which novel transgenic plants with modified properties can be generated are known to the person skilled in the art. For such genetic manipulations, nucleic acid molecules which allow mutagenesis or sequence alteration by recombination of DNA sequences can be introduced into plasmids. With the aid of standard methods, it is possible, for example, to undertake base exchanges, remove part sequences or add natural or synthetic sequences. To connect the DNA fragments to each other, adapters or linkers may be added to the fragments.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product.

To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Such sequences are known to those skilled in the art (see, for example, Braun et al., EMBO J. 11 (1992), 3219-3227). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants.

Thus, transgenic plants can be obtained whose properties are altered by overexpression, suppression or inhibition of homologous (=natural) genes or gene sequences or expression of heterologous (=foreign) genes or gene sequences.

It is preferred to employ the compounds (I) according to the invention in transgenic crops which are resistant to growth regulators such as, for example, dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, glyphosates, glufosinates or benzoylisoxazoles and analogous active compounds.

When the active compounds of the invention are employed in transgenic crops, not only do the effects towards harmful plants to be observed in other crops occur, but frequently also effects which are specific to the application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also relates to the use of the compounds of the general formula (I) according to the invention and/or their salts as herbicides for controlling harmful plants in crops of useful plants or ornamentals, optionally in transgenic crop plants.

Preference is given to the use in cereals, here preferably corn, wheat, barley, rye, oats, millet or rice, by the pre- or post-emergence method.

Preference is also given to the use in soybeans by the pre- or post-emergence method.

The use according to the invention for the control of harmful plants or for growth regulation of plants also includes the case in which the active compound of the general formula (I) or its salt is not formed from a precursor substance (“prodrug”) until after application on the plant, in the plant or in the soil.

The invention also provides the use of one or more compounds of the general formula (I) or salts thereof or of a composition according to the invention (as defined below) (in a method) for controlling harmful plants or for regulating the growth of plants which comprises applying an effective amount of one or more compounds of the general formula (I) or salts thereof onto the plants (harmful plants, if appropriate together with the useful plants), plant seeds, the soil in which or on which the plants grow or the area under cultivation.

The invention also provides a herbicidal and/or plant growth-regulating composition, characterized in that the composition comprises

(a) one or more compounds of the general formula (I) and/or salts thereof, as defined above, preferably in one of the embodiments identified as preferred or particularly preferred, in particular one or more compounds of the formulae (I.1) to (I.36) and/or salts thereof, in each case as defined above, and
(b) one or more further substances selected from groups (i) and/or (ii):
(i) one or more further agrochemically active substances, preferably selected from the group consisting of insecticides, acaricides, nematicides, further herbicides (i.e. those not corresponding to the formula (I) defined above), fungicides, safeners, fertilizers and/or further growth regulators,
(ii) one or more formulation auxiliaries customary in crop protection.

Here, the further agrochemically active substances of component (i) of a composition according to the invention are preferably selected from the group of substances mentioned in “The Pesticide Manual”, 16th edition, The British Crop Protection Council and the Royal Soc. of Chemistry, 2012.

A herbicidal or plant growth-regulating composition according to the invention comprises preferably one, two, three or more formulation auxiliaries (ii) customary in crop protection selected from the group consisting of surfactants, emulsifiers, dispersants, film-formers, thickeners, inorganic salts, dusting agents, carriers solid at 25° C. and 1013 mbar, preferably adsorptive granulated inert materials, wetting agents, antioxidants, stabilizers, buffer substances, antifoam agents, water, organic solvents, preferably organic solvents miscible with water in any ratio at 25° C. and 1013 mbar.

The compounds (I) according to the invention can be used in the form of wettable powders, emulsifiable concentrates, sprayable solutions, dusting products or granules in the customary formulations. The invention therefore also provides herbicidal and plant growth-regulating compositions which comprise compounds of the general formula (I) and/or salts thereof.

The compounds of the general formula (I) and/or salts thereof can be formulated in various ways according to which biological and/or physicochemical parameters are required. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes.

These individual formulation types and the formulation assistants, such as inert materials, surfactants, solvents and further additives, are known to the person skilled in the art and are described, for example, in: Watkins, “Handbook of Insecticide Dust Diluents and Carriers”, 2nd Ed., Darland Books, Caldwell N.J.; H.v. Olphen, “Introduction to Clay Colloid Chemistry”, 2nd ed., J. Wiley & Sons, N.Y.; C. Marsden, “Solvents Guide”, 2nd ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [Interface-active Ethylene Oxide Adducts], Wiss. Verlagsgesellschaft, Stuttgart 1976; Winnacker-Kuchler, “Chemische Technologie” [Chemical Technology], volume 7, C. Hanser Verlag Munich, 4th Ed. 1986.

Wettable powders are preparations which can be dispersed uniformly in water and, in addition to the active compound, apart from a diluent or inert substance, also comprise surfactants of the ionic and/or nonionic type (wetting agents, dispersants), for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the herbicidal active compounds are finely ground, for example in customary apparatuses such as hammer mills, blower mills and air-jet mills, and simultaneously or subsequently mixed with the formulation auxiliaries.

Emulsifiable concentrates are produced by dissolving the active compound in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonate salts, for example calcium dodecylbenzenesulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters, or polyoxyethylene sorbitan esters, for example polyoxyethylene sorbitan fatty acid esters.

Dusting products are obtained by grinding the active compound with finely distributed solids, for example talc, natural clays, such as kaolin, bentonite and pyrophyllite, or diatomaceous earth.

Suspension concentrates may be water- or oil-based. They may be prepared, for example, by wet-grinding by means of commercial bead mills and optional addition of surfactants as have, for example, already been listed above for the other formulation types.

Emulsions, for example oil-in-water emulsions (EW), can be produced, for example, by means of stirrers, colloid mills and/or static mixers using aqueous organic solvents and optionally surfactants as have, for example, already been listed above for the other formulation types.

Granules can be prepared either by spraying the active compound onto granular inert material capable of adsorption or by applying active compound concentrates to the surface of carrier substances, such as sand, kaolinites or granular inert material, by means of adhesives, for example polyvinyl alcohol, sodium polyacrylate or else mineral oils. Suitable active compounds can also be granulated in the manner customary for the production of fertilizer granules—if desired as a mixture with fertilizers.

Water-dispersible granules are produced generally by the customary processes such as spray-drying, fluidized-bed granulation, pan granulation, mixing with high-speed mixers and extrusion without solid inert material.

For the production of pan, fluidized-bed, extruder and spray granules, see e.g. processes in “Spray-Drying Handbook” 3rd Ed. 1979, G. Goodwin Ltd., London; J. E. Browning, “Agglomeration”, Chemical and Engineering 1967, pages 147 ff; “Perry's Chemical Engineer's Handbook”, 5th Ed., McGraw Hill, New York 1973, p. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical preparations, preferably herbicidal or plant growth-regulating compositions, of the present invention preferably comprise a total amount of from 0.1 to 99% by weight, preferably 0.5 to 95% by weight, particularly preferably 1 to 90% by weight, especially preferably 2 to 80% by weight, of active compounds of the general formula (I) and their salts.

In wettable powders, the active compound concentration is, for example, about 10% to 90% by weight, the remainder to 100% by weight consisting of customary formulation constituents. In emulsifiable concentrates, the active compound concentration may be about 1% to 90% and preferably 5% to 80% by weight. Formulations in the form of dusts comprise 1% to 30% by weight of active compound, preferably usually 5% to 20% by weight of active compound; sprayable solutions contain about 0.05% to 80% by weight, preferably 2% to 50% by weight of active compound. In the case of water-dispersible granules, the active compound content depends partially on whether the active compound is in liquid or solid form and on which granulation auxiliaries, fillers, etc., are used. In the water-dispersible granules, the content of active compound is, for example, between 1 and 95% by weight, preferably between 10 and 80% by weight.

In addition, the active compound formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity. Examples of formulation auxiliaries are described inter alia in “Chemistry and Technology of Agrochemical Formulations”, ed. D. A. Knowles, Kluwer Academic Publishers (1998).

The compounds of the general formula (I) or salts thereof can be used as such or in the form of their preparations (formulations) in a combination with other pesticidally active substances, for example insecticides, acaricides, nematicides, herbicides, fungicides, safeners, fertilizers and/or growth regulators, for example in the form of a finished formulation or of a tank mix. The combination formulations can be prepared on the basis of the abovementioned formulations, while taking account of the physical properties and stabilities of the active compounds to be combined.

Active compounds which can be employed in combination with the compounds of the general formula (I) according to the invention in mixture formulations or in a tank mix are, for example, known active compounds based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II, protoporphyrinogen oxidase, as described, for example, in Weed Research 26 (1986) 441-445 or “The Pesticide Manual”, 16th edition, The British Crop Protection Council and the Royal Soc. of Chemistry, 2012 and literature cited therein.

Of particular interest is the selective control of harmful plants in crops of useful plants and ornamentals. Although the compounds (I) according to the invention have already demonstrated very good to adequate selectivity in a large number of crops, in principle, in some crops and in particular also in the case of mixtures with other, less selective herbicides, phytotoxicities on the crop plants may occur. In this connection, combinations of compounds (I) according to the invention are of particular interest which comprise the compounds (I) or their combinations with other herbicides or pesticides and safeners. The safeners, which are used in an antidotically effective amount, reduce the phytotoxic side effects of the herbicides/pesticides employed, for example in economically important crops, such as cereals (wheat, barley, rye, corn, rice, millet), sugarbeet, sugarcane, oilseed rape, cotton and soybeans, preferably cereals.

The weight ratios of herbicide (mixture) to safener depend generally on the herbicide application rate and the efficacy of the safener in question and may vary within wide limits, for example in the range from 200:1 to 1:200, preferably 100:1 to 1:100, in particular 20:1 to 1:20.

Analogously to the compounds (I) or mixtures thereof, the safeners can be formulated with further herbicides/pesticides and be provided and employed as a finished formulation or tank mix with the herbicides.

For application, the herbicide or herbicide/safener formulations present in commercial form are, if appropriate, diluted in a customary manner, for example in the case of wettable powders, emulsifiable concentrates, dispersions and water-dispersible granules with water. Dust-type preparations, granules for soil application or granules for scattering and sprayable solutions are not normally diluted further with other inert substances prior to application.

The application rate of the compounds of the general formula (I) and/or their salts is affected to a certain extent by external conditions such as temperature, humidity, etc. Here, the application rate may vary within wide limits. For the application as a herbicide for controlling harmful plants, the total amount of compounds of the general formula (I) and their salts is preferably in the range from 0.001 to 10.0 kg/ha, with preference in the range from 0.005 to 5 kg/ha, more preferably in the range from 0.01 to 1.5 kg/ha, particularly preferably in the range from 0.05 to 1 kg/ha. This applies both to the pre-emergence or the post-emergence application.

When compounds of the general formula (I) and/or their salts are used as plant growth regulator, for example as culm stabilizer for crop plants like those mentioned above, preferably cereal plants, such as wheat, barley, rye, triticale, millet, rice or corn, the total application rate is preferably in the range of from 0.001 to 2 kg/ha, preferably in the range of from 0.005 to 1 kg/ha, in particular in the range of from 10 to 500 g/ha, very particularly preferably in the range from 20 to 250 g/ha. This applies both to the pre-emergence and the post-emergence application.

The application as culm stabilizer may take place at various stages of the growth of the plants.

Preferred is, for example, the application after the tillering phase, at the beginning of the longitudinal growth.

As an alternative, application as plant growth regulator is also possible by treating the seed, which includes various techniques for dressing and coating seed. Here, the application rate depends on the particular techniques and can be determined in preliminary tests.

Active compounds which can be employed in combination with the compounds of the general formula (I) according to the invention in compositions according to the invention (for example in mixed formulations or in the tank mix) are, for example, known active compounds which are based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II or protoporphyrinogen oxidase, as are described in, for example, Weed Research 26 (1986) 441-445 or “The Pesticide Manual”, 16th edition, The British Crop Protection Council and the Royal Soc. of Chemistry, 2012 and the literature cited therein. Known herbicides or plant growth regulators which can be combined with the compounds of the invention are, for example, the following, where said active compounds are designated either with their “common name” in accordance with the International Organization for Standardization (ISO) or with the chemical name or with the code number. They always encompass all the use forms, for example acids, salts, esters and also all isomeric forms such as stereoisomers and optical isomers, even if they are not mentioned explicitly.

Examples of such herbicidal mixing partners are:

acetochlor, acifluorfen, acifluorfen-sodium, aclonifen, alachlor, allidochlor, alloxydim, alloxydim-sodium, ametryn, amicarbazone, amidochlor, amidosulfuron, 4-amino-3-chloro-6-(4-chloro-2-fluoro-3-methylphenyl)-5-fluoropyridine-2-carboxylic acid, aminocyclopyrachlor, aminocyclopyrachlor-potassium, aminocyclopyrachlor-methyl, aminopyralid, amitrole, ammonium sulfamate, anilofos, asulam, atrazine, azafenidin, azimsulfuron, beflubutamid, benazolin, benazolin-ethyl, benfluralin, benfuresate, bensulfuron, bensulfuron-methyl, bensulide, bentazone, benzobicyclon, benzofenap, bicyclopyron, bifenox, bilanafos, bilanafos-sodium, bispyribac, bispyribac-sodium, bromacil, bromobutide, bromofenoxim, bromoxynil, bromoxynil-butyrate, -potassium, -heptanoate and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, chlorfenac, chlorfenac-sodium, chlorfenprop, chlorflurenol, chlorflurenol-methyl, chloridazon, chlorimuron, chlorimuron-ethyl, chlorophthalim, chlorotoluron, chlorthal-dimethyl, chlorsulfuron, cinidon, cinidon-ethyl, cinmethylin, cinosulfuron, clacyfos, clethodim, clodinafop, clodinafop-propargyl, clomazone, clomeprop, clopyralid, cloransulam, cloransulam-methyl, cumyluron, cyanamide, cyanazine, cycloate, cyclopyrimorate, cyclosulfamuron, cycloxydim, cyhalofop, cyhalofop-butyl, cyprazine, 2,4-D, 2,4-D-butotyl, -butyl, -dimethylammonium, -diolamine, -ethyl, 2-ethylhexyl, -isobutyl, -isooctyl, -isopropylammonium, -potassium, -triisopropanolammonium and -trolamine, 2,4-DB, 2,4-DB-butyl, -dimethylammonium, isooctyl, -potassium and -sodium, daimuron (dymron), dalapon, dazomet, n-decanol, desmedipham, detosyl-pyrazolate (DTP), dicamba, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimethametryn, dimethenamid, dimethenamid-P, dimetrasulfuron, dinitramine, dinoterb, diphenamid, diquat, diquat-dibromid, dithiopyr, diuron, DNOC, endothal, EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-9600, F-5231, i.e. N-[2-chloro-4-fluoro-5-[4-(3-fluoropropyl)-4,5-dihydro-5-oxo-1H-tetrazol-1-yl]phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-P-ethyl, fenoxasulfone, fenquinotrione, fentrazamide, flamprop, flamprop-M-isopropyl, flamprop-M-methyl, flazasulfuron, florasulam, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyrsulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium, -sodium and -trimesium, H-9201, i.e. 0-(2,4-dimethyl-6-nitrophenyl)O-ethyl isopropylphosphoramidothioate, halauxifen, halauxifen-methyl, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl (2,4-dichlorophenoxy)acetate, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, lactofen, lenacil, linuron, MCPA, MCPA-butotyl, -dimethylammonium, -2-ethylhexyl, -isopropylammonium, -potassium and -sodium, MCPB, MCPB-methyl, -ethyl and -sodium, mecoprop, mecoprop-sodium, and -butotyl, mecoprop-P, mecoprop-P-butotyl, -dimethylammonium, -2-ethylhexyl and -potassium, mefenacet, mefluidide, mesosulfuron, mesosulfuron-methyl, mesotrione, methabenzthiazuron, metam, metamifop, metamitron, metazachlor, metazosulfuron, methabenzthiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinate, monolinuron, monosulfuron, monosulfuron-ester, MT-5950, i.e. N-[3-chloro-4-(1-methylethyl)phenyl]-2-methylpentanamide, NGGC-011, napropamide, NC-310, i.e. 4-(2,4-dichlorobenzoyl)-1-methyl-5-benzyloxypyrazole, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxyfluorfen, paraquat, paraquat dichloride, pebulate, pendimethalin, penoxsulam, pentachlorophenol, pentoxazone, pethoxamid, petroleum oils, phenmedipham, picloram, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, sethoxydim, siduron, simazine, simetryn, SL-261, sulcotrion, sulfentrazone, sulfometuron, sulfometuron-methyl, sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, 2,3,6-TBA, TCA (trifluoroacetic acid), TCA-sodium, tebuthiuron, tefuryltrione, tembotrione, tepraloxydim, terbacil, terbucarb, terbumeton, terbuthylazin, terbutryn, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methyl, tritosulfuron, urea sulfate, vernolate, XDE-848, ZJ-0862, i.e. 3,4-dichloro-N-{2-[(4,6-dimethoxypyrimidin-2-yl)oxy]benzyl}aniline, and the following compounds:

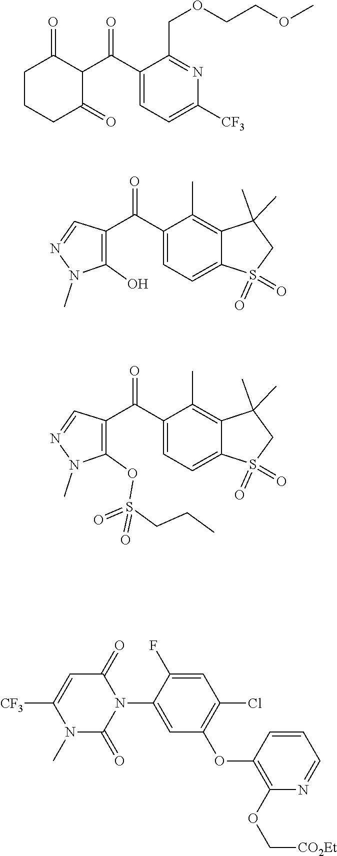 [SMILES: COCCOCc1nc(C(F)(F)F)ccc1C(=O)C1C(=O)CCCC1=O ; Cc1c(C(=O)c2cnn(C)c2O)ccc2c1C(C)(C)CS2(=O)=O ; CCCS(=O)(=O)Oc1c(C(=O)c2ccc3c(c2C)C(C)(C)CS3(=O)=O)cnn1C ; CCOC(=O)COc1ncccc1Oc1cc(-n2c(=O)cc(C(F)(F)F)n(C)c2=O)c(F)cc1Cl]

Examples of plant growth regulators as possible mixing partners are:

acibenzolar, acibenzolar-S-methyl, 5-aminolevulinic acid, ancymidol, 6-benzylaminopurine, brassinolide, catechol, chlormequat chloride, cloprop, cyclanilide, 3-(cycloprop-1-enyl)propionic acid, daminozide, dazomet, n-decanol, dikegulac, dikegulac-sodium, endothal, endothal-dipotassium, -disodium, and mono(N,N-dimethylalkylammonium), ethephon, flumetralin, flurenol, flurenol-butyl, flurprimidol, forchlorfenuron, gibberellic acid, inabenfide, indole-3-acetic acid (IAA), 4-indol-3-ylbutyric acid, isoprothiolane, probenazole, jasmonic acid, jasmonic acid methyl ester, maleic hydrazide, mepiquat chloride, 1-methylcyclopropene, 2-(1-naphthyl)acetamide, 1-naphthylacetic acid, 2-naphthyloxyacetic acid, nitrophenolate mixture, 4-oxo-4[(2-phenylethyl)amino]butyric acid, paclobutrazole, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, uniconazole-P.

Useful combination partners for the compounds of the general formula (I) according to the invention also include, for example, the following safeners:
S1) Compounds from the group of heterocyclic carboxylic acid derivatives:
S1^{a}) Compounds of the dichlorophenylpyrazoline-3-carboxylic acid type (S1^{a}), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874;
S1^{b}) Derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described in EP-A-333131 and EP-A-269806;
S1^{c}) Derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described, for example, in EP-A-268554;
S1^{d}) Compounds of the triazolecarboxylic acid type (S1^{d}), preferably compounds such as fenchlorazole(-ethyl ester), i.e. ethyl 1-(2,4-dichlorophenyl)-5-trichloromethyl-1H-1,2,4-triazole-3-carboxylate (51-7), and related compounds, as described in EP-A-174562 and EP-A-346620;
S1^{e}) Compounds of the 5-benzyl- or 5-phenyl-2-isoxazoline-3-carboxylic acid or of the 5,5-diphenyl-2-isoxazoline-3-carboxylic acid type (S1^{d}), preferably compounds such as ethyl 5-(2,4-dichlorobenzyl)-2-isoxazoline-3-carboxylate (S1-8) or ethyl 5-phenyl-2-isoxazoline-3-carboxylate (51-9) and related compounds as described in WO-A-91/08202, or 5,5-diphenyl-2-isoxazolinecarboxylic acid (51-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13) as described in patent application WO-A-95/07897.
S2) Compounds from the group of the 8-quinolinoxy derivatives (S2):
S2^{a}) Compounds of the 8-quinolinoxyacetic acid type (S2^{a}), preferably 1-methylhexyl (5-chloro-8-quinolinoxy)acetate (“cloquintocet-mexyl”) (S2-1), 1,3-dimethylbut-1-yl (5-chloro-8-quinolinoxy)acetate (S2-2), 4-allyloxybutyl (5-chloro-8-quinolinoxy)acetate (S2-3), 1-allyloxyprop-2-yl (5-chloro-8-quinolinoxy)acetate (S2-4), ethyl (5-chloro-8-quinolinoxy)acetate (S2-5), methyl (5-chloro-8-quinolinoxy)acetate (S2-6), allyl (5-chloro-8-quinolinoxy)acetate (S2-7), 2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86750, EP-A-94349 and EP-A-191736 or EP-A-0 492 366, and also (5-chloro-8-quinolinoxy)acetic acid (S2-10), hydrates and salts thereof, for example the lithium, sodium, potassium, calcium, magnesium, aluminum, iron, ammonium, quaternary ammonium, sulfonium or phosphonium salts thereof, as described in WO-A-2002/34048;
S2^{b}) Compounds of the (5-chloro-8-quinolinoxy)malonic acid type (S2^{b}), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
S3) Active compounds of the dichloroacetamide type (S3), which are frequently used as pre-emergence safeners (soil-acting safeners), for example
   “dichlormid” (N,N-diallyl-2,2-dichloroacetamide) (S3-1),
   “R-29148” (3-dichloroacetyl-2,2,5-trimethyl-1,3-oxazolidine) from Stauffer (S3-2),
   “R-28725” (3-dichloroacetyl-2,2-dimethyl-1,3-oxazolidine) from Stauffer (S3-3),
   “benoxacor” (4-dichloroacetyl-3,4-dihydro-3-methyl-2H-1,4-benzoxazine) (S3-4),
   “PPG-1292” (N-allyl-N-[(1,3-dioxolan-2-yl)methyl]dichloroacetamide) from PPG Industries (S3-5),
   “DKA-24” (N-allyl-N-[(allylaminocarbonyl)methyl]dichloroacetamide) from Sagro-Chem (S3-6),
   “AD-67” or “MON 4660” (3-dichloroacetyl-1-oxa-3-azaspiro[4.5]decane) from Nitrokemia or Monsanto (S3-7),
   “TI-35” (1-dichloroacetylazepane) from TRI-Chemical RT (S3-8),
   “diclonon” (dicyclonon) or “BAS145138” or “LAB145138” (S3-9) ((RS)-1-dichloroacetyl-3,3,8a-trimethylperhydropyrrolo[1,2-a]pyrimidin-6-one) from BASF,
   “furilazole” or “MON 13900” ((RS)-3-dichloroacetyl-5-(2-furyl)-2,2-dimethyloxazolidine) (53-10), and the (R) isomer thereof (53-11).
S4) Compounds from the class of the acylsulfonamides (S4):
S4^{a})N-Acylsulfonamides of the formula (S4^{a}) and salts thereof, as described in WO-A-97/45016,

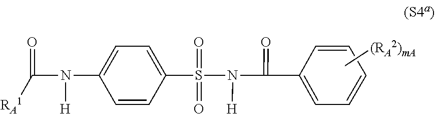

in which
R_{A}¹ represents (C₁-C₆)-alkyl, (C₃-C₆)-cycloalkyl, where the 2 latter radicals are substituted by v_{A} substituents from the group of halogen, (C₁-C₄)-alkoxy, (C₁-C₆)-haloalkoxy and (C₁-C₄)-alkylthio and, in the case of cyclic radicals, also by (C₁-C₄)-alkyl and (C₁-C₄)-haloalkyl;
R_{A}² represents halogen, (C₁-C₄)-alkyl, (C₁-C₄)-alkoxy, CF₃;
m_{A} represents 1 or 2;
x_{A} represents 0, 1, 2 or 3;
S4^{b}) Compounds of the 4-(benzoylsulfamoyl)benzamide type of the formula (S4^{b}) and salts thereof, as described in WO-A-99/16744,

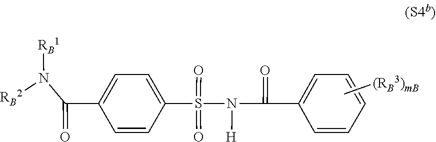

in which
R_{B}¹, R_{B}² independently of one another represent hydrogen, (C₁-C₆)-alkyl, (C₃-C₆)-cycloalkyl, (C₃-C₆)-alkenyl, (C₃-C₆)-alkynyl,
R_{B}³ represents halogen, (C₁-C₄)-alkyl, (C₁-C₄)-haloalkyl or (C₁-C₄)-alkoxy and
m_{B} represents 1 or 2,
   e.g. those in which
   R_{B}¹=cyclopropyl, R_{B}²=hydrogen and (R_{B}³)=2-OMe (“cyprosulfamide”, S4-1),
   R_{B}¹=cyclopropyl, R_{B}²=hydrogen and (R_{B}³)=5-Cl-2-OMe (S4-2),
   R_{B}¹=ethyl, R_{B}²=hydrogen and (R_{B}³)=2-OMe (S4-3),
   R_{B}¹=isopropyl, R_{B}²=hydrogen and (R_{B}³)=5-Cl-2-OMe (S4-4) and
   R_{B}¹=isopropyl, R_{B}²=hydrogen and (R_{B}³)=2-OMe (S4-5);
S4^{c}) Compounds from the class of the benzoylsulfamoylphenylureas of the formula (S4^{c}), as described in EP-A-365484,

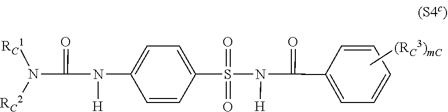

in which
R_{C}¹, R_{C}² independently of one another represent hydrogen, (C₁-C₈)-alkyl, (C₃-C₈)-cycloalkyl, (C₃-C₆)-alkenyl, (C₃-C₆)-alkynyl,
R_{C}³ represents halogen, (C₁-C₄)-alkyl, (C₁-C₄)-alkoxy, CF₃ and
m_{C} represents 1 or 2;
for example
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea;
S4^{d}) Compounds of the N-phenylsulfonylterephthalamide type of the formula (S4^{d}) and salts thereof, which are known, for example, from CN 101838227,

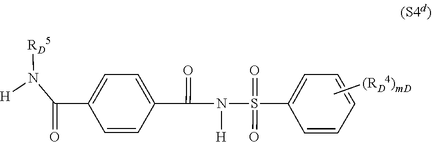

in which
R_{D}⁴ is halogen, (C₁-C₄)-alkyl, (C₁-C₄)-alkoxy, CF₃;
m_{D} is 1 or 2;
R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl.
S5) Active compounds from the class of the hydroxyaromatics and the aromatic-aliphatic carboxylic acid derivatives (S5), for example ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.
S6) Active compounds from the class of the 1,2-dihydroquinoxalin-2-ones (S6), for example 1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.
S7) Compounds from the class of the diphenylmethoxyacetic acid derivatives (S7), e.g. methyl diphenylmethoxyacetate (CAS Reg. No. 41858-19-9) (S7-1), ethyl diphenylmethoxyacetate or diphenylmethoxyacetic acid, as described in WO-A-98/38856.
S8) Compounds of the formula (S8), as described in WO-A-98/27049,

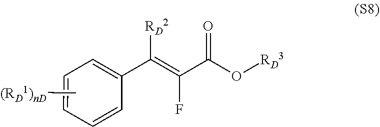

in which the symbols and indices are defined as follows:
R_{D}¹ represents halogen, (C₁-C₄)-alkyl, (C₁-C₄)-haloalkyl, (C₁-C₄)-alkoxy, (C₁-C₄)-haloalkoxy,
R_{D}² represents hydrogen or (C₁-C₄)-alkyl,
R_{D}³ represents hydrogen, (C₁-C₈)-alkyl, (C₂-C₄)-alkenyl, (C₂-C₄)-alkynyl or aryl, where each of the abovementioned carbon-containing radicals is unsubstituted or substituted by one or more, preferably up to three identical or different radicals from the group consisting of halogen and alkoxy; or salts thereof,
   n_{D} represents an integer from 0 to 2.
S9) Active compounds from the class of the 3-(5-tetrazolylcarbonyl)-2-quinolones (S9), for example 1,2-dihydro-4-hydroxy-1-ethyl-3-(5-tetrazolylcarbonyl)-2-quinolone (CAS Reg. No.: 219479-18-2), 1,2-dihydro-4-hydroxy-1-methyl-3-(5-tetrazolylcarbonyl)-2-quinolone (CAS Reg. No. 95855-00-8), as described in WO-A-1999/000020.
S10) Compounds of the formulae (S10^{a}) or (S10^{b}) as described in WO-A-2007/023719 and WO-A-2007/023764

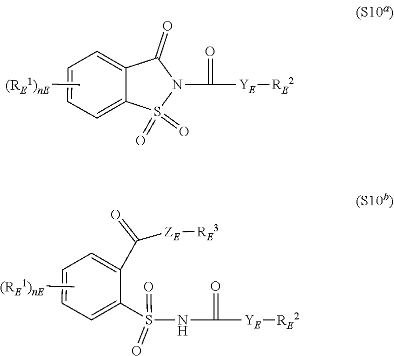

in which
R_{E}¹ represents halogen, (C₁-C₄)-alkyl, methoxy, nitro, cyano, CF₃, OCF₃,
Y_{E}, Z_{E} independently of one another represent O or S,
n_{E} represents an integer from 0 to 4,
R_{E}² represents (C₁-C₁₆)-alkyl, (C₂-C₆)-alkenyl, (C₃-C₆)-cycloalkyl, aryl; benzyl, halobenzyl,
R_{E}³ represents hydrogen or (C₁-C₆)-alkyl.
S11) Active compounds of the oxyimino compounds type (S11), which are known as seed-dressing agents, for example
   “oxabetrinil” ((Z)-1,3-dioxolan-2-ylmethoxyimino(phenyl)acetonitrile) (S11-1), which is known as a seed-dressing safener for millet/sorghum against metolachlor damage,
   “fluxofenim” (1-(4-chlorophenyl)-2,2,2-trifluoro-1-ethanone O-(1,3-dioxolan-2-ylmethyl)oxime) (S11-2), which is known as a seed-dressing safener for millet/sorghum against metolachlor damage, and
   “cyometrinil” or “CGA-43089” ((Z)-cyanomethoxyimino(phenyl)acetonitrile) (S11-3), which is known as a seed-dressing safener for millet/sorghum against metolachlor damage.
S12) Active compounds from the class of the isothiochromanones (S12), for example methyl [(3-oxo-1H-2-benzothiopyran-4(3H)-ylidene)methoxy]acetate (CAS Reg. No. 205121-04-6) (S12-1) and related compounds from WO-A-1998/13361.
S13) One or more compounds from group (S13):
   “naphthalic anhydride” (1,8-naphthalenedicarboxylic anhydride) (513-1), which is known as a seed-dressing safener for corn against thiocarbamate herbicide damage,
   “fenclorim” (4,6-dichloro-2-phenylpyrimidine) (S13-2), which is known as a safener for pretilachlor in sown rice,
   “flurazole” (benzyl 2-chloro-4-trifluoromethyl-1,3-thiazole-5-carboxylate) (S13-3), which is known as a seed-dressing safener for millet/sorghum against alachlor and metolachlor damage,
   “CL 304415” (CAS Reg. No. 31541-57-8) (4-carboxy-3,4-dihydro-2H-1-benzopyran-4-acetic acid) (S13-4) from American Cyanamid, which is known as a safener for corn against damage by imidazolinones,
   “MG 191” (CAS Reg. No. 96420-72-3) (2-dichloromethyl-2-methyl-1,3-dioxolane) (S13-5) from Nitrokemia, which is known as a safener for corn,
   “MG 838” (CAS Reg. No. 133993-74-5) (2-propenyl 1-oxa-4-azaspiro[4.5]decane-4-carbodithioate) (S13-6) from Nitrokemia
   “disulfoton” (0,0-diethyl S-2-ethylthioethyl phosphorodithioate) (S13-7),
   “dietholate” (0,0-diethyl O-phenyl phosphorothioate) (S13-8),
   “mephenate” (4-chlorophenyl methylcarbamate) (S13-9).
S14) Active compounds which, in addition to herbicidal action against harmful plants, also have safener action on crop plants such as rice, for example
   “dimepiperate” or “MY-93” (S-1-methyl 1-phenylethylpiperidine-1-carbothioate), which is known as a safener for rice against damage by the herbicide molinate,
   “daimuron” or “SK 23” (1-(1-methyl-1-phenylethyl)-3-p-tolylurea), which is known as a safener for rice against damage by the herbicide imazosulfuron,
   “cumyluron”=“JC-940” (3-(2-chlorophenylmethyl)-1-(1-methyl-1-phenylethyl)urea, see JP-A-60087254), which is known as a safener for rice against damage by some herbicides,
   “methoxyphenone” or “NK 049” (3,3′-dimethyl-4-methoxybenzophenone), which is known as a safener for rice against damage by some herbicides,
   “CSB” (1-bromo-4-(chloromethylsulfonyl)benzene) from Kumiai, (CAS Reg. No. 54091-06-4), which is known as a safener against damage by some herbicides in rice.
S15) Compounds of the formula (S15) or tautomers thereof

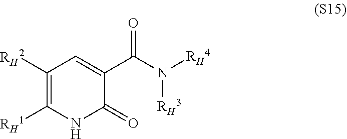

as described in WO-A-2008/131861 and WO-A-2008/131860
in which
R_{H}¹ is a (C₁-C₆)-haloalkyl radical and
R_{H}² is hydrogen or halogen and
R_{H}³, R_{H}⁴ independently of one another are hydrogen, (C₁-C₁₆)-alkyl, (C₂-C₁₆)-alkenyl or (C₂-C₁₆)-alkynyl,
   where each of the 3 last-mentioned radicals is unsubstituted or substituted by one or more radicals from the group of halogen, hydroxy, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
   or (C₃-C₆)-cycloalkyl, (C₄-C₆)-cycloalkenyl, (C₃-C₆)-cycloalkyl fused on one side of the ring to a 4- to 6-membered saturated or unsaturated carbocyclic ring,
   or (C₄-C₆)-cycloalkenyl fused on one side of the ring to a 4- to 6-membered saturated or unsaturated carbocyclic ring,
   where each of the 4 last-mentioned radicals is unsubstituted or substituted by one or more radicals from the group of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
   or
R_{H}³ is (C₁-C₄)-alkoxy, (C₂-C₄)-alkenyloxy, (C₂-C₆)-alkynyloxy or (C₂-C₄)-haloalkoxy and
R_{H}⁴ is hydrogen or (C₁-C₄)-alkyl or
R_{H}³ and R_{H}⁴ together with the directly attached nitrogen atom represent a four- to eight-membered heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio.
S16) Active compounds which are used primarily as herbicides but also have safener action on crop plants, for example
   (2,4-dichlorophenoxy)acetic acid (2,4-D),
   (4-chlorophenoxy)acetic acid,
   (R,S)-2-(4-chloro-o-tolyloxy)propionic acid (mecoprop),
   4-(2,4-dichlorophenoxy)butyric acid (2,4-DB),
   (4-chloro-o-tolyloxy)acetic acid (MCPA),
   4-(4-chloro-o-tolyloxy)butyric acid,
   4-(4-chlorophenoxy)butyric acid,
   3,6-dichloro-2-methoxybenzoic acid (dicamba),
   1-(ethoxycarbonyl)ethyl 3,6-dichloro-2-methoxybenzoate (lactidichlor-ethyl).

Preferred safeners in combination with the compounds of the general formula (I) according to the invention and/or salts thereof, in particular with the compounds of the formulae (I.1) to (I.34) and/or salts thereof, are: cloquintocet-mexyl, cyprosulfamide, fenchlorazole ethyl ester, isoxadifen-ethyl, mefenpyr-diethyl, fenclorim, cumyluron, S4-1 and S4-5, and particularly preferred safeners are: cloquintocet-mexyl, cyprosulfamide, isoxadifen-ethyl and mefenpyr-diethyl.

### BIOLOGICAL EXAMPLES

a. Post-Emergence Herbicidal Action and Crop Plant Compatibility

Seeds of monocotyledonous and dicotyledonous weeds and crop plants were placed in sandy loam in plastic or wood-fiber pots, covered with soil and cultivated in a greenhouse under controlled growth conditions. 2 to 3 weeks after sowing, the test plants were treated at the one-leaf stage. The compounds of the invention, formulated in the form of wettable powders (WP) or as emulsion concentrates (EC), were then sprayed onto the green parts of the plants as aqueous suspension or emulsion with addition of 0.5% additive at a water application rate of 600 l/ha (converted). After the test plants had been kept in the greenhouse under optimum growth conditions for about 3 weeks, the activity of the preparations was rated visually in comparison to untreated controls. For example, 100% activity=the plants have died, 0% activity=like control plants.

Tables A1 to A15 below show the effects of selected compounds of the general formula (I) according to Tables I.1 to I.36 on various harmful plants and at an application rate corresponding to 20 g/ha and less, which were obtained by the experimental procedure mentioned above.

TABLE A1

| Compound | Alopecurus myosuroides | Application rate |
|---|---|---|
| Example No. | (efficacy in %) | [g/ha] |
| I.1-1 | 80 | 20 |
| I.1-71 | 80 | 5 |
| I.10-1 | 80 | 5 |
| I.10-26 | 80 | 5 |
| I.10-71 | 80 | 5 |
| I.10-72 | 80 | 20 |
| I.10-115 | 80 | 20 |
| I.10-176 | 80 | 5 |
| I.15-1 | 80 | 5 |
| I.15-23 | 100 | 20 |
| I.15-26 | 90 | 20 |
| I.15-71 | 90 | 5 |
| I.15-72 | 90 | 5 |
| I.15-115 | 80 | 5 |
| I.15-176 | 80 | 5 |
| I.15-280 | 90 | 5 |
| I.15-2 | 100 | 20 |
| I.16-1 | 100 | 20 |
| I.16-2 | 100 | 20 |
| I.16-23 | 100 | 20 |
| I.16-421 | 100 | 20 |
| I.15-421 | 100 | 20 |
| I.16-176 | 100 | 20 |
| I.16-422 | 100 | 20 |
| I.16-424 | 100 | 20 |
| I.16-71 | 100 | 20 |
| I.16-115 | 100 | 20 |
| I.15-422 | 100 | 20 |
| I.14-1 | 90 | 20 |
| I.14-2 | 100 | 20 |
| I.14-422 | 100 | 20 |
| I.14-115 | 100 | 20 |
| I.35-1 | 100 | 20 |
| I.35-23 | 100 | 20 |
| I.31-23 | 90 | 20 |
| I.31-1 | 90 | 20 |
| I.1-286 | 100 | 20 |
| I.1-176 | 100 | 20 |
| I.1-115 | 100 | 20 |
| I.36-1 | 100 | 20 |
| I.36-23 | 100 | 20 |
| I.36-176 | 100 | 20 |
| I.36-286 | 100 | 20 |
| I.15-368 | 100 | 20 |
| I.15-366 | 100 | 20 |
| I.15-367 | 100 | 20 |

TABLE A2

| Compound | Avena fatua | Application rate |
|---|---|---|
| Example No. | (efficacy in %) | [g/ha] |
| I.1-1 | 80 | 20 |
| I.1-71 | 80 | 20 |
| I.10-1 | 80 | 20 |
| I.10-71 | 80 | 20 |
| I.15-1 | 90 | 5 |
| I.15-23 | 80 | 5 |
| I.15-26 | 90 | 20 |
| I.15-71 | 80 | 20 |
| I.15-72 | 80 | 5 |
| I.15-115 | 90 | 20 |
| I.15-176 | 90 | 20 |
| I.15-424 | 100 | 20 |
| I.15-2 | 90 | 20 |
| I.16-1 | 90 | 20 |
| I.16-2 | 100 | 20 |
| I.16-23 | 100 | 20 |
| I.16-421 | 100 | 20 |
| I.15-421 | 100 | 20 |
| I.16-176 | 90 | 20 |
| I.16-422 | 100 | 20 |
| I.16-424 | 90 | 20 |
| I.16-71 | 90 | 20 |
| I.16-115 | 80 | 20 |
| I.15-422 | 80 | 20 |
| I.14-1 | 80 | 20 |
| I.14-2 | 100 | 20 |
| I.14-422 | 90 | 20 |
| I.14-115 | 80 | 20 |
| I.35-1 | 100 | 20 |
| I.35-23 | 90 | 20 |
| I.1-286 | 80 | 20 |
| I.1-176 | 90 | 20 |
| I.1-115 | 80 | 20 |
| I.36-1 | 100 | 20 |
| I.36-176 | 80 | 20 |
| I.36-286 | 80 | 20 |
| I.15-368 | 100 | 20 |
| I.15-366 | 90 | 20 |
| I.15-367 | 90 | 20 |

TABLE A3

| Compound | Digitaria sanguinalis | Application rate |
|---|---|---|
| Example No. | (efficacy in %) | [g/ha] |
| I.1-1 | 100 | 20 |
| I.1-71 | 100 | 5 |
| I.10-1 | 80 | 5 |
| I.10-26 | 100 | 20 |
| I.10-71 | 90 | 5 |
| I.10-72 | 80 | 5 |
| I.10-115 | 100 | 20 |
| I.10-176 | 100 | 20 |
| I.15-1 | 100 | 5 |
| I.15-6 | 100 | 5 |
| I.15-23 | 100 | 5 |
| I.15-26 | 100 | 5 |
| I.15-31 | 100 | 5 |
| I.15-41 | 100 | 5 |
| I.15-71 | 100 | 5 |
| I.15-72 | 100 | 5 |
| I.15-115 | 100 | 20 |
| I.15-154 | 100 | 20 |
| I.15-176 | 100 | 5 |
| I.15-201 | 100 | 5 |
| I.15-211 | 100 | 5 |
| I.15-280 | 100 | 5 |
| I.15-286 | 100 | 5 |
| I.15-405 | 100 | 5 |
| I.15-424 | 100 | 5 |
| I.15-2 | 100 | 20 |
| I.16-1 | 100 | 20 |
| I.16-2 | 100 | 20 |
| I.16-23 | 100 | 20 |
| I.16-421 | 100 | 20 |
| I.15-421 | 100 | 20 |
| I.16-176 | 100 | 20 |
| I.16-422 | 100 | 20 |
| I.16-424 | 100 | 20 |
| I.16-71 | 100 | 20 |
| I.16-115 | 100 | 20 |
| I.15-422 | 100 | 20 |
| I.14-1 | 100 | 20 |
| I.14-2 | 100 | 20 |
| I.14-23 | 100 | 20 |
| I.14-422 | 100 | 20 |
| I.14-115 | 100 | 20 |
| I.35-1 | 100 | 20 |
| I.35-23 | 100 | 20 |
| I.31-23 | 100 | 20 |
| I.31-1 | 100 | 20 |
| I.1-176 | 100 | 20 |
| I.1-115 | 100 | 20 |
| I.36-1 | 100 | 20 |
| I.36-23 | 100 | 20 |
| I.36-176 | 100 | 20 |
| I.36-286 | 100 | 20 |
| I.15-368 | 100 | 20 |
| I.15-366 | 100 | 20 |
| I.15-367 | 100 | 20 |

TABLE A4

| Compound | Echinochloa crus-galli | Application rate |
|---|---|---|
| Example No. | (efficacy in %) | [g/ha] |
| I.1-1 | 100 | 20 |
| I.1-71 | 100 | 5 |
| I.10-1 | 100 | 5 |
| I.10-26 | 100 | 20 |
| I.10-71 | 100 | 5 |
| I.10-72 | 100 | 5 |
| I.10-115 | 100 | 20 |
| I.10-176 | 100 | 5 |
| I.15-1 | 100 | 5 |
| I.15-6 | 100 | 5 |
| I.15-23 | 100 | 5 |
| I.15-26 | 100 | 5 |
| I.15-41 | 100 | 5 |
| I.15-71 | 100 | 5 |
| I.15-72 | 100 | 5 |
| I.15-115 | 100 | 20 |
| I.15-176 | 100 | 5 |
| I.15-201 | 100 | 5 |
| I.15-211 | 100 | 5 |
| I.15-280 | 80 | 5 |
| I.15-286 | 100 | 5 |
| I.15-288 | 100 | 5 |
| I.15-301 | 100 | 5 |
| I.15-350 | 100 | 5 |
| I.15-405 | 100 | 5 |
| I.15-424 | 100 | 5 |
| I.15-2 | 100 | 20 |
| I.16-1 | 100 | 20 |
| I.16-2 | 100 | 20 |
| I.16-23 | 100 | 20 |
| I.16-421 | 100 | 20 |
| I.15-421 | 100 | 20 |
| I.16-176 | 100 | 20 |
| I.16-422 | 100 | 20 |
| I.16-424 | 100 | 20 |
| I.16-71 | 100 | 20 |
| I.16-115 | 100 | 20 |
| I.15-422 | 100 | 20 |
| I.14-1 | 100 | 20 |
| I.14-2 | 100 | 20 |
| I.14-23 | 100 | 20 |
| I.14-422 | 100 | 20 |
| I.14-115 | 100 | 20 |
| I.35-1 | 100 | 20 |
| I.35-23 | 100 | 20 |
| I.31-23 | 100 | 20 |
| I.31-1 | 100 | 20 |
| I.1-286 | 100 | 20 |
| I.1-176 | 100 | 20 |
| I.1-115 | 100 | 20 |
| I.36-1 | 100 | 20 |
| I.36-23 | 100 | 20 |
| I.36-176 | 100 | 20 |
| I.36-286 | 100 | 20 |
| I.15-368 | 100 | 20 |
| I.15-366 | 100 | 20 |
| I.15-367 | 100 | 20 |

TABLE A5

| Compound | Lolium rigidum | Application rate |
|---|---|---|
| Example No. | (efficacy in %) | [g/ha] |
| I.1-1 | 80 | 20 |
| I.1-71 | 100 | 5 |
| I.10-26 | 80 | 20 |
| I.10-71 | 80 | 20 |
| I.10-72 | 80 | 20 |
| I.15-1 | 100 | 20 |
| I.15-23 | 80 | 5 |
| I.15-26 | 80 | 5 |
| I.15-71 | 100 | 20 |
| I.15-72 | 100 | 20 |
| I.15-115 | 90 | 20 |
| I.15-2 | 90 | 20 |
| I.16-1 | 90 | 20 |
| I.16-2 | 100 | 20 |
| I.16-23 | 100 | 20 |
| I.16-421 | 90 | 20 |
| I.15-421 | 100 | 20 |
| I.16-176 | 80 | 20 |
| I.16-422 | 90 | 20 |
| I.16-424 | 80 | 20 |
| I.16-71 | 90 | 20 |
| I.16-115 | 100 | 20 |
| I.15-422 | 100 | 20 |
| I.14-1 | 80 | 20 |
| I.14-2 | 90 | 20 |
| I.14-422 | 80 | 20 |
| I.35-1 | 80 | 20 |
| I.31-1 | 80 | 20 |
| I.1-286 | 80 | 20 |
| I.1-176 | 80 | 20 |
| I.1-115 | 80 | 20 |
| I.36-1 | 90 | 20 |
| I.36-23 | 90 | 20 |
| I.36-176 | 80 | 20 |
| I.36-286 | 80 | 20 |
| I.15-368 | 90 | 20 |
| I.15-366 | 90 | 20 |
| I.15-367 | 80 | 20 |

TABLE A6

| Compound | Setaria viridis | Application rate |
|---|---|---|
| Example No. | (efficacy in %) | [g/ha] |
| I.1-1 | 100 | 5 |
| I.1-71 | 100 | 5 |
| I.10-1 | 80 | 5 |
| I.10-26 | 100 | 5 |
| I.10-71 | 100 | 5 |
| I.10-72 | 90 | 5 |
| I.10-115 | 100 | 5 |
| I.10-176 | 100 | 5 |
| I.15-1 | 100 | 5 |
| I.15-23 | 100 | 5 |
| I.15-26 | 100 | 5 |
| I.15-71 | 100 | 5 |
| I.15-72 | 100 | 5 |
| I.15-115 | 100 | 20 |
| I.15-176 | 100 | 5 |
| I.15-280 | 100 | 5 |
| I.15-2 | 100 | 20 |
| I.16-1 | 100 | 20 |
| I.16-2 | 100 | 20 |
| I.16-23 | 100 | 20 |
| I.16-421 | 100 | 20 |
| I.15-421 | 100 | 20 |
| I.16-176 | 100 | 20 |
| I.16-422 | 100 | 20 |
| I.16-424 | 100 | 20 |
| I.16-71 | 100 | 20 |
| I.16-115 | 100 | 20 |
| I.15-422 | 100 | 20 |
| I.14-1 | 100 | 20 |
| I.14-2 | 100 | 20 |
| I.14-23 | 100 | 20 |
| I.14-422 | 100 | 20 |
| I.14-115 | 100 | 20 |
| I.35-1 | 100 | 20 |
| I.35-23 | 100 | 20 |
| I.31-23 | 100 | 20 |
| I.31-1 | 100 | 20 |
| I.1-176 | 100 | 20 |
| I.1-115 | 100 | 20 |
| I.36-1 | 100 | 20 |
| I.36-23 | 100 | 20 |
| I.36-176 | 100 | 20 |
| I.36-286 | 100 | 20 |
| I.15-368 | 100 | 20 |
| I.15-366 | 100 | 20 |
| I.15-367 | 100 | 20 |

TABLE A7

| Compound | Abutilon theophrasti | Application rate |
|---|---|---|
| Example No. | (efficacy in %) | [g/ha] |
| I.1-1 | 100 | 20 |
| I.1-71 | 100 | 5 |
| I.10-1 | 80 | 5 |
| I.10-26 | 100 | 5 |
| I.10-71 | 100 | 5 |
| I.10-72 | 100 | 5 |
| I.10-115 | 100 | 5 |
| I.10-176 | 100 | 20 |
| I.15-1 | 100 | 5 |
| I.15-6 | 100 | 5 |
| I.15-23 | 100 | 5 |
| I.15-26 | 100 | 5 |
| I.15-31 | 100 | 5 |
| I.15-41 | 100 | 5 |
| I.15-71 | 100 | 5 |
| I.15-72 | 100 | 5 |
| I.15-115 | 100 | 5 |
| I.15-154 | 100 | 5 |
| I.15-166 | 100 | 5 |
| I.15-176 | 100 | 5 |
| I.15-201 | 100 | 5 |
| I.15-211 | 100 | 5 |
| I.15-280 | 100 | 5 |
| I.15-286 | 100 | 5 |
| I.15-288 | 100 | 5 |
| I.15-301 | 100 | 5 |
| I.15-350 | 100 | 5 |
| I.15-424 | 100 | 5 |
| I.15-2 | 100 | 20 |
| I.16-1 | 100 | 20 |
| I.16-2 | 100 | 20 |
| I.16-23 | 100 | 20 |
| I.16-421 | 100 | 20 |
| I.15-421 | 100 | 20 |
| I.16-176 | 100 | 20 |
| I.16-422 | 100 | 20 |
| I.16-424 | 100 | 20 |
| I.16-71 | 100 | 20 |
| I.16-115 | 100 | 20 |
| I.15-422 | 100 | 20 |
| I.14-1 | 100 | 20 |
| I.14-2 | 100 | 20 |
| I.14-23 | 100 | 20 |
| I.14-422 | 100 | 20 |
| I.35-1 | 100 | 20 |
| I.35-23 | 100 | 20 |
| I.31-23 | 100 | 20 |
| I.31-1 | 100 | 20 |
| I.1-286 | 100 | 20 |
| I.1-176 | 100 | 20 |
| I.1-115 | 100 | 20 |
| I.36-1 | 100 | 20 |
| I.36-23 | 100 | 20 |
| I.36-176 | 100 | 20 |
| I.36-286 | 100 | 20 |
| I.15-368 | 100 | 20 |
| I.15-366 | 100 | 20 |
| I.15-367 | 100 | 20 |

TABLE A8

| Compound | Amaranthus retroflexus | Application rate |
|---|---|---|
| Example No. | (efficacy in %) | [g/ha] |
| I.1-1 | 100 | 20 |
| I.1-71 | 100 | 5 |
| I.10-1 | 80 | 5 |
| I.10-26 | 100 | 5 |
| I.10-71 | 100 | 5 |
| I.10-72 | 100 | 5 |
| I.10-115 | 100 | 5 |
| I.10-176 | 100 | 20 |
| I.15-1 | 100 | 5 |
| I.15-6 | 100 | 5 |
| I.15-23 | 100 | 5 |
| I.15-26 | 100 | 5 |
| I.15-31 | 100 | 5 |
| I.15-41 | 100 | 5 |
| I.15-71 | 100 | 5 |
| I.15-72 | 100 | 5 |
| I.15-115 | 100 | 5 |
| I.15-154 | 100 | 5 |
| I.15-166 | 100 | 5 |
| I.15-176 | 100 | 5 |
| I.15-201 | 100 | 5 |
| I.15-211 | 100 | 5 |
| I.15-280 | 100 | 5 |
| I.15-286 | 100 | 5 |
| I.15-288 | 100 | 5 |
| I.15-301 | 100 | 5 |
| I.15-350 | 100 | 5 |
| I.15-424 | 100 | 5 |
| I.15-2 | 100 | 20 |
| I.16-1 | 100 | 20 |
| I.16-2 | 100 | 20 |
| I.16-23 | 100 | 20 |
| I.16-421 | 100 | 20 |
| I.15-421 | 100 | 20 |
| I.16-176 | 100 | 20 |
| I.16-422 | 100 | 20 |
| I.16-424 | 100 | 20 |
| I.16-71 | 100 | 20 |
| I.16-115 | 100 | 20 |
| I.15-422 | 100 | 20 |
| I.14-1 | 100 | 20 |
| I.14-2 | 100 | 20 |
| I.14-23 | 100 | 20 |
| I.14-422 | 100 | 20 |
| I.14-115 | 100 | 20 |
| I.35-1 | 100 | 20 |
| I.35-23 | 100 | 20 |
| I.31-23 | 100 | 20 |
| I.31-1 | 100 | 20 |
| I.1-286 | 100 | 20 |
| I.1-176 | 100 | 20 |
| I.1-115 | 100 | 20 |
| I.36-1 | 100 | 20 |
| I.36-23 | 100 | 20 |
| I.36-176 | 100 | 20 |
| I.36-286 | 100 | 20 |
| I.15-368 | 100 | 20 |
| I.15-366 | 100 | 20 |
| I.15-367 | 100 | 20 |

TABLE A9

| Compound | Matricaria inodora | Application rate |
|---|---|---|
| Example No. | (efficacy in %) | [g/ha] |
| I.1-1 | 100 | 5 |
| I.1-71 | 100 | 5 |
| I.10-1 | 90 | 5 |
| I.10-26 | 100 | 20 |
| I.10-71 | 80 | 5 |
| I.10-72 | 100 | 20 |
| I.10-115 | 90 | 5 |
| I.10-176 | 100 | 20 |
| I.15-1 | 100 | 5 |
| I.15-23 | 90 | 5 |
| I.15-26 | 900 | 5 |
| I.15-31 | 100 | 5 |
| I.15-71 | 100 | 20 |
| I.15-72 | 90 | 5 |
| I.15-115 | 100 | 5 |
| I.15-176 | 100 | 20 |
| I.15-280 | 90 | 5 |
| I.15-286 | 90 | 5 |
| I.15-288 | 90 | 5 |
| I.15-350 | 80 | 5 |
| I.15-405 | 80 | 5 |
| I.15-424 | 100 | 5 |
| I.15-2 | 100 | 20 |
| I.16-1 | 100 | 20 |
| I.16-2 | 100 | 20 |
| I.16-23 | 100 | 20 |
| I.16-421 | 100 | 20 |
| I.15-421 | 100 | 20 |
| I.16-176 | 100 | 20 |
| I.16-422 | 100 | 20 |
| I.16-424 | 100 | 20 |
| I.16-71 | 100 | 20 |
| I.16-115 | 100 | 20 |
| I.15-422 | 100 | 20 |
| I.14-1 | 100 | 20 |
| I.14-2 | 100 | 20 |
| I.14-23 | 100 | 20 |
| I.14-422 | 90 | 20 |
| I.14-115 | 100 | 20 |
| I.35-1 | 100 | 20 |
| I.35-23 | 100 | 20 |
| I.31-23 | 100 | 20 |
| I.31-1 | 100 | 20 |
| I.1-286 | 100 | 20 |
| I.1-176 | 100 | 20 |
| I.1-115 | 100 | 20 |
| I.36-1 | 100 | 20 |
| I.36-23 | 100 | 20 |
| I.36-176 | 90 | 20 |
| I.36-286 | 90 | 20 |
| I.15-368 | 100 | 20 |
| I.15-366 | 100 | 20 |
| I.15-367 | 100 | 20 |

TABLE A10

| Compound | Pharbitis purpurea | Application rate |
|---|---|---|
| Example No. | (efficacy in %) | [g/ha] |
| I.1-1 | 100 | 5 |
| I.1-71 | 100 | 5 |
| I.10-1 | 80 | 5 |
| I.10-26 | 100 | 5 |
| I.10-71 | 100 | 5 |
| I.10-72 | 100 | 5 |
| I.10-115 | 100 | 5 |
| I.10-176 | 90 | 5 |
| I.15-1 | 100 | 5 |
| I.15-6 | 100 | 5 |
| I.15-23 | 100 | 5 |
| I.15-26 | 100 | 5 |
| I.15-31 | 100 | 5 |
| I.15-41 | 100 | 5 |
| I.15-71 | 100 | 5 |
| I.15-72 | 100 | 5 |
| I.15-115 | 100 | 5 |
| I.15-154 | 100 | 5 |
| I.15-176 | 100 | 5 |
| I.15-201 | 100 | 5 |
| I.15-211 | 80 | 5 |
| I.15-280 | 100 | 5 |
| I.15-286 | 100 | 5 |
| I.15-288 | 100 | 5 |
| I.15-301 | 100 | 5 |
| I.15-350 | 100 | 5 |
| I.15-405 | 100 | 5 |
| I.15-424 | 100 | 5 |
| I.15-2 | 100 | 20 |
| I.16-1 | 100 | 20 |
| I.16-2 | 100 | 20 |
| I.16-23 | 100 | 20 |
| I.16-421 | 100 | 20 |
| I.15-421 | 100 | 20 |
| I.16-176 | 100 | 20 |
| I.16-422 | 100 | 20 |
| I.16-424 | 100 | 20 |
| I.16-71 | 100 | 20 |
| I.16-115 | 100 | 20 |
| I.15-422 | 100 | 20 |
| I.14-1 | 100 | 20 |
| I.14-2 | 100 | 20 |
| I.14-23 | 100 | 20 |
| I.14-422 | 100 | 20 |
| I.14-115 | 100 | 20 |
| I.35-1 | 100 | 20 |
| I.35-23 | 100 | 20 |
| I.31-23 | 100 | 20 |
| I.31-1 | 100 | 20 |
| I.1-286 | 100 | 20 |
| I.1-176 | 100 | 20 |
| I.1-115 | 100 | 20 |
| I.36-1 | 100 | 20 |
| I.36-23 | 100 | 20 |
| I.36-176 | 100 | 20 |
| I.36-286 | 100 | 20 |
| I.15-368 | 100 | 20 |
| I.15-366 | 100 | 20 |
| I.15-367 | 100 | 20 |

TABLE A11

| Compound | Polygonum convolvulus | Application rate |
|---|---|---|
| Example No. | (efficacy in %) | [g/ha] |
| I.1-1 | 100 | 5 |
| I.1-71 | 100 | 5 |
| I.10-1 | 100 | 5 |
| I.10-26 | 100 | 5 |
| I.10-71 | 100 | 5 |
| I.10-72 | 100 | 5 |
| I.10-115 | 100 | 5 |
| I.10-176 | 100 | 5 |
| I.15-1 | 100 | 5 |
| I.15-23 | 100 | 5 |
| I.15-26 | 100 | 5 |
| I.15-71 | 100 | 5 |
| I.15-72 | 100 | 5 |
| I.15-115 | 100 | 5 |
| I.15-176 | 100 | 5 |
| I.15-280 | 100 | 5 |
| I.15-2 | 100 | 20 |
| I.16-1 | 100 | 20 |
| I.16-2 | 100 | 20 |
| I.16-23 | 100 | 20 |
| I.16-421 | 100 | 20 |
| I.15-421 | 100 | 20 |
| I.16-176 | 90 | 20 |
| I.16-422 | 100 | 20 |
| I.16-424 | 100 | 20 |
| I.16-71 | 100 | 20 |
| I.16-115 | 100 | 20 |
| I.15-422 | 100 | 20 |
| I.14-1 | 100 | 20 |
| I.14-23 | 100 | 20 |
| I.14-422 | 100 | 20 |
| I.14-115 | 100 | 20 |
| I.35-1 | 100 | 20 |
| I.36-1 | 100 | 20 |
| I.36-23 | 90 | 20 |
| I.36-176 | 100 | 20 |
| I.36-286 | 100 | 20 |

TABLE A12

| Compound | Stellaria media | Application rate |
|---|---|---|
| Example No. | (efficacy in %) | [g/ha] |
| I.1-1 | 90 | 5 |
| I.1-71 | 100 | 5 |
| I.10-1 | 100 | 5 |
| I.10-26 | 100 | 5 |
| I.10-71 | 100 | 5 |
| I.10-72 | 90 | 5 |
| I.10-115 | 100 | 5 |
| I.10-176 | 100 | 5 |
| I.15-1 | 100 | 5 |
| I.15-23 | 100 | 5 |
| I.15-26 | 100 | 5 |
| I.15-71 | 100 | 5 |
| I.15-72 | 100 | 5 |
| I.15-115 | 100 | 5 |
| I.15-176 | 100 | 5 |

TABLE A13

| Compound | Viola tricolor | Application rate |
|---|---|---|
| Example No. | (efficacy in %) | [g/ha] |
| I.1-1 | 100 | 5 |
| I.1-71 | 100 | 5 |
| I.10-1 | 100 | 5 |
| I.10-26 | 100 | 5 |
| I.10-71 | 100 | 5 |
| I.10-72 | 100 | 5 |
| I.10-115 | 100 | 5 |
| I.10-176 | 100 | 5 |
| I.15-1 | 100 | 5 |
| I.15-23 | 100 | 5 |
| I.15-26 | 100 | 5 |
| I.15-71 | 100 | 5 |
| I.15-72 | 100 | 5 |
| I.15-115 | 100 | 5 |
| I.15-176 | 100 | 5 |
| I.15-280 | 100 | 5 |
| I.15-2 | 100 | 20 |
| I.16-1 | 100 | 20 |
| I.16-2 | 100 | 20 |
| I.16-23 | 100 | 20 |
| I.16-421 | 100 | 20 |
| I.15-421 | 100 | 20 |
| I.16-176 | 100 | 20 |
| I.16-422 | 100 | 20 |
| I.16-424 | 100 | 20 |
| I.16-71 | 100 | 20 |
| I.16-115 | 100 | 20 |
| I.15-422 | 100 | 20 |
| I.14-1 | 100 | 20 |
| I.14-2 | 100 | 20 |
| I.14-23 | 100 | 20 |
| I.14-422 | 100 | 20 |
| I.14-115 | 100 | 20 |
| I.35-1 | 100 | 20 |
| I.35-23 | 100 | 20 |
| I.31-23 | 100 | 20 |
| I.31-1 | 100 | 20 |
| I.1-286 | 100 | 20 |
| I.1-176 | 100 | 20 |
| I.1-115 | 100 | 20 |
| I.36-1 | 100 | 20 |
| I.36-23 | 100 | 20 |
| I.36-176 | 100 | 20 |
| I.36-286 | 100 | 20 |
| I.15-368 | 100 | 20 |
| I.15-366 | 100 | 20 |
| I.15-367 | 100 | 20 |

TABLE A14

| Compound | Veronica persica | Application rate |
|---|---|---|
| Example No. | (efficacy in %) | [g/ha] |
| I.1-1 | 100 | 5 |
| I.1-71 | 100 | 5 |
| I.10-1 | 90 | 20 |
| I.10-26 | 80 | 5 |
| I.10-71 | 90 | 5 |
| I.10-72 | 80 | 20 |
| I.10-115 | 100 | 20 |
| I.10-176 | 80 | 20 |
| I.15-1 | 100 | 5 |
| I.15-6 | 100 | 5 |
| I.15-23 | 100 | 5 |
| I.15-26 | 100 | 5 |
| I.15-31 | 100 | 5 |
| I.15-41 | 100 | 5 |
| I.15-71 | 100 | 5 |
| I.15-72 | 100 | 5 |
| I.15-115 | 100 | 5 |
| I.15-154 | 100 | 5 |
| I.15-166 | 100 | 5 |
| I.15-176 | 90 | 5 |
| I.15-211 | 100 | 5 |
| I.15-201 | 100 | 5 |
| I.15-280 | 100 | 5 |
| I.15-286 | 100 | 5 |
| I.15-288 | 100 | 5 |
| I.15-301 | 100 | 5 |
| I.15-350 | 100 | 5 |
| I.15-405 | 100 | 5 |
| I.15-424 | 100 | 5 |
| I.15-2 | 100 | 20 |
| I.16-1 | 100 | 20 |
| I.16-2 | 100 | 20 |
| I.16-23 | 100 | 20 |
| I.16-421 | 100 | 20 |
| I.15-421 | 100 | 20 |
| I.16-176 | 100 | 20 |
| I.16-422 | 100 | 20 |
| I.16-424 | 100 | 20 |
| I.16-71 | 100 | 20 |
| I.16-115 | 100 | 20 |
| I.15-422 | 100 | 20 |
| I.14-1 | 100 | 20 |
| I.14-2 | 100 | 20 |
| I.14-23 | 100 | 20 |
| I.14-422 | 100 | 20 |
| I.14-115 | 100 | 20 |
| I.35-1 | 100 | 20 |
| I.35-23 | 100 | 20 |
| I.31-23 | 100 | 20 |
| I.31-1 | 100 | 20 |
| I.1-286 | 100 | 20 |
| I.1-176 | 100 | 20 |
| I.1-115 | 100 | 20 |
| I.36-1 | 100 | 20 |
| I.36-23 | 100 | 20 |
| I.36-176 | 90 | 20 |
| I.36-286 | 100 | 20 |
| I.15-368 | 100 | 20 |
| I.15-366 | 100 | 20 |
| I.15-367 | 100 | 20 |

TABLE A15

| Compound | Hordeum murinum | Application rate |
|---|---|---|
| Example No. | (efficacy in %) | [g/ha] |
| I.1-1 | 80 | 5 |
| I.1-71 | 80 | 20 |
| I.10-1 | 90 | 20 |
| I.10-71 | 80 | 20 |
| I.10-72 | 80 | 20 |
| I.10-115 | 80 | 20 |
| I.10-176 | 80 | 20 |
| I.15-1 | 100 | 20 |
| I.15-23 | 80 | 5 |
| I.15-26 | 100 | 20 |
| I.15-71 | 100 | 20 |
| I.15-72 | 90 | 5 |
| I.15-115 | 100 | 20 |
| I.15-176 | 100 | 20 |

Tables A16 to A19 below show the crop plant compatibilities of selected compounds of the general formula (I) according to Tables I.1 to I.36 at an application rate corresponding to 5 g/ha or 20 g/ha, which were obtained in experiments according to the experimental procedure mentioned above. Here, the observed effects on selected crop plants are stated in comparison to the untreated controls (values in %).

TABLE A16

| Compound | Oryza sativa | Application rate |
|---|---|---|
| Example No. | (efficacy in %) | [g/ha] |
| I.10-71 | 20 | 5 |
| I.10-72 | 20 | 5 |
| I.10-115 | 20 | 5 |
| I.10-176 | 20 | 5 |
| I.15-6 | 20 | 5 |
| I.15-31 | 20 | 5 |
| I.15-41 | 20 | 5 |
| I.15-154 | 0 | 5 |
| I.15-176 | 20 | 5 |
| I.15-201 | 0 | 5 |
| I.15-211 | 0 | 5 |
| I.15-286 | 20 | 5 |
| I.15-288 | 0 | 5 |
| I.15-301 | 20 | 5 |
| I.15-350 | 20 | 5 |
| I.16-421 | 20 | 5 |
| I.16-422 | 20 | 5 |
| I.16-115 | 20 | 5 |
| I.14-1 | 0 | 5 |
| I.14-2 | 10 | 20 |
| I.14-23 | 10 | 20 |
| I.14-422 | 20 | 20 |
| I.14-115 | 20 | 20 |
| I.31-23 | 20 | 20 |
| I.31-1 | 20 | 20 |
| I.1-286 | 0 | 5 |
| I.1-176 | 20 | 5 |
| I.1-115 | 20 | 5 |
| I.36-176 | 20 | 5 |

TABLE A17

| Compound | Zea mays | Application rate |
|---|---|---|
| Example No. | (efficacy in %) | [g/ha] |
| I.10-71 | 20 | 5 |
| I.15-176 | 20 | 5 |
| I.15-201 | 20 | 5 |
| I.14-1 | 10 | 5 |
| I.14-2 | 20 | 5 |
| I.14-23 | 10 | 20 |
| I.14-115 | 20 | 5 |

TABLE A18

| Compound | Brassica napis | Application rate |
|---|---|---|
| Example No. | (efficacy in %) | [g/ha] |
| I.10-1 | 20 | 5 |
| I.15-154 | 0 | 5 |
| I.15-166 | 0 | 5 |
| I.15-201 | 0 | 5 |
| I.15-286 | 20 | 5 |

TABLE A19

| Compound | Triticum aestivum | Application rate |
|---|---|---|
| Example No. | (efficacy in %) | [g/ha] |
| I.10-72 | 20 | 20 |
| I.15-6 | 20 | 5 |
| I.15-41 | 20 | 5 |
| I.15-115 | 20 | 5 |
| I.15-154 | 0 | 5 |
| I.15-166 | 0 | 5 |
| I.15-176 | 20 | 5 |
| I.15-211 | 0 | 5 |
| I.15-280 | 20 | 5 |
| I.15-350 | 20 | 5 |

As the results show, compounds of the general formula (I) according to the invention, in post-emergence treatment, have good herbicidal activity against harmful plants such as Abutilon theophrasti, Alopecurus myosuroides, Amaranthus retroflexus, Avena fatua, Digitaria sanguinalis, Echinochloa crus-galli, Hordeum murinum, Lolium rigidum, Matricaria inodora, Pharbitis purpurea, Polygonum convolvulus, Setaria viridis, Stellaria media, Veronica persica and Viola tricolor at an application rate of 0.02 kg of active substance or less per hectare, and good crop plant compatibility with organisms such as Oryza sativa, Zea mays, Brassica napus and Triticum aestivum at an application rate of 0.02 kg or less per hectare.

### B. Pre-Emergence Herbicidal Action and Crop Plant Compatibility

Seeds of monocotyledonous and dicotyledonous weed plants and crop plants were placed in plastic or organic planting pots and covered with soil. The compounds of the invention, formulated in the form of wettable powders (WP) or as emulsion concentrates (EC), were then applied to the surface of the covering soil as aqueous suspension or emulsion with addition of 0.5% additive at a water application rate equivalent to 600 l/ha (converted). After the treatment, the pots were placed in a greenhouse and kept under good growth conditions for the test plants. After about 3 weeks, the effect of the preparations was scored visually in comparison with untreated controls as percentages. For example, 100% activity=the plants have died, 0% activity=like control plants.

Tables B1 to B13 below show the effects of selected compounds of the general formula (I) according to Tables I.1 to I.36 on various harmful plants and at an application rate corresponding to 80 g/ha or less, which were obtained by the experimental procedure mentioned above.

TABLE B1

| Compound | Alopecurus myosuroides | Application rate |
|---|---|---|
| Example No. | (efficacy in %) | [g/ha] |
| I.15-6 | 90 | 20 |
| I.15-31 | 100 | 80 |
| I.15-41 | 100 | 80 |
| I.15-154 | 100 | 80 |
| I.15-166 | 90 | 20 |
| I.15-201 | 100 | 80 |
| I.15-211 | 100 | 80 |
| I.15-280 | 90 | 20 |
| I.15-286 | 100 | 80 |
| I.15-288 | 100 | 80 |
| I.15-301 | 100 | 80 |
| I.15-350 | 100 | 80 |
| I.15-405 | 100 | 80 |
| I.15-424 | 80 | 20 |

TABLE B2

| Compound | Avena fatua | Application rate |
|---|---|---|
| Example No. | (efficacy in %) | [g/ha] |
| I.15-6 | 100 | 80 |
| I.15-31 | 100 | 80 |
| I.15-41 | 90 | 80 |
| I.15-154 | 100 | 80 |
| I.15-166 | 100 | 80 |
| I.15-201 | 100 | 80 |
| I.15-211 | 80 | 80 |
| I.15-280 | 80 | 20 |
| I.15-286 | 100 | 80 |
| I.15-288 | 100 | 80 |
| I.15-301 | 100 | 80 |
| I.15-350 | 100 | 80 |
| I.15-405 | 100 | 80 |
| I.15-424 | 100 | 80 |

TABLE B3

| Compound | Digitaria sanguinalis | Application rate |
|---|---|---|
| Example No. | (efficacy in %) | [g/ha] |
| I.15-6 | 100 | 20 |
| I.15-31 | 100 | 20 |
| I.15-41 | 100 | 20 |
| I.15-154 | 100 | 20 |
| I.15-166 | 100 | 20 |
| I.15-201 | 100 | 20 |
| I.15-211 | 100 | 20 |
| I.15-280 | 100 | 20 |
| I.15-286 | 100 | 20 |
| I.15-288 | 100 | 20 |
| I.15-301 | 100 | 20 |
| I.15-350 | 100 | 20 |
| I.15-405 | 100 | 20 |
| I.15-424 | 100 | 20 |

TABLE B4

| Compound | Echinochloa crus-galli | Application rate |
|---|---|---|
| Example No. | (efficacy in %) | [g/ha] |
| I.15-6 | 100 | 80 |
| I.15-31 | 100 | 20 |
| I.15-41 | 100 | 80 |
| I.15-154 | 100 | 80 |
| I.15-166 | 80 | 20 |
| I.15-201 | 100 | 80 |
| I.15-211 | 80 | 20 |
| I.15-280 | 90 | 20 |
| I.15-286 | 80 | 20 |
| I.15-288 | 80 | 20 |
| I.15-301 | 80 | 20 |
| I.15-350 | 100 | 80 |
| I.15-405 | 80 | 20 |
| I.15-424 | 90 | 20 |

TABLE B5

| Compound | Lolium rigidum | Application rate |
|---|---|---|
| Example No. | (efficacy in %) | [g/ha] |
| I.15-6 | 100 | 80 |
| I.15-31 | 100 | 80 |
| I.15-41 | 100 | 80 |
| I.15-154 | 100 | 80 |
| I.15-166 | 100 | 80 |
| I.15-201 | 100 | 80 |
| I.15-211 | 100 | 80 |
| I.15-280 | 100 | 80 |
| I.15-286 | 100 | 80 |
| I.15-288 | 100 | 80 |
| I.15-301 | 100 | 80 |
| I.15-350 | 100 | 80 |
| I.15-405 | 100 | 80 |
| I.15-424 | 100 | 80 |

TABLE B6

| Compound | Setaria viridis | Application rate |
|---|---|---|
| Example No. | (efficacy in %) | [g/ha] |
| I.15-6 | 100 | 20 |
| I.15-31 | 100 | 20 |
| I.15-41 | 100 | 20 |
| I.15-154 | 100 | 20 |
| I.15-166 | 100 | 20 |
| I.15-201 | 100 | 20 |
| I.15-211 | 100 | 20 |
| I.15-280 | 100 | 20 |
| I.15-286 | 100 | 20 |
| I.15-288 | 100 | 20 |
| I.15-301 | 100 | 20 |
| I.15-350 | 100 | 20 |
| I.15-405 | 100 | 20 |
| I.15-424 | 100 | 20 |

TABLE B7

| Compound | Abutilon theophrasti | Application rate |
|---|---|---|
| Example No. | (efficacy in %) | [g/ha] |
| I.15-6 | 100 | 20 |
| I.15-31 | 100 | 20 |
| I.15-41 | 100 | 20 |
| I.15-154 | 100 | 20 |
| I.15-166 | 100 | 20 |
| I.15-201 | 100 | 20 |
| I.15-211 | 100 | 20 |
| I.15-280 | 100 | 20 |
| I.15-286 | 100 | 20 |
| I.15-288 | 100 | 20 |
| I.15-301 | 100 | 20 |
| I.15-350 | 100 | 20 |
| I.15-405 | 100 | 20 |
| I.15-424 | 100 | 20 |

TABLE B8

| Compound | Amaranthus retroflexus | Application rate |
|---|---|---|
| Example No. | (efficacy in %) | [g/ha] |
| I.15-6 | 100 | 20 |
| I.15-31 | 100 | 20 |
| I.15-41 | 100 | 20 |
| I.15-154 | 100 | 20 |
| I.15-166 | 100 | 20 |
| I.15-201 | 100 | 20 |
| I.15-211 | 100 | 20 |
| I.15-280 | 100 | 20 |
| I.15-286 | 100 | 20 |
| I.15-288 | 100 | 20 |
| I.15-301 | 100 | 20 |
| I.15-350 | 100 | 20 |
| I.15-405 | 100 | 20 |
| I.15-424 | 100 | 20 |

TABLE B9

| Compound | Matricaria inodora | Application rate |
|---|---|---|
| Example No. | (efficacy in %) | [g/ha] |
| I.15-6 | 100 | 20 |
| I.15-31 | 100 | 20 |
| I.15-41 | 100 | 20 |
| I.15-154 | 100 | 20 |
| I.15-166 | 100 | 20 |
| I.15-201 | 100 | 20 |
| I.15-211 | 100 | 20 |
| I.15-280 | 100 | 20 |
| I.15-286 | 100 | 20 |
| I.15-288 | 100 | 20 |
| I.15-301 | 100 | 20 |
| I.15-350 | 100 | 20 |
| I.15-405 | 100 | 20 |
| I.15-424 | 100 | 20 |

TABLE B10

| Compound | Pharbitis purpurea | Application rate |
|---|---|---|
| Example No. | (efficacy in %) | [g/ha] |
| I.15-6 | 100 | 20 |
| I.15-31 | 100 | 20 |
| I.15-41 | 100 | 20 |
| I.15-154 | 100 | 80 |
| I.15-166 | 100 | 20 |
| I.15-201 | 100 | 20 |
| I.15-211 | 80 | 20 |
| I.15-280 | 90 | 20 |
| I.15-286 | 100 | 20 |
| I.15-288 | 100 | 20 |
| I.15-301 | 100 | 20 |
| I.15-350 | 100 | 20 |
| I.15-405 | 100 | 20 |
| I.15-424 | 100 | 20 |

TABLE B11

| Compound | Polygonum convolvulus | Application rate |
|---|---|---|
| Example No. | (efficacy in %) | [g/ha] |
| I.15-6 | 100 | 20 |
| I.15-31 | 100 | 20 |
| I.15-41 | 100 | 20 |
| I.15-154 | 100 | 20 |
| I.15-166 | 100 | 20 |
| I.15-201 | 100 | 20 |
| I.15-211 | 100 | 20 |
| I.15-280 | 100 | 20 |
| I.15-286 | 100 | 20 |
| I.15-288 | 100 | 20 |
| I.15-301 | 100 | 20 |
| I.15-350 | 100 | 20 |
| I.15-405 | 90 | 20 |
| I.15-424 | 100 | 20 |

TABLE B12

| Compound | Viola tricolor | Application rate |
|---|---|---|
| Example No. | (efficacy in %) | [g/ha] |
| I.15-6 | 100 | 20 |
| I.15-31 | 100 | 20 |
| I.15-41 | 100 | 20 |
| I.15-154 | 100 | 20 |
| I.15-166 | 100 | 20 |
| I.15-201 | 100 | 20 |
| I.15-211 | 100 | 20 |
| I.15-280 | 100 | 20 |
| I.15-286 | 100 | 20 |
| I.15-288 | 100 | 20 |
| I.15-301 | 100 | 20 |
| I.15-350 | 100 | 20 |
| I.15-405 | 100 | 20 |
| I.15-424 | 100 | 20 |

TABLE B13

| Compound | Veronica persica | Application rate |
|---|---|---|
| Example No. | (efficacy in %) | [g/ha] |
| I.15-6 | 100 | 20 |
| I.15-31 | 100 | 20 |
| I.15-41 | 100 | 20 |
| I.15-154 | 100 | 20 |
| I.15-166 | 100 | 20 |
| I.15-201 | 100 | 20 |
| I.15-211 | 100 | 20 |
| I.15-280 | 100 | 20 |
| I.15-286 | 100 | 20 |
| I.15-288 | 100 | 20 |
| I.15-301 | 80 | 20 |
| I.15-350 | 100 | 20 |
| I.15-405 | 100 | 20 |
| I.15-424 | 100 | 20 |

Tables B14 to B16 below show the crop plant compatibilities of selected compounds of the general formula (I) according to Tables I.1 to I.36 at an application rate corresponding to 20 g/ha, which were obtained in experiments according to the experimental procedure mentioned above. Here, the observed effects on selected crop plants are stated in comparison to the untreated controls (values in %).

TABLE B14

| Compound | Zea mays | Application rate |
|---|---|---|
| Example No. | (efficacy in %) | [g/ha] |
| I.15-6 | 0 | 20 |
| I.15-154 | 0 | 20 |
| I.15-201 | 0 | 20 |
| I.15-211 | 20 | 20 |
| I.15-286 | 20 | 20 |
| I.15-301 | 0 | 20 |
| I.15-350 | 0 | 20 |
| I.15-405 | 0 | 20 |

TABLE B15

| Compound | Glycine max | Application rate |
|---|---|---|
| Example No. | (efficacy in %) | [g/ha] |
| I.15-41 | 20 | 20 |
| I.15-280 | 0 | 20 |

TABLE B16

| Compound | Triticum aestivum | Application rate |
|---|---|---|
| Example No. | (efficacy in %) | [g/ha] |
| I.15-6 | 10 | 20 |
| I.15-154 | 10 | 20 |
| I.15-166 | 20 | 20 |
| I.15-211 | 0 | 20 |
| I.15-280 | 10 | 20 |
| I.15-405 | 20 | 20 |
| I.15-424 | 10 | 20 |

As the results show, compounds of the general formula (I) according to the invention, in pre-emergence treatment, have good herbicidal activity against harmful plants, for example against harmful plants such as Abutilon theophrasti, Alopecurus myosuroides, Amaranthus retroflexus, Avena fatua, Digitaria sanguinalis, Echinochloa crus-galli, Lolium rigidum, Matricaria inodora, Pharbitis purpurea, Polygonum convolvulus, Setaria viridis, Veronica persica and Viola tricolor at an application rate of 0.08 kg of active substance or less per hectare, and good crop plant compatibility with organisms such as Zea mays, Glycine max and Triticum aestivum at an application rate of 0.02 kg per hectare.

1. A substituted N-phenyluracil of formula (I) or salt thereof 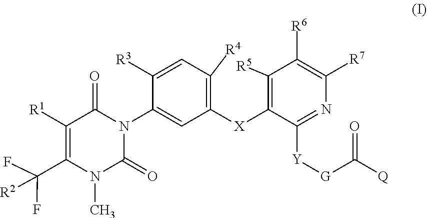
in which
R¹ represents hydrogen, (C₁-C₈)-haloalkyl,
R² represents hydrogen, fluorine, chlorine, bromine, trifluoromethyl, (C₁-C₈)-alkoxy,
R³ represents hydrogen, halogen, (C₁-C₈)-alkoxy,
R⁴ represents halogen, cyano, NO₂, C(O)NH₂, C(S)NH₂, (C₁-C₈)-haloalkyl, (C₂-C₈)-alkynyl,
R⁵, R⁶ and R⁷ independently of one another represent hydrogen, halogen, cyano, (C₁-C₈)-alkyl, (C₁-C₈)-haloalkyl, (C₁-C₈)-alkoxy, (C₁-C₈)-haloalkoxy,
G represents straight-chain or branched (C₁-C₈)-alkylene,
Q represents a radical of formula 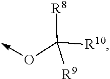
R⁸ represents hydrogen, (C₁-C₈)-alkyl, (C₁-C₈)-haloalkyl, aryl, aryl-(C₁-C₈)-alkyl, heteroaryl, (C₂-C₈)-alkynyl, (C₂-C₈)-alkenyl, C(O)R¹³, C(O)OR¹³, (C₁-C₈)-alkoxy-(C₁-C₈)-alkyl,
R⁹ represents hydrogen or (C₁-C₈)-alkyl,
R¹⁰ represents cyano, NO₂, heteroaryl, heteroaryl-(C₁-C₈)-alkyl, heterocyclyl, heterocyclyl-(C₁-C₈)-alkyl, R¹¹R¹²N—(C₁-C₈)-alkyl, R¹³O—(C₁-C₈)-alkyl, cyano-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, OR¹³, NR¹¹R¹², SR¹⁴, S(O)R¹⁴, SO₂R¹⁴, R¹⁴S—(C₁-C₈)-alkyl, R¹⁴(O)S—(C₁-C₈)-alkyl, R¹⁴O₂S—(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-alkyl]-bis-(aryl)silyl-(C₁-C₈)-alkyl, tris-[(C₁-C₈)-alkyl]silyl, bis-hydroxyboryl-(C₁-C₈)-alkyl, bis-[(C₁-C₈)-alkoxy]boryl-(C₁-C₈)-alkyl, tetramethyl-1,3,2-dioxaborolan-2-yl, tetramethyl-1,3,2-dioxaborolan-2-yl-(C₁-C₈)-alkyl, nitro-(C₁-C₈)-alkyl, C(O)R¹⁴, bis-(C₁-C₈)-alkoxymethyl, bis-(C₁-C₈)-alkoxymethyl-(C₁-C₈)-alkyl, or
R⁸ and R¹⁰ together with the carbon atom to which they are attached form fully saturated or partially saturated 3- to 10-membered monocyclic or bicyclic heterocyclyl optionally having further substitution,
R¹¹ and R¹² are identical or different and independently of one another represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, COR¹³, SO₂R¹⁴, heterocyclyl, (C₁-C₈)-alkoxycarbonyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, heterocyclyl-(C₁-C₈)-alkyl, or
R¹¹ and R¹² together with the nitrogen atom to which they are attached form a fully saturated or partially saturated 3- to 10-membered monocyclic or bicyclic ring optionally interrupted by heteroatoms and optionally having further substitution,
R¹³ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkoxy-(C₁-C₈)-haloalkyl, (C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkoxy-(C₁-C₈)-alkyl, aryl, aryl-(C₁-C₈)-alkyl, aryl-(C₁-C₈)-alkoxy-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, bis-[(C₁-C₈)-alkyl]amino-(C₂-C₆)-alkyl, (C₁-C₈)-alkyl-amino-(C₂-C₆)-alkyl, aryl-(C₁-C₈)-alkyl-amino-(C₂-C₆)-alkyl, R¹⁴S—(C₁-C₈)-alkyl, R¹⁴(O)S—(C₁-C₈)-alkyl, R¹⁴O₂S—(C₁-C₈)-alkyl, hydroxycarbonyl-(C₁-C₈)-alkyl, heterocyclyl, heterocyclyl-(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-alkyl]-bis-(aryl)silyl-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, aryloxy-(C₁-C₈)-alkyl, heteroaryloxy-(C₁-C₈)-alkyl, (C₁-C₈)-alkoxycarbonyl,
R¹⁴ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]amino, (C₁-C₈)-alkyl-amino, aryl-(C₁-C₈)-amino, aryl-(C₁-C₆)-alkyl-amino, aryl-[(C₁-C₈)-alkyl]amino, (C₃-C₈)-cycloalkyl-amino, (C₃-C₈)-cycloalkyl-[(C₁-C₈)-alkyl]amino; N-azetidinyl, N-pyrrolidinyl, N-piperidinyl, N-morpholinyl,
and
X and Y independently of one another represent O (oxygen) or S (sulfur).

2. The compound of formula (I) as claimed in claim 1 and/or salt thereof, wherein
R¹ represents hydrogen, (C₁-C₇)-haloalkyl,
R² represents hydrogen, fluorine, chlorine, bromine, trifluoromethyl, (C₁-C₇)-alkoxy,
R³ represents hydrogen, halogen, (C₁-C₇)-alkoxy,
R⁴ represents halogen, cyano, NO₂, C(O)NH₂, C(S)NH₂, (C₁-C₇)-haloalkyl, (C₂-C₇)-alkynyl,
R⁵, R⁶ and R⁷ independently of one another represent hydrogen, halogen, cyano, (C₁-C₇)-alkyl, (C₁-C₇)-haloalkyl, (C₁-C₇)-alkoxy, (C₁-C₇)-haloalkoxy,
G represents straight-chain or branched (C₁-C₇)-alkylene,
Q represents a radical of formula 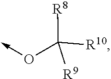
R⁸ represents hydrogen, (C₁-C₇)-alkyl, (C₁-C₇)-haloalkyl, aryl, aryl-(C₁-C₇)-alkyl, heteroaryl, (C₂-C₇)-alkynyl, (C₂-C₇)-alkenyl, C(O)R¹³, C(O)OR¹³, (C₁-C₇)-alkoxy-(C₁-C₇)-alkyl,
R⁹ represents hydrogen or (C₁-C₆)-alkyl,
R¹⁰ represents cyano, NO₂, heteroaryl, heteroaryl-(C₁-C₇)-alkyl, heterocyclyl, heterocyclyl-(C₁-C₇)-alkyl, R¹¹R¹²N—(C₁-C₇)-alkyl, R¹³O—(C₁-C₇)-alkyl, cyano-(C₁-C₇)-alkyl, (C₁-C₇)-alkylcarbonyloxy-(C₁-C₇)-alkyl, (C₃-C₇)-cycloalkylcarbonyloxy-(C₁-C₇)-alkyl, arylcarbonyloxy-(C₁-C₇)-alkyl, heteroarylcarbonyloxy-(C₁-C₇)-alkyl, heterocyclylcarbonyloxy-(C₁-C₇)-alkyl, OR¹³, NR¹¹R¹², SR¹⁴, S(O)R¹⁴, SO₂R¹⁴, R¹⁴S—(C₁-C₇)-alkyl, R¹⁴(O)S—(C₁-C₇)-alkyl, R¹⁴O₂S—(C₁-C₇)-alkyl, tris-[(C₁-C₇)-alkyl]silyl-(C₁-C₇)-alkyl, bis-[(C₁-C₇)-alkyl](aryl)silyl(C₁-C₇)-alkyl, [(C₁-C₇)-alkyl]-bis-(aryl)silyl-(C₁-C₇)-alkyl, tris-[(C₁-C₇)-alkyl]silyl, bis-hydroxyboryl-(C₁-C₇)-alkyl, bis-[(C₁-C₇)-alkoxy]boryl-(C₁-C₇)-alkyl, tetramethyl-1,3,2-dioxaborolan-2-yl, tetramethyl-1,3,2-dioxaborolan-2-yl-(C₁-C₇)-alkyl, nitro-(C₁-C₇)-alkyl, C(O)R¹⁴, bis-(C₁-C₇)-alkoxymethyl, bis-(C₁-C₇)-alkoxymethyl-(C₁-C₇)-alkyl, or
R⁸ and R¹⁰ together with the carbon atom to which they are attached form fully saturated or partially saturated 3- to 10-membered monocyclic or bicyclic heterocyclyl optionally having further substitution,
R¹¹ and R¹² are identical or different and independently of one another represent hydrogen, (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₃-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-haloalkoxy-(C₁-C₇)-alkyl, (C₁-C₇)-alkylthio-(C₁-C₇)-alkyl, (C₁-C₇)-haloalkylthio-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, (C₃-C₇)-cycloalkyl-(C₁-C₇)-alkyl, (C₄-C₁₀)-cycloalkenyl-(C₁-C₇)-alkyl, COR¹³, SO₂R¹⁴, heterocyclyl, (C₁-C₇)-alkoxycarbonyl, bis-[(C₁-C₇)-alkyl]aminocarbonyl-(C₁-C₇)-alkyl, (C₁-C₇)-alkyl-aminocarbonyl-(C₁-C₇)-alkyl, aryl-(C₁-C₇)-alkyl-aminocarbonyl-(C₁-C₇)-alkyl, aryl-(C₁-C₇)-alkoxycarbonyl, heteroaryl-(C₁-C₇)-alkoxycarbonyl, (C₂-C₇)-alkenyloxycarbonyl, (C₂-C₇)-alkynyloxycarbonyl, heterocyclyl-(C₁-C₇)-alkyl, or
R¹¹ and R¹² together with the nitrogen atom to which they are attached form a fully saturated or partially saturated 3- to 10-membered monocyclic or bicyclic ring optionally interrupted by heteroatoms and optionally having further substitution,
R¹³ represents hydrogen, (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₃-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-haloalkoxy-(C₁-C₇)-alkyl, (C₁-C₇)-alkoxy-(C₁-C₇)-haloalkyl, (C₁-C₇)-alkoxy-(C₁-C₇)-alkoxy-(C₁-C₇)-alkyl, (C₁-C₇)-alkoxy-(C₁-C₇)-alkoxy-(C₁-C₇)-alkoxy-(C₁-C₇)-alkyl, (C₁-C₇)-alkoxy-(C₁-C₇)-alkoxy-(C₁-C₇)-alkoxy-(C₁-C₇)-alkoxy-(C₁-C₇)-alkyl, aryl, aryl-(C₁-C₇)-alkyl, aryl-(C₁-C₇)-alkoxy-(C₁-C₇)-alkyl, heteroaryl, heteroaryl-(C₁-C₇)-alkyl, (C₃-C₇)-cycloalkyl-(C₁-C₇)-alkyl, (C₄-C₁₀)-cycloalkenyl-(C₁-C₇)-alkyl, bis-[(C₁-C₇)-alkyl]aminocarbonyl-(C₁-C₇)-alkyl, (C₁-C₇)-alkyl-aminocarbonyl-(C₁-C₇)-alkyl, aryl-(C₁-C₇)-alkyl-aminocarbonyl-(C₁-C₇)-alkyl, bis-[(C₁-C₇)-alkyl]amino-(C₂-C₅)-alkyl, (C₁-C₇)-alkyl-amino-(C₂-C₅)-alkyl, aryl-(C₁-C₇)-alkyl-amino-(C₂-C₅)-alkyl, R¹⁴S—(C₁-C₇)-alkyl, R¹⁴(O)S—(C₁-C₇)-alkyl, R¹⁴O_{2S}—(C₁-C₇)-alkyl, hydroxycarbonyl-(C₁-C₇)-alkyl, heterocyclyl, heterocyclyl-(C₁-C₇)-alkyl, tris-[(C₁-C₇)-alkyl]silyl-(C₁-C₇)-alkyl, bis-[(C₁-C₇)-alkyl](aryl)silyl(C₁-C₇)-alkyl, [(C₁-C₇)-alkyl]-bis-(aryl)silyl-(C₁-C₇)-alkyl, (C₁-C₇)-alkylcarbonyloxy-(C₁-C₇)-alkyl, (C₃-C₇)-cycloalkylcarbonyloxy-(C₁-C₇)-alkyl, arylcarbonyloxy-(C₁-C₇)-alkyl, heteroarylcarbonyloxy-(C₁-C₇)-alkyl, heterocyclylcarbonyloxy-(C₁-C₇)-alkyl, aryloxy-(C₁-C₇)-alkyl, heteroaryloxy-(C₁-C₇)-alkyl, (C₁-C₇)-alkoxycarbonyl,
R¹⁴ represents hydrogen, (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₃-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, heterocyclyl-(C₁-C₇)-alkyl, (C₃-C₇)-cycloalkyl-(C₁-C₇)-alkyl, (C₄-C₁₀)-cycloalkenyl-(C₁-C₇)-alkyl, bis-[(C₁-C₇)-alkyl]amino, (C₁-C₇)-alkyl-amino, aryl-(C₁-C₇)-amino, aryl-(C₁-C₄)-alkyl-amino, aryl-[(C₁-C₇)-alkyl]amino, (C₃-C₇)-cycloalkyl-amino, (C₃-C₇)-cycloalkyl-[(C₁-C₇)-alkyl]amino; N-azetidinyl, N-pyrrolidinyl, N-piperidinyl, N-morpholinyl, and
X and Y independently of one another represent O (oxygen) or S (sulfur).

3. The compound of formula (I) as claimed in claim 1 and/or salt thereof, wherein
R¹ represents hydrogen,
R² represents hydrogen, fluorine, chlorine, bromine, trifluoromethyl, (C₁-C₆)-alkoxy,
R³ represents hydrogen, halogen, (C₁-C₆)-alkoxy,
R⁴ represents halogen, cyano, NO₂, C(O)NH₂, C(S)NH₂, (C₁-C₆)-haloalkyl, (C₂-C₆)-alkynyl,
R⁵, R⁶ and R⁷ independently of one another represent hydrogen, halogen, cyano, (C₁-C₆)-alkyl, (C₁-C₆)-haloalkyl, (C₁-C₆)-alkoxy, (C₁-C₆)-haloalkoxy,
G represents straight-chain or branched (C₁-C₆)-alkylene,
Q represents a radical of formula 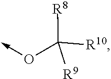
R⁸ represents hydrogen, (C₁-C₆)-alkyl, (C₁-C₆)-haloalkyl, aryl, aryl-(C₁-C₆)-alkyl, heteroaryl, (C₂-C₆)-alkynyl, (C₂-C₆)-alkenyl, C(O)R¹³, C(O)OR¹³, (C₁-C₆)-alkoxy-(C₁-C₆)-alkyl,
R⁹ represents hydrogen or (C₁-C₄)-alkyl,
R¹⁰ represents cyano, NO₂, heteroaryl, heteroaryl-(C₁-C₆)-alkyl, heterocyclyl, heterocyclyl-(C₁-C₆)-alkyl, R¹¹R¹²N—(C₁-C₆)-alkyl, R¹³O—(C₁-C₆)-alkyl, cyano-(C₁-C₆)-alkyl, (C₁-C₆)-alkylcarbonyloxy-(C₁-C₆)-alkyl, (C₃-C₆)-cycloalkylcarbonyloxy-(C₁-C₆)-alkyl, arylcarbonyloxy-(C₁-C₆)-alkyl, heteroarylcarbonyloxy-(C₁-C₆)-alkyl, heterocyclylcarbonyloxy-(C₁-C₆)-alkyl, OR¹³, NR¹¹R¹², SR¹⁴, S(O)R¹⁴, SO₂R¹⁴, R¹⁴S—(C₁-C₆)-alkyl, R¹⁴(O)S—(C₁-C₆)-alkyl, R¹⁴O₂S—(C₁-C₆)-alkyl, tris-[(C₁-C₆)-alkyl]silyl-(C₁-C₆)-alkyl, bis-[(C₁-C₆)-alkyl](aryl)silyl(C₁-C₆)-alkyl, [(C₁-C₆)-alkyl]-bis-(aryl)silyl-(C₁-C₆)-alkyl, tris-[(C₁-C₆)-alkyl]silyl, bis-hydroxyboryl-(C₁-C₆)-alkyl, bis-[(C₁-C₆)-alkoxy]boryl-(C₁-C₆)-alkyl, tetramethyl-1,3,2-dioxaborolan-2-yl, tetramethyl-1,3,2-dioxaborolan-2-yl-(C₁-C₆)-alkyl, nitro-(C₁-C₆)-alkyl, C(O)R¹³, bis-(C₁-C₆)-alkoxymethyl, bis-(C₁-C₆)-alkoxymethyl-(C₁-C₆)-alkyl,
R⁸ and R¹⁰ together with the carbon atom to which they are attached form fully saturated or partially saturated 3- to 10-membered monocyclic or bicyclic heterocyclyl optionally having further substitution,
R¹¹ and R¹² are identical or different and independently of one another represent hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-haloalkoxy-(C₁-C₆)-alkyl, (C₁-C₆)-alkylthio-(C₁-C₆)-alkyl, (C₁-C₆)-haloalkylthio-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, COR¹³, SO₂R¹⁴, heterocyclyl, (C₁-C₆)-alkoxycarbonyl, bis-[(C₁-C₆)-alkyl]aminocarbonyl-(C₁-C₆)-alkyl, (C₁-C₆)-alkyl-aminocarbonyl-(C₁-C₆)-alkyl, aryl-(C₁-C₆)-alkyl-aminocarbonyl-(C₁-C₆)-alkyl, aryl-(C₁-C₆)-alkoxycarbonyl, heteroaryl-(C₁-C₆)-alkoxycarbonyl, (C₂-C₆)-alkenyloxycarbonyl, (C₂-C₆)-alkynyloxycarbonyl, heterocyclyl-(C₁-C₆)-alkyl, or
R¹¹ and R¹² together with the nitrogen atom to which they are attached form a fully saturated or partially saturated 3- to 10-membered monocyclic or bicyclic ring optionally interrupted by heteroatoms and optionally having further substitution,
R¹³ represents hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-haloalkoxy-(C₁-C₆)-alkyl, (C₁-C₆)-alkoxy-(C₁-C₆)-haloalkyl, (C₁-C₆)-alkoxy-(C₁-C₆)-alkoxy-(C₁-C₆)-alkyl, (C₁-C₆)-alkoxy-(C₁-C₆)-alkoxy-(C₁-C₆)-alkoxy-(C₁-C₆)-alkyl, (C₁-C₆)-alkoxy-(C₁-C₆)-alkoxy-(C₁-C₆)-alkoxy-(C₁-C₆)-alkoxy-(C₁-C₆)-alkyl, aryl, aryl-(C₁-C₆)-alkyl, aryl-(C₁-C₆)-alkoxy-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, bis-[(C₁-C₆)-alkyl]aminocarbonyl-(C₁-C₆)-alkyl, (C₁-C₆)-alkyl-aminocarbonyl-(C₁-C₆)-alkyl, aryl-(C₁-C₆)-alkyl-aminocarbonyl-(C₁-C₆)-alkyl, bis-[(C₁-C₆)-alkyl]amino-(C₂-C₄)-alkyl, (C₁-C₆)-alkyl-amino-(C₂-C₄)-alkyl, aryl-(C₁-C₆)-alkyl-amino-(C₂-C₄)-alkyl, R¹⁴S—(C₁-C₆)-alkyl, R¹⁴(O)S—(C₁-C₆)-alkyl, R¹⁴O₂S—(C₁-C₆)-alkyl, hydroxycarbonyl-(C₁-C₆)-alkyl, heterocyclyl, heterocyclyl-(C₁-C₆)-alkyl, tris-[(C₁-C₆)-alkyl]silyl-(C₁-C₆)-alkyl, bis-[(C₁-C₆)-alkyl](aryl)silyl(C₁-C₆)-alkyl, [(C₁-C₆)-alkyl]-bis-(aryl)silyl-(C₁-C₆)-alkyl, (C₁-C₆)-alkylcarbonyloxy-(C₁-C₆)-alkyl, (C₃-C₆)-cycloalkylcarbonyloxy-(C₁-C₆)-alkyl, arylcarbonyloxy-(C₁-C₆)-alkyl, heteroarylcarbonyloxy-(C₁-C₆)-alkyl, heterocyclylcarbonyloxy-(C₁-C₆)-alkyl, aryloxy-(C₁-C₆)-alkyl, heteroaryloxy-(C₁-C₆)-alkyl, (C₁-C₆)-alkoxycarbonyl,
R¹⁴ represents hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, heterocyclyl-(C₁-C₆)-alkyl, (C₃-C₆)-cycloalkyl-(C₁-C₆)-alkyl, (C₄-C₁₀)-cycloalkenyl-(C₁-C₆)-alkyl, bis-[(C₁-C₆)-alkyl]amino, (C₁-C₆)-alkyl-amino, aryl-(C₁-C₆)-amino, aryl-(C₁-C₂)-alkyl-amino, aryl-[(C₁-C₆)-alkyl]amino, (C₃-C₆)-cycloalkyl-amino, (C₃-C₆)-cycloalkyl-[(C₁-C₆)-alkyl]amino, N-azetidinyl, N-pyrrolidinyl, N-piperidinyl, N-morpholinyl,
and
X and Y independently of one another represent O (oxygen) or S (sulfur).

4. The compound of formula (I) as claimed in claim 1 and/or salt thereof, wherein
R¹ represents hydrogen,
R² represents hydrogen, fluorine, chlorine, bromine, trifluoromethyl, methoxy, ethoxy, prop-1-yloxy, but-1-yloxy,
R³ represents hydrogen, fluorine, chlorine, bromine, methoxy, ethoxy, prop-1-yloxy, prop-2-yloxy, but-1-yloxy, but-2-yloxy, 2-methylprop-1-yloxy, 1,1-dimethyleth-1-yloxy,
R⁴ represents fluorine, chlorine, bromine, cyano, NO₂, C(O)NH₂, C(S)NH₂, trifluoromethyl, difluoromethyl, pentafluoroethyl, ethynyl, propyn-1-yl, 1-butyn-1-yl, pentyn-1-yl, hexyn-1-yl,
R⁵, R⁶ and R⁷ independently of one another represent hydrogen, fluorine, chlorine, bromine, iodine, cyano, methyl, ethyl, prop-1-yl, 1-methylethyl, but-1-yl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, difluoromethyl, pentafluoroethyl, 2,2-difluoroethyl, 2,2,2-trifluoroethyl, methoxy, ethoxy, prop-1-yloxy, prop-2-yloxy, but-1-yloxy, but-2-yloxy, 2-methylprop-1-yloxy, 1,1-dimethyleth-1-yloxy, difluoromethoxy, trifluoromethoxy, pentafluoroethoxy, 2,2-difluoroethoxy, 2,2,2-trifluoroethoxy,
G represents methylene, (methyl)methylene, (ethyl)methylene, (prop-1-yl)methylene, (prop-2-yl)methylene, (but-1-yl)methylene, (but-2-yl)methylene, (pent-1-yl)methylene, (pent-2-yl)methylene, (pent-3-yl)methylene, (dimethyl)methylene, (diethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, 1,1-dimethylethyl-1-ene, 2,2-dimethylethyl-1-ene, 1-ethylethyl-1-ene, 2-ethylethyl-1-ene, 1-(prop-1-yl)ethyl-1-ene, 2-(prop-1-yl)ethyl-1-ene, 1-(prop-2-yl)ethyl-1-ene, 2-(prop-2-yl)ethyl-1-ene, 1,1,2-trimethylethyl-1-ene, 1,2,2-trimethylethyl-1-ene, 1,1,2,2-tetramethylethyl-1-ene, n-pentylene, 1-methylbutyl-1-ene, 2-methylbutyl-1-ene, 3-methylbutyl-1-ene, 4-methylbutyl-1-ene, 1,1-dimethylpropyl-1-ene, 2,2-dimethylpropyl-1-ene, 3,3-dimethylpropyl-1-ene, 1,2-dimethylpropyl-1-ene, 1,3-dimethylpropyl-1-ene, 1-ethylpropyl-1-ene, n-hexylene, 1-methylpentyl-1-ene, 2-methylpentyl-1-ene, 3-methylpentyl-1-ene, 4-methylpentyl-1-ene, 1,1-dimethylbutyl-1-ene, 1,2-dimethylbutyl-1-ene, 1,3-dimethylbutyl-1-ene, 2,2-dimethylbutyl-1-ene, 2,3-dimethylbutyl-1-ene, 3,3-dimethylbutyl-1-ene, 1-ethylbutyl-1-ene, 2-ethylbutyl-1-ene, 1,1,2-trimethylpropyl-1-ene, 1,2,2-trimethylpropyl-1-ene, 1-ethyl-1-methylpropyl-1-ene, 1-ethyl-2-methylpropyl-1-ene,
X and Y independently of one another represent O (oxygen) or S (sulfur)
and
Q represents one of the moieties Q-1 to Q-54, Q-56 to Q-57, Q-60 to Q-89, Q-91 to Q-129, Q-131 to Q-139, Q-141 to Q-144, Q-146 to Q-180, Q-182 to Q-185, Q-193 to Q-195, Q-200 to Q-208, Q-210 to Q-370, Q-395 to Q-440 specifically mentioned below:

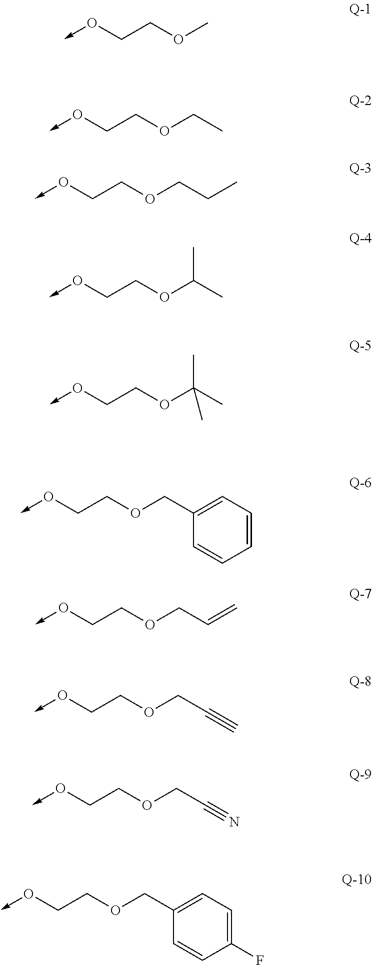

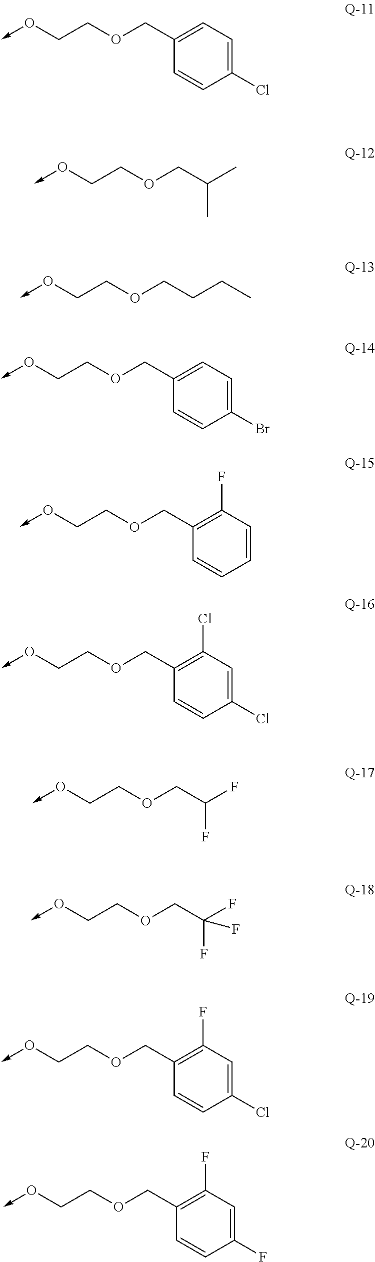

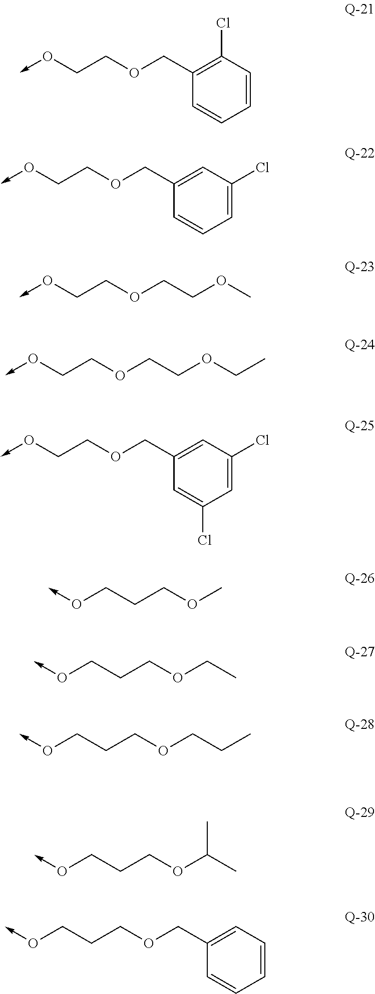

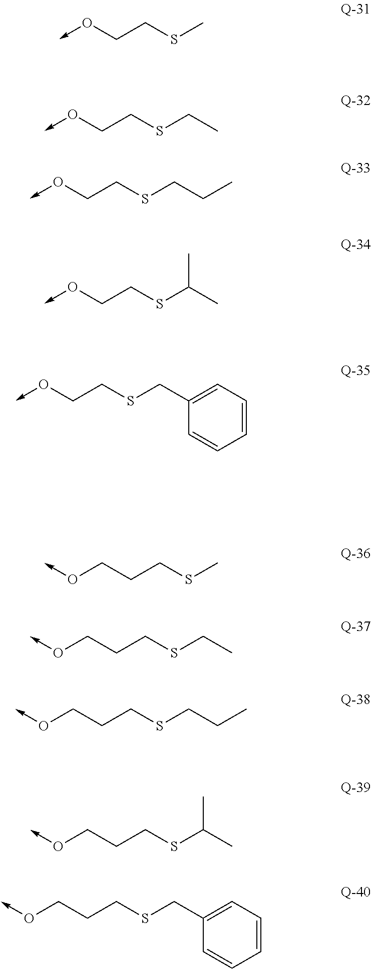

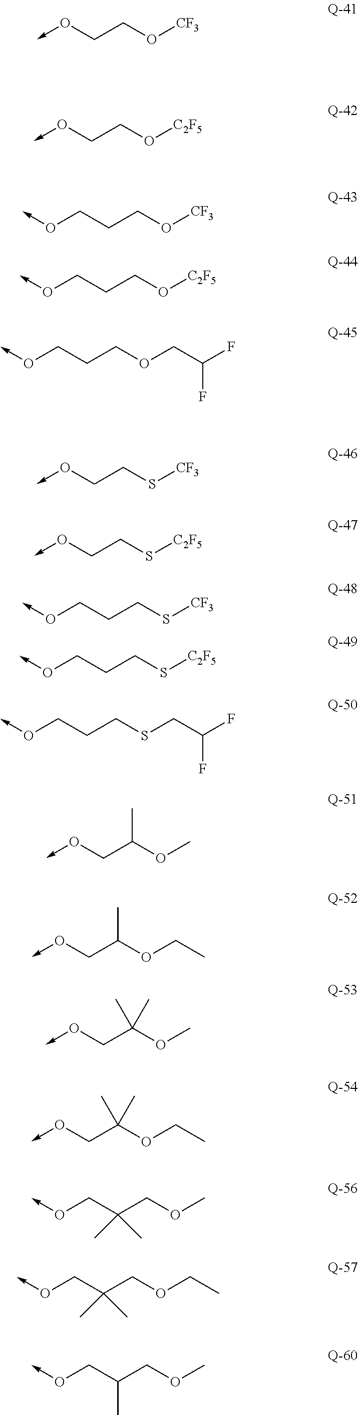

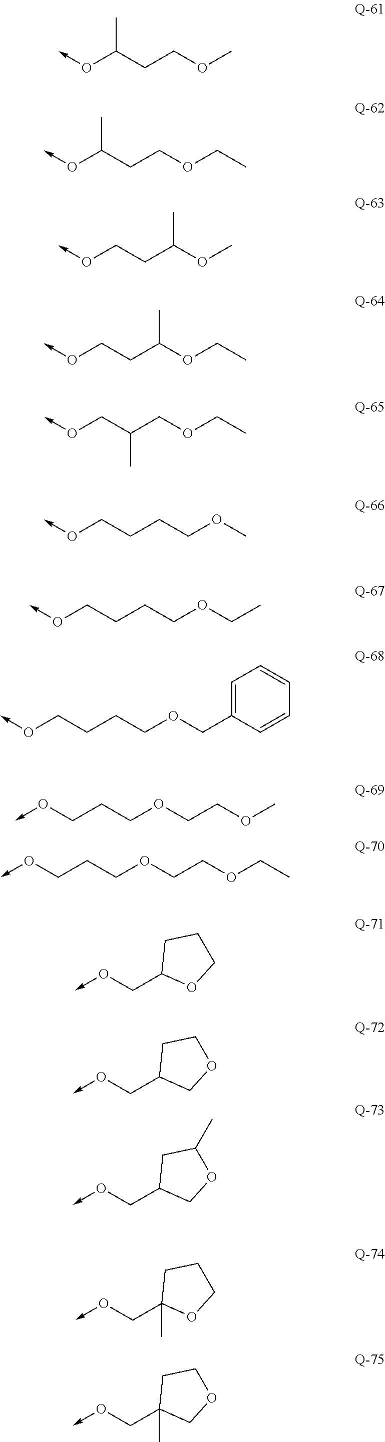

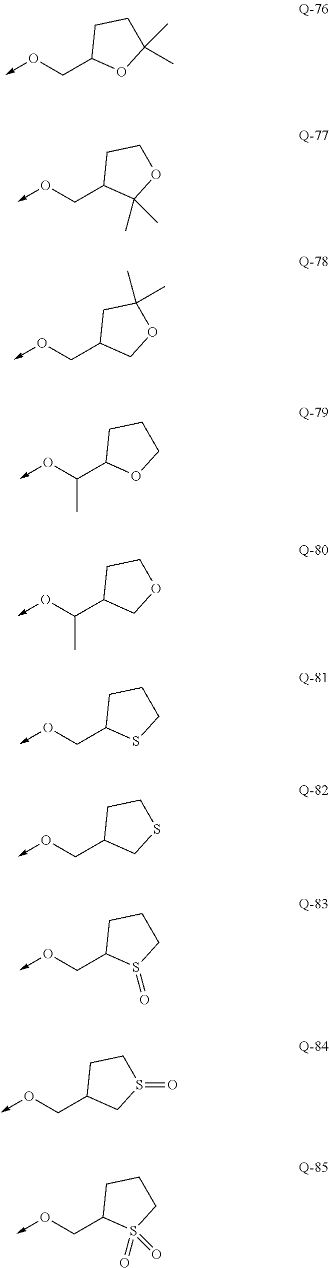

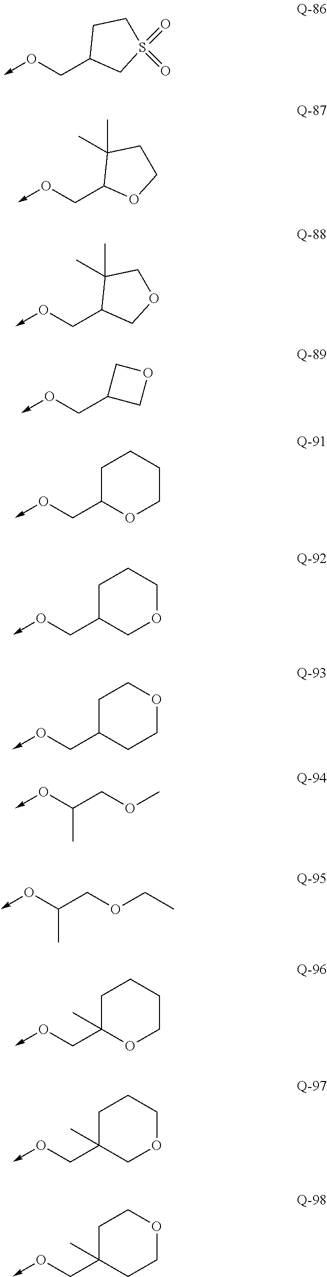

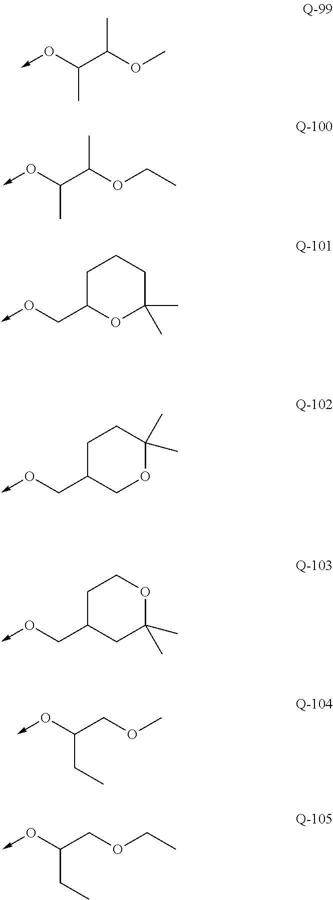

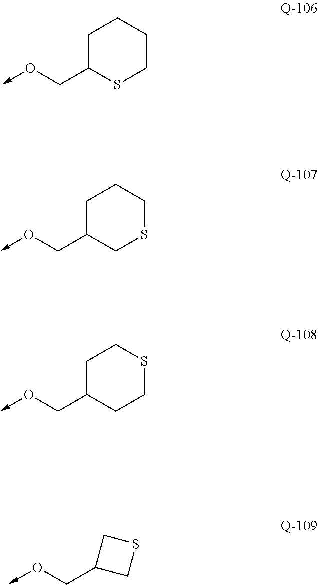

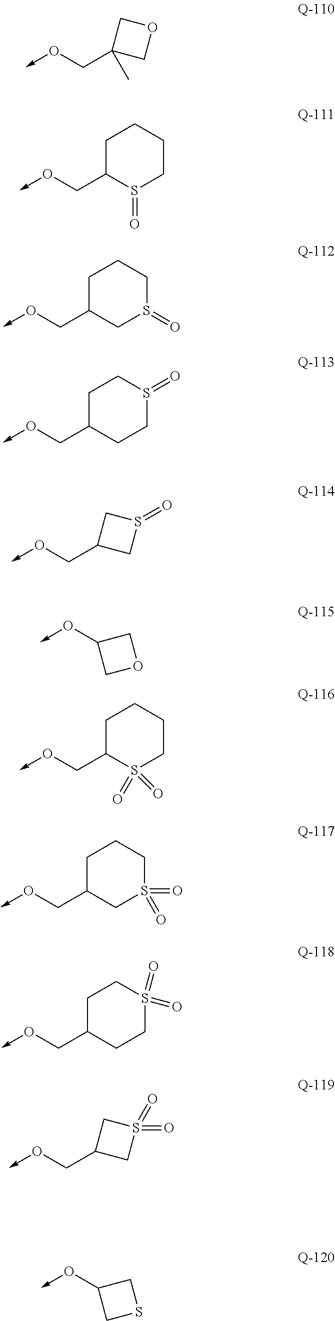

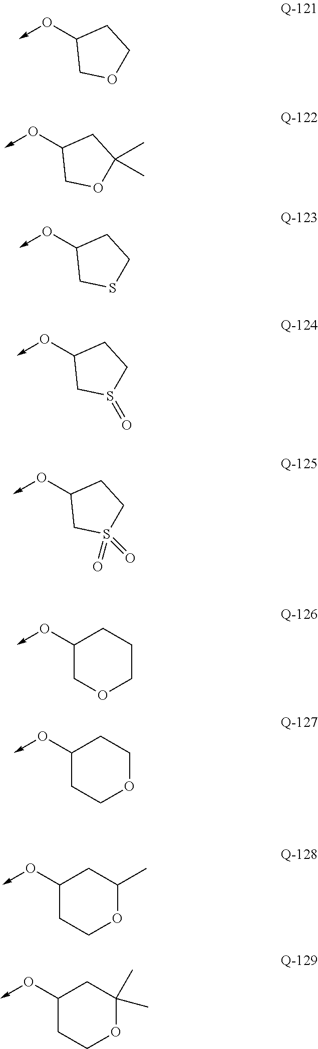

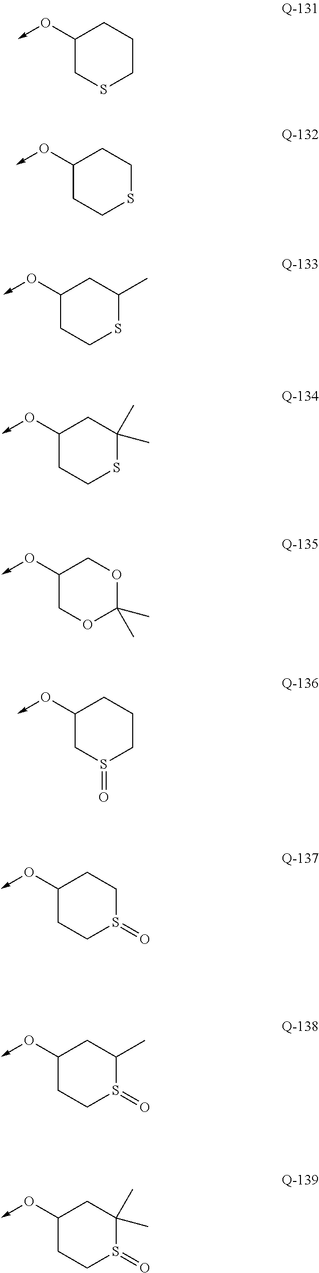

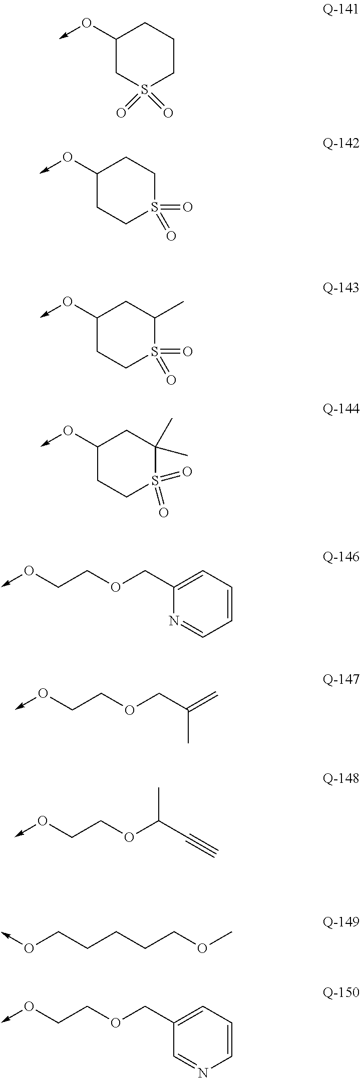

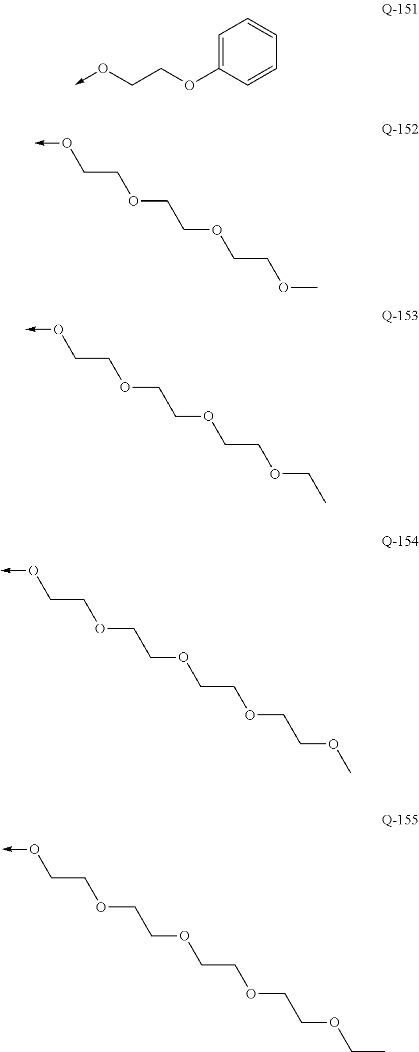

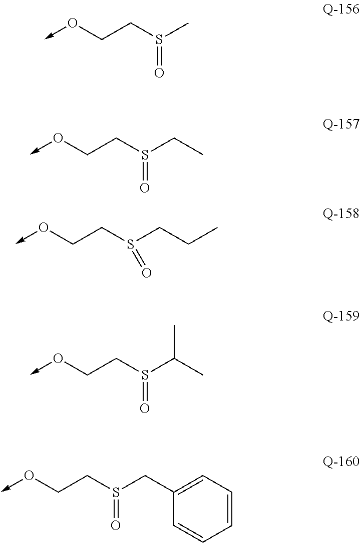

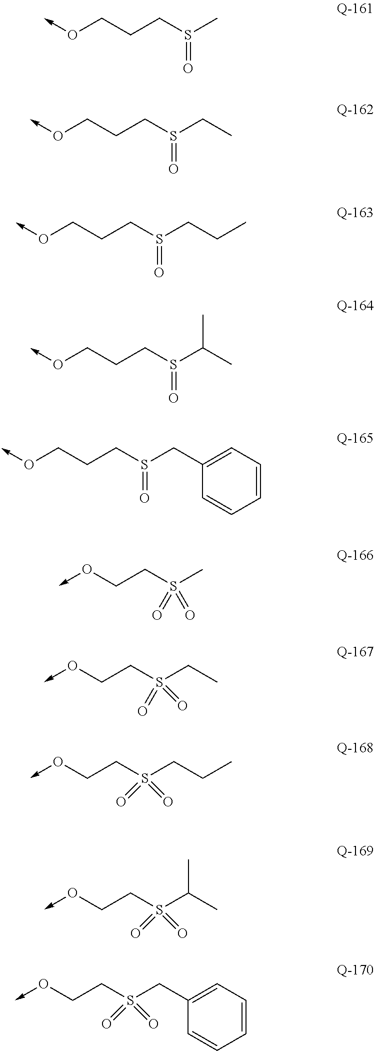

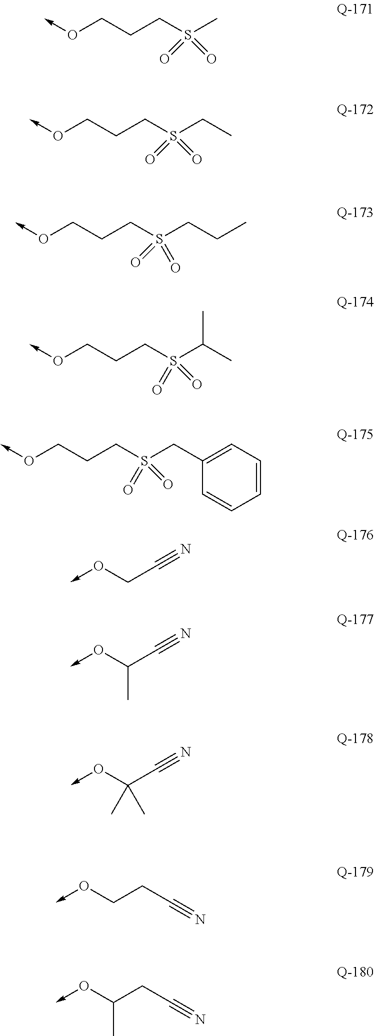

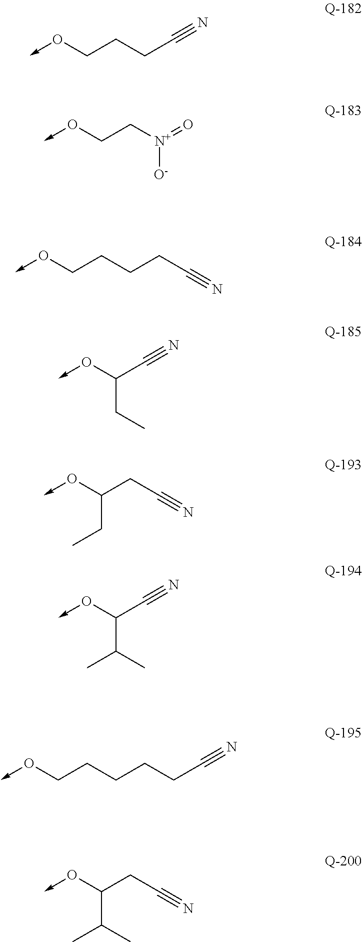

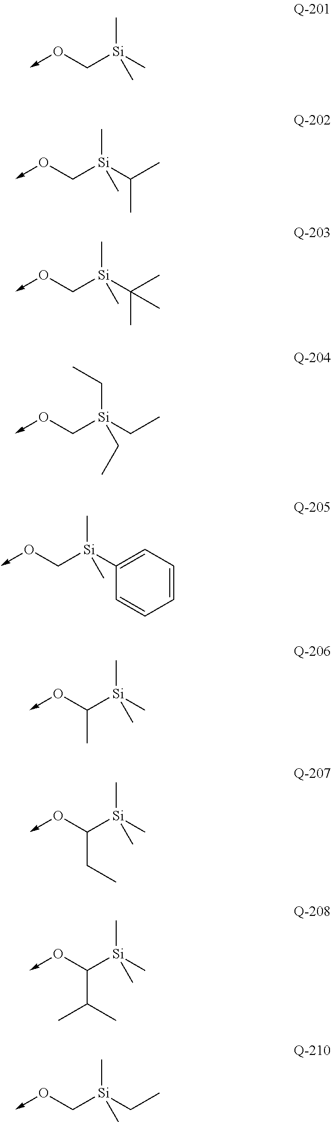

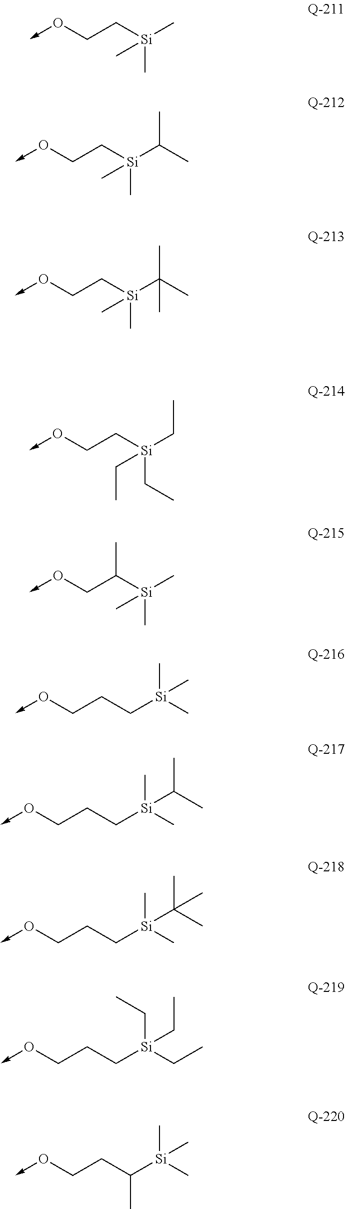

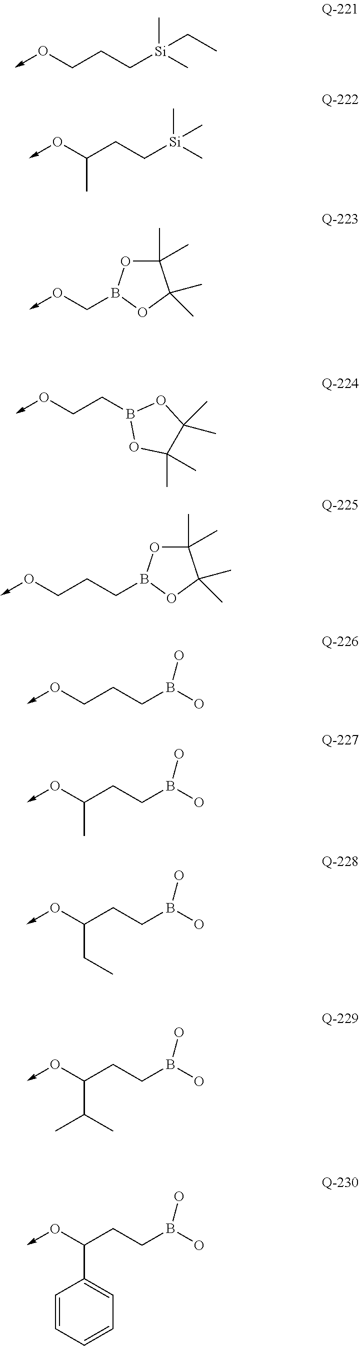

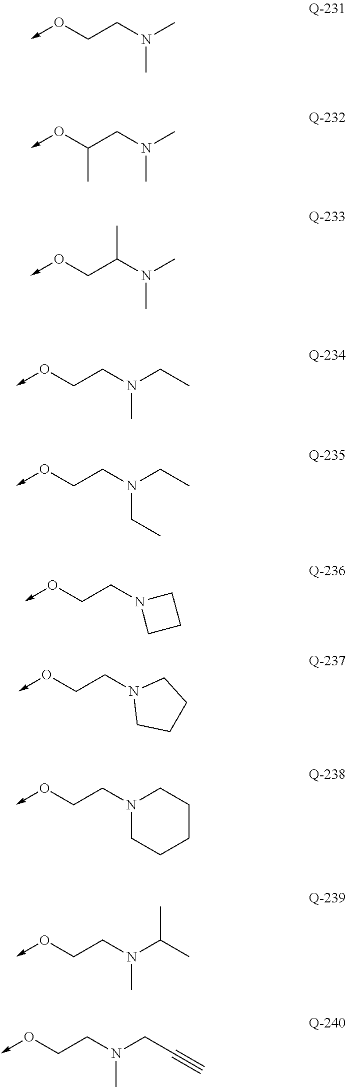

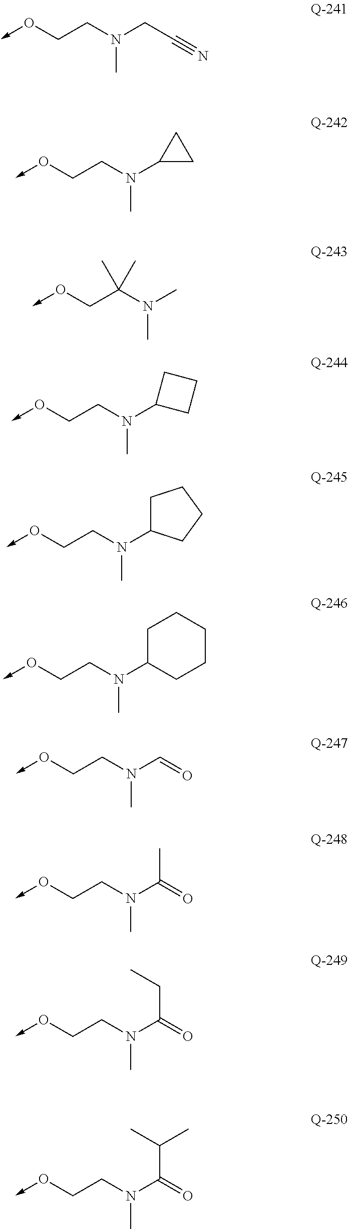

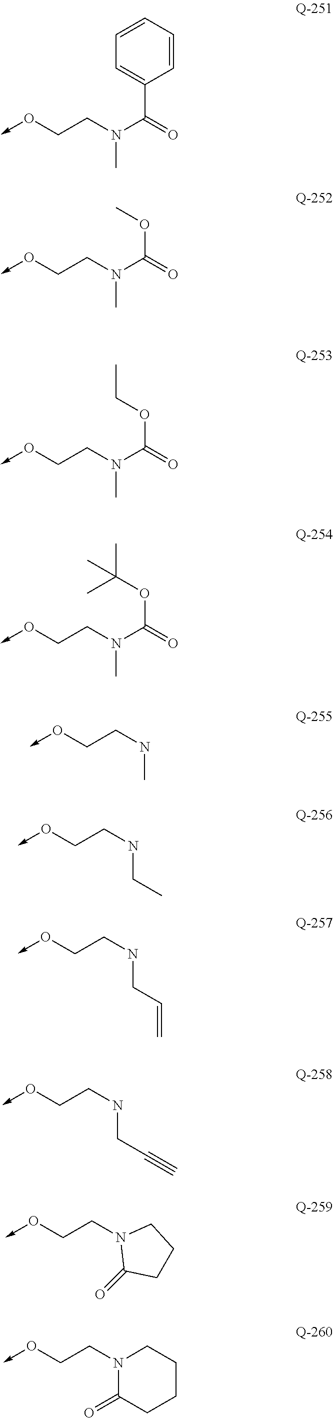

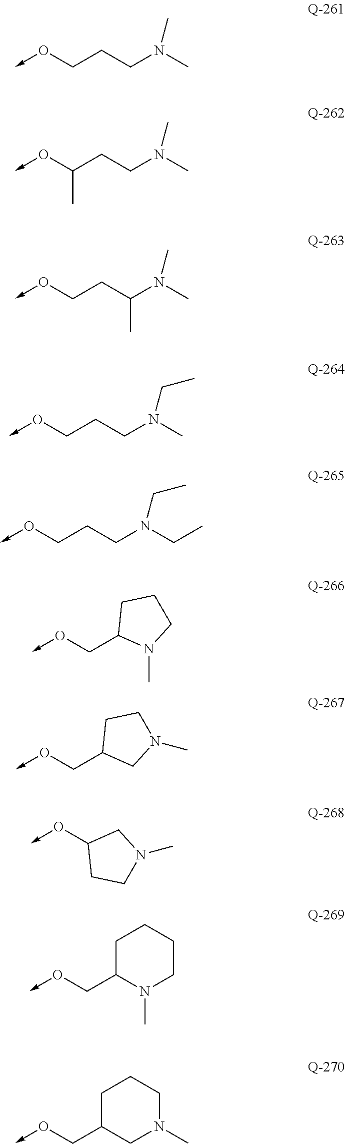

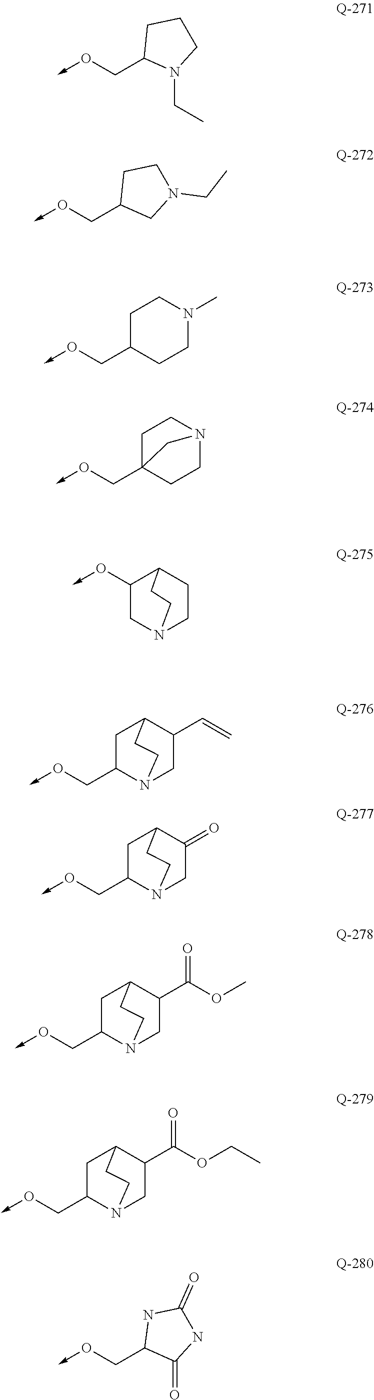

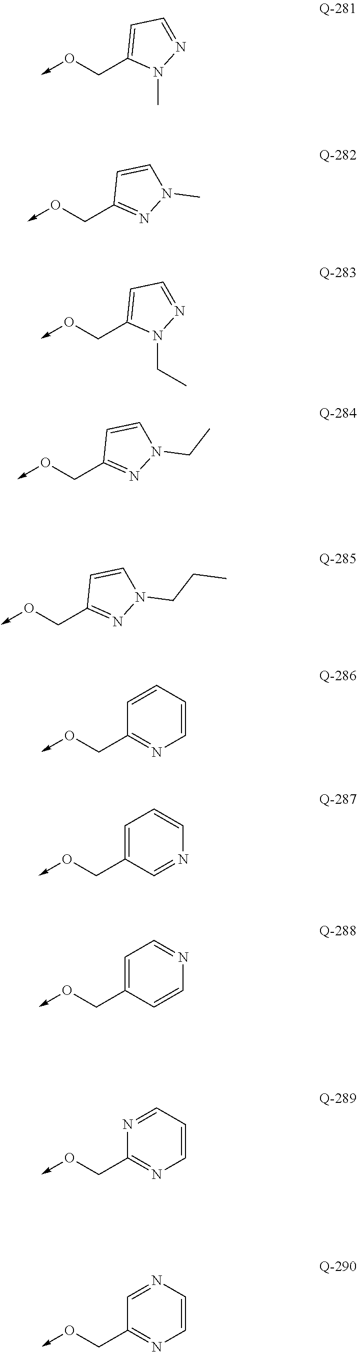

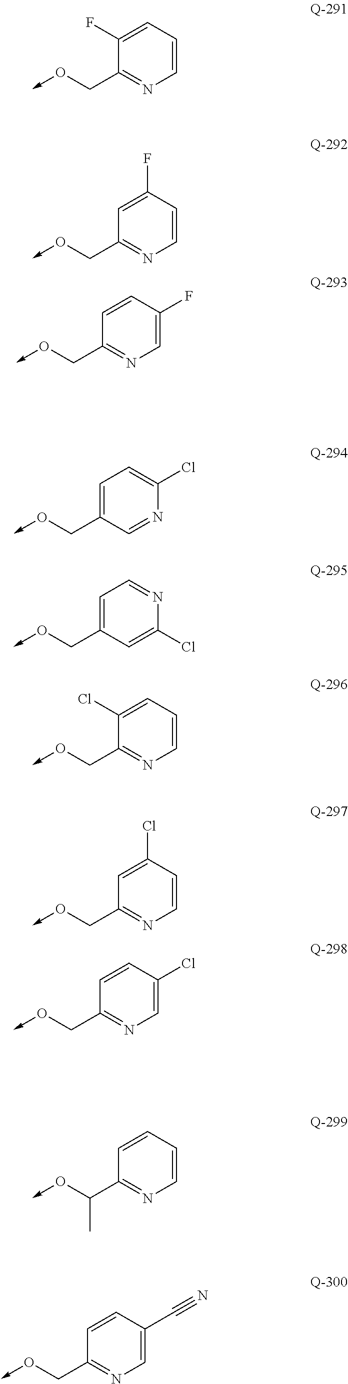

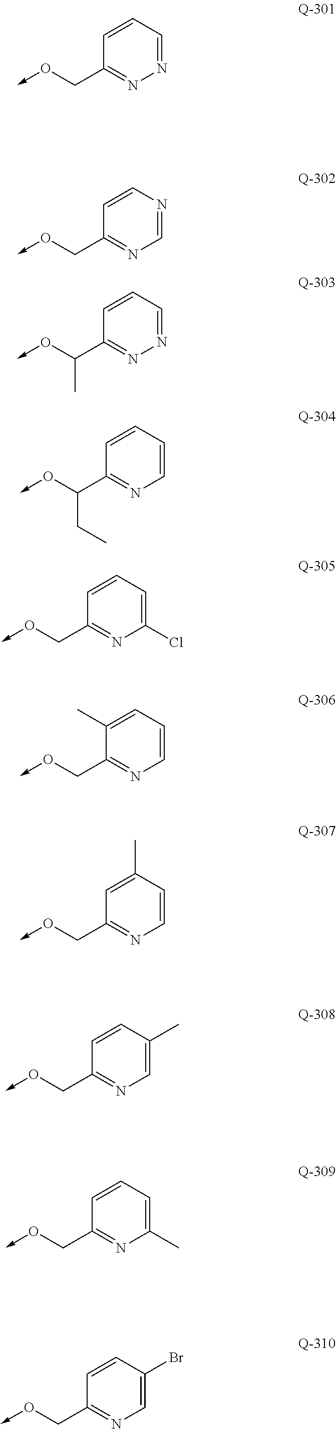

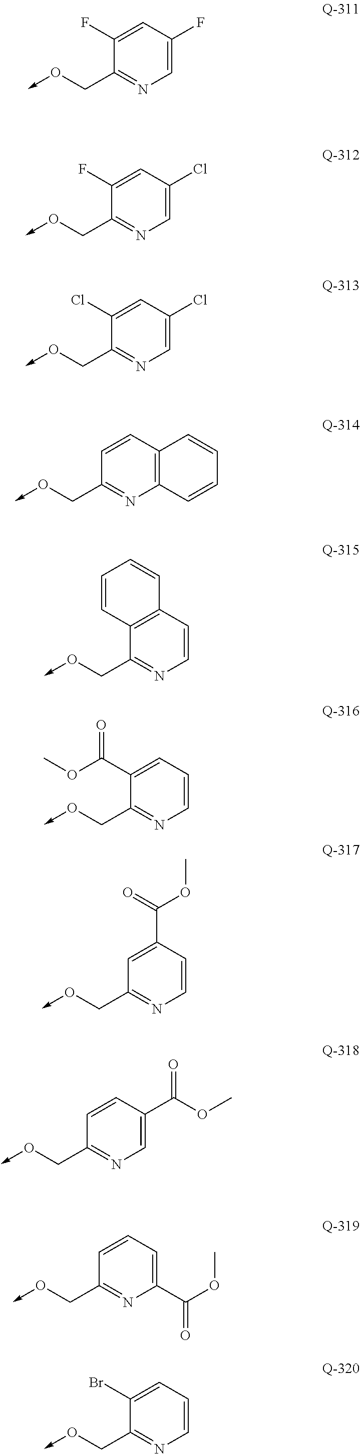

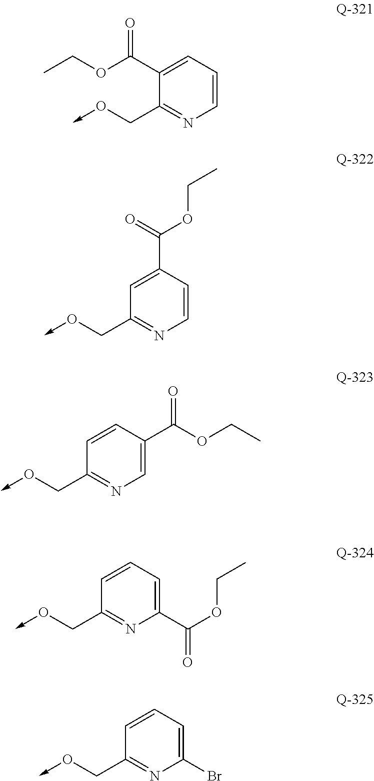

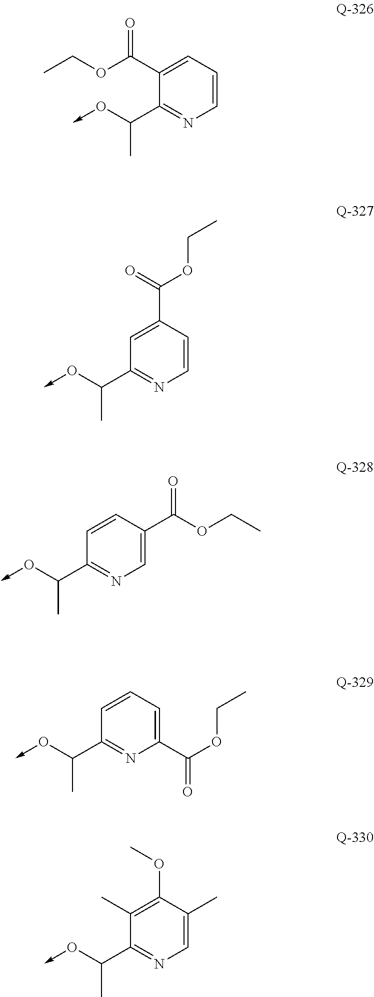

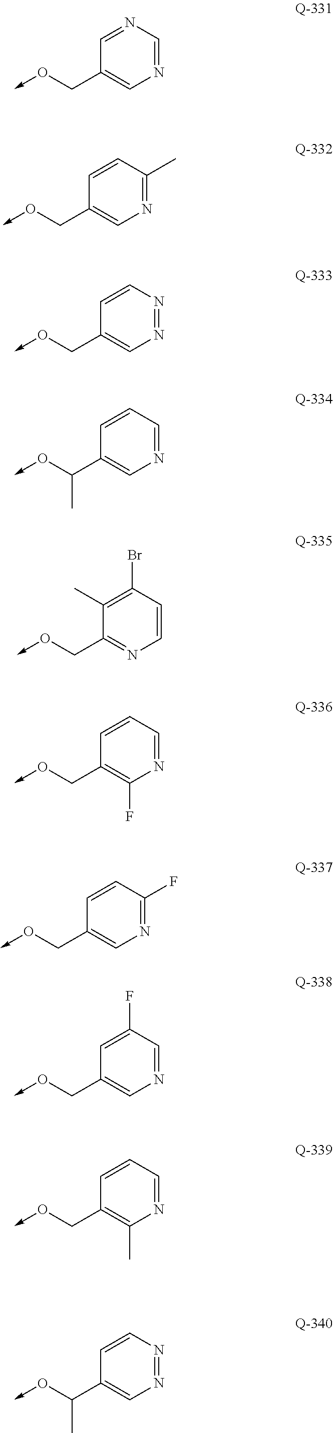

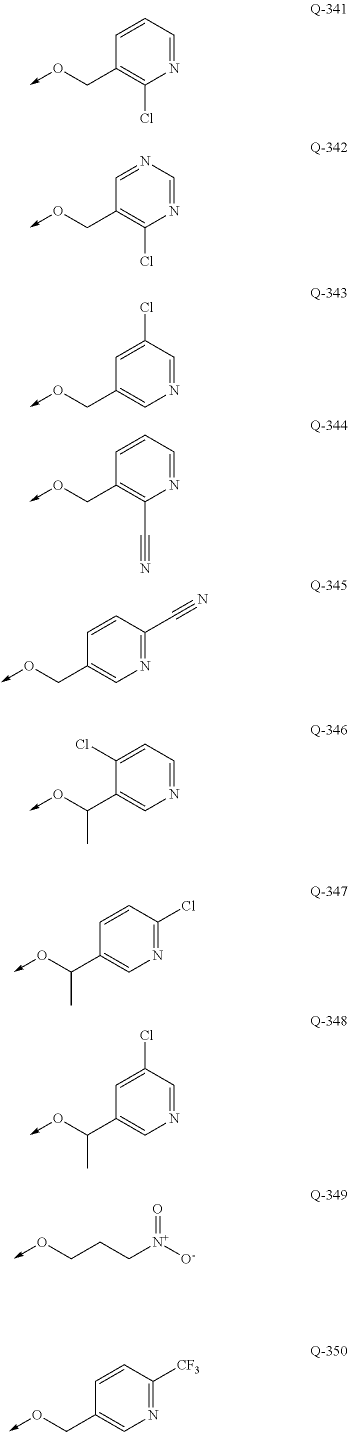

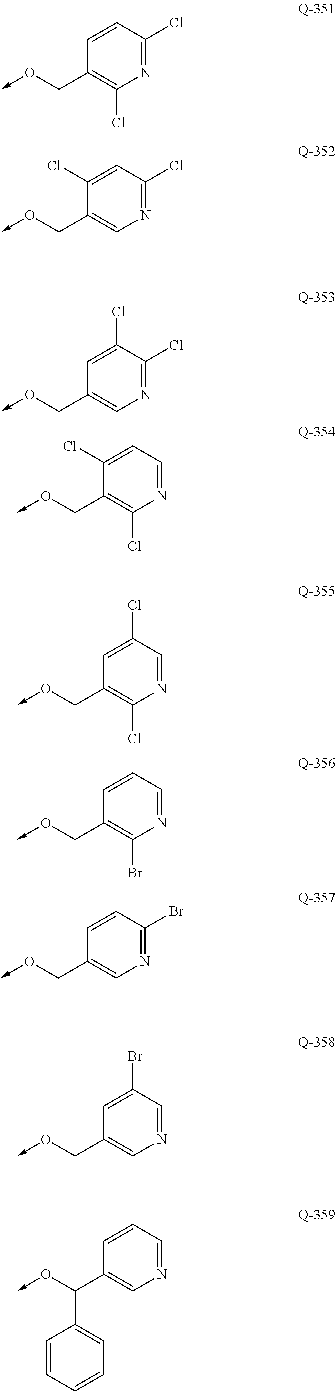

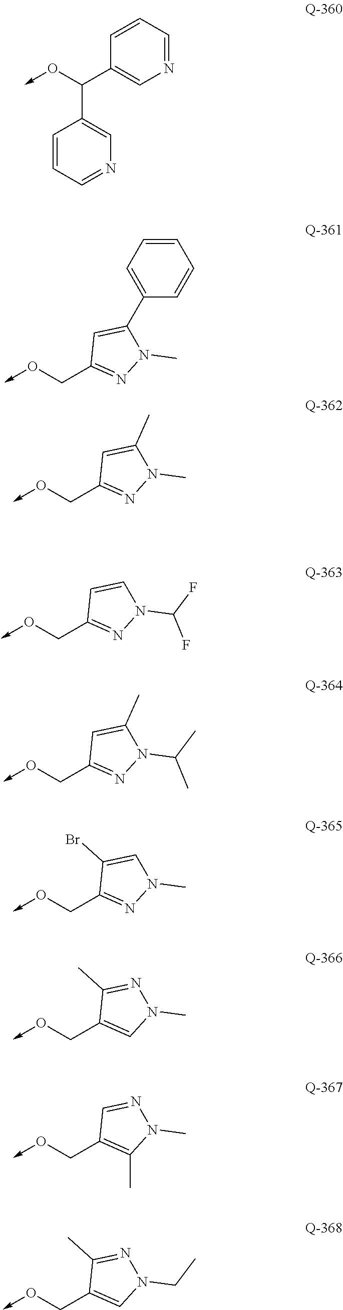

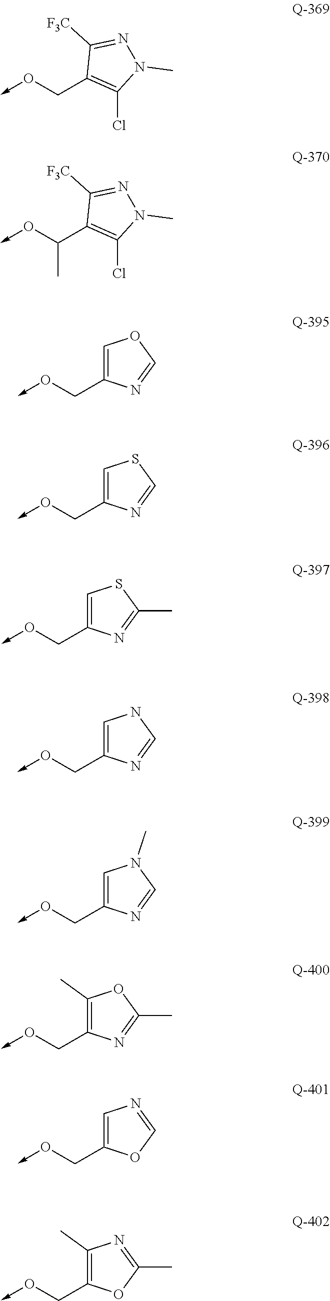

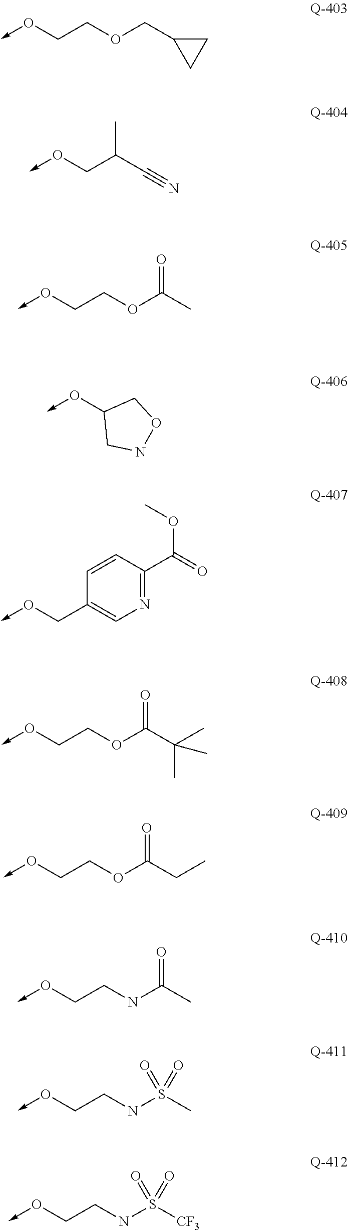

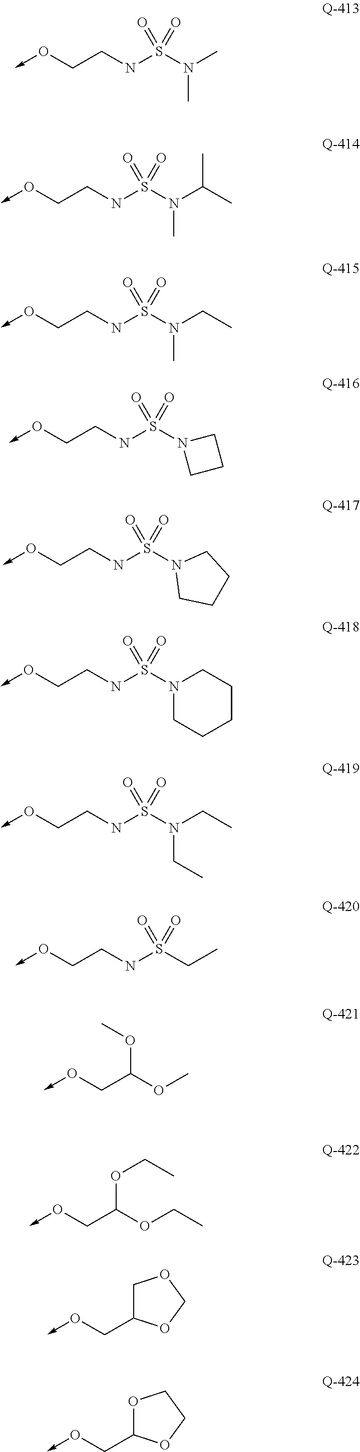

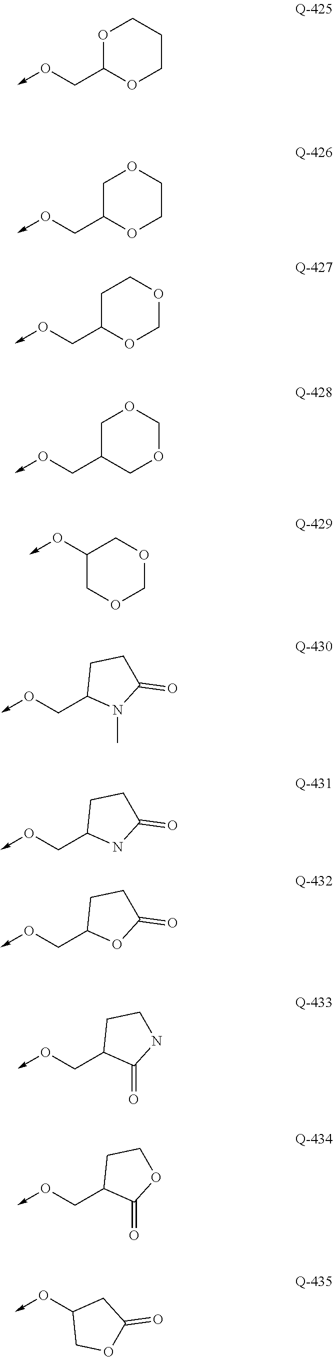

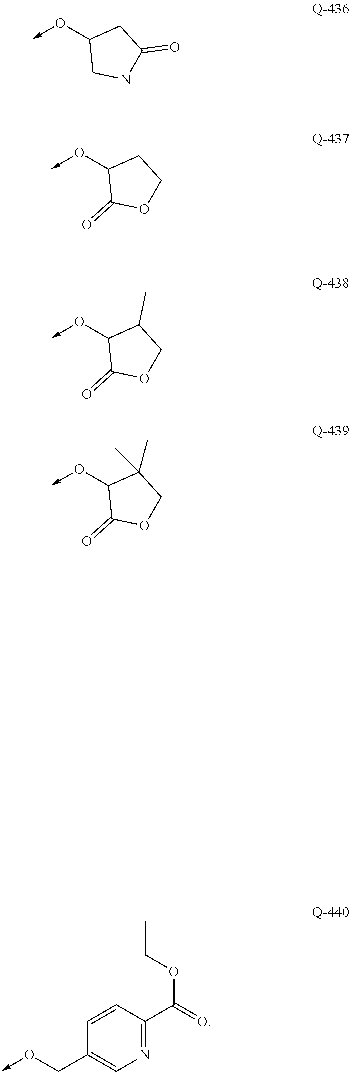

5. The compound of formula (I) as claimed in claim 4 and/or salt thereof, wherein
R¹ represents hydrogen,
R² represents fluorine,
R³ represents hydrogen, fluorine, chlorine, bromine, methoxy,
R⁴ represents fluorine, chlorine, bromine, cyano, NO₂, C(O)NH₂, C(S)NH₂, trifluoromethyl, ethynyl, propyn-1-yl,
R⁵, R⁶, R⁷ independently of one another represent hydrogen, fluorine, chlorine, bromine, iodine, cyano, methyl, ethyl, trifluoromethyl, difluoromethyl, methoxy, ethoxy, difluoromethoxy, trifluoromethoxy,
G represents methylene, (methyl)methylene, (ethyl)methylene, (dimethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, 1,1-dimethylethyl-1-ene, 2,2-dimethylethyl-1-ene, 1-ethylethyl-1-ene, 2-ethylethyl-1-ene, 1-(prop-1-yl)ethyl-1-ene, 2-(prop-1-yl)ethyl-1-ene, 1-(prop-2-yl)ethyl-1-ene, 2-(prop-2-yl)ethyl-1-ene, n-pentylene, 1-methylbutyl-1-ene, 2-methylbutyl-1-ene, 3-methylbutyl-1-ene, 4-methylbutyl-1-ene, 1,1-dimethylpropyl-1-ene, 2,2-dimethylpropyl-1-ene, 3,3-dimethylpropyl-1-ene, 1,2-dimethylpropyl-1-ene, 1,3-dimethylpropyl-1-ene, 1-ethylpropyl-1-ene, n-hexylene,
X and Y independently of one another represent O (oxygen) or S (sulfur)
and
Q represents one of the moieties Q-1 to Q-54, Q-56 to Q-57, Q-60 to Q-89, Q-91 to Q-129, Q-131 to Q-139, Q-141 to Q-144, Q-146 to Q-180, Q-182 to Q-185, Q-193 to Q-195, Q-200 to Q-208, Q-210 to Q-370, Q-395 to Q-440.

6. The compound of formula (I) as claimed in claim 4 and/or salt thereof, wherein
R¹ represents hydrogen,
R² represents fluorine,
R³ represents fluorine,
R⁴ represents chlorine, bromine, cyano, NO₂, C(O)NH₂, C(S)NH₂,
R⁵, R⁶ and R⁷ independently of one another represent hydrogen, fluorine, chlorine, bromine, cyano, methyl, trifluoromethyl, methoxy, trifluoromethoxy,
G represents methylene, (methyl)methylene, (ethyl)methylene, (dimethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, n-pentylene, n-hexylene,
X and Y independently of one another represent O (oxygen) or S (sulfur)
and
Q represents one of the moieties Q-1 to Q-54, Q-56 to Q-57, Q-60 to Q-89, Q-91 to Q-129, Q-131 to Q-139, Q-141 to Q-144, Q-146 to Q-180, Q-182 to Q-185, Q-193 to Q-195, Q-200 to Q-208, Q-210 to Q-370, Q-395 to Q-440.

7. The compound of formula (I) as claimed in claim 4 and/or salt thereof, wherein
R¹ represents hydrogen,
R² represents fluorine,
R³ represents fluorine,
R⁴ represents chlorine, bromine, cyano, NO₂,
R⁵ represents hydrogen,
R⁶ represents hydrogen, fluorine,
R⁷ represents hydrogen,
G represents methylene,
X represents O (oxygen) or S (sulfur),
Y represents O (oxygen),
and
Q represents one of the moieties Q-1 to Q-35, Q-41, Q-42, Q-71 to Q-80, Q-115, Q-120, Q-152 to Q-155, Q-166 to Q-170, Q-176 to Q-206, Q-211 to Q-214, Q-280 to Q-358, Q-362 to Q-370, Q-405, Q-408 to Q-410, Q-421 to Q-429.

8. The compound of formula (I) as claimed in claim 4 and/or salt thereof, wherein
R¹ represents hydrogen,
R² represents fluorine,
R³ represents fluorine,
R⁴ represents chlorine, bromine, cyano, NO₂,
R⁵ represents hydrogen,
R⁶ represents hydrogen, fluorine,
R⁷ represents hydrogen,
G represents methylene,
X represents O (oxygen) or S (sulfur),
Y represents O (oxygen),
and
Q represents one of the moieties Q-1, Q-2, Q-6, Q-23, Q-26, Q-31, Q-41, Q-71, Q-72, Q-115, Q-154, Q-166, Q-176, Q-201, Q-211, Q-280, Q-286, Q-288, Q-301, Q-350, Q-366, Q-367, Q-368, Q-405, Q-421, Q-422, Q-424.

9. A product comprising one or more compounds of formula (I) as defined in claim 1 and/or salts thereof as herbicide and/or plant growth regulator, optionally in crops of useful plants and/or ornamentals.

10. An herbicidal and/or plant growth-regulating composition, wherein the composition comprises one or more compounds of formula (I) as defined in claim 1 and/or salts thereof, and one or more further substances selected from groups (i) and/or (ii), with
(i) one or more further agrochemically active substances, optionally selected from the group consisting of insecticides, acaricides, nematicides, further herbicides, fungicides, safeners, fertilizers and/or further growth regulators,
(ii) one or more formulation auxiliaries customary in crop protection.

11. A method for controlling one or more harmful plants or for regulating the growth of one or more plants, comprising applying an effective amount
of one or more compounds of formula (I), as defined in claim 1 and/or salts thereof, or
of a composition thereof,
to the plants, seed of plants, soil in which or on which the plants grow or an area under cultivation.
